(19) United States
(12) Patent Application Publication
(10) Pub. No.: US 2025/0340872 A1
(43) Pub. Date: Nov. 6, 2025

(54) COMPOUNDS AND METHODS FOR REDUCING TAU EXPRESSION

(71) Applicant: Ionis Pharmaceuticals, Inc., Carlsbad, CA (US)

(72) Inventor: Susan M. Freier, San Diego, CA (US)

(73) Assignee: Ionis Pharmaceuticals, Inc., Carlsbad, CA (US)

(21) Appl. No.: 18/699,132

(22) PCT Filed: Oct. 7, 2022
(86) PCT No.: PCT/US2022/077740

§ 371 Date: Apr. 5, 2024

Related U.S. Application Data
(60) Provisional application No. 63/253,516, filed on Oct. 7, 2021.
(60) Provisional application No. 63/317,743, filed on Mar. 8, 2022.

(51) Int. Cl. C12N 15/113

(52) U.S. Cl. CPC C12N 15/113; C12N 2310/14; C12N 2310/315; C12N 2310/321; C12N 2310/322; C12N 2310/3231

(57) ABSTRACT

Provided are RNAi agents, methods, and pharmaceutical compositions for reducing the amount or activity of tau RNA in a cell or animal, and in certain instances reducing the amount of tau protein in a cell or animal. Such RNAi agents, methods, and pharmaceutical compositions are useful to ameliorate at least one symptom or hallmark of a neurodegenerative disease, including a tauopathy, Alzheimer's disease, fronto-temporal dementia (FTD), FTDP-17, progressive supranuclear palsy (PSP), chronic traumatic encephalopathy (CTE), corticobasal ganglionic degeneration (CBD), epilepsy, or Dravet's Syndrome.

### SEQUENCE LISTING

The present application is being filed along with a Sequence Listing in electronic format. The Sequence Listing is provided as a file entitled BIOL0438WOSEQ.xml created Oct. 3, 2022, which is 1.68 MB in size. The information in the electronic format of the sequence listing is incorporated herein by reference in its entirety.

### FIELD

Provided are RNAi agents, methods, and pharmaceutical compositions for reducing the amount or activity of tau RNA in a cell or animal, and in certain instances reducing the amount of tau protein in a cell or animal. Such agents, methods, and pharmaceutical compositions are useful to ameliorate at least one symptom or hallmark of a neurodegenerative disease. Such neurodegenerative diseases include tauopathies, Alzheimer's disease (AD), frontotemporal dementia (FTD), FTDP-17, progressive supranuclear palsy (PSP), chronic traumatic encephalopathy (CTE), corticobasal ganglionic degeneration (CBD), epilepsy, or Dravet's Syndrome. Such symptoms or hallmarks include loss of memory, loss of motor function, and increase in the number and/or volume of neurofibrillary inclusions.

### BACKGROUND

The primary function of tau is to bind to and stabilize microtubules, which are important structural components of the cytoskeleton involved in mitosis, cytokinesis, and vesicular transport. Tau is found in multiple tissues but is particularly abundant in the axons of neurons. In humans, there are six isoforms of tau that are generated by alternative splicing of exons 2, 3, and 10. Splicing of exons 2 and 3 leads to inclusion of zero, one, or two 29 amino acid acidic domains and is termed 0N, 1N, or 2N tau, respectively. The influence of these domains on tau function is not fully clear, though may play a role in interactions with the plasma membrane. Inclusion of exon 10 leads to inclusion of the microtubule binding domain encoded by exon 10. Since there are 3 microtubule binding domains elsewhere in tau, this tau isoform (with exon 10 included) is termed 4R tau, where ‘R’ refers to the number of repeats of microtubule binding domains. Tau without exon 10 is termed 3R tau. Since more microtubule binding domains (4R compared with 3R) increases the binding to microtubules, 4R tau presumably significantly increases microtubule binding and assembly. The ratio of 3R/4R tau is developmentally regulated, with fetal tissues expressing exclusively 3R tau and adult human tissues expressing approximately equal levels of 3R/4R tau. Deviations from the normal ratio of 3R/4R tau are characteristic of neurodegenerative FTD tauopathies. It is not known how changing the 3R/4R tau ratio at a later stage in the adult animal will affect tau pathogenesis.

Serine-threonine directed phosphorylation regulates the microtubule binding ability of tau. Hyperphosphorylation promotes detachment of tau from microtubules. Other post translational modifications of tau have been described, however the significance of these is unclear. Phosphorylation of tau is also developmentally regulated with higher phosphorylation in fetal tissues and much lower phosphorylation in the adult. One characteristic of neurodegenerative disorders is aberrantly increased tau phosphorylation. The microtubule network is involved in many important processes within the cell including structural integrity needed for maintaining morphology of cells and operating transport machinery. Since binding of tau to microtubules stabilizes microtubules, tau is likely to be a key mediator of some of these processes and disruption of normal tau in neurodegenerative diseases may disrupt some of these key cellular processes.

One of the early indicators that tau may be important in neurodegenerative syndromes was the recognition that tau is a key component of neurofibrillary inclusions in Alzheimer's disease. In fact, neurofibrillary inclusions are aggregates of hyperphosphorylated tau protein. Along with amyloid beta containing plaques, neurofibrillary inclusions are a hallmark of Alzheimer's disease and correlate significantly with cognitive impairment. 95% of tau accumulations in AD are found in neuronal processes and is termed neuritic dystrophy. The process(es) whereby this microtubule associated protein becomes disengaged from microtubules and forms accumulations of proteins and how this relates to neuronal toxicity is not well understood.

Neuronal tau inclusions are a pathological characteristic of not only Alzheimer's disease, but also a subset of frontotemporal dementia (FTD), PSP, and CBD. The link between tau and neurodegeneration was solidified by the discovery that mutations in the tau gene cause a subset of FTD. These genetic data have also highlighted the importance of the 3R:4R ratio of tau. Many of the tau mutations that cause FTD lead to a change in tau splicing, which leads to preferential inclusion of exon 10, and thus to increased 4R tau. The overall tau levels are normal. Whether the tau isoform change or the amino acid change or both cause neurodegeneration remains unknown. Recent data suggest that PSP may also be associated with an increased 4R:3R tau ratio.

To help understand the influence of tau ratios on neurodegeneration, a mouse model based on one of the splicing tau mutations (N279K) has been generated using a minigene that includes the tau promoter and the flanking intronic sequences of exon 10. As in humans, these mice demonstrate increased levels of 4R tau compared with transgenics expressing WT tau and develop behavioral and motor abnormalities as well as accumulations of aggregated tau in the brain and spinal cord.

Tau protein has been associated with multiple diseases of the brain including Alzheimer's disease, FTD, PSP, CBD, dementia pugilistica, parkinsonism linked to chromosome, Lytico-Bodig disease, tangle-predominant dementia, ganglioglioma, gangliocytoma, meningioangiomatosis, subacute sclerosing panencephalitis, lead encephalopathy, tuberous sclerosis, Hallervorden-Spatz disease, Pick's disease, argyrophilic grain disease, corticobasal degeneration or frontotemporal lobar degeneration and others. Tau-associated disorders such as AD are the most common cause of dementia in the elderly. AD affects an estimated 15 million people worldwide and 40% of the population above 85 years of age. AD is characterized by two pathological hallmarks: tau neurofibrillary inclusions (NFT) and amyloid-β (Aβ) plaques.

There is currently a lack of acceptable options for treating such neurodegenerative diseases. It is therefore an object herein to provide methods for the treatment of such diseases.

### SUMMARY

Provided herein are RNAi agents, methods and pharmaceutical compositions for reducing the amount or activity of tau RNA, and in certain embodiments reducing the amount of tau protein in a cell or animal. In certain embodiments, the animal has a neurodegenerative disease. In certain embodiments, the neurodegenerative disease is a tauopathy, Alzheimer's disease, frontotemporal dementia (FTD), FTDP-17, progressive supranuclear palsy (PSP), chronic traumatic encephalopathy (CTE), corticobasal ganglionic degeneration (CBD), epilepsy, or Dravet's Syndrome. In certain embodiments, RNAi agents useful for reducing expression of tau RNA are oligomeric duplexes.

Also provided are methods useful for ameliorating at least one symptom or hallmark of a neurodegenerative disease. In certain embodiments, the neurodegenerative disease is a tauopathy, Alzheimer's disease, frontotemporal dementia (FTD), FTDP-17, progressive supranuclear palsy (PSP), chronic traumatic encephalopathy (CTE), corticobasal ganglionic degeneration (CBD), epilepsy, or Dravet's Syndrome. In certain embodiments, the neurodegenerative disease is AD or FTD. In certain embodiments, the symptom or hallmark includes loss of memory, loss of motor function, and increase in the number and/or volume of neurofibrillary inclusions.

### DETAILED DESCRIPTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” or a “2′-O-methoxyethyl” means a 2′-O(CH₂)₂OCH₃ group in place of the 2′-OH group of a furanosyl sugar moiety. A “2′-MOE sugar moiety” or a “2′-O-methoxyethyl sugar moiety” means a sugar moiety with a 2′-O(CH₂)₂OCH₃ group in place of the 2′-OH group of a furanosyl sugar moiety. Unless otherwise indicated, a 2′-MOE sugar moiety is in the β-D-ribosyl configuration. “MOE” means O-methoxyethyl.

As used herein, “2′-MOE nucleoside” or “2′-O(CH₂)₂OCH₃ nucleoside” means a nucleoside comprising a 2′-MOE sugar moiety.

As used herein, “2′-OMe” means a 2′-OCH₃ group in place of the 2′-OH group of a furanosyl sugar moiety. A “2′-O-methyl sugar moiety” or “2′-OMe sugar moiety” or “2′-O-methylribosyl sugar moiety” means a sugar moiety with a 2′-OCH₃ group in place of the 2′-OH group of a furanosyl (e.g., ribosyl) sugar moiety. Unless otherwise indicated, a 2′-OMe sugar moiety is in the β-D-ribosyl configuration.

As used herein, “2′-OMe nucleoside” or “2′-OMe modified nucleoside” means a nucleoside comprising a 2′-OMe sugar moiety.

As used herein, “2′-F” means a 2′-F group in place of the 2′-OH group of a furanosyl sugar moiety. A “2′-fluoro sugar moiety” or “2′-F sugar moiety” or “2′-fluororibosyl sugar moiety” means a sugar moiety with a 2′-F group in place of the 2′-OH group of a furanosyl sugar moiety. Unless otherwise indicated, a 2′-F sugar moiety is in the β-D-ribosyl configuration.

As used herein, “2′-F nucleoside” or “2′-F modified nucleoside” means a nucleoside comprising a 2′-F modified sugar moiety.

As used herein, “xylo 2′-F” means a 2′-F sugar moiety in the β-D-xylosyl configuration.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “3′ target site” refers to the 3′-most nucleotide of a target nucleic acid which is complementary to an antisense oligonucleotide, when the antisense oligonucleotide is hybridized to the target nucleic acid.

As used herein, “5′ target site” refers to the 5′-most nucleotide of a target nucleic acid which is complementary to an antisense oligonucleotide, when the antisense oligonucleotide is hybridized to the target nucleic acid.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “administering” or “administration” means providing a pharmaceutical agent or composition to a subject.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is the modulating of splicing of a target pre-mRNA.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include, but are not limited to, antisense RNAi oligonucleotides.

As used herein, “antisense RNAi oligonucleotide” means an oligonucleotide comprising a region that is complementary to a target sequence, and which includes at least one chemical modification suitable for RNAi-mediated nucleic acid reduction.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, the symptom or hallmark is loss of memory, loss of motor function, and increase in the number and/or volume of neurofibrillary inclusions. The progression or severity of indicators may be determined by subjective or objective measures, which are known to those skilled in the art.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the furanosyl sugar moiety is a ribosyl sugar moiety. In certain embodiments, the bicyclic sugar moiety does not comprise a furanosyl moiety.

As used herein, “blunt” or “blunt ended” in reference to an oligomeric duplex means that there are no terminal unpaired nucleotides (i.e., no overhanging nucleotides). One or both ends of an oligomeric duplex can be blunt.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) of cerebrospinal fluid and is biocompatible with CSF.

As used herein, “cell-targeting moiety” means a conjugate group or portion of a conjugate group that is capable of binding to a particular cell type or particular cell types.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions. As used herein, “complementary nucleobases” means nucleobases that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Certain modified nucleobases that pair with natural nucleobases or with other modified nucleobases are known in the art and are not considered complementary nucleobases as defined herein unless indicated otherwise. For example, inosine can pair, but is not considered complementary, with adenosine, cytosine, or uracil. Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or nucleic acid at each nucleobase of the shorter of the two oligonucleotides, or at each nucleoside if the oligonucleotides are the same length.

As used herein, “complementary region” in reference to an oligonucleotide is the range of nucleobases of the oligonucleotide that is complementary with a second oligonucleotide or target nucleic acid. The “complementary region” of an oligonucleotide means that at least 70% of the nucleobases of that region and the nucleobases of another nucleic acid or one or more regions thereof are capable of hydrogen bonding with one another when the nucleobase sequence of the oligonucleotide and the other nucleic acid are aligned in opposing directions.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising cEt modified sugar.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides.

As used herein, “diluent” means an ingredient in a composition that lacks pharmacological activity, but is pharmaceutically necessary or desirable. For example, the diluent in an injected composition can be a liquid, e.g., aCSF, PBS, or saline solution.

As used herein, “double-stranded” in reference to a region or an oligonucleotide means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “duplex” or “duplex region” means the structure formed by two oligonucleotides or portions thereof that are hybridized to one another.

As used herein, “hotspot region” is a range of nucleobases on a target nucleic acid that is amenable to antisense agent-mediated, and in particular RNAi agent-mediated, reduction of the amount or activity of the target nucleic acid.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an oligomeric duplex and a nucleic acid target. In certain embodiments, complementary nucleic acid molecules include, but are not limited to, an antisense oligonucleotide and a nucleic acid target.

As used herein, “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “lipid nanoparticle” or “LNP” is a vesicle comprising a lipid layer encapsulating a pharmaceutically active molecule, such as a nucleic acid molecule, e.g., an RNAi agent or a plasmid from which an RNAi agent is transcribed. LNPs are described in, for example, U.S. Pat. Nos. 6,858,225, 6,815,432, 8,158,601, and 8,058,069, the entire contents of each of which are hereby incorporated herein by reference.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned.

As used herein, “modified nucleoside” means a nucleoside comprising a modified nucleobase and/or a modified sugar moiety.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “motif” means the pattern of unmodified and/or modified sugar moieties, nucleobases, and/or internucleoside linkages, in an oligonucleotide.

As used herein, “neurodegenerative disease” means a condition marked by progressive loss of function or structure, including loss of neuronal function and death of neurons. In certain embodiments, the neurodegenerative disease is a tauopathy, Alzheimer's disease, frontotemporal dementia (FTD), FTDP-17, progressive supranuclear palsy (PSP), chronic traumatic encephalopathy (CTE), corticobasal ganglionic degeneration (CBD), epilepsy, or Dravet's Syndrome.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases.

As used herein, “nucleobase sequence” means the order of contiguous nucleobases in a target nucleic acid or oligonucleotide independent of any sugar or internucleoside linkage modification.

As used herein, “nucleoside” means a compound, or a fragment of a compound, comprising a nucleobase and a sugar moiety. The nucleobase and sugar moiety are each, independently, unmodified or modified.

As used herein, “nucleoside overhang” refers to unpaired nucleotides at either or both ends of an oligomeric duplex formed by hybridization two oligonucleotides.

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

As used herein, “oligomeric duplex” means a duplex formed by two oligomeric compounds having complementary nucleobase sequences.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “prodrug” means an inactive or less active form of a compound which, when administered to a subject, is metabolized to form the active, or more active, compound. In certain embodiments, a prodrug comprises a cell-targeting moiety and at least one active compound.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNAi (ssRNAi), and microRNA, including microRNA mimics. RNAi agents may comprise conjugate groups and/or terminal groups. In certain embodiments, an RNAi agent modulates the amount, activity, and/or activity of a target nucleic acid. The term RNAi agent excludes antisense compounds that act through RNase H.

As used herein, “sense compound” means a sense oligonucleotide and optionally one or more additional features, such as a conjugate group.

As used herein, “sense oligonucleotide” means an oligonucleotide, including the oligonucleotide portion of an sense compound, that is capable of hybridizing to an antisense oligonucleotide. Sense oligonucleotides include but are not limited to sense RNAi oligonucleotides.

As used herein, “standard in vitro assay” means the assay described in Example 2 and reasonable variations thereof.

As used herein, “stereorandom” or “stereorandom chiral center” in the context of a population of molecules of identical molecular formula means a chiral center that is not controlled during synthesis, or enriched following synthesis, for a particular absolute stereochemical configuration. The stereochemical configuration of a chiral center is considered random when it is the result of a synthetic method that is not designed to control the stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. In certain embodiments, the stereorandom chiral center is not racemic because one absolute configuration predominates following synthesis, e.g., due to the action of non-chiral reagents near the enriched stereochemistry of an adjacent sugar moiety. In certain embodiments, a stereorandom chiral center is at the phosphorous atom of a stereorandom phosphorothioate or mesyl phosphoroamidate internucleoside linkage. In certain embodiments, a stereorandom chiral center is a stereorandom phosphorothioate internucleoside linkage.

As used herein, “stabilized phosphate group” means a 5′-phosphate analog that is metabolically more stable than a 5′-phosphate as naturally occurs on DNA or RNA.

As used herein, “sugar moiety” means an unmodified sugar moiety or a modified sugar moiety. As used herein, “unmodified sugar moiety” means a 2′-OH(H) β-D-ribosyl moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom” or “hallmark” means any physical feature or test result that indicates the existence or extent of a disease or disorder. In certain embodiments, a symptom is apparent to a subject or to a medical professional examining or testing the subject. In certain embodiments, a hallmark is apparent upon invasive diagnostic testing, including, but not limited to, post-mortem tests. In certain embodiments, a hallmark is apparent on a brain MRI scan. In certain embodiments, symptoms and hallmarks include loss of memory, loss of motor function, and/or increase in the number and/or volume of neurofibrillary inclusions.

As used herein, “target nucleic acid” and “target RNA” mean a nucleic acid that an oligomeric compound is designed to affect. Target RNA means an mRNA transcript and includes pre-mRNA and mRNA unless otherwise specified.

As used herein, “target region” means a portion of a target nucleic acid to which an oligomeric compound is designed to hybridize.

“Tau-associated disease” means any disease or disorder associated with any tau nucleic acid or expression product thereof. Such diseases may include a neurodegenerative disease. Such neurodegenerative diseases may include tauopathies, Alzheimer's disease, frontotemporal dementia (FTD), FTDP-17, progressive supranuclear palsy (PSP), chronic traumatic encephalopathy (CTE), corticobasal ganglionic degeneration (CBD), epilepsy, and Dravet's Syndrome.

“Tau RNA” means any messenger RNA (mRNA) expression product of a DNA sequence encoding tau.

“Tau nucleic acid” means any nucleic acid encoding tau. For example, in certain embodiments, a tau nucleic acid includes a DNA sequence encoding tau, an RNA sequence transcribed from DNA encoding tau (including genomic DNA comprising introns and exons), and an mRNA sequence encoding tau. “Tau mRNA” means an mRNA encoding a tau protein. Tau nucleic acid may also be referred to herein as mammalian microtubule-associated protein tau (MAPT), including human microtubule-associated protein tau (MAPT).

“Tau protein” means the polypeptide expression product of a tau nucleic acid.

As used herein, “terminal group” means a chemical group or group of atoms that is covalently linked to a terminus of an oligonucleotide.

As used herein, “treating” means improving a subject's disease or condition by administering an oligomeric agent or oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to an animal. For example, a therapeutically effective amount improves a symptom of a disease.

### CERTAIN EMBODIMENTS

The present disclosure provides the following non-limiting numbered embodiments:

Embodiment 1. An oligomeric compound, wherein the oligomeric compound comprises a modified oligonucleotide consisting of 12 to 50 linked nucleosides, wherein the nucleobase sequence of the modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of any of the nucleobase sequences of any of SEQ ID NOs: 11-39, 69-112, 157-204, 253-290, 329-375, 423-452, 483-516, 551-580, 611-650, 691-721, 753-898, 1045-1443, and wherein the modified oligonucleotide is an antisense oligonucleotide.

Embodiment 2. The oligomeric compound of embodiment 1, wherein the nucleobase sequence of the modified oligonucleotide comprises the nucleobase sequence of any of SEQ ID NOs: 11-39, 69-112, 157-204, 253-290, 329-375, 423-452, 483-516, 551-580, 611-650, 691-721, 753-898, 1045-1443.

Embodiment 3. The oligomeric compound of embodiment 1, wherein the nucleobase sequence of the modified oligonucleotide consists of nucleobase sequence of any SEQ ID NOs: 11-39, 69-112, 157-204, 253-290, 329-375, 423-452, 483-516, 551-580, 611-650, 691-721, 753-898, 1045-1443.

Embodiment 4. The oligomeric compound of any of embodiments 1-3, wherein the nucleobase sequence of the modified oligonucleotide comprises or consists of the nucleobase sequence selected from SEQ ID NOs: 329, 330, 391, 694, 696, 721, 759, 774, 787, 848, 850, 855, 857-858, 860-861, 863, 884, 886, 890, 891, 1045, 1050, 1115, 1116, 1142, 1157, 1159, 1161, 1166-1167, 1229-1330, 1343, 1360, 1364-1365, 1402, 1430-1431.

Embodiment 5. The oligomeric compound of any of embodiments 1-4, wherein the nucleobase sequence of the modified oligonucleotide is at least 90%, at least 95%, or 100% complementary to an equal length portion of a tau nucleic acid, wherein the tau nucleic acid has the nucleobase sequence of SEQ ID NO: 1 or SEQ ID NO: 2.

Embodiment 6. The oligomeric compound of any of embodiments 1-15, wherein the modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 29, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 7. The oligomeric compound of any of embodiments 1-6, wherein the modified oligonucleotide consists of 23 linked nucleosides.

Embodiment 8. The oligomeric compound of any of embodiments 1-7, wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, at least 21, at least 22, or 23 contiguous nucleobases of an equal length portion of nucleobases of 110-142 of SEQ ID NO: 1;
an equal length portion of nucleobases of 1754-1783 SEQ ID NO: 1;
an equal length portion of nucleobases of 2332-2362 SEQ ID NO: 1; or
an equal length portion of nucleobases of 6523-6552 SEQ ID NO: 1.

Embodiment 9. The oligomeric compound of any of embodiments 1-8, wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, at least 21, at least 22, or 23 contiguous nucleobases of
SEQ ID NOs: 721, 850, 1115, or 1116;
SEQ ID NOs: 885, 1142, or 1360;
SEQ ID NOs: 329, 1045, or 1343; and
SEQ ID NOs: 890, 1330, or 1431.

Embodiment 10. The oligomeric compound of any of embodiments 1-8, wherein the nucleobase sequence of the modified oligonucleotide comprises or consists of the nucleobase sequence selected from
SEQ ID NOs: 721, 850, 1115, or 1116;
SEQ ID NOs: 885, 1142, or 1360;
SEQ ID NOs: 329, 1045, or 1343; and
SEQ ID NOs: 890, 1330, or 1431.

Embodiment 11. The oligomeric compound of any of embodiments 1-10, wherein at least one nucleoside of the modified oligonucleotide comprises a modified sugar moiety.

Embodiment 12. The oligomeric compound of embodiment 11, wherein the modified sugar moiety comprises a bicyclic sugar moiety.

Embodiment 13. The oligomeric compound of embodiment 12, wherein the bicyclic sugar moiety comprises a 2′-4′ bridge, wherein the 2′-4′ bridge is selected from —O—CH₂— and —O—CH(CH₃)—.

Embodiment 14. The oligomeric compound of embodiment 11, wherein the modified sugar moiety comprises a non-bicyclic modified sugar moiety.

Embodiment 15. The oligomeric compound of embodiment 14, wherein the non-bicyclic modified sugar moiety is a 2′-MOE sugar moiety, a 2′-OMe sugar moiety, or a 2′-F sugar moiety.

Embodiment 16. The oligomeric compound of any of embodiments 1-15, wherein at least one nucleoside of the modified oligonucleotide comprises a sugar surrogate.

Embodiment 17. The oligomeric compound of embodiment 16, wherein the sugar surrogate is selected from morpholino, modified morpholino, glycol nucleic acid (GNA), hexitol nucleic acid (HNA), fluoro-hexitol nucleic acid (F-HNA), and peptide nucleic acid (PNA).

Embodiment 18. The oligomeric compound of any of embodiments 1-17, wherein the modified oligonucleotide comprises at least one modified internucleoside linkage.

Embodiment 19. The oligomeric compound of embodiment 18, wherein at least one modified internucleoside linkage is a phosphorothioate internucleoside linkage.

Embodiment 20. The oligomeric compound of embodiment 19, wherein at least one modified internucleoside linkage is a mesyl phosphoramidate internucleoside linkage.

Embodiment 21. The oligomeric compound of embodiment 20, wherein each internucleoside linkage is a phosphorothioate internucleoside linkage.

Embodiment 22. The oligomeric compound of any of embodiments 1-18, wherein each internucleoside linkage of the modified oligonucleotide is independently selected from a phosphodiester internucleoside linkage and a phosphorothioate internucleoside linkage.

Embodiment 23. The oligomeric compound of any of embodiments 1-18, wherein each internucleoside linkage of the modified oligonucleotide is independently selected from a phosphodiester internucleoside linkage, a phosphorothioate internucleoside linkage, and a mesyl phosphoramidate internucleoside linkage.

Embodiment 24. The oligomeric compound of any of embodiments 1-18, wherein each internucleoside linkage of the modified oligonucleotide is independently selected from a phosphorothioate internucleoside linkage and a mesyl phosphoramidate internucleoside linkage.

Embodiment 25. The oligomeric compound of any of embodiments 1-24, wherein the modified oligonucleotide has an internucleoside linkage motif of ssooooooooooooooooooss, wherein “s” is a phosphorothioate internucleoside linkage and “o” is a phosphodiester internucleoside linkage.

Embodiment 26. The oligomeric compound of any of embodiments 1-25, wherein the modified oligonucleotide comprises at least one modified nucleobase.

Embodiment 27. The oligomeric compound of embodiment 26, wherein the modified nucleobase is 5-methylcytosine.

Embodiment 28. The oligomeric compound of embodiment 27, wherein each cytosine is a 5-methylcytosine.

Embodiment 29. An oligomeric compound of any of embodiments 1-28, wherein the modified oligonucleotide has a sugar motif (5′ to 3′) of yfyfyfyfyfyfyfyfyfyfyyy or yfyyyfyyyyyyyfyfyyyyyyy, wherein each “y” represents a 2′-OMe sugar moiety and each “f” represents a 2′-F sugar moiety.

Embodiment 30. The oligomeric compound of any one of embodiments 1-29, wherein the oligomeric compound comprises a conjugate group.

Embodiment 31. The oligomeric compound of embodiment 30, wherein the conjugate group comprises a conjugate moiety and a conjugate linker.

Embodiment 32. The oligomeric compound of embodiment 31, wherein the conjugate moiety is a lipophilic group.

Embodiment 33. The oligomeric compound of embodiment 31, wherein the conjugate moiety is selected from a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

Embodiment 34. The oligomeric compound of any of embodiments 31-33, wherein the conjugate linker consists of a single bond.

Embodiment 35. The oligomeric compound of any of embodiments 31-33, wherein the conjugate linker is cleavable.

Embodiment 36. The oligomeric compound of any of embodiments 1-335, comprising a terminal group.

Embodiment 37. An oligomeric duplex, comprising a first oligomeric compound comprising a first modified oligonucleotide and a second oligomeric compound comprising a second modified oligonucleotide, wherein the first oligomeric compound is an oligomeric compound of any of embodiments 1-36.

Embodiment 38. The oligomeric duplex of embodiment 37, wherein the second modified oligonucleotide consists of 8 to 80 linked nucleosides, and wherein the nucleobase sequence of the second modified oligonucleotide comprises a complementary region of at least 8 nucleobases that is at least 90% complementary to an equal length portion of the first modified oligonucleotide.

Embodiment 39. An oligomeric duplex comprising:
a first oligomeric compound comprising a first modified oligonucleotide consisting of 15 to 30 linked nucleosides, wherein the nucleobase sequence of the first modified oligonucleotide comprises at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 11-39, 69-112, 157-204, 253-290, 329-375, 423-452, 483-516, 551-580, 611-650, 691-721, 753-898, 1045-1443; and
a second oligomeric compound comprising a second modified oligonucleotide consisting of 15 to 29 linked nucleosides, wherein the nucleobase sequence of the second modified oligonucleotide comprises a complementary region of at least 8 nucleobases that is at least 90% complementary to an equal length portion of the first modified oligonucleotide.

Embodiment 40. An oligomeric duplex comprising:
a first oligomeric compound comprising a first modified oligonucleotide consisting of 15 to 30 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 11-39, 69-112, 157-204, 253-290, 329-375, 423-452, 483-516, 551-580, 611-650, 691-721, 753-898, 1045-1443; and
a second oligomeric compound comprising a second modified oligonucleotide consisting of 15 to 29 linked nucleosides, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or 21 contiguous nucleobases of the nucleobase sequence of any of SEQ ID NOs: 40-68, 113-156, 205-252, 291-328, 376-422, 453-482, 517-550, 581-610, 651-690, 722-752, 899-1044, 1444-1842, wherein the nucleobase sequence of the second modified oligonucleotide is at least 90% complementary to an equal length portion of the first modified oligonucleotide.

Embodiment 41. An oligomeric duplex comprising:
a first oligomeric compound comprising a first modified oligonucleotide consisting of 23 linked nucleosides, wherein the nucleobase sequence of the first modified oligonucleotide consists of the nucleobase sequence of any of SEQ ID NOs: 11-39, 69-112, 157-204, 253-290, 329-375, 423-452, 483-516, 551-580, 611-650, 691-721, 753-898, 1045-1443; and
a second oligomeric compound comprising a second modified oligonucleotide consisting of 21 linked nucleosides, wherein the nucleobase sequence of the second modified oligonucleotide consists of the nucleobase sequence of any of SEQ ID NOs: 40-68, 113-156, 205-252, 291-328, 376-422, 453-482, 517-550, 581-610, 651-690, 722-752, 899-1044, 1444-1842, and wherein the nucleobase sequence of the second modified oligonucleotide is at least 90% complementary to an equal length portion of the first modified oligonucleotide.

Embodiment 42. The oligomeric duplex of any of embodiments 39-41, wherein the first modified oligonucleotide comprises a 5′-stabilized phosphate group.

Embodiment 43. The oligomeric duplex of embodiment 42, wherein the 5′-stabilized phosphate group comprises a cyclopropyl phosphonate or a vinyl phosphonate.

Embodiment 44. The oligomeric duplex of any of embodiments 39-43, wherein the first modified oligonucleotide comprises a glycol nucleic acid (GNA) sugar surrogate.

Embodiment 45. The oligomeric duplex of any of embodiments 39-44, wherein the first modified oligonucleotide comprises a 2′-NMA sugar moiety.

Embodiment 46. The oligomeric duplex of any of embodiments 39-45, wherein at least one nucleoside of the second modified oligonucleotide comprises a modified sugar moiety.

Embodiment 47. The oligomeric duplex of embodiment 39-46, wherein the modified sugar moiety of the second modified oligonucleotide comprises a bicyclic sugar moiety.

Embodiment 48. The oligomeric duplex of embodiment 39-47, wherein the bicyclic sugar moiety of the second modified oligonucleotide comprises a 2′-4′ bridge selected from —O—CH₂— and —O—CH(CH₃)—.

Embodiment 49. The oligomeric duplex of embodiment 39-48, wherein the modified sugar moiety of the second modified oligonucleotide comprises a non-bicyclic modified sugar moiety.

Embodiment 50. The oligomeric duplex of embodiment 49, wherein the non-bicyclic modified sugar moiety of the second modified oligonucleotide is a 2′-MOE sugar moiety, a 2′-F sugar moiety, or 2′-OMe sugar moiety.

Embodiment 51. The oligomeric duplex of any of embodiments 39-50, wherein at least one nucleoside of the second modified oligonucleotide comprises a sugar surrogate.

Embodiment 52. The oligomeric duplex of any of embodiments 39-51, wherein the second modified oligonucleotide comprises at least one modified internucleoside linkage.

Embodiment 53. The oligomeric duplex of embodiment 52, wherein the at least one modified internucleoside linkage of the second modified oligonucleotide is a phosphorothioate internucleoside linkage.

Embodiment 54. The oligomeric duplex of any of embodiments 39-52, wherein the second modified oligonucleotide comprises at least one phosphodiester internucleoside linkage.

Embodiment 55. The oligomeric duplex of any of embodiments 39-52, wherein each internucleoside linkage of the second modified oligonucleotide is independently selected from a phosphodiester internucleoside linkage or a phosphorothioate internucleoside linkage.

Embodiment 56. The oligomeric duplex of any of embodiments 39-52, wherein the internucleoside linkage motif of the first modified oligonucleotide is ssooooooooooooooooooss and the internucleoside linkage motif of the second modified oligonucleotide is ssooooooooooooooooss, wherein each “o” represents a phosphodiester internucleoside linkage and each “s” represents a phosphorothioate internucleoside linkage.

Embodiment 57. The oligomeric duplex of any of embodiments 39-56, wherein the second modified oligonucleotide comprises at least one modified nucleobase.

Embodiment 58. The oligomeric duplex of embodiment 57, wherein the modified nucleobase of the second modified oligonucleotide is 5-methylcytosine.

Embodiment 59. The oligomeric duplex of any of embodiments 39-58, wherein the second modified oligonucleotide comprises a conjugate group.

Embodiment 60. The oligomeric duplex of embodiment 59, wherein the conjugate group comprises a conjugate linker and a conjugate moiety.

Embodiment 61. The oligomeric duplex of embodiment 59 or 60, wherein the conjugate group is attached to the second modified oligonucleotide at the 5′-end of the second modified oligonucleotide.

Embodiment 62. The oligomeric duplex of embodiment 59 or 60, wherein the conjugate group is attached to the second modified oligonucleotide at the 3′-end of the second modified oligonucleotide.

Embodiment 63. The oligomeric duplex of any of embodiments 59-62, wherein the conjugate group comprises a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

Embodiment 64. The oligomeric duplex of any of embodiments 39-63, wherein the second modified oligonucleotide comprises a terminal group.

Embodiment 65. The oligomeric duplex of embodiment 64, wherein the terminal group is an abasic sugar moiety.

Embodiment 66. The oligomeric duplex of any of embodiments 39-65, wherein the second modified oligonucleotide consists of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 67. The oligomeric duplex of any of embodiments 39-66, wherein the first modified oligonucleotide consists of 23 linked nucleosides and the second modified oligonucleotide consists of 21 linked nucleosides.

Embodiment 68. The oligomeric duplex of embodiment 67, wherein the modified oligonucleotide of the first oligomeric compound has a sugar motif (from 5′ to 3′) of yfyfyfyfyfyfyfyfyfyfyyy and the second modified oligonucleotide has a sugar motif (from 5′ to 3′) of fyfyfyfyfyfyfyfyfyfyf, wherein each “y” represents a 2′-OMe sugar moiety and each “f” represents a 2′-F sugar moiety.

Embodiment 69. The oligomeric duplex of claim 67, wherein the modified oligonucleotide of the first oligomeric compound has a sugar motif (from 5′ to 3′) of: yfyyyfyyyyyyyfyfyyyyyyy and the second modified oligonucleotide has a sugar motif (from 5′ to 3′) of yyyyyyfyfffyyyyyyyyyy, wherein each “y” represents a 2′-OMe sugar moiety and each “f” represents a 2′-F sugar moiety.

Embodiment 70. An antisense agent, wherein the antisense agent is the oligomeric duplex of any of embodiments 37-69.

Embodiment 71. The antisense agent of embodiment 70, wherein the antisense agent is an RNAi agent capable of reducing the amount of tau nucleic acid through the activation of RISC/Ago2.

Embodiment 72. A chirally enriched population of oligomeric duplexes of embodiments 37-69, wherein the population is enriched for modified oligonucleotides comprising at least one particular phosphorothioate internucleoside linkage having a particular stereochemical configuration.

Embodiment 73. The chirally enriched population of embodiment 72, wherein the population is enriched for modified oligonucleotides having a particular, independently selected stereochemical configuration at each phosphorothioate internucleoside linkage.

Embodiment 74. The chirally enriched population of embodiment 73, wherein the population is enriched for modified oligonucleotides having the (Rp) configuration at one particular phosphorothioate internucleoside linkage and the (Sp) configuration at each of the remaining phosphorothioate internucleoside linkages.

Embodiment 75. A population of oligomeric duplexes comprising modified oligonucleotides of any of embodiments 37-69, wherein all of the phosphorothioate internucleoside linkages of the modified oligonucleotides are stereorandom.

Embodiment 76. A pharmaceutical composition comprising the oligomeric compound of any of embodiments 1-36, the oligomeric duplex of any of embodiments 37-69, the antisense agent of any of embodiments 70-71, or the population of any of embodiments 72-75, and a pharmaceutically acceptable diluent or carrier.

Embodiment 77. The pharmaceutical composition of embodiment 76, wherein the pharmaceutically acceptable diluent is phosphate buffered saline or artificial cerebrospinal fluid.

Embodiment 78. The pharmaceutical composition of embodiment 777, wherein the pharmaceutical composition consists essentially of the oligomeric compound, the oligomeric duplex, the antisense agent, or the population, and phosphate buffered saline or artificial cerebrospinal fluid.

Embodiment 79. A method comprising administering to an animal the oligomeric compound of any of embodiments 1-36, the oligomeric duplex of any of embodiments 37-69, the antisense agent of any of embodiments 70-71, the population of any of embodiments 72-75, or the pharmaceutical composition of any of embodiments 76-78.

Embodiment 80. The method of embodiment 79, wherein the animal has a tau-associated disease.

Embodiment 81. The method of embodiment 79 or embodiment 80, wherein the disease associated with tau is a tauopathy, Alzheimer's disease, frontotemporal dementia (FTD), frontotemporal dementia with parkinsonism-17 (FTDP-17), progressive supranuclear palsy (PSP), chronic traumatic encephalopathy (CTE), corticobasal ganglionic degeneration (CBD), epilepsy, or Dravet's Syndrome.

Embodiment 82. A method of treating a tau-associated disease comprising administering to an individual having or at risk for developing the tau-associated disease a therapeutically effective amount of the oligomeric compound of any of embodiments 1-36, the oligomeric duplex of any of embodiments 37-69, the antisense agent of any of embodiments 70-71, the population of any of embodiments 72-75, or the pharmaceutical composition according to any of embodiments 76-78 and thereby treating the tau-associated disease.

Embodiment 83. The method of embodiment 82, wherein the tau-associated disease is a tauopathy, Alzheimer's disease, frontotemporal dementia (FTD), frontotemporal dementia with parkinsonism-17 (FTDP-17), progressive supranuclear palsy (PSP), chronic traumatic encephalopathy (CTE), corticobasal ganglionic degeneration (CBD), epilepsy, or Dravet's Syndrome.

Embodiment 84. The method of embodiment 82 or embodiment 83, wherein the tau-associated disease is a tauopathy.

Embodiment 85. The method of embodiment 82 or embodiment 83, wherein the tau-associated disease is Alzheimer's disease.

Embodiment 86. The method of embodiment 82 or embodiment 83, wherein the tau-associated disease is frontotemporal dementia (FTD.

Embodiment 87. The method of embodiment 82 or embodiment 83, wherein the tau-associated disease is FTDP-17.

Embodiment 88. The method of embodiment 82 or embodiment 83, wherein the tau-associated disease is progressive supranuclear palsy (PSP).

Embodiment 89. The method of embodiment 82 or embodiment 83, wherein the tau-associated disease is chronic traumatic encephalopathy (CTE).

Embodiment 90. The method of embodiment 82 or embodiment 83, wherein the tau-associated disease is corticobasal ganglionic degeneration (CBD).

Embodiment 91. The method of embodiment 82 or embodiment 83, wherein the tau-associated disease is epilepsy.

Embodiment 92. The method of embodiment 82 or embodiment 83, wherein the tau-associated disease is Dravet's Syndrome.

Embodiment 93. The method of any of embodiments 83-92, wherein at least one symptom or hallmark of the tau-associated disease is ameliorated.

Embodiment 94. The method of embodiment 93, wherein the symptom or hallmark is loss of memory, loss of motor function, and/or an increase in the number and/or volume of neurofibrillary inclusions.

Embodiment 95. A method of reducing tau in a cell comprising contacting the cell with the oligomeric compound of any of embodiments 1-36, the oligomeric duplex of any of embodiments 37-69, the antisense agent of any of embodiments 70-71, the population of any of embodiments 72-75, or the pharmaceutical composition according to any of embodiments 76-78.

Embodiment 96. The method of embodiment 95, wherein the cell is a central nervous system cell.

Embodiment 97. The method of any of embodiments 95-96, wherein the cell is a human cell.

Embodiment 98. Use of the oligomeric compound of any of embodiments 1-36, the oligomeric duplex of any of embodiments 37-69, the antisense agent of any of embodiments 70-71, the population of any of embodiments 72-75, or the pharmaceutical composition according to any of embodiments 76-78, for treating a tau-associated disease.

Embodiment 99. Use of the oligomeric compound of any of embodiments 1-36, the oligomeric duplex of any of embodiments 37-69, the antisense agent of any of embodiments 70-71, the population of any of embodiments 72-75, or the pharmaceutical composition according to any of embodiments 76-78, for the manufacture of a medicament for treating a tau-associated disease.

Embodiment 100. The use of embodiments 98 or embodiment 99, wherein the tau-associated disease is a tauopathy, Alzheimer's disease, frontotemporal dementia (FTD), frontotemporal dementia with parkinsonism-17 (FTDP-17), progressive supranuclear palsy (PSP), chronic traumatic encephalopathy (CTE), corticobasal ganglionic degeneration (CBD), epilepsy, or Dravet's Syndrome.

Embodiment 101. The use of any of embodiments 98-100, wherein at least one symptom or hallmark is ameliorated.

Embodiment 102. The use of embodiment 101, wherein the at least one symptom or hallmark is loss of memory, loss of motor function, or an increase in the number and/or volume of neurofibrillary inclusions.

Embodiment 103. The use of any of embodiments 98-102, wherein the use of the oligomeric compound, the oligomeric duplex, the antisense agent, the population, or the pharmaceutical composition improves motor function, reduces the amount or volume of alpha-synuclein aggregates, reduces or delays neurodegeneration, improves cognitive function, or delays the onset or progression of dementia.

### I. Certain Oligonucleotides

Provided herein are oligomeric agents comprising antisense oligonucleotides complementary to a tau nucleic acid and optionally, sense oligonucleotides complementary to the antisense oligonucleotides. Antisense oligonucleotides and sense oligonucleotides typically comprise at least one modified nucleoside and/or at least one modified internucleoside linkage. Certain modified nucleosides and modified internucleoside linkages suitable for use in antisense oligonucleotides and/or sense oligonucleotides are described below.

### A. Certain Modified Nucleosides

Modified nucleosides comprise a modified sugar moiety or a modified nucleobase or both a modified sugar moiety and a modified nucleobase. Modified nucleosides comprising the following modified sugar moieties and/or the following modified nucleobases may be incorporated into antisense oligonucleotides and/or sense oligonucleotides.

### 1. Certain Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited, to substituents at the 2′, 3′, 4′, and/or 5′ positions. In certain embodiments, the furanosyl sugar moiety is a ribosyl sugar moiety. In certain embodiments, one or more acyclic substituent of non-bicyclic modified sugar moieties is branched.

In certain embodiments, non-bicyclic modified sugar moieties comprise a substituent group at the 2′-position. Examples of substituent groups suitable for the 2′-position of modified sugar moieties include but are not limited to: —F, —OCH₃ (“OMe” or “O-methyl”), and —O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), 2′-O(CH₂)₂O(CH₂)₂N(CH₃)₂ (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, O(CH₂)₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl. In certain embodiments, a 2′-substituted non-bicyclic modified nucleoside comprises a sugar moiety comprising a non-bridging 2′-substituent group selected from F, OCH₃, O(CH₂)₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”). In certain embodiments, a 2′-substituted sugar moiety of a modified nucleoside comprises a 2′-substituent group selected from: F, OCF₃, OCH₃, OCH₂CH₂OCH₃, O(CH₂)₂SCH₃, O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, a 2′-substituted sugar moiety of a modified nucleoside comprises 2′-substituent group selected from F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. Modified furanosyl sugar moieties described herein are in the β-D-ribosyl isomeric configuration unless otherwise specified.

In certain embodiments, non-bicyclic modified sugar moieties comprise a substituent group at the 4′-position. Examples of substituent groups suitable for the 4′-position of modified sugar moieties include but are not limited to alkoxy (e.g., methoxy), alkyl, and those described in Manoharan et al., WO 2015/106128.

In certain embodiments, non-bicyclic modified sugar moieties comprise a substituent group at the 3′-position. Examples of substituent groups suitable for the 3′-position of modified sugar moieties include but are not limited to alkoxy (e.g., methoxy), alkyl (e.g., methyl, ethyl).

In certain embodiments, non-bicyclic modified sugar moieties comprise a substituent group at the 5′-position. Examples of substituent groups suitable for the 5′-position of modified sugar moieties include but are not limited to vinyl, alkoxy (e.g., methoxy), alkyl (e.g., methyl (R or S), ethyl).

In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In naturally occurring nucleic acids, sugars are linked to one another 3′ to 5′. In certain embodiments, oligonucleotides include one or more nucleoside or sugar moiety linked at an alternative position, for example at the 2′ or inverted 5′ to 3′. For example, where the linkage is at the 2′ position, the 2′-substituent groups may instead be at the 3′-position.

Certain modified sugar moieties comprise a bridging sugar substituent that forms a second ring resulting in a bicyclic sugar moiety. Nucleosides comprising such bicyclic sugar moieties have been referred to as bicyclic nucleosides (BNAs), locked nucleosides, or conformationally restricted nucleotides (CRN). Certain such compounds are described in US Patent Publication No. 2013/0190383; and PCT publication WO 2013/036868. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include, but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙO—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US 2008/0039618; and Migawa et al., US 2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

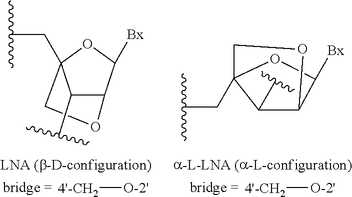

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mal Cane Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include, but are not limited to, hexitol nucleic acid (“HNA”), anitol nucleic acid (“ANA”), manitol nucleic acid (“MNA”) (see e.g., Leumann, C J. Bioorg. & Med. Chem. 2002, 10, 841-854), fluoro hexitol nucleic acid (F-HNA):

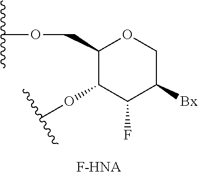

(“F-HNA”, see e.g., Swayze et al., U.S. Pat. No. 8,088,904; Swayze et al., U.S. Pat. No. 8,440,803; and Swayze et al., U.S. Pat. No. 9,005,906) F-HNA can also be referred to as a F-THP or 3′-fluoro tetrahydropyran, and nucleosides comprising additional modified THP compounds having the formula:

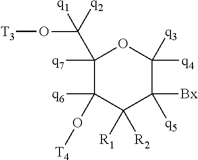

wherein, independently, for each of said modified THP nucleoside:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

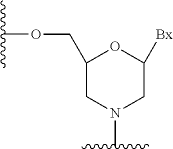

In certain embodiments, morpholinos may be modified, for example, by adding or altering various substituent groups from the above morpholino structure. Such sugar surrogates are referred to herein as “modified morpholinos.” In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the RNAi oligonucleotides are described in, for example, in Nielsen et al., Science, 1991, 254, 1497-1500.

In certain embodiments, sugar surrogates are the “unlocked” sugar structure of UNA (unlocked nucleic acid) nucleosides. UNA is a nucleoside wherein any of the bonds of the sugar moiety has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

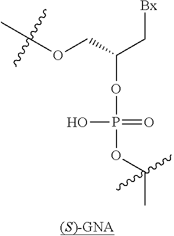

where Bx represents any nucleobase.

Many other modified sugar moieties and sugar surrogates are known in the art that can be used in modified nucleosides.

### 2. Certain Modified Nucleobases

In certain embodiments, oligonucleotides comprise one or more nucleoside comprising a modified nucleobase. In certain embodiments, oligonucleotides comprise one or more inosine nucleosides (i.e., nucleosides comprising a hypoxantine nucleobase).

In certain embodiments, modified nucleobases are selected from: 5-substituted pyrimidines, 6-azapyrimidines, alkyl or alkynyl substituted pyrimidines, alkyl substituted purines, and N-2, N-6, and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases, as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO 2021/030778, incorporated by reference herein. In certain embodiments, a modified internucleoside linkage comprises the formula:

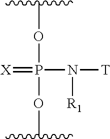

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

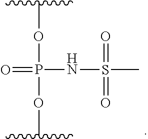 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

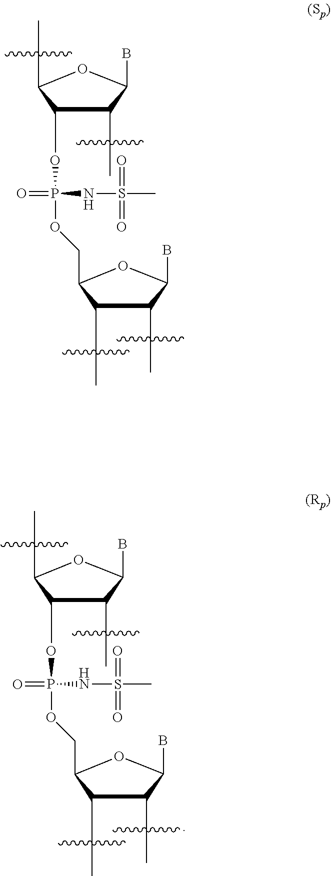

Representative internucleoside linkages having a chiral center include but are not limited to alkylphosphonates, mesyl phosphoramidates, and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate or mesyl phosphoramidate linkage. Nonetheless, each individual phosphorothioate or mesyl phosphoramidate of each individual oligonucleotide molecule has a defined stereoconfiguration. In certain embodiments, populations of modified oligonucleotides are enriched for modified oligonucleotides comprising one or more particular phosphorothioate or mesyl phosphoramidate internucleoside linkages in a particular, independently selected stereochemical configuration. In certain embodiments, the particular configuration of the particular phosphorothioate or mesyl phosphoramidate linkage is present in at least 65% of the molecules in the population. In certain embodiments, the particular configuration of the particular phosphorothioate or mesyl phosphoramidate linkage is present in at least 70% of the molecules in the population. In certain embodiments, the particular configuration of the particular phosphorothioate or mesyl phosphoramidate linkage is present in at least 80% of the molecules in the population. In certain embodiments, the particular configuration of the particular phosphorothioate or mesyl phosphoramidate linkage is present in at least 90% of the molecules in the population. In certain embodiments, the particular configuration of the particular phosphorothioate or mesyl phosphoramidate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate or mesyl phosphoramidate in the (Sp) configuration. In certain embodiments, a population of modified oligonucleotides is enriched for modified oligonucleotides having at least one phosphorothioate or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

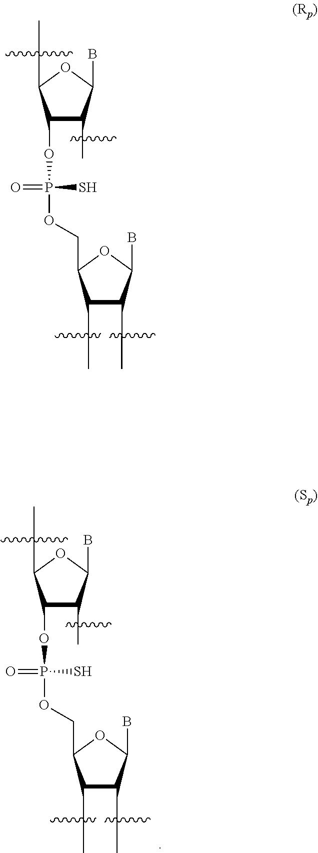

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, oligonucleotides (such as antisense oligonucleotides and/or sense oligonucleotides) comprise one or more inverted nucleoside, as shown below:

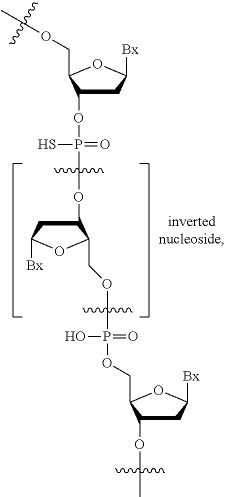

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

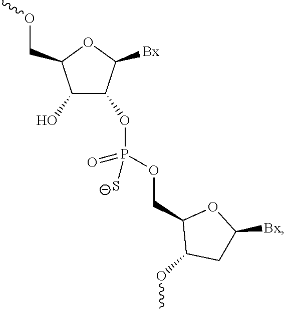

wherein each Bx represents any nucleobase.

### B. Antisense Oligonucleotides

In certain embodiments, antisense oligonucleotides comprise a number of linked nucleosides, wherein certain nucleosides and/or linkages are modified.

### 1. Certain Lengths

In certain embodiments, antisense oligonucleotides (including modified oligonucleotides) can have any of a variety of ranges of lengths. In certain embodiments, antisense oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, provided that X≤Y. For example, in certain embodiments, antisense oligonucleotides consist of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 29, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

In certain embodiments, antisense oligonucleotides consist of 12-30 linked nucleosides. In certain embodiments, antisense oligonucleotides consist of 15-30 linked nucleosides. In certain embodiments, antisense oligonucleotides consist of 17-25 linked nucleosides. In certain embodiments, antisense oligonucleotides consist of 17-23 linked nucleosides. In certain embodiments, antisense oligonucleotides consist of 17-21 linked nucleosides. In certain embodiments, antisense oligonucleotides consist of 18-30 linked nucleosides. In certain embodiments, antisense oligonucleotides consist of 19-29 linked nucleosides. In certain embodiments, antisense oligonucleotides consist of 20-30 linked nucleosides. In certain embodiments, antisense oligonucleotides consist of 21-30 linked nucleosides. In certain embodiments, antisense oligonucleotides consist of 23-30 linked nucleosides. In certain embodiments, antisense oligonucleotides consist of 18-25 linked nucleosides. In certain embodiments, antisense oligonucleotides consist of 20-22 linked nucleosides. In certain embodiments, antisense oligonucleotides consist of 21-23 linked nucleosides. In certain embodiments, antisense oligonucleotides consist of 23-24 linked nucleosides. In certain embodiments, antisense oligonucleotides consist of 20 linked nucleosides. In certain embodiments, antisense oligonucleotides consist of 21 linked nucleosides. In certain embodiments, antisense oligonucleotides consist of 22 linked nucleosides. In certain embodiments, antisense oligonucleotides consist of 23 linked nucleosides.

### 2. Certain Sugar Motifs

In certain embodiments, the sugar moiety of at least one nucleoside of an antisense oligonucleotide is a modified sugar moiety.

In certain such embodiments, at least one nucleoside comprises a 2′-OMe modified sugar moiety. In certain embodiments, at least 2 nucleosides comprise 2′-OMe modified sugar moieties. In certain embodiments, at least 5 nucleosides comprise 2′-OMe modified sugar moieties. In certain embodiments, at least 8 nucleosides comprise 2′-OMe modified sugar moieties. In certain embodiments, at least 10 nucleosides comprise 2′-OMe modified sugar moieties. In certain embodiments, at least 12 nucleosides comprise 2′-OMe modified sugar moieties. In certain embodiments, at least 14 nucleosides comprise 2′-OMe modified sugar moieties. In certain embodiments, at least 15 nucleosides comprise 2′-OMe modified sugar moieties. In certain embodiments, at least 17 nucleosides comprise 2′-OMe modified sugar moieties. In certain such embodiments, at least 18 nucleosides comprise 2′-OMe modified sugar moieties. In certain such embodiments, at least 20 nucleosides comprise 2′-OMe modified sugar moieties. In certain such embodiments, at least 21 nucleosides comprise 2′-OMe modified sugar moieties.

In certain embodiments, at least one nucleoside comprises a 2′-F modified sugar. In certain embodiments, at least 2 nucleosides comprise 2′-F modified sugar moieties. In certain embodiments, at least 3 nucleosides comprise 2′-F modified sugar moieties. In certain embodiments, at least 4 nucleosides comprise 2′-F modified sugar moieties. In certain embodiments, one, but not more than one nucleoside comprises a 2′-F modified sugar. In certain embodiments, 1 or 2 nucleosides comprise 2′-F modified sugar moieties. In certain embodiments, 1-3 nucleosides comprise 2′-F modified sugar moieties. In certain embodiments, at least 1-4 nucleosides comprise 2′-F modified sugar moieties. In certain embodiments, antisense oligonucleotides have a block of 2-4 contiguous 2′-F modified nucleosides. In certain embodiments, 4 nucleosides of an antisense oligonucleotide are 2′-F modified nucleosides and 3 of those 2′-F modified nucleosides are contiguous. In certain such embodiments the remainder of the nucleosides are 2′-OMe modified. In certain embodiments, antisense oligonucleotides have a sugar motif of (5′ to 3′): yfyfyfyfyfyfyfyfyfyfyyy or yfyyyfyyyyyyyfyfyyyyyyy, wherein each “y” represents a 2′-OMe sugar moiety and each “f” represents a 2′-fluororibosyl sugar.

In certain embodiments, one nucleoside of an antisense oligonucleotide is a UNA.

In certain embodiments, one nucleoside of an antisense oligonucleotide is a GNA.

In certain embodiments, 1-4 nucleosides of an antisense oligonucleotide is/are DNA. In certain such embodiments, the 1-4 DNA nucleosides are at one or both ends of the antisense oligonucleotide.

### 3. Certain Internucleoside Linkage Motifs

In certain embodiments, at least one linkage of the antisense oligonucleotide is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain such embodiments, the modified linkage is a phosphorothioate linkage. In certain embodiments, the remaining linkages are all unmodified phosphodiester linkages. In certain embodiments the internucleoside linkage motif is ssooooooooooooooooooss, wherein each “s” represents a phosphorothioate linkage and each “o” represents a phosphodiester linkage.

In certain embodiments, at least one linkage of the antisense oligonucleotide is an inverted linkage.

### C. Sense Oligonucleotides

In certain embodiments, sense oligonucleotides comprise a number of linked nucleosides, wherein certain nucleosides and/or linkages are modified.

### 1. Certain Lengths

In certain embodiments, sense oligonucleotides (including modified oligonucleotides) can have any of a variety of ranges of lengths. In certain embodiments, sense oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, sense oligonucleotides consist of 10 to 25, 10 to 30, 10 to 50, 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

In certain embodiments, sense oligonucleotides consist of 12-30 linked nucleosides. In certain embodiments, sense oligonucleotides consist of 15-29 linked nucleosides. In certain embodiments, sense oligonucleotides consist of 17-25 linked nucleosides. In certain embodiments, sense oligonucleotides consist of 17-23 linked nucleosides. In certain embodiments, sense oligonucleotides consist of 17-21 linked nucleosides. In certain embodiments, sense oligonucleotides consist of 18-30 linked nucleosides. In certain embodiments, sense oligonucleotides consist of 20-30 linked nucleosides. In certain embodiments, sense oligonucleotides consist of 21-30 linked nucleosides. In certain embodiments, sense oligonucleotides consist of 23-30 linked nucleosides. In certain embodiments, sense oligonucleotides consist of 18-25 linked nucleosides. In certain embodiments, sense oligonucleotides consist of 20-22 linked nucleosides. In certain embodiments, sense oligonucleotides consist of 21-23 linked nucleosides. In certain embodiments, sense oligonucleotides consist of 23-24 linked nucleosides. In certain embodiments, sense oligonucleotides consist of 20 linked nucleosides. In certain embodiments, sense oligonucleotides consist of 21 linked nucleosides. In certain embodiments, sense oligonucleotides consist of 22 linked nucleosides. In certain embodiments, sense oligonucleotides consist of 23 linked nucleosides. In certain embodiments, sense oligonucleotides consist of 25 linked nucleosides.

### 2. Certain Sugar Motifs

In certain embodiments, the sugar moiety of at least one nucleoside of a sense oligonucleotides is a modified sugar moiety.

In certain such embodiments, at least one nucleoside comprises a 2′-OMe modified sugar moiety. In certain embodiments, at least 2 nucleosides comprise 2′-OMe modified sugar moieties. In certain embodiments, at least 5 nucleosides comprise 2′-OMe modified sugar moieties. In certain embodiments, at least 8 nucleosides comprise 2′-OMe modified sugar moieties. In certain embodiments, at least 10 nucleosides comprise 2′-OMe modified sugar moieties. In certain embodiments, at least 12 nucleosides comprise 2′-OMe modified sugar moieties. In certain embodiments, at least 14 nucleosides comprise 2′-OMe modified sugar moieties. In certain embodiments, at least 15 nucleosides comprise 2′-OMe modified sugar moieties. In certain embodiments, at least 17 nucleosides comprise 2′-OMe modified sugar moieties. In certain such embodiments, at least 18 nucleosides comprise 2′-OMe modified sugar moieties. In certain such embodiments, at least 20 nucleosides comprise 2′-OMe modified sugar moieties. In certain such embodiments, at least 21 nucleosides comprise 2′-OMe modified sugar moieties.

In certain embodiments, at least one nucleoside comprises a 2′-F modified sugar moiety. In certain embodiments, at least 2 nucleosides comprise 2′-F modified sugar moieties. In certain embodiments, at least 3 nucleosides comprise 2′-F modified sugar moieties. In certain embodiments, at least 4 nucleosides comprise 2′-F modified sugar moieties. In certain embodiments, one, but not more than nucleoside comprises a 2′-F modified sugar moiety. In certain embodiments, 1 or 2 nucleosides comprise 2′-F modified sugar moieties. In certain embodiments, 1-3 nucleosides comprise 2′-F modified sugar moieties. In certain embodiments, at least 1-4 nucleosides comprise 2′-F modified sugar moieties. In certain embodiments, sense oligonucleotides have a block of 2-4 contiguous 2′-F modified nucleosides. In certain embodiments, 4 nucleosides of an sense oligonucleotide are 2′-F modified nucleosides and 3 of those 2′-F modified nucleosides are contiguous. In certain such embodiments the remainder of the nucleosides are 2′OMe modified. In certain embodiments, sense oligonucleotides have a sugar motif of (5′ to 3′): fyfyfyfyfyfyfyfyfyfyf or yyyyyyfyfffyyyyyyyyyy, wherein each “y” represents a 2′-OMe sugar moiety and each “f” represents a 2′-fluororibosyl sugar.

In certain embodiments, one nucleoside of an sense oligonucleotide is a UNA.

In certain embodiments, one nucleoside of an sense oligonucleotide is a GNA.

In certain embodiments, 1-4 nucleosides of an sense oligonucleotide is/are DNA. In certain such embodiments, the 1-4 DNA nucleosides are at one or both ends of the sense oligonucleotide.

### 3. Certain Internucleoside Linkages

In certain embodiments, at least one linkage of the sense oligonucleotides is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain such embodiments, the modified linkage is a phosphorothioate linkage. In certain embodiments, the remaining linkages are all unmodified phosphodiester linkages. In certain embodiments the internucleoside linkage motif is ssooooooooooooooooss, wherein each “s” represents a phosphorothioate linkage and each “o” represents a phosphodiester linkage.

In certain embodiments, at least one linkage of the sense oligonucleotides is an inverted linkage.

### II. Oligomeric Duplexes

In certain embodiments, an oligomeric compound described herein comprising an oligonucleotide, having a nucleobase sequence complementary to that of a target nucleic acid, is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a first modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides. In certain embodiments, the two oligonucleotides have at least one mismatch relative to one another. In certain embodiments, the oligomeric duplex is an antisense agent.

In certain embodiments, an oligomeric duplex comprises:
a first oligomeric compound comprising a first modified oligonucleotide consisting of 15 to 30 linked nucleosides wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 11-39, 69-112, 157-204, 253-290, 329-375, 423-452, 483-516, 551-580, 611-650, 691-721, 753-898, 1045-1443; and
a second oligomeric compound comprising a second modified oligonucleotide consisting of 15 to 29 linked nucleosides wherein the nucleobase sequence of the second modified oligonucleotide comprises a complementary region of at least 12 nucleobases that is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second oligomeric compound is a sense compound. In certain embodiments, the second modified oligonucleotide is a sense RNAi oligonucleotide. In certain embodiments, the nucleobase sequence of the second modified oligonucleotide comprises a complementary region of at least 9, at least 10, at least 11, at least 12, at least 13, at least 14, at least 15, at least 16, at least 17, at least 18, at least 19, at least 20, or 21 nucleobases that is at least 90% complementary to the nucleobase sequence of an equal portion of the first modified oligonucleotide. In certain embodiments, the nucleobase sequence of the second modified oligonucleotide comprises a complementary region of at least 8, at least 9, at least 10, at least 11, at least 12, at least 13, at least 14, at least 15, at least 16, at least 17, at least 18, at least 19, at least 20, or 21 nucleobases that is at least 95% complementary to the nucleobase sequence of an equal portion of the first modified oligonucleotide. In certain embodiments, the nucleobase sequence of the second modified oligonucleotide comprises a complementary region of at least 8, at least 9, at least 10, at least 11, at least 12, at least 13, at least 14, at least 15, at least 16, at least 17, at least 18, at least 19, at least 20, or 21 nucleobases that is 100% complementary to the nucleobase sequence of an equal portion of the first modified oligonucleotide.

In certain embodiments, an oligomeric duplex comprises:
a first oligomeric compound comprising a first modified oligonucleotide consisting of 15 to 30 linked nucleosides wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 11-39, 69-112, 157-204, 253-290, 329-375, 423-452, 483-516, 551-580, 611-650, 691-721, 753-898, 1045-1443; and
a second oligomeric compound comprising a second modified oligonucleotide consisting of 15 to 29 linked nucleosides wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of the nucleobase sequence of any of SEQ ID NOs: 40-68, 113-156, 205-252, 291-328, 376-422, 453-482, 517-550, 581-610, 651-690, 722-752, 899-1044, 1444-1842, wherein the nucleobase sequence of the second modified oligonucleotide is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second oligomeric compound is a sense compound. In certain embodiments, the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent.

In certain embodiments, an oligomeric duplex comprises:
a first oligomeric compound comprising a first modified oligonucleotide consisting of 23 linked nucleosides, wherein the nucleobase sequence of the first modified oligonucleotide consists of the nucleobase sequence of any of SEQ ID NOs: 11-39, 69-112, 157-204, 253-290, 329-375, 423-452, 483-516, 551-580, 611-650, 691-721, 753-898, 1045-1443; and
a second oligomeric compound comprising a second modified oligonucleotide consisting of 21 linked nucleosides, wherein the nucleobase sequence of the second modified oligonucleotide consists of the nucleobase sequence of any of SEQ ID NOs: 40-68, 113-156, 205-252, 291-328, 376-422, 453-482, 517-550, 581-610, 651-690, 722-752, 899-1044, 1444-1842, wherein the nucleobase sequence of the second modified oligonucleotide is at least 90% complementary to an equal length portion of the first modified oligonucleotide.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second oligomeric compound is a sense compound. In certain embodiments, the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent.

In certain embodiments, an oligomeric duplex comprises a first oligomeric compound comprising a first modified oligonucleotide consisting of 15 to 30 linked nucleosides and a second oligomeric compound comprising a second modified oligonucleotide consisting of 15 to 29 linked nucleosides, wherein the nucleobase sequence of the first modified oligonucleotide and the nucleobase sequence of the second modified oligonucleotide each comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the following pairs of nucleobase sequences recited in SEQ ID NOs: 11/40, 12/41, 13/42, 14/43, 15/44, 16/45, 17/46, 18/47, 19/48, 20/49, 21/50, 22/51, 23/52, 24/53, 25/54, 26/55, 27/56, 28/57, 29/58, 30/59, 31/60, 32/61, 33/62, 34/63, 35/64, 36/65, 37/66, 38/67, 39/68, 69/113, 70/114, 71/115, 72/116, 73/117, 74/118, 75/119, 76/120, 77/121, 78/122, 79/123, 80/124, 81/125, 82/126, 83/127, 84/128, 85/129, 86/130, 87/131, 88/132, 89/133, 90/134, 91/135, 92/136, 93/137, 94/138, 95/139, 96/140, 97/141, 98/142, 99/143, 100/144, 101/145, 102/146, 103/147, 104/148, 105/149, 106/150, 107/151, 108/152, 109/153, 110/154, 111/155, 112/156, 157/205, 158/206, 159/207, 160/208, 161/209, 162/210, 163/211, 164/212, 165/213, 166/214, 167/215, 168/216, 169/217, 170/218, 171/219, 172/220, 173/221, 174/222, 175/223, 176/224, 177/225, 178/226, 179/227, 180/228, 181/229, 182/230, 183/231, 184/232, 185/233, 186/234, 187/235, 188/236, 189/237, 190/238, 191/239, 192/240, 193/241, 194/242, 195/243, 196/244, 197/245, 198/246, 199/247, 200/248, 201/249, 202/250, 203/251, 204/252, 253/291, 254/292, 255/293, 256/294, 257/295, 258/296, 259/297, 260/298, 261/299, 262/300, 263/301, 264/302, 265/303, 266/304, 267/305, 268/306, 269/307, 270/308, 271/309, 272/310, 273/311, 274/312, 275/313, 276/314, 277/315, 278/316, 279/317, 280/318, 281/319, 282/320, 283/321, 284/322, 285/323, 286/324, 287/325, 288/326, 289/327, 290/328, 329/376, 330/377, 331/378, 332/379, 333/380, 334/381, 335/382, 336/383, 337/384, 338/385, 339/386, 340/387, 341/388, 342/389, 343/390, 344/391, 345/392, 346/393, 347/394, 348/395, 349/396, 350/397, 351/398, 352/399, 353/400, 354/401, 355/402, 356/403, 357/404, 358/405, 359/406, 360/407, 361/408, 362/409, 363/410, 364/411, 365/412, 366/413, 367/414, 368/415, 369/416, 370/417, 371/418, 372/419, 373/420, 374/421, 375/422, 423/453, 424/454, 425/455, 426/456, 427/457, 428/458, 429/459, 430/460, 431/461, 432/462, 433/463, 434/464, 435/465, 436/466, 437/467, 438/468, 439/469, 440/470, 441/471, 442/472, 443/473, 444/474, 445/475, 446/476, 447/477, 448/478, 449/479, 450/480, 451/481, 452/482, 483/517, 484/518, 485/519, 486/520, 487/521, 488/522, 489/523, 490/524, 491/525, 492/526, 493/527, 494/528, 495/529, 496/530, 497/531, 498/532, 499/533, 500/534, 501/535, 502/536, 503/537, 504/538, 505/539, 506/540, 507/541, 508/542, 509/543, 510/544, 511/545, 512/546, 513/547, 514/548, 515/549, 516/550, 551/581, 552/582, 553/583, 554/584, 555/585, 556/586, 557/587, 558/588, 559/589, 560/590, 561/591, 562/592, 563/593, 564/594, 565/595, 566/596, 567/597, 568/598, 569/599, 570/600, 571/601, 572/602, 573/603, 574/604, 575/605, 576/606, 577/607, 578/608, 579/609, 580/610, 611/651, 612/652, 613/653, 614/654, 615/655, 616/656, 617/657, 618/658, 619/659, 620/660, 621/661, 622/662, 623/663, 624/664, 625/665, 626/666, 627/667, 628/668, 629/669, 630/670, 631/671, 632/672, 633/673, 634/674, 635/675, 636/676, 637/677, 638/678, 639/679, 640/680, 641/681, 642/682, 643/683, 644/684, 645/685, 646/686, 647/687, 648/688, 649/689, 650/690, 691/722, 692/723, 693/724, 694/725, 695/726, 696/727, 697/728, 698/729, 699/730, 700/731, 701/732, 702/733, 703/734, 704/735, 705/736, 706/737, 707/738, 708/739, 709/740, 710/741, 711/742, 712/743, 713/744, 714/745, 715/746, 716/747, 717/748, 718/749, 719/750, 720/751, 721/752, 753/899, 754/900, 755/901, 756/902, 757/903, 758/904, 759/905, 760/906, 761/907, 762/908, 763/909, 764/910, 765/911, 766/912, 767/913, 768/914, 769/915, 770/916, 771/917, 772/918, 773/919, 774/920, 775/921, 776/922, 777/923, 778/924, 779/925, 780/926, 781/927, 782/928, 783/929, 784/930, 785/931, 786/932, 787/933, 788/934, 789/935, 790/936, 791/937, 792/938, 793/939, 794/940, 795/941, 796/942, 797/943, 798/944, 799/945, 800/946, 901/947, 802/948, 803/949, 804/950, 805/951, 806/952, 807/953, 808/854, 809/955, 810/956, 811/957, 812/958, 813/959, 814/960, 815/961, 816/962, 817/963, 818/964, 819/965, 820/966, 821/967, 822/968, 823/969, 824/970, 825/971, 826/972, 827/973, 828/974, 829/975, 830/976, 831/977, 832/978, 833/979, 834/980, 835/981, 836/982, 837/983, 838/984, 839/985, 840/986, 841/987, 842/988, 843/989, 844/990, 845/991, 846/992, 847/993, 848/994, 849/995, 850/996, 851/997, 852/998, 853/999, 854/1000, 855/1001, 856/1002, 857/1003, 858/1004, 859/1005, 860/1006, 861/1007, 862/1008, 863/1009, 864/1010, 865/1011, 866/1012, 867/1013, 868/1014, 869/1015, 870/1016, 871/1017, 872/1018, 873/1019, 874/1020, 875/1021, 876/1022, 877/1023, 878/1024, 879/1025, 880/1026, 881/1027, 882/1028, 883/1029, 884/1030, 885/1031, 886/1032, 887/1033, 888/1034, 889/1035, 890/1036, 891/1037, 892/1038, 893/1039, 894/1040, 895/1041, 896/1042, 897/1043, 898/1044, 1045/1444, 1046/1445, 1047/1446, 1048/1447, 1049/1448, 1050/1449, 1051/1450, 1052/1451, 1053/1452, 1054/1453, 1055/1454, 1056/1455, 1057/1456, 1058/1457, 1059/1458, 1060/1459, 1061/1460, 1062/1461, 1063/1462, 1064/1463, 1065/1464, 1066/1465, 1067/1466, 1068/1467, 1069/1468, 1070/1469, 1071/1470, 1072/1471, 1073/1472, 1074/1473, 1075/1474, 1076/1475, 1077/1476, 1078/1477, 1079/1478, 1080/1479, 1081/1480, 1082/1481, 1083/1482, 1084/1483, 1085/1484, 1086/1485, 1087/1486, 1088/1487, 1089/1488, 1090/1489, 1091/1490, 1092/1491, 1093/1492, 1094/1493, 1095/1494, 1096/1495, 1097/1496, 1098/1497, 1099/1498, 1100/1499, 1101/1500, 1102/1501, 1103/1502, 1104/1503, 1105/1504, 1106/1505, 1107/1506, 1108/1507, 1109/1508, 1110/1509, 1111/1510, 1112/1511, 1113/1512, 1114/1513, 1115/1514, 1116/1515, 1117/1516, 1118/1517, 1119/1518, 1120/1519, 1121/1520, 1122/1521, 1123/1522, 1124/1523, 1125/1524, 1126/1525, 1127/1526, 1128/1527, 1129/1528, 1130/1529, 1131/1530, 1132/1531, 1133/1532, 1134, 1533, 1135/1534, 1136/1535, 1137/1536, 1138/1537, 1139/1538, 1140/1539, 1141/1540, 1142/1541, 1143/1542, 1144/1543, 1145/1544, 1146/1545, 1147/1546, 1148/1547, 1149/1548, 1150/1549, 1151/1550, 1152/1551, 1153/1552, 1154/1553, 1155/1554, 1156/1555, 1157/1556, 1158/1557, 1159/1558, 1160/1559, 1161/1560, 1162/1561, 1163/1562, 1164/1563, 1165/1564, 1166/1565, 1167/1566, 1168/1567, 1169/1568, 1170/1569, 1171/1570, 1172/1571, 1173/1572, 1174/1573, 1175/1574, 1176/1575, 1177/1576, 1178/1577, 1179/1578, 1180/1579, 1181/1580, 1182/1581, 1183/1582, 1184/1583, 1185/1584, 1186/1585, 1187/1586, 1188/1587, 1189/1588, 1190/1589, 1191/1590, 1192/1591, 1193/1592, 1194/1593, 1195/1594, 1196/1595, 1197/1596, 1198/1597, 1199/1598, 1200/1599, 1201/1600, 1202/1601, 1203/1602, 1204/1603, 1205/1604, 1206/1605, 1207/1608, 1208/1607, 1209/1608, 1210/1609, 1211/1610, 1212/1611, 1213/1612, 1214/1613, 1215/1614, 1216/1615, 1217/1616, 1218/1617, 1219/1618, 1220/1619, 1221/1620, 1222/1621, 1223/1622, 1224/1623, 1225/1624, 1226/1625, 1227/1626, 1228/1627, 1229/1628, 1230/1629, 1231/1630, 1232/1631, 1233/1632, 1234/1633, 1235/1634, 1236/1635, 1237/1636, 1238/1637, 1239/1638, 1240/1639, 1241/1340, 1242/1641, 1243/1642, 1244/1643, 1245/1644, 1246/1645, 1247/1646, 1248/1647, 1249/1648, 1250/1649, 1251/1650, 1252/1651, 1253/1652, 1254/1653, 1255/1654, 1256/1655, 1257/1656, 1258/1657, 1259/1658, 1260/1659, 1261/1660, 1262/1661, 1263/1662, 1264/1663, 1265/1664, 1266/1665, 1267/1666, 1268/1667, 1269/1668, 1270/1669, 1271/1670, 1272/1671, 1273/1672, 1274/1673, 1275/1674, 1276/1675, 1277/1676, 1278/1677, 1279/1678, 1280/1679, 1281/1680, 1282/1681, 1283/1682, 1284/1683, 1285/1684, 1286/1685, 1287/1686, 1288/1687, 1289/1688, 1290/1689, 1291/1690, 1292/1691, 1293/1692, 1294/1693, 1295/1694, 1296/1695, 1297/1696, 1298/1697, 1299/1698, 1300/1699, 1301/1700, 1302/1701, 1303/1702, 1304/1703, 1305/1704, 1306/1705, 1307/1706, 1308/1707, 1309/1708, 1310/1709, 1311/1710, 1312/1711, 1313/1712, 1314/1713, 1315/1714, 1316/1715, 1317/1716, 1318/1717, 1319/1718, 1320/1719, 1321/1720, 1322/1721, 1323/1722, 1324/1723, 1325/1724, 1326/1725, 1327/1726, 1328/1727, 1329/1728, 1330/1729, 1331/1730, 1332/1731, 1333/1732, 1334/1733, 1335/1734, 1336/1735, 1337/1736, 1338/1737, 1339/1738, 1340/1739, 1341/1740, 1342/1741, 1343/1742, 1344/1743, 1345/1744, 1346/1745, 1347/1746, 1348/1747, 1349/1748, 1350/1749, 1351/1750, 1352/1751, 1353/1752, 1354/1753, 1355/1754, 1356/1755, 1357/1756, 1358/1757, 1359/1758, 1360/1759, 1361/1760, 1362/1761, 1363/1762, 1364/1763, 1365/1764, 1366/1765, 1367/1766, 1368/1767, 1369/1768, 1370/1769, 1371/1770, 1372/1771, 1373/1772, 1374/1773, 1375/1774, 1376/1775, 1377/1776, 1378/1777, 1379/1778, 1380/1779, 1381/1780, 1382/1781, 1383/1782, 1384/1783, 1385/1784, 1386/1785, 1387/1786, 1388/1787, 1389/1788, 1390/1789, 1391/1790, 1392/1791, 1393/1792, 1394/1793, 1395/1794, 1396/1795, 1397/1796, 1398/1797, 1399/1798, 1400/1799, 1401/1800, 1402/1801, 1403/1802, 1404/1803, 1405/1804, 1406/1805, 1407/1806, 1408/1807, 1409/1808, 1410/1809, 1411/1810, 1412/1811, 1413/1812, 1414/1813, 1415/1814, 1416/1815, 1417/1816, 1418/1817, 1419/1818, 1420/1819, 1421/1820, 1422/1821, 1423/1822, 1424/1823, 1425/1824, 1426/1825, 1427/1826, 1428/1827, 1429/1828, 1430/1829, 1431/1830, 1432/1831, 1433/1832, 1434/1833, 1435/1834, 1436/1835, 1437/1836, 1438/1837, 1439/1838, 1440/1839, 1441/1840, 1442/1841, 1443/1842, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second oligomeric compound is a sense compound. In certain embodiments, the second modified oligonucleotide is a sense RNAi oligonucleotide.

In certain embodiments, an oligomeric duplex comprises a first oligomeric compound comprising a first modified oligonucleotide consisting of 15 to 30 linked nucleosides and a second oligomeric compound comprising a second modified oligonucleotide consisting of 15 to 29 linked nucleosides, wherein the nucleobase sequences of the first modified oligonucleotide and second modified oligonucleotide comprise any of the following pairs of nucleobase sequences recited in SEQ ID NOs: 11/40, 12/41, 13/42, 14/43, 15/44, 16/45, 17/46, 18/47, 19/48, 20/49, 21/50, 22/51, 23/52, 24/53, 25/54, 26/55, 27/56, 28/57, 29/58, 30/59, 31/60, 32/61, 33/62, 34/63, 35/64, 36/65, 37/66, 38/67, 39/68, 69/113, 70/114, 71/115, 72/116, 73/117, 74/118, 75/119, 76/120, 77/121, 78/122, 79/123, 80/124, 81/125, 82/126, 83/127, 84/128, 85/129, 86/130, 87/131, 88/132, 89/133, 90/134, 91/135, 92/136, 93/137, 94/138, 95/139, 96/140, 97/141, 98/142, 99/143, 100/144, 101/145, 102/146, 103/147, 104/148, 105/149, 106/150, 107/151, 108/152, 109/153, 110/154, 111/155, 112/156, 157/205, 158/206, 159/207, 160/208, 161/209, 162/210, 163/211, 164/212, 165/213, 166/214, 167/215, 168/216, 169/217, 170/218, 171/219, 172/220, 173/221, 174/222, 175/223, 176/224, 177/225, 178/226, 179/227, 180/228, 181/229, 182/230, 183/231, 184/232, 185/233, 186/234, 187/235, 188/236, 189/237, 190/238, 191/239, 192/240, 193/241, 194/242, 195/243, 196/244, 197/245, 198/246, 199/247, 200/248, 201/249, 202/250, 203/251, 204/252, 253/291, 254/292, 255/293, 256/294, 257/295, 258/296, 259/297, 260/298, 261/299, 262/300, 263/301, 264/302, 265/303, 266/304, 267/305, 268/306, 269/307, 270/308, 271/309, 272/310, 273/311, 274/312, 275/313, 276/314, 277/315, 278/316, 279/317, 280/318, 281/319, 282/320, 283/321, 284/322, 285/323, 286/324, 287/325, 288/326, 289/327, 290/328, 329/376, 330/377, 331/378, 332/379, 333/380, 334/381, 335/382, 336/383, 337/384, 338/385, 339/386, 340/387, 341/388, 342/389, 343/390, 344/391, 345/392, 346/393, 347/394, 348/395, 349/396, 350/397, 351/398, 352/399, 353/400, 354/401, 355/402, 356/403, 357/404, 358/405, 359/406, 360/407, 361/408, 362/409, 363/410, 364/411, 365/412, 366/413, 367/414, 368/415, 369/416, 370/417, 371/418, 372/419, 373/420, 374/421, 375/422, 423/453, 424/454, 425/455, 426/456, 427/457, 428/458, 429/459, 430/460, 431/461, 432/462, 433/463, 434/464, 435/465, 436/466, 437/467, 438/468, 439/469, 440/470, 441/471, 442/472, 443/473, 444/474, 445/475, 446/476, 447/477, 448/478, 449/479, 450/480, 451/481, 452/482, 483/517, 484/518, 485/519, 486/520, 487/521, 488/522, 489/523, 490/524, 491/525, 492/526, 493/527, 494/528, 495/529, 496/530, 497/531, 498/532, 499/533, 500/534, 501/535, 502/536, 503/537, 504/538, 505/539, 506/540, 507/541, 508/542, 509/543, 510/544, 511/545, 512/546, 513/547, 514/548, 515/549, 516/550, 551/581, 552/582, 553/583, 554/584, 555/585, 556/586, 557/587, 558/588, 559/589, 560/590, 561/591, 562/592, 563/593, 564/594, 565/595, 566/596, 567/597, 568/598, 569/599, 570/600, 571/601, 572/602, 573/603, 574/604, 575/605, 576/606, 577/607, 578/608, 579/609, 580/610, 611/651, 612/652, 613/653, 614/654, 615/655, 616/656, 617/657, 618/658, 619/659, 620/660, 621/661, 622/662, 623/663, 624/664, 625/665, 626/666, 627/667, 628/668, 629/669, 630/670, 631/671, 632/672, 633/673, 634/674, 635/675, 636/676, 637/677, 638/678, 639/679, 640/680, 641/681, 642/682, 643/683, 644/684, 645/685, 646/686, 647/687, 648/688, 649/689, 650/690, 691/722, 692/723, 693/724, 694/725, 695/726, 696/727, 697/728, 698/729, 699/730, 700/731, 701/732, 702/733, 703/734, 704/735, 705/736, 706/737, 707/738, 708/739, 709/740, 710/741, 711/742, 712/743, 713/744, 714/745, 715/746, 716/747, 717/748, 718/749, 719/750, 720/751, 721/752, 753/899, 754/900, 755/901, 756/902, 757/903, 758/904, 759/905, 760/906, 761/907, 762/908, 763/909, 764/910, 765/911, 766/912, 767/913, 768/914, 769/915, 770/916, 771/917, 772/918, 773/919, 774/920, 775/921, 776/922, 777/923, 778/924, 779/925, 780/926, 781/927, 782/928, 783/929, 784/930, 785/931, 786/932, 787/933, 788/934, 789/935, 790/936, 791/937, 792/938, 793/939, 794/940, 795/941, 796/942, 797/943, 798/944, 799/945, 800/946, 901/947, 802/948, 803/949, 804/950, 805/951, 806/952, 807/953, 808/854, 809/955, 810/956, 811/957, 812/958, 813/959, 814/960, 815/961, 816/962, 817/963, 818/964, 819/965, 820/966, 821/967, 822/968, 823/969, 824/970, 825/971, 826/972, 827/973, 828/974, 829/975, 830/976, 831/977, 832/978, 833/979, 834/980, 835/981, 836/982, 837/983, 838/984, 839/985, 840/986, 841/987, 842/988, 843/989, 844/990, 845/991, 846/992, 847/993, 848/994, 849/995, 850/996, 851/997, 852/998, 853/999, 854/1000, 855/1001, 856/1002, 857/1003, 858/1004, 859/1005, 860/1006, 861/1007, 862/1008, 863/1009, 864/1010, 865/1011, 866/1012, 867/1013, 868/1014, 869/1015, 870/1016, 871/1017, 872/1018, 873/1019, 874/1020, 875/1021, 876/1022, 877/1023, 878/1024, 879/1025, 880/1026, 881/1027, 882/1028, 883/1029, 884/1030, 885/1031, 886/1032, 887/1033, 888/1034, 889/1035, 890/1036, 891/1037, 892/1038, 893/1039, 894/1040, 895/1041, 896/1042, 897/1043, 898/1044, 1045/1444, 1046/1445, 1047/1446, 1048/1447, 1049/1448, 1050/1449, 1051/1450, 1052/1451, 1053/1452, 1054/1453, 1055/1454, 1056/1455, 1057/1456, 1058/1457, 1059/1458, 1060/1459, 1061/1460, 1062/1461, 1063/1462, 1064/1463, 1065/1464, 1066/1465, 1067/1466, 1068/1467, 1069/1468, 1070/1469, 1071/1470, 1072/1471, 1073/1472, 1074/1473, 1075/1474, 1076/1475, 1077/1476, 1078/1477, 1079/1478, 1080/1479, 1081/1480, 1082/1481, 1083/1482, 1084/1483, 1085/1484, 1086/1485, 1087/1486, 1088/1487, 1089/1488, 1090/1489, 1091/1490, 1092/1491, 1093/1492, 1094/1493, 1095/1494, 1096/1495, 1097/1496, 1098/1497, 1099/1498, 1100/1499, 1101/1500, 1102/1501, 1103/1502, 1104/1503, 1105/1504, 1106/1505, 1107/1506, 1108/1507, 1109/1508, 1110/1509, 1111/1510, 1112/1511, 1113/1512, 1114/1513, 1115/1514, 1116/1515, 1117/1516, 1118/1517, 1119/1518, 1120/1519, 1121/1520, 1122/1521, 1123/1522, 1124/1523, 1125/1524, 1126/1525, 1127/1526, 1128/1527, 1129/1528, 1130/1529, 1131/1530, 1132/1531, 1133/1532, 1134, 1533, 1135/1534, 1136/1535, 1137/1536, 1138/1537, 1139/1538, 1140/1539, 1141/1540, 1142/1541, 1143/1542, 1144/1543, 1145/1544, 1146/1545, 1147/1546, 1148/1547, 1149/1548, 1150/1549, 1151/1550, 1152/1551, 1153/1552, 1154/1553, 1155/1554, 1156/1555, 1157/1556, 1158/1557, 1159/1558, 1160/1559, 1161/1560, 1162/1561, 1163/1562, 1164/1563, 1165/1564, 1166/1565, 1167/1566, 1168/1567, 1169/1568, 1170/1569, 1171/1570, 1172/1571, 1173/1572, 1174/1573, 1175/1574, 1176/1575, 1177/1576, 1178/1577, 1179/1578, 1180/1579, 1181/1580, 1182/1581, 1183/1582, 1184/1583, 1185/1584, 1186/1585, 1187/1586, 1188/1587, 1189/1588, 1190/1589, 1191/1590, 1192/1591, 1193/1592, 1194/1593, 1195/1594, 1196/1595, 1197/1596, 1198/1597, 1199/1598, 1200/1599, 1201/1600, 1202/1601, 1203/1602, 1204/1603, 1205/1604, 1206/1605, 1207/1608, 1208/1607, 1209/1608, 1210/1609, 1211/1610, 1212/1611, 1213/1612, 1214/1613, 1215/1614, 1216/1615, 1217/1616, 1218/1617, 1219/1618, 1220/1619, 1221/1620, 1222/1621, 1223/1622, 1224/1623, 1225/1624, 1226/1625, 1227/1626, 1228/1627, 1229/1628, 1230/1629, 1231/1630, 1232/1631, 1233/1632, 1234/1633, 1235/1634, 1236/1635, 1237/1636, 1238/1637, 1239/1638, 1240/1639, 1241/1340, 1242/1641, 1243/1642, 1244/1643, 1245/1644, 1246/1645, 1247/1646, 1248/1647, 1249/1648, 1250/1649, 1251/1650, 1252/1651, 1253/1652, 1254/1653, 1255/1654, 1256/1655, 1257/1656, 1258/1657, 1259/1658, 1260/1659, 1261/1660, 1262/1661, 1263/1662, 1264/1663, 1265/1664, 1266/1665, 1267/1666, 1268/1667, 1269/1668, 1270/1669, 1271/1670, 1272/1671, 1273/1672, 1274/1673, 1275/1674, 1276/1675, 1277/1676, 1278/1677, 1279/1678, 1280/1679, 1281/1680, 1282/1681, 1283/1682, 1284/1683, 1285/1684, 1286/1685, 1287/1686, 1288/1687, 1289/1688, 1290/1689, 1291/1690, 1292/1691, 1293/1692, 1294/1693, 1295/1694, 1296/1695, 1297/1696, 1298/1697, 1299/1698, 1300/1699, 1301/1700, 1302/1701, 1303/1702, 1304/1703, 1305/1704, 1306/1705, 1307/1706, 1308/1707, 1309/1708, 1310/1709, 1311/1710, 1312/1711, 1313/1712, 1314/1713, 1315/1714, 1316/1715, 1317/1716, 1318/1717, 1319/1718, 1320/1719, 1321/1720, 1322/1721, 1323/1722, 1324/1723, 1325/1724, 1326/1725, 1327/1726, 1328/1727, 1329/1728, 1330/1729, 1331/1730, 1332/1731, 1333/1732, 1334/1733, 1335/1734, 1336/1735, 1337/1736, 1338/1737, 1339/1738, 1340/1739, 1341/1740, 1342/1741, 1343/1742, 1344/1743, 1345/1744, 1346/1745, 1347/1746, 1348/1747, 1349/1748, 1350/1749, 1351/1750, 1352/1751, 1353/1752, 1354/1753, 1355/1754, 1356/1755, 1357/1756, 1358/1757, 1359/1758, 1360/1759, 1361/1760, 1362/1761, 1363/1762, 1364/1763, 1365/1764, 1366/1765, 1367/1766, 1368/1767, 1369/1768, 1370/1769, 1371/1770, 1372/1771, 1373/1772, 1374/1773, 1375/1774, 1376/1775, 1377/1776, 1378/1777, 1379/1778, 1380/1779, 1381/1780, 1382/1781, 1383/1782, 1384/1783, 1385/1784, 1386/1785, 1387/1786, 1388/1787, 1389/1788, 1390/1789, 1391/1790, 1392/1791, 1393/1792, 1394/1793, 1395/1794, 1396/1795, 1397/1796, 1398/1797, 1399/1798, 1400/1799, 1401/1800, 1402/1801, 1403/1802, 1404/1803, 1405/1804, 1406/1805, 1407/1806, 1408/1807, 1409/1808, 1410/1809, 1411/1810, 1412/1811, 1413/1812, 1414/1813, 1415/1814, 1416/1815, 1417/1816, 1418/1817, 1419/1818, 1420/1819, 1421/1820, 1422/1821, 1423/1822, 1424/1823, 1425/1824, 1426/1825, 1427/1826, 1428/1827, 1429/1828, 1430/1829, 1431/1830, 1432/1831, 1433/1832, 1434/1833, 1435/1834, 1436/1835, 1437/1836, 1438/1837, 1439/1838, 1440/1839, 1441/1840, 1442/1841, 1443/1842, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair.

In certain embodiments, an oligomeric duplex comprises a first oligomeric compound comprising a first modified oligonucleotide consisting of 19 to 30 linked nucleosides and a second oligomeric compound comprising a second modified oligonucleotide consisting of 15 to 29 linked nucleosides, wherein the nucleobase sequences of the first modified oligonucleotide and second modified oligonucleotide comprise any of the following pairs of nucleobase sequences recited in SEQ ID NOs: 329/376, 330/377, 691/722, 694/725, 696/727, 721/752, 759/905, 774/920, 787/933, 848/994, 850/996, 855/1001, 857/1003, 858/1004, 860/1006, 861/1007, 863/1009, 864/1010, 866/1012, 890/1036, 891/1037, 1045/1444, 1050/1449, 1115/1514, 1116/1515, 1142/1541, 1157/1556, 1159/1558, 1161/1560, 1166/1565, 1167/1566, 1229/1628, 1230/1629, 1343/1742, 1360/1759, 1364/1763, 1365/1764, 1402/1801, 1430/1829, 1431/1830, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second oligomeric compound is a sense compound. In certain embodiments, the second modified oligonucleotide is a sense RNAi oligonucleotide.

In certain embodiments, an oligomeric duplex comprises a first oligomeric compound comprising a first modified oligonucleotide consisting of 23 linked nucleosides and a second oligomeric compound comprising a second modified oligonucleotide consisting of 21 linked nucleosides, wherein the nucleobase sequences of the first modified oligonucleotide and second modified oligonucleotide consist of any of the following pairs of nucleobase sequences recited in SEQ ID NOs: 11/40, 12/41, 13/42, 14/43, 15/44, 16/45, 17/46, 18/47, 19/48, 20/49, 21/50, 22/51, 23/52, 24/53, 25/54, 26/55, 27/56, 28/57, 29/58, 30/59, 31/60, 32/61, 33/62, 34/63, 35/64, 36/65, 37/66, 38/67, 39/68, 69/113, 70/114, 71/115, 72/116, 73/117, 74/118, 75/119, 76/120, 77/121, 78/122, 79/123, 80/124, 81/125, 82/126, 83/127, 84/128, 85/129, 86/130, 87/131, 88/132, 89/133, 90/134, 91/135, 92/136, 93/137, 94/138, 95/139, 96/140, 97/141, 98/142, 99/143, 100/144, 101/145, 102/146, 103/147, 104/148, 105/149, 106/150, 107/151, 108/152, 109/153, 110/154, 111/155, 112/156, 157/205, 158/206, 159/207, 160/208, 161/209, 162/210, 163/211, 164/212, 165/213, 166/214, 167/215, 168/216, 169/217, 170/218, 171/219, 172/220, 173/221, 174/222, 175/223, 176/224, 177/225, 178/226, 179/227, 180/228, 181/229, 182/230, 183/231, 184/232, 185/233, 186/234, 187/235, 188/236, 189/237, 190/238, 191/239, 192/240, 193/241, 194/242, 195/243, 196/244, 197/245, 198/246, 199/247, 200/248, 201/249, 202/250, 203/251, 204/252, 253/291, 254/292, 255/293, 256/294, 257/295, 258/296, 259/297, 260/298, 261/299, 262/300, 263/301, 264/302, 265/303, 266/304, 267/305, 268/306, 269/307, 270/308, 271/309, 272/310, 273/311, 274/312, 275/313, 276/314, 277/315, 278/316, 279/317, 280/318, 281/319, 282/320, 283/321, 284/322, 285/323, 286/324, 287/325, 288/326, 289/327, 290/328, 329/376, 330/377, 331/378, 332/379, 333/380, 334/381, 335/382, 336/383, 337/384, 338/385, 339/386, 340/387, 341/388, 342/389, 343/390, 344/391, 345/392, 346/393, 347/394, 348/395, 349/396, 350/397, 351/398, 352/399, 353/400, 354/401, 355/402, 356/403, 357/404, 358/405, 359/406, 360/407, 361/408, 362/409, 363/410, 364/411, 365/412, 366/413, 367/414, 368/415, 369/416, 370/417, 371/418, 372/419, 373/420, 374/421, 375/422, 423/453, 424/454, 425/455, 426/456, 427/457, 428/458, 429/459, 430/460, 431/461, 432/462, 433/463, 434/464, 435/465, 436/466, 437/467, 438/468, 439/469, 440/470, 441/471, 442/472, 443/473, 444/474, 445/475, 446/476, 447/477, 448/478, 449/479, 450/480, 451/481, 452/482, 483/517, 484/518, 485/519, 486/520, 487/521, 488/522, 489/523, 490/524, 491/525, 492/526, 493/527, 494/528, 495/529, 496/530, 497/531, 498/532, 499/533, 500/534, 501/535, 502/536, 503/537, 504/538, 505/539, 506/540, 507/541, 508/542, 509/543, 510/544, 511/545, 512/546, 513/547, 514/548, 515/549, 516/550, 551/581, 552/582, 553/583, 554/584, 555/585, 556/586, 557/587, 558/588, 559/589, 560/590, 561/591, 562/592, 563/593, 564/594, 565/595, 566/596, 567/597, 568/598, 569/599, 570/600, 571/601, 572/602, 573/603, 574/604, 575/605, 576/606, 577/607, 578/608, 579/609, 580/610, 611/651, 612/652, 613/653, 614/654, 615/655, 616/656, 617/657, 618/658, 619/659, 620/660, 621/661, 622/662, 623/663, 624/664, 625/665, 626/666, 627/667, 628/668, 629/669, 630/670, 631/671, 632/672, 633/673, 634/674, 635/675, 636/676, 637/677, 638/678, 639/679, 640/680, 641/681, 642/682, 643/683, 644/684, 645/685, 646/686, 647/687, 648/688, 649/689, 650/690, 691/722, 692/723, 693/724, 694/725, 695/726, 696/727, 697/728, 698/729, 699/730, 700/731, 701/732, 702/733, 703/734, 704/735, 705/736, 706/737, 707/738, 708/739, 709/740, 710/741, 711/742, 712/743, 713/744, 714/745, 715/746, 716/747, 717/748, 718/749, 719/750, 720/751, 721/752, 753/899, 754/900, 755/901, 756/902, 757/903, 758/904, 759/905, 760/906, 761/907, 762/908, 763/909, 764/910, 765/911, 766/912, 767/913, 768/914, 769/915, 770/916, 771/917, 772/918, 773/919, 774/920, 775/921, 776/922, 777/923, 778/924, 779/925, 780/926, 781/927, 782/928, 783/929, 784/930, 785/931, 786/932, 787/933, 788/934, 789/935, 790/936, 791/937, 792/938, 793/939, 794/940, 795/941, 796/942, 797/943, 798/944, 799/945, 800/946, 901/947, 802/948, 803/949, 804/950, 805/951, 806/952, 807/953, 808/854, 809/955, 810/956, 811/957, 812/958, 813/959, 814/960, 815/961, 816/962, 817/963, 818/964, 819/965, 820/966, 821/967, 822/968, 823/969, 824/970, 825/971, 826/972, 827/973, 828/974, 829/975, 830/976, 831/977, 832/978, 833/979, 834/980, 835/981, 836/982, 837/983, 838/984, 839/985, 840/986, 841/987, 842/988, 843/989, 844/990, 845/991, 846/992, 847/993, 848/994, 849/995, 850/996, 851/997, 852/998, 853/999, 854/1000, 855/1001, 856/1002, 857/1003, 858/1004, 859/1005, 860/1006, 861/1007, 862/1008, 863/1009, 864/1010, 865/1011, 866/1012, 867/1013, 868/1014, 869/1015, 870/1016, 871/1017, 872/1018, 873/1019, 874/1020, 875/1021, 876/1022, 877/1023, 878/1024, 879/1025, 880/1026, 881/1027, 882/1028, 883/1029, 884/1030, 885/1031, 886/1032, 887/1033, 888/1034, 889/1035, 890/1036, 891/1037, 892/1038, 893/1039, 894/1040, 895/1041, 896/1042, 897/1043, 898/1044, 1045/1444, 1046/1445, 1047/1446, 1048/1447, 1049/1448, 1050/1449, 1051/1450, 1052/1451, 1053/1452, 1054/1453, 1055/1454, 1056/1455, 1057/1456, 1058/1457, 1059/1458, 1060/1459, 1061/1460, 1062/1461, 1063/1462, 1064/1463, 1065/1464, 1066/1465, 1067/1466, 1068/1467, 1069/1468, 1070/1469, 1071/1470, 1072/1471, 1073/1472, 1074/1473, 1075/1474, 1076/1475, 1077/1476, 1078/1477, 1079/1478, 1080/1479, 1081/1480, 1082/1481, 1083/1482, 1084/1483, 1085/1484, 1086/1485, 1087/1486, 1088/1487, 1089/1488, 1090/1489, 1091/1490, 1092/1491, 1093/1492, 1094/1493, 1095/1494, 1096/1495, 1097/1496, 1098/1497, 1099/1498, 1100/1499, 1101/1500, 1102/1501, 1103/1502, 1104/1503, 1105/1504, 1106/1505, 1107/1506, 1108/1507, 1109/1508, 1110/1509, 1111/1510, 1112/1511, 1113/1512, 1114/1513, 1115/1514, 1116/1515, 1117/1516, 1118/1517, 1119/1518, 1120/1519, 1121/1520, 1122/1521, 1123/1522, 1124/1523, 1125/1524, 1126/1525, 1127/1526, 1128/1527, 1129/1528, 1130/1529, 1131/1530, 1132/1531, 1133/1532, 1134, 1533, 1135/1534, 1136/1535, 1137/1536, 1138/1537, 1139/1538, 1140/1539, 1141/1540, 1142/1541, 1143/1542, 1144/1543, 1145/1544, 1146/1545, 1147/1546, 1148/1547, 1149/1548, 1150/1549, 1151/1550, 1152/1551, 1153/1552, 1154/1553, 1155/1554, 1156/1555, 1157/1556, 1158/1557, 1159/1558, 1160/1559, 1161/1560, 1162/1561, 1163/1562, 1164/1563, 1165/1564, 1166/1565, 1167/1566, 1168/1567, 1169/1568, 1170/1569, 1171/1570, 1172/1571, 1173/1572, 1174/1573, 1175/1574, 1176/1575, 1177/1576, 1178/1577, 1179/1578, 1180/1579, 1181/1580, 1182/1581, 1183/1582, 1184/1583, 1185/1584, 1186/1585, 1187/1586, 1188/1587, 1189/1588, 1190/1589, 1191/1590, 1192/1591, 1193/1592, 1194/1593, 1195/1594, 1196/1595, 1197/1596, 1198/1597, 1199/1598, 1200/1599, 1201/1600, 1202/1601, 1203/1602, 1204/1603, 1205/1604, 1206/1605, 1207/1608, 1208/1607, 1209/1608, 1210/1609, 1211/1610, 1212/1611, 1213/1612, 1214/1613, 1215/1614, 1216/1615, 1217/1616, 1218/1617, 1219/1618, 1220/1619, 1221/1620, 1222/1621, 1223/1622, 1224/1623, 1225/1624, 1226/1625, 1227/1626, 1228/1627, 1229/1628, 1230/1629, 1231/1630, 1232/1631, 1233/1632, 1234/1633, 1235/1634, 1236/1635, 1237/1636, 1238/1637, 1239/1638, 1240/1639, 1241/1340, 1242/1641, 1243/1642, 1244/1643, 1245/1644, 1246/1645, 1247/1646, 1248/1647, 1249/1648, 1250/1649, 1251/1650, 1252/1651, 1253/1652, 1254/1653, 1255/1654, 1256/1655, 1257/1656, 1258/1657, 1259/1658, 1260/1659, 1261/1660, 1262/1661, 1263/1662, 1264/1663, 1265/1664, 1266/1665, 1267/1666, 1268/1667, 1269/1668, 1270/1669, 1271/1670, 1272/1671, 1273/1672, 1274/1673, 1275/1674, 1276/1675, 1277/1676, 1278/1677, 1279/1678, 1280/1679, 1281/1680, 1282/1681, 1283/1682, 1284/1683, 1285/1684, 1286/1685, 1287/1686, 1288/1687, 1289/1688, 1290/1689, 1291/1690, 1292/1691, 1293/1692, 1294/1693, 1295/1694, 1296/1695, 1297/1696, 1298/1697, 1299/1698, 1300/1699, 1301/1700, 1302/1701, 1303/1702, 1304/1703, 1305/1704, 1306/1705, 1307/1706, 1308/1707, 1309/1708, 1310/1709, 1311/1710, 1312/1711, 1313/1712, 1314/1713, 1315/1714, 1316/1715, 1317/1716, 1318/1717, 1319/1718, 1320/1719, 1321/1720, 1322/1721, 1323/1722, 1324/1723, 1325/1724, 1326/1725, 1327/1726, 1328/1727, 1329/1728, 1330/1729, 1331/1730, 1332/1731, 1333/1732, 1334/1733, 1335/1734, 1336/1735, 1337/1736, 1338/1737, 1339/1738, 1340/1739, 1341/1740, 1342/1741, 1343/1742, 1344/1743, 1345/1744, 1346/1745, 1347/1746, 1348/1747, 1349/1748, 1350/1749, 1351/1750, 1352/1751, 1353/1752, 1354/1753, 1355/1754, 1356/1755, 1357/1756, 1358/1757, 1359/1758, 1360/1759, 1361/1760, 1362/1761, 1363/1762, 1364/1763, 1365/1764, 1366/1765, 1367/1766, 1368/1767, 1369/1768, 1370/1769, 1371/1770, 1372/1771, 1373/1772, 1374/1773, 1375/1774, 1376/1775, 1377/1776, 1378/1777, 1379/1778, 1380/1779, 1381/1780, 1382/1781, 1383/1782, 1384/1783, 1385/1784, 1386/1785, 1387/1786, 1388/1787, 1389/1788, 1390/1789, 1391/1790, 1392/1791, 1393/1792, 1394/1793, 1395/1794, 1396/1795, 1397/1796, 1398/1797, 1399/1798, 1400/1799, 1401/1800, 1402/1801, 1403/1802, 1404/1803, 1405/1804, 1406/1805, 1407/1806, 1408/1807, 1409/1808, 1410/1809, 1411/1810, 1412/1811, 1413/1812, 1414/1813, 1415/1814, 1416/1815, 1417/1816, 1418/1817, 1419/1818, 1420/1819, 1421/1820, 1422/1821, 1423/1822, 1424/1823, 1425/1824, 1426/1825, 1427/1826, 1428/1827, 1429/1828, 1430/1829, 1431/1830, 1432/1831, 1433/1832, 1434/1833, 1435/1834, 1436/1835, 1437/1836, 1438/1837, 1439/1838, 1440/1839, 1441/1840, 1442/1841, 1443/1842, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair.

In certain embodiments, an oligomeric duplex comprises a first oligomeric compound comprising a first modified oligonucleotide consisting of 15 to 30 linked nucleosides and a second oligomeric compound comprising a second modified oligonucleotide consisting of 15 to 29 linked nucleosides, wherein the nucleobase sequences of the first modified oligonucleotide and second modified oligonucleotide consists of any of the following pairs of nucleobase sequences recited in SEQ ID NOs: 329/376, 330/377, 691/722, 694/725, 696/727, 721/752, 759/905, 774/920, 787/933, 848/994, 850/996, 855/1001, 857/1003, 858/1004, 860/1006, 861/1007, 863/1009, 864/1010, 866/1012, 890/1036, 891/1037, 1045/1444, 1050/1449, 1115/1514, 1116/1515, 1142/1541, 1157/1556, 1159/1558, 1161/1560, 1166/1565, 1167/1566, 1229/1628, 1230/1629, 1343/1742, 1360/1759, 1364/1763, 1365/1764, 1402/1801, 1430/1829, 1431/1830, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair.

In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second oligomeric compound is a sense compound. In certain embodiments, the second modified oligonucleotide is a sense RNAi oligonucleotide.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH2- and —O—CH(CH3)-, and a non-bicyclic sugar moiety, such as a 2′-MOE sugar moiety, a 2′-F sugar moiety, a 2′-OMe sugar moiety, or a 2′-NMA sugar moiety. In certain embodiments, at least one nucleoside of the first modified oligonucleotide and/or the second modified oligonucleotide can comprise an unmodified 2′-deoxyribosyl sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe. In certain embodiments, one or more 2′-F sugar moieties have a configuration other than 2′-β-D-ribosyl. In certain embodiments, one or more 2′-F sugar moieties is in the 2′-β-D-xylosyl configuration.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, modified morpholino, hexitol nucleic acid (HNA), fluro-hexitol nucleic acid (FHNA), peptide nucleic acid (PNA), glycol nucleic acid (GNA), and unlocked nucleic acid (UNA). In certain embodiments, at least one nucleoside of the first modified oligonucleotide comprises a sugar surrogate, which can be a GNA.

In any of the oligomeric duplexes described herein, the first modified oligonucleotide has a sugar motif (from 5′ to 3′) of yfyfyfyfyfyfyfyfyfyfyyy or yfyyyfyyyyyyyfyfyyyyyyy, wherein each “y” represents a 2′-OMe sugar moiety and each “f” represents a 2′-F sugar moiety. In any of the oligomeric duplexes described herein, the second modified oligonucleotide has a sugar motif (from 5′ to 3′) of fyfyfyfyfyfyfyfyfyfyf or yyyyyyfyfffyyyyyyyyyy, wherein each “y” represents a 2′-OMe sugar moiety and each “f” represents a 2′-F sugar moiety.

In any of the oligomeric duplexes described herein, the modified oligonucleotide of the first oligomeric compound has a sugar motif (from 5′ to 3′) of: yfyfyfyfyfyfyfyfyfyfyyy and the second modified oligonucleotide has a sugar motif (from 5′ to 3′) of fyfyfyfyfyfyfyfyfyfyf, wherein each “y” represents a 2′-OMe sugar moiety and each “f” represents a 2′-F sugar moiety. In any of the oligomeric duplexes described herein, the modified oligonucleotide of the first oligomeric compound has a sugar motif (from 5′ to 3′) of yfyyyfyyyyyyyfyfyyyyyyy and the second modified oligonucleotide has a sugar motif (from 5′ to 3′) of yyyyyyfyfffyyyyyyyyyy, wherein each “y” represents a 2′-OMe sugar moiety and each “f” represents a 2′-F sugar moiety.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage. In certain embodiments, the modified internucleoside linkage is a mesyl phosphoramidate internucleoside linkage. In certain embodiments, at least one of the first or second internucleoside linkages from the 5′ end and/or the 3′ end of the first modified oligonucleotide comprises a mesyl phosphoramidate internucleoside linkage. In certain embodiments, at least one of the first or second internucleoside linkages from the 5′ end and/or the 3′ end of the second modified oligonucleotide comprises a mesyl phosphoramidate internucleoside linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester, a phosphorothioate, or a mesyl phosphoramidate internucleoside linkage.

In any of the oligomeric duplexes described herein, the internucleoside linkage motif of the first modified oligonucleotide can be ssooooooooooooooooooss, wherein each “o” represents a phosphodiester internucleoside linkage and each “s” represents a phosphorothioate internucleoside linkage. In any of the oligomeric duplexes described herein, the internucleoside linkage motif of the second modified oligonucleotide can be ssooooooooooooooooss, wherein each “o” represents a phosphodiester internucleoside linkage and each “s” represents a phosphorothioate internucleoside linkage.

In any of the oligomeric duplexes described herein, at least one nucleobase of the first modified oligonucleotide and/or the second modified oligonucleotide can be a modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In any of the oligomeric duplexes described herein, the second modified oligonucleotide can comprise a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, an antisense agent comprises an antisense compound, which comprises an oligomeric compound or an oligomeric duplex described herein. In certain embodiments, an antisense agent, which can comprise an oligomeric compound or an oligomeric duplex described herein, is an RNAi agent capable of reducing the amount of Tau nucleic acid through the activation of RISC/Ago2.

Certain embodiments provide an oligomeric agent comprising two or more oligomeric duplexes. In certain embodiments, an oligomeric agent comprises two or more of any of the oligomeric duplexes described herein. In certain embodiments, an oligomeric agent comprises two or more of the same oligomeric duplex, which can be any of the oligomeric duplexes described herein. In certain embodiments, the two or more oligomeric duplexes are linked together. In certain embodiments, the two or more oligomeric duplexes are covalently linked together. In certain embodiments, the second modified oligonucleotides of two or more oligomeric duplexes are covalently linked together. In certain embodiments, the second modified oligonucleotides of two or more oligomeric duplexes are covalently linked together at their 3′ ends. In certain embodiments, the two or more oligomeric duplexes are covalently linked together by a glycol linker, such as a tetraethylene glycol linker.

### Certain Terminal Groups

In certain embodiments, oligomeric compounds comprise a terminal group. In certain such embodiments, oligomeric compounds comprise a phosphorus-containing group at the 5′-end of the antisense oligonucleotide and/or the sense oligonucleotide. In certain embodiments, the terminal group is a phosphate stabilized phosphate group. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos) or 5′-deoxy-5′-C-malonyl. When the 5′-end phosphorus-containing group is 5′-end vinylphosphonate, the 5′VP can be either 5′-E-VP isomer (i.e., trans-vinylphosphate), 5′-Z—VP isomer (i.e., cis-vinylphosphate), or mixtures thereof. Although such phosphate group can be attached to either the antisense oligonucleotide or the sense oligonucleotide, it will typically be attached to the antisense oligonucleotide as that has been shown to improve activity of certain RNAi agents. See, e.g., Prakash et al., Nucleic Acids Res., 43(6):2993-3011, 2015; Elkayam, et al., Nucleic Acids Res., 45(6):3528-3536, 2017; Parmar, et al. ChemBioChem, 17(11)₉₈₅-989; 2016; Harastzi, et al., Nucleic Acids Res., 45(13):7581-7592, 2017. In certain embodiments, the phosphate stabilizing group is 5′-cyclopropyl phosphonate. See e.g., WO 2018/027106.

### Certain Conjugated Oligomeric Compounds

In certain embodiments, the oligomeric compounds comprise one or more conjugate groups. Conjugate groups consist of one or more conjugate moiety and a conjugate linker which links the conjugate moiety to an oligonucleotide of an oligomeric compound. Conjugate groups may be attached to either or both ends and/or at any internal position of an oligonucleotide. In certain embodiments, conjugate groups modify one or more properties of oligomeric compound, including, but not limited to, pharmacodynamics, pharmacokinetics, stability, binding, absorption, tissue distribution, cellular distribution, cellular uptake, charge and clearance.

Conjugation of one or more carbohydrate moieties to an oligomeric compound can optimize one or more properties of the oligomeric compound. In certain embodiments, the carbohydrate moiety is attached to a modified subunit of the oligomeric compound. For example, the ribose sugar of one or more ribonucleotide subunits of an oligomeric compound can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds.

### A. Certain Specific Conjugate Groups

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, i, 923-937), a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

### 2. Conjugate Linkers

Conjugate moieties are attached to an oligomeric compound through conjugate linkers. In certain embodiments, a conjugate group is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units, such as ethylene glycol, nucleosides, or amino acid units.

In certain embodiments, a conjugate linker comprises a pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include, but are not limited to, electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include, but are not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include, but are not limited to, substituted or unsubstituted C₁-C₁₀ alkyl, substituted or unsubstituted C₂-C₁₀ alkenyl, or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl.

In certain embodiments, conjugate linkers comprise 1-5 linker-nucleosides. In certain embodiments, such linker-nucleosides are modified nucleosides. In certain embodiments, such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which a oligomeric compound comprises two oligonucleotides each consisting of a specified number or range of linked nucleosides and the antisense oligonucleotide having a specified percent complementarity to a reference nucleic acid, and the oligomeric compound also comprises a conjugate group comprising a conjugate linker comprising linker-nucleosides, those linker-nucleosides are not counted toward the length of the oligonucleotides of an oligomeric compound and are not used in determining the percent complementarity of the antisense oligonucleotide with the reference nucleic acid. Unless otherwise indicated, conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligomeric compound. For example, in certain circumstances, oligomeric compounds comprising a particular conjugate moiety are better taken up by a particular cell type, but once the oligomeric compound has been taken up, it is desirable that the conjugate group be cleaved to release the unconjugated or parent oligomeric compound. Thus, certain conjugates may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among an amide, an ester, an ether, one or both esters of a phosphodiester, a phosphate ester, a carbamate, and a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Certain Cell-Targeting Conjugate Moieties

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

### Certain Motifs

Oligomeric duplexes can be described by motif or by specific features. In certain embodiments, an oligomeric duplex having a motif or specific feature described herein is an antisense agent.

In certain embodiments, the oligomeric duplexes described herein comprise:
(a) a sense oligonucleotide having:
   (i) a length of 21 nucleosides;
   (ii) a conjugate attached to the 3′-end or the 5′-end; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
and
(b) an antisense oligonucleotide having:
   (i) a length of 23 nucleosides;
   (ii) 2′-OMe modifications at positions 1, 3, 5, 9, 11 to 13, 15, 17, 19, 21, and 23, and 2′-F modifications at positions 2, 4, 6 to 8, 10, 14, 16, 18, 20, and 22 (counting from the 5′ end); and
   (iii) phosphorothioate internucleoside linkages between nucleoside positions 21 and 22, and between nucleoside positions 22 and 23 (counting from the 5′ end);
   wherein the two nucleosides at the 3′end of the antisense oligonucleotide are overhanging nucleosides, and the end of the oligomeric duplex constituting the 5′-end of the antisense oligonucleotide and the 3′-end of the sense oligonucleotide is blunt (i.e., neither oligonucleotide has overhang nucleoside at that end and instead the hybridizing region of the sense oligonucleotide includes the 3′-most nucleoside of the sense oligonucleotide and that nucleoside hybridizes with the 5′-most nucleoside of the antisense oligonucleotide).

In certain embodiments, the oligomeric duplexes described herein comprise:
(a) a sense oligonucleotide having:
   (i) a length of 21 nucleosides;
   (ii) a conjugate attached to the 3′-end or the 5′-end;
   (iii) 2′-F modifications at positions 1, 3, 5, 7, 9 to 11, 13, 17, 19, and 21, and 2′-OMe modifications at positions 2, 4, 6, 8, 12, 14, 16, 18, and 20 (counting from the 5′ end); and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 23 nucleosides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the oligomeric duplex includes a two nucleoside overhang at the 3′end of the antisense oligonucleotide, and a blunt end at the 5′-end of the antisense oligonucleotide.

In certain embodiments, the oligomeric duplexes described herein comprise:
(a) a sense oligonucleotide having:
   (i) a length of 21 nucleosides;
   (ii) a conjugate attached to the 3′-end or the 5′-end;
   (iii) 2′-OMe modifications at positions 1 to 6, 8, 10, and 12 to 21, and 2′-F modifications at positions 7 and 9, and a deoxynucleoside at position 11 (counting from the 5′ end); and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 23 nucleosides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the oligomeric duplex has a two nucleoside overhang at the 3′end of the antisense oligonucleotide, and a blunt end at the 5′-end of the antisense oligonucleotide.

In certain embodiments, the oligomeric duplexes described herein comprise:
(a) a sense oligonucleotide having:
   (i) a length of 21 nucleosides;
   (ii) a conjugate attached to the 3′-end or the 5′-end;
   (iii) 2′-F modifications at positions 1, 3, 5, 7, 9, 11, 13, 15, 17, 19, and 21, and 2′-OMe modifications at positions 2, 4, 6, 8, 10, 12, 14, 16, 18, and 20 (counting from the 5′ end); and
   (iv) phosphorothioate internucleoside linkages between nucleoside positions 1 and 2, between nucleoside positions 2 and 3, between nucleoside positions 19 and 20, and between nucleoside positions 20 and 21 (counting from the 5′ end);
and
(b) an antisense oligonucleotide having:
   (i) a length of 23 nucleosides;
   (ii) 2′-OMe modifications at positions 1, 3, 5, 7, 9, 11, 13, 15, 17, 19, and 21 to 23, and 2′-F modifications at positions 2, 4, 6, 8, 10, 12, 14, 16, 18, and 20 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the oligomeric duplex has a two nucleoside overhang at the 3′end of the antisense oligonucleotide, and a blunt end at the 5′-end of the antisense oligonucleotide.

In certain embodiments, the oligomeric duplexes described herein comprise:
(a) a sense oligonucleotide having:
   (i) a length of 21 nucleosides;
   (ii) a conjugate attached to the 3′-end or the 5′-end;
   (iii) 2′-OMe modifications at positions 1 to 6, 8, and 12 to 21, and 2′-F modifications at positions 7, and 9 to 11; and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 23 nucleosides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the oligomeric duplex has a two nucleotide overhang at the 3′end of the antisense oligonucleotide, and a blunt end at the 5′-end of the antisense oligonucleotide.

In certain embodiments, the oligomeric duplexes described herein comprise:
(a) a sense oligonucleotide having:
   (i) a length of 21 nucleosides;
   (ii) a conjugate attached to the 3′-end or the 5′-end;
   (iii) 2′-OMe modifications at positions 1 to 6, 8, and 12 to 21, and 2′-F modifications at positions 7, and 9 to 11; and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 23 nucleosides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleoside linkages between nucleoside positions 1 and 2, between nucleoside positions 2 and 3, between nucleotide positions 21 and 22, and between nucleoside positions 22 and 23 (counting from the 5′ end);
   wherein the oligomeric duplex has a two nucleoside overhang at the 3′end of the antisense oligonucleotide, and a blunt end at the 5′-end of the antisense oligonucleotide.

In certain embodiments, the oligomeric duplexes described herein comprise:
(a) a sense oligonucleotide having:
   (i) a length of 21 nucleosides;
   (ii) a conjugate attached to the 3′-end or the 5′-end;
   (iii) 2′-OMe modifications at positions 1 to 6, 8, and 12 to 21, and 2′-F modifications at positions 7, and 9 to 11 (counting from the 5′ end); and
   (iv) phosphorothioate internucleoside linkages between nucleoside positions 1 and 2, between nucleoside positions 2 and 3, between nucleoside positions 19 and 20, and between nucleoside positions 20 and 21; and
(b) an antisense oligonucleotide having:
   (i) a length of 23 nucleosides;
   (ii) 2′-OMe modifications at positions 1, 3 to 5, 7 to 13, 15, and 17 to 23, and 2′F modifications at positions 2, 6, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the oligomeric duplex includes a two nucleoside overhang at the 3′end of the antisense oligonucleotide, and a blunt end at the 5′-end of the antisense oligonucleotide. In certain embodiments, the oligomeric duplexes described herein comprise:
(a) a sense oligonucleotide having:
   (i) a length of 19 nucleosides;
   (ii) a conjugate attached to the 3′-end or the 5′-end;
   (iii) 2′-OMe modifications at positions 1 to 4, 6, and 10 to 19, and 2′-F modifications at positions 5, and 7 to 9; and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 21 nucleosides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleoside linkages between nucleoside positions 1 and 2, between nucleoside positions 2 and 3, between nucleoside positions 19 and 20, and between nucleoside positions 20 and 21 (counting from the 5′ end);
   wherein the oligomeric duplex has a two nucleoside overhang at the 3′end of the antisense oligonucleotide, and a blunt end at the 5′-end of the antisense oligonucleotide.

In certain embodiments, the antisense oligonucleotide of the oligomeric duplex has a stabilized phosphate group at the 5′ end of thereof.

In certain embodiments, the oligomeric duplex comprises a sense oligonucleotide consisting of 21 nucleosides and an antisense oligonucleotide consisting of 23 nucleosides, wherein the sense oligonucleotide contains at least one motif of three contiguous 2′-F modified nucleosides at positions 9, 10, 11 from the 5′-end; the antisense oligonucleotide contains at least one motif of three 2′-O-methyl modifications on three consecutive nucleosides at positions 11, 12, 13 from the 5′ end, wherein one end of the oligomeric duplex is blunt, while the other end comprises a 2 nucleotide overhang. In certain embodiments, the 2 nucleotide overhang is at the 3′-end of the antisense oligonucleotide.

In certain embodiments, when the 2 nucleotide overhang is at the 3′-end of the antisense oligonucleotide, there may be two phosphorothioate internucleoside linkages between the terminal three nucleotides, wherein two of the three nucleotides are the overhang nucleotides, and the third nucleotide is a paired nucleotide next to the overhang nucleotide.

In certain embodiments, the oligomeric duplex additionally has two phosphorothioate internucleoside linkages between the terminal three nucleotides at both the 5′-end of the sense oligonucleotide and at the 5′-end of the antisense oligonucleotide. In certain embodiments, every nucleoside in the sense oligonucleotide and the antisense oligonucleotide of the oligomeric duplex is a modified nucleoside. In certain embodiments, each nucleoside is independently modified with a 2′-O-methyl or 2′-fluoro, e.g. in an alternating motif. Optionally, the oligomeric duplex comprises a conjugate.

In certain embodiments, every nucleotide in the sense oligonucleotide and antisense oligonucleotide of the oligomeric duplex, including the nucleotides that are part of the motifs, may be modified. Each nucleotide may be modified with the same or different modification, which can include one or more alteration of one or both of the non-linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

In certain embodiments, each nucleoside of the sense oligonucleotide and antisense oligonucleotide is independently modified with LNA, cEt, UNA, HNA, CeNA, 2′-MOE, 2′-OMe, 2′-O-allyl, 2′-C-allyl, 2′-deoxy, 2′-hydroxyl, or 2′-fluoro. The oligomeric duplex can contain more than one modification. In one embodiment, each nucleoside of the sense oligonucleotide and antisense oligonucleotide is independently modified with 2′-O-methyl or 2′-F. In certain embodiments, the modification is a 2′-NMA modification.

The term “alternating motif” as used herein refers to a motif having one or more modifications, each modification occurring on alternating nucleosides of one oligonucleotide. The alternating nucleoside may refer to one per every other nucleoside or one per every three nucleosides, or a similar pattern. For example, if A, B and C each represent one type of modification to the nucleoside, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleoside, the alternating pattern, i.e., modifications on every other nucleoside, may be the same, but each of the sense oligonucleotide or antisense oligonucleotide can be selected from several possibilities of modifications within the alternating motif such as “ABABAB . . . ”, “ACACAC . . . ” “BDBDBD . . . ” or “CDCDCD . . . ,” etc.

In certain embodiments, the modification pattern for the alternating motif on the sense oligonucleotide relative to the modification pattern for the alternating motif on the antisense oligonucleotide is shifted. The shift may be such that the group of modified nucleotide of the sense oligonucleotide corresponds to a group of differently modified nucleotides of the antisense oligonucleotide and vice versa. For example, the sense oligonucleotide when paired with the antisense oligonucleotide in the oligomeric duplex, the alternating motif in the sense oligonucleotide may start with “ABABAB” from 5′-3′ of the oligonucleotide and the alternating motif in the antisense oligonucleotide may start with “BABABA” from 5′-3′ of the oligonucleotide within the duplex region. As another example, the alternating motif in the sense oligonucleotide may start with “AABBAABB” from 5′-3′ of the oligonucleotide and the alternating motif in the antisense oligonucleotide may start with “BBAABBAA” from 5′-3′ of the oligonucleotide within the duplex region, so that there is a complete or partial shift of the modification 10 patterns between the sense oligonucleotide and the antisense oligonucleotide.

In certain embodiments, the oligomeric duplex comprising the pattern of the alternating motif of 2′-O-methyl modification and 2′-F modification on the sense oligonucleotide initially has a shift relative to the pattern of the alternating motif of 2′-O-methyl modification and 2′-F modification on the antisense oligonucleotide initially, i.e., the 2′-O-methyl modified nucleotide on the sense oligonucleotide base pairs with a 2′-F modified nucleotides on the antisense oligonucleotide and vice versa. The 1 position of the sense oligonucleotide may start with the 2′-F modification, and the 1 position of the antisense oligonucleotide may start with a 2′-O-methyl modification.

The introduction of one or more motifs of three identical modifications on three consecutive nucleotides to the sense oligonucleotide and/or antisense oligonucleotide interrupts the initial modification pattern present in the sense oligonucleotide and/or antisense oligonucleotide. This interruption of the modification pattern of the sense and/or antisense oligonucleotide by introducing one or more motifs of three identical modifications on three consecutive nucleotides to the sense and/or antisense oligonucleotide surprisingly enhances the gene silencing activity to the target gene. In one embodiment, when the motif of three identical modifications on three consecutive 25 nucleotides is introduced to any of the oligonucleotides, the modification of the nucleotide next to the motif is a different modification than the modification of the motif. For example, the portion of the sequence containing the motif is “ . . . NaYYYNb . . . ,” where “Y” represents the modification of the motif of three identical modifications on three consecutive nucleotide, and “Na” and “Nb” represent a modification to the nucleotide next to the motif “YYY” that is different than the modification of Y, and where Na and Nb can be the same or different modifications. Alternatively, Na and/or Nb may be present or absent when there is a wing modification present.

In certain embodiments, the sense oligonucleotide may be represented by formula (I):

(I)
5′ nₚ-Nₐ-(XXX)i-N_{b}-YYY-N_{b}-(ZZZ)ᵣNₐ-n_{q} 3′

wherein:
i and j are each independently 0 or 1;
p and q are each independently 0-6;
each Nₐ independently represents 0-25 linked nucleosides comprising at least two differently modified nucleosides;
each N_{b} independently represents 0-10 linked nucleosides;
each nₚ and n_{q} independently represent an overhanging nucleoside;
wherein N_{b} and Y do not have the same modification; and
XXX, YYY and ZZZ each independently represent modified nucleosides where each X nucleoside has the same modification; each Y nucleoside has the same modification; and each Z nucleoside has the same modification. In certain embodiments, each Y comprises a 2′-F modification.

In certain embodiments, the Nₐ and N_{b} comprise modifications of alternating patterns.

In certain embodiments, the YYY motif occurs at or near the cleavage site of the target nucleic acid. For example, when the oligomeric duplex has a duplex region of 17-23 nucleotides in length, the YYY motif can occur at or near the vicinity of the cleavage site (e.g., can occur at positions 6, 7, 8; 7, 8, 9; 8, 9, 10; 9, 10, 11; 10, 11, 12; or 11, 12, 13) of the sense oligonucleotide, the count starting from the 1^{st} nucleotide from the 5′-end; or optionally, the count starting at the 1^{st} paired nucleotide within the duplex region, from the 5′-end.

In certain embodiments, the antisense oligonucleotide of the oligomeric duplex may be represented by the formula:

(II)
5′ n_{q}-Nₐ′-(Z′Z′Z′)ₖ-N_{b}′-Y′Y′Y′-N_{b}′-(X′X′X′)ₗ-N′ₐ-
nₚ 3′

wherein:
k and l are each independently 0 or 1;
p′ and q′ are each independently 0-6;
each Nₐ′ independently represents 0-25 linked nucleotides comprising at least two differently modified nucleotides;
each N_{b}′ independently represents 0-10 linked nucleotides;
each nₚ′ and n_{q}′ independently represent an overhanging nucleoside;
wherein N_{b}′ and Y′ do not have the same modification; and
X′X′X′, Y′Y′Y′ and Z′Z′Z′ each independently represent modified nucleosides where each X′ nucleoside has the same modification; each Y′ nucleoside has the same modification; and each Z′ nucleoside has the same modification. In certain embodiments, each Y′ comprises a 2′-F modification. In certain embodiments, each Y′ comprises a 2′-OMe modification.

In certain embodiments, the Nₐ′ and/or N_{b}′ comprise modifications of alternating patterns.

In certain embodiments, the Y′Y′Y′ motif occurs at or near the cleavage site of the target nucleic acid. For example, when the oligomeric duplex has a duplex region of 17-23 nucleotides in length, the Y′Y′Y′ motif can occur at positions 9, 10, 11; 10, 11, 12; 11, 12, 13; 12, 13, 14; or 13, 14, 15 of the antisense oligonucleotide, with the count starting from the 1 nucleotide from the 5′-end; or, optionally, the count starting at the 1^{st} paired nucleotide within the duplex region, from the 5′-end. Preferably, the Y′Y′Y′ motif occurs at positions 11, 12, 13.

In certain embodiments, k is 1 and l is 0, or k is 0 and l is 1, or both k and l are 1.

The antisense oligonucleotide can therefore be represented by the following formulas:

(IIb)
5′ n_{q}′-Nₐ′-Z′Z′Z′-N_{b}′-Y′Y′Y′-Nₐ′-nₚ′ 3′;
(IIc)
5′ n_{q}′-Nₐ′-Y′Y′Y′-N_{b}′-X′X′X′-nₚ′ 3′;
or
(IId)
5′ n_{q}′-Nₐ′-Z′Z′Z′-N_{b}′-Y′Y′Y′-N_{b}′-X′X′X′-Nₐ′-nₚ′ 3′.

When the antisense oligonucleotide is represented by formula IIb, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense oligonucleotide is represented by formula IIc, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense oligonucleotide is represented by formula IId, N_{b}′ represents 0-10, 0-7, 0-5, 0-4, 0-2, or 0 linked nucleosides. Each Nₐ′ independently represents 2-20, 2-15, or 2-10 linked nucleosides. Preferably, N_{b}′ is 0, 1, 2, 3, 4, 5, or 6.

In certain embodiments, k is 0 and l is 0 and the antisense oligonucleotide may be represented by the formula:

(Ia)
5′ nₚ′-Na′-Y′Y′Y′-Nₐ′-n_{q}′ 3′.

When the antisense oligonucleotide is represented by formula IIa, each Nₐ′ independently represents 2-20, 2-15, or 2-10 linked nucleosides.

Each X′, Y′, and Z′ may be the same or different from each other.

Each nucleoside of the sense oligonucleotide and antisense oligonucleotide may be independently modified with LNA, UNA, cEt, HNA, CeNA, 2′-methoxyethyl, 2′-O-methyl, 2′-O-allyl, 2′-C-allyl, 2′-hydroxyl, or 2′-fluoro. For example, each nucleoside of the sense oligonucleotide and antisense oligonucleotide is independently modified with, 2′-O-methyl or 2′-fluoro. Each X, Y, Z, X′, Y′, and Z′, in particular, may represent a 2′-O-methyl modification or 2′-fluoro modification. In certain embodiments, the modification is a 2′-NMA modification.

In certain embodiments, the sense oligonucleotide of the oligomeric duplex may contain YYY motif occurring at 9, 10, and 11 positions of the oligonucleotide when the duplex region is 21 nucleotides, the count starting from the 1^{st} nucleotide from the 5′-end, or optionally, the count starting at the 1^{st} paired nucleotide within the duplex region, from the 5′-end; and Y represents 2′-F modification. The sense oligonucleotide may additionally contain XXX motif or ZZZ motifs as wing modifications at the opposite end of the duplex region; and XXX and ZZZ each independently represents a 2′-O-methyl modification or 2′-fluoro modification.

In certain embodiments, the antisense oligonucleotide may contain Y′Y′Y′ motif occurring at positions 11, 12, 13 of the oligonucleotide, the count starting from the 1^{st} nucleotide from the 5′-end, or optionally, the count starting at the 1^{st} paired nucleotide within the duplex region, from the 5′-end; and Y′ represents 2′-O-methyl modification. The antisense oligonucleotide may additionally contain X′X′X′ motif or Z′Z′Z′ motif as wing modifications at the opposite end of the duplex region; and X′X′X′ or Z′Z′Z′ each independently represents a 2′-O-methyl modification or 2′-fluoro modification.

The sense oligonucleotide represented by any one of the above formulas Ia, Ib, Ic, and Id forms a duplex with an antisense oligonucleotide being represented by any one of the formulas IIa, IIb, IIc, and IId, respectively.

Accordingly, the oligomeric duplexes described herein may comprise a sense oligonucleotide and an antisense oligonucleotide, each oligonucleotide having 14 to 30 nucleotides, the oligomeric duplex represented by formula (ITT):

Sense:
5′ nₚ-Nₐ-(XXX)ᵢ-N_{b}-YYY-N_{b}-(ZZZ)ⱼ-Nₐ-n_{q} 3′
Antisense:
3′ nₚ′-Nₐ′-(X′X′X′)ₖ-N_{b}′-Y′Y′Y′-N_{b}′-(Z′Z′Z′)ₗ-Nₐ′-
n_{q}′ 5′

wherein:
i, j, k, and l are each independently 0 or 1;
p, p′, q, and q′ are each independently 0-6;
each Nₐ and Nₐ′ independently represents 0-25 linked nucleosides, each sequence comprising at least two differently modified nucleotides;
each N_{b} and N_{b}′ independently represents 0-10 linked nucleosides;
wherein each nₚ′, nₚ, n_{q}′ and n_{q}, each of which may or may not be present, independently represents an overhang nucleotide; and
XXX, YYY, X′X′X′, Y′Y′Y′, and Z′Z′Z′ each independently represent one motif of three identical modifications on three consecutive nucleotides.

In certain embodiments, i is 0 and j is 0; or i is 1 and j is 0; or i is 0 and j is 1; or both i and j are 0; or both i and j are 1. In another embodiment, k is 0 and l is 0; or k is 1 and l is 0, or k is 0 and l is 1; or both k and l are 0; or both k and 1 are 1.

Exemplary combinations of the sense oligonucleotide and antisense oligonucleotide forming a oligomeric duplex include the formulas below:

(IIIa)
5′ nₚ-Nₐ-YYY-Nₐ-n_{q} 3′
3′ nₚ′-Nₐ′-Y′Y′Y′-Nₐ′n_{q}′ 5′
(IIIb)
5′ nₚ-Nₐ-YYY-N_{b}-ZZZ-Nₐ-n_{q} 3′
3′ nₚ′-Nₐ′-Y′Y′Y′-N_{b}′-Z′Z′Z′-Nₐ′n_{q}′ 5′
(IIIc)
5′ nₚ-Nₐ-XXX-N_{b}-YYY-Nₐ-n_{q} 3′
3′ nₚ′-Nₐ′-X′X′X′-N_{b}′-Y′Y′Y′-Nₐ′-n_{q}′ 5′
(IIId)
5′ nₚ-Nₐ-XXX-N_{b}-YYY-N_{b}-ZZZ-Nₐ-n_{q} 3′
3′ nₚ′-Nₐ′-X′X′X′-N_{b}′-Y′Y′Y′-N_{b}′-Z′Z′Z′-Nₐ-n_{q}′ 5′

When the oligomeric duplex is represented with formula IIIa, each Nₐ independently represents 2-20, 2-15, or 2-10 linked nucleosides.

When the oligomeric duplex is represented with formula IIIb, each N_{b} independently represents 1-10, 1-7, 1-5, or 1-4 linked nucleosides. Each Nₐ independently represents 2-20, 2-15, or 2-10 linked nucleosides.

When the oligomeric duplex is represented with formula IIIc, each N_{b}, N_{b}′ independently represents 0-10, 0-7, 0-10, 0-7, 0-5, 0-4, 0-2, or 0 linked nucleosides. Each Nₐ independently represents 2-20, 2-15, or 2-10 linked nucleosides.

When the oligomeric duplex is represented with formula IIId, each N_{b}, N_{b}′ independently represents 0-10, 0-7, 0-10, 0-7, 0-5, 0-4, 0-2, or 0 linked nucleosides. Each Nₐ, Nₐ′ independently 2-20, 2-15, or 2-10 linked nucleosides. Each Nₐ, Nₐ′, N_{b}, N_{b}′ independently comprises modifications of alternating pattern.

Each of X, Y, and Z in formulas III, IIIa, IIIb, IIIc, and IIId may be the same or different from each other.

When the oligomeric duplex is represented by formula III, IIIa, IIIb, IIIc, and/or IIId, at least one of the Y nucleotides may form a base pair with one of the Y′ nucleotides. Alternatively, at least two of the Y nucleotides may form base pairs with the corresponding Y′ nucleotides; or all three of the Y nucleotides may form base pairs with the corresponding Y′ nucleotides.

When the oligomeric duplex is represented by formula IIIb or IIId, at least one of the Z nucleotides may form a base pair with one of the Z′ nucleotides. Alternatively, at least two of the Z nucleotides may form base pairs with the corresponding Z′ nucleotides; or all three of the Z nucleotides may form base pairs with the corresponding Z′ nucleotides.

When the oligomeric duplex is represented by formula IIIc or IIId, at least one of the X nucleotides may form a base pair with one of the X′ nucleotides. Alternatively, at least two of the X nucleotides may form base pairs with the corresponding X′ nucleotides; or all three of the X nucleotides may form base pairs with the corresponding X′ nucleotides.

In certain embodiments, the modification of the Y nucleotide is different than the modification on the Y′ nucleotide, the modification on the Z nucleotide is different than the modification on the Z′ nucleotide, and/or the modification on the X nucleotide is different than the modification on the X′ nucleotide.

In certain embodiments, when the oligomeric duplex is represented by the formula IIId, the Nₐ modifications are 2′-O-methyl or 2′-fluoro modifications. In another embodiment, when the oligomeric duplex is represented by formula IIId, the Nₐ modifications are 2′-O-methyl or 2′-fluoro modifications and nₚ′>0 and at least one nₚ′ is linked to a neighboring nucleotide via phosphorothioate linkage. In other embodiments, when the oligomeric duplex is represented by formula IIId, the Nₐ modifications are 2′-O-methyl or 2′-fluoro modifications, nₚ′>0 and at least one nₚ′ is linked to a neighboring nucleotide via phosphorothioate linkage, and the sense oligonucleotide is conjugated to one or more cell targeting group attached through a bivalent or trivalent branched linker. In certain embodiments, when the oligomeric duplex is represented by formula IIId, the Nₐ modifications are 2′-O-methyl or 2′-fluoro modifications, nₚ′>0 and at least one nₚ′ is linked to a neighboring nucleotide via phosphorothioate linkage, the sense oligonucleotide comprises at least one phosphorothioate linkage and the sense oligonucleotide is conjugated to one or more cell targeting group attached through a bivalent or trivalent branched linker.

In certain embodiments, when the oligomeric duplex is represented by the formula IIIa, the Nₐ modifications are 2′-O-methyl or 2′-fluoro modifications and nₚ′>0 and at least one nₚ′ is linked to a neighboring nucleotide via phosphorothioate linkage, the sense oligonucleotide comprises at least one phosphorothioate linkage and the sense oligonucleotide is conjugated to one or more cell targeting group attached through a bivalent or trivalent branched linker.

In certain embodiments, the modification is a 2′-NMA modification.

### Antisense Activity

In certain embodiments, oligomeric compounds and oligomeric duplexes are capable of hybridizing to a target nucleic acid, resulting in at least one antisense activity; such oligomeric compounds and oligomeric duplexes are antisense agents. In certain antisense activities, an antisense agent or a portion of an antisense agent is loaded into an RNA-induced silencing complex (RISC), ultimately resulting in cleavage of the target nucleic acid. For example, certain antisense compounds result in cleavage of the target nucleic acid by Argonaute. Antisense agents having antisense oligonucleotides that are loaded into RISC are RNAi agents. RNAi agents may be double-stranded (siRNA or dsRNAi) or single-stranded (ssRNA).

In certain embodiments, RNAi agents are capable of RISC-mediated modulation of a target nucleic acid in a cell. In certain embodiments, such compounds reduce or inhibit the amount or activity of a target nucleic acid by 25% or more in the standard in vitro assay described in Example 2. In certain embodiments, RNAi agents selectively affect more than one target nucleic acid. Such RNAi agents comprise a nucleobase sequence that hybridizes to more than one target nucleic acid, resulting in more than one desired antisense activity. In certain embodiments, an RNAi agent does not hybridize to one or more non-target nucleic acid or does not hybridize to one or more non-target nucleic acid in such a way that results in significant undesired antisense activity.

Antisense activities may be observed directly or indirectly. In certain embodiments, observation or detection of an RNAi activity involves observation or detection of a change in an amount of a target nucleic acid or protein encoded by such target nucleic acid, a change in the ratio of splice variants of a nucleic acid or protein and/or a phenotypic change in a cell or animal.

### Certain Target Nucleic Acids

RNAi agents comprise or consist of an antisense oligonucleotide comprising a region that is complementary to a target nucleic acid. In certain embodiments, the target nucleic acid is an endogenous RNA molecule. In certain embodiments, the target nucleic acid encodes a protein.

### A. Target/Duplex Complementarity

In certain embodiments, oligomeric agents or oligomeric compounds comprise or consist of an antisense oligonucleotide comprising a region that is complementary to a target nucleic acid. In certain embodiments, antisense oligonucleotides are 99%, 95%, 90%, 85%, or 80% complementary to the target nucleic acid. In certain embodiments, antisense oligonucleotides are at least 80% complementary to the target nucleic acid over the entire length of the antisense oligonucleotides and comprise a region that is 100% or fully complementary to a target nucleic acid. In certain embodiments, the region of full complementarity is from 6 to 20, 10 to 18, or 18 to 20 nucleobases in length.

In certain embodiments, antisense oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the antisense oligonucleotides is improved.

In certain embodiments, antisense oligonucleotides comprise a region complementary to the target nucleic acid. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 contiguous nucleosides. In certain embodiments, the complementary region constitutes 70%, 80%, 85%, 90%, 95% of the nucleosides of the antisense oligonucleotide. In certain embodiments, the complementary region constitutes all of the nucleosides of the antisense oligonucleotide. In certain embodiments, the complementary region of the antisense oligonucleotide is at least 99%, 95%, 90%, 85%, or 80% complementary to the target nucleic acid. In certain embodiments, the complementary region of the antisense oligonucleotide is 100% complementary to the target nucleic acid.

In certain embodiments, oligomeric duplexes comprise a sense oligonucleotide. In such embodiments, sense oligonucleotides comprise a region complementary to the antisense oligonucleotide. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 contiguous nucleotides. In certain embodiments, the complementary region constitutes 70%, 80%, 85%, 90%, 95% of the nucleosides of the sense oligonucleotide. In certain embodiments, the complementary region constitutes all of the nucleosides of the sense oligonucleotide. In certain embodiments, the complementary region of the sense oligonucleotide is at least 99%, 95%, 90%, 85%, or 80% complementary to the antisense oligonucleotide. In certain embodiments, the complementary region of the sense oligonucleotide is 100% complementary to the antisense oligonucleotide.

The complementary region of a sense oligonucleotide hybridizes with the antisense oligonucleotide to form a duplex region. In certain embodiments, such duplex region consists of 7 hybridized pairs of nucleosides (one of each pair being on the antisense oligonucleotide and the other of each pair being on the sense oligonucleotide). In certain embodiments, a duplex region comprises least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 hybridized pairs. In certain embodiments, each nucleoside of antisense oligonucleotide is paired in the duplex region (i.e., the antisense oligonucleotide has no overhanging nucleosides). In certain embodiments, the antisense oligonucleotide includes unpaired nucleosides at the 3′-end and/or the 5′end (overhanging nucleosides). In certain embodiments, each nucleoside of sense oligonucleotide is paired in the duplex region (i.e., the sense oligonucleotide has no overhanging nucleosides). In certain embodiments, the sense oligonucleotide includes unpaired nucleosides at the 3′-end and/or the 5′end (overhanging nucleosides). In certain embodiments, duplexes formed by the antisense oligonucleotide and the sense oligonucleotide do not include any overhangs at one or both ends. Such ends without overhangs are referred to as blunt. In certain embodiments wherein the antisense oligonucleotide has overhanging nucleosides, one or more of those overhanging nucleosides are complementary to the target nucleic acid. In certain embodiments wherein the antisense oligonucleotide has overhanging nucleosides, one or more of those overhanging nucleosides are not complementary to the target nucleic acid.

### B. Tau

In certain embodiments, RNAi agents comprise or consist of an antisense oligonucleotide comprising a region that is complementary to a target nucleic acid, wherein the target nucleic acid is tau RNA. In each of the embodiments described above, the RNAi agent may target tau RNA. In certain embodiments, the RNAi agent is an oligomeric duplex.

In certain embodiments, the tau nucleic acid has the sequence set forth in SEQ ID NO: 1 (GENBANK Accession No. NM_001377265.1) or SEQ ID NO:2 (GENBANK Accession No. NT_010783.14 truncated from nucleotides 2624000 to 2761000). In certain embodiments, contacting a cell with an oligomeric duplex comprising an oligomeric compound complementary to SEQ ID NO: 1 reduces the amount of tau RNA, and in certain embodiments reduces the amount of tau protein. In certain embodiments, contacting a cell with an oligomeric duplex comprising an oligomeric compound complementary to SEQ ID NO: 1 results in reduced aggregation of tau protein.

In certain embodiments, contacting a cell in an animal with an RNAi agent comprising an oligonucleotide complementary to SEQ ID NO: 1 ameliorates one or more symptoms of a neurodegenerative disease. In certain embodiments, the symptom is loss of memory, loss of motor function, or increase in the number and/or volume of neurofibrillary inclusions. In certain embodiments, contacting a cell in an animal with RNAi agent comprising an oligonucleotide complementary to SEQ ID NO: 1 results in maintaining or improving memory, maintaining or improving motor function, and/or maintenance or reduction in the number and/or volume of neurofibrillary inclusions.

In certain embodiments, the RNAi agent consists of an antisense oligonucleotide. In certain embodiments, the RNAi agent comprises a conjugate group. In certain embodiments, the RNAi agent is an oligomeric duplex comprising an antisense RNA oligomeric compound and a sense RNAi oligomeric compound. In certain embodiments, the oligomeric duplex comprises more than one conjugate group.

### C. Certain Target Nucleic Acids in Certain Tissues

In certain embodiments, oligomeric compounds comprise or consist of an antisense oligonucleotide comprising a region that is complementary to a target nucleic acid, wherein the target nucleic acid is expressed in a pharmacologically relevant tissue. In certain embodiments, oligomeric duplexes comprise an antisense oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system (CNS). Such tissues include the cortex, spinal cord, and the hippocampus.

### D. Certain Methods and Uses

Certain embodiments provided herein relate to methods of inhibiting tau RNA expression or activity, which can be useful for treating or ameliorating a tau-associated disease. In certain embodiments, the tau-associated disease is a tauopathy, Alzheimer's disease, fronto-temporal dementia (FTD), FTDP-17, progressive supranuclear palsy (PSP), chronic traumatic encephalopathy (CTE), corticobasal ganglionic degeneration (CBD), epilepsy, or Dravet's Syndrome.

In certain embodiments, a method comprises administering to a subject an oligomeric compound, or an oligomeric duplex, any of which having a nucleobase sequence complementary to tau RNA. In certain embodiments, the subject has or is at risk for developing a tau-associated disease. In certain embodiments, the subject has or is at risk for developing a tauopathy, Alzheimer's disease, fronto-temporal dementia (FTD), FTDP-17, progressive supranuclear palsy (PSP), chronic traumatic encephalopathy (CTE), corticobasal ganglionic degeneration (CBD), epilepsy, or Dravet's Syndrome. In certain embodiments, the oligomeric compound or oligomeric duplex is an antisense agent. In certain embodiments, the subject has or is at risk for developing Alzheimer's disease.

In certain embodiments, a method of treating a tau-associated disease comprises administering to a subject an oligomeric compound, an oligomeric duplex, or an antisense agent, any of which having a nucleobase sequence complementary to tau RNA. In certain embodiments, the subject has or is at risk for developing a tauopathy, Alzheimer's disease, fronto-temporal dementia (FTD), FTDP-17, progressive supranuclear palsy (PSP), chronic traumatic encephalopathy (CTE), corticobasal ganglionic degeneration (CBD), epilepsy, or Dravet's Syndrome. In certain embodiments, the oligomeric compound or oligomeric duplex is an antisense agent. In certain embodiments, the subject has or is at risk for developing Alzheimer's disease. In certain embodiments, the oligomeric compound or oligomeric duplex is an antisense agent. In certain embodiments, the at least one symptom or hallmark is loss of memory, loss of motor function, and increase in the number and/or volume of neurofibrillary inclusions. In certain embodiments, administration of the oligomeric compound, the oligomeric duplex, or the antisense agent to the subject reduces or delays the onset or progression of loss of memory, loss of motor function, and increase in the number and/or volume of neurofibrillary inclusions.

In certain embodiments, a method of reducing expression of tau in a cell comprises contacting the cell with an oligomeric compound, an oligomeric duplex, or an antisense agent, any of which having a nucleobase sequence complementary to tau RNA. In certain embodiments, the cell is central nervous system cell. In certain embodiments, the cell is a human cell.

Certain embodiments are drawn to an oligomeric compound, an oligomeric duplex, or an antisense agent, any of which having a nucleobase sequence complementary to tau RNA, for use in treating a tau-associated disease associated or for use in the manufacture of a medicament for treating a tau-associated disease. In certain embodiments, the tau-associated disease is a tauopathy, Alzheimer's disease, fronto-temporal dementia (FTD), FTDP-17, progressive supranuclear palsy (PSP), chronic traumatic encephalopathy (CTE), corticobasal ganglionic degeneration (CBD), epilepsy, or Dravet's Syndrome. In certain embodiments, the tau-associated disease is Alzheimer's Disease.

In any of the methods or uses described herein, the oligomeric compound, the oligomeric duplex, or the antisense agent can be any described herein.

### E. Certain Pharmaceutical Compositions

The oligomeric compounds, oligomeric duplexes, or antisense agents described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered. In certain embodiments, the oligomeric compound or oligomeric duplex is an RNAi agent.

Certain embodiments provide pharmaceutical compositions comprising one or more oligomeric compounds, oligomeric duplexes, or antisense agents, or a salt thereof. In certain such embodiments, the pharmaceutical composition comprises a suitable pharmaceutically acceptable diluent or carrier. In certain embodiments, a pharmaceutical composition comprises a sterile saline solution and one or more oligomeric compounds, oligomeric duplexes, or antisense agents. In certain embodiments, such pharmaceutical composition consists of a sterile saline solution and one or more oligomeric compounds, oligomeric duplexes, or antisense agents. In certain embodiments, the sterile saline is pharmaceutical grade saline. In certain embodiments, a pharmaceutical composition comprises one or more oligomeric compounds, oligomeric duplexes, or antisense agents and sterile water. In certain embodiments, a pharmaceutical composition consists of one oligomeric compound, oligomeric duplex, antisense agent and sterile water. In certain embodiments, the sterile water is pharmaceutical grade water. In certain embodiments, a pharmaceutical composition comprises one or more oligomeric compounds, oligomeric duplexes, or antisense agents and phosphate-buffered saline (PBS). In certain embodiments, a pharmaceutical composition consists of one or more oligomeric compounds, oligomeric duplexes, or antisense agents and sterile PBS. In certain embodiments, the sterile PBS is pharmaceutical grade PBS. In certain embodiments, such pharmaceutical composition consists of cerebrospinal fluid (CSF) and one or more oligomeric compounds, oligomeric duplexes, or antisense agents. In certain embodiments, the oligomeric duplexes or antisense agents comprises a sense oligonucleotide and an antisense oligonucleotide. Compositions and methods for the formulation of pharmaceutical compositions are dependent upon a number of criteria, including, but not limited to, route of administration, extent of disease, or dose to be administered.

In certain embodiments, the CSF is artificial CSF (aCSF). In certain embodiments, a pharmaceutical composition consists of one or more oligomeric compounds, oligomeric duplexes, or antisense agents and artificial cerebrospinal fluid. In certain embodiments, a pharmaceutical composition consists essentially of one or more oligomeric compounds, oligomeric duplexes, or antisense agents and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compounds, oligomeric duplexes, or antisense agents, and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds, oligomeric duplexes, or antisense agents may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising one or more oligomeric compounds, oligomeric duplexes, or antisense agents provided herein encompass any pharmaceutically acceptable salts, esters, or salts of such esters, which, upon administration to an animal, including a human, is capable of providing (directly or indirectly) the biologically active metabolite or residue thereof. Accordingly, for example, the disclosure is also drawn to pharmaceutically acceptable salts of compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts.

A prodrug can include the incorporation of additional nucleosides at one or both ends of oligomeric compound, oligomeric duplex, or antisense agent, which are cleaved by endogenous nucleases within the body, to form the active compound.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound, an oligomeric duplex, or an antisense agent provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

In certain embodiments, oligomeric compounds, oligomeric duplexes, or antisense agents are in aqueous solution with sodium. In certain embodiments, oligomeric compounds, oligomeric duplexes, or antisense agents are in aqueous solution with potassium. In certain embodiments, oligomeric compounds, oligomeric duplexes, or antisense agents are in PBS. In certain embodiments, oligomeric compounds, oligomeric duplexes, or antisense agents are in water. In certain embodiments, oligomeric compounds, oligomeric duplexes, or antisense agents are in aCSF. In certain such embodiments, the pH of the solution is adjusted with NaOH and/or HCl to achieve a desired pH.

### Certain Hotspot Regions

### 1. Nucleobases 1754-1783 of SEQ ID NO: 1

In certain embodiments, nucleobases 1754-1783 of SEQ ID NO: 1 comprise a hotspot region. In certain embodiments, oligomeric duplexes comprise antisense oligonucleotides complementary to a portion of nucleobases 1754-1783 of SEQ ID NO: 1. The antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length.

The nucleobase sequences of SEQ ID NOs: 1115, 1116, 850, and 721 are complementary to a portion of nucleobases 1754-1783 of SEQ ID NO: 1. The nucleobase sequences of Compound Nos: 1702622, 1702625, 1703582, and 1613217 are complementary to a portion of nucleobases 1754-1783 of SEQ ID NO: 1.

In certain embodiments, oligomeric duplexes comprising antisense oligonucleotides complementary to a portion of nucleobases 1754-1783 of SEQ ID NO: 1 achieve at least 43% reduction of MAPT RNA in a standard in vitro assay. In certain embodiments, oligomeric duplexes comprising antisense oligonucleotides complementary to a portion of nucleobases 1754-1783 of SEQ ID NO: 1 achieve an average of 69% reduction of MAPT RNA in a standard in vitro assay.

In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety. In certain embodiments, the antisense oligonucleotide has a sugar motif of 5′-yfyyyfyyyyyyyfyfyyyyyyy-3′, wherein each “y” represents a 2′-OMe sugar moiety, and each “f” represents a 2′-F sugar moiety.

### 2. Nucleobases 2332-2362 of SEQ ID NO: 1

In certain embodiments, nucleobases 2332-2362 of SEQ ID NO: 1 comprise a hotspot region. In certain embodiments, oligomeric duplexes comprise antisense oligonucleotides complementary to a portion of nucleobases 2332-2362 of SEQ ID NO: 1. The antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length.

The nucleobase sequences of SEQ ID NOs: 855, 1142, and 1360 are complementary to a portion of nucleobases 2332-2362 of SEQ ID NO: 1. The nucleobase sequences of Compound Nos: 1703618, 1702718, and 1703621 are complementary to a portion of nucleobases 2332-2362 of SEQ ID NO: 1.

In certain embodiments, oligomeric duplexes comprising antisense oligonucleotides complementary to a portion of nucleobases 2332-2362 of SEQ ID NO: 1 achieve at least 47% reduction of MAPT RNA in a standard in vitro assay. In certain embodiments, oligomeric duplexes comprising antisense oligonucleotides complementary to a portion of nucleobases 2332-2362 of SEQ ID NO: 1 achieve an average of 64% reduction of MAPT RNA in a standard in vitro assay.

In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyyyfyyyyyyyfyfyyyyyyy-3′, wherein each “y” represents a 2′-OMe sugar moiety, and each “f” represents a 2′-F sugar moiety.

### 3. Nucleobases 110-142 of SEQ ID NO: 1

In certain embodiments, nucleobases 110-142 of SEQ ID NO: 1 comprise a hotspot region. In certain embodiments, oligomeric duplexes comprise antisense oligonucleotides complementary to a portion of nucleobases 110-142 of SEQ ID NO: 1. The antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length.

The nucleobase sequences of SEQ ID NOs: 1360, 329, and 1045 are complementary to a portion of nucleobases 110-142 of SEQ ID NO: 1. The nucleobase sequences of Compound Nos: 1612977, 1702343, and 1703531 are complementary to a portion of nucleobases 110-142 of SEQ ID NO: 1.

In certain embodiments, oligomeric duplexes comprising antisense oligonucleotides complementary to a portion of nucleobases 110-142 of SEQ ID NO: 1 achieve at least 48% reduction of MAPT RNA in a standard in vitro assay. In certain embodiments, oligomeric duplexes comprising antisense oligonucleotides complementary to a portion of nucleobases 110-142 of SEQ ID NO: 1 achieve an average of 63% reduction of MAPT RNA in a standard in vitro assay.

In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety. In certain embodiments, the antisense oligonucleotide has a sugar motif of 5′-yfyyyfyyyyyyyfyfyyyyyyy-3′, wherein each “y” represents a 2′-OMe sugar moiety, and each “f” represents a 2′-F sugar moiety.

### 4. Nucleobases 6523-6552 of SEQ ID NO: 1

In certain embodiments, nucleobases 6523-6552 of SEQ ID NO: 1 comprise a hotspot region. In certain embodiments, oligomeric duplexes comprise antisense oligonucleotides complementary to a portion of nucleobases 6523-6552 of SEQ ID NO: 1. The antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length.

The nucleobase sequences of SEQ ID NOs: 890, 1330, and 1431 are complementary to a portion of nucleobases 6523-6552 of SEQ ID NO: 1. The nucleobase sequences of Compound Nos: 1703939, 1703471, and 1703942 are complementary to a portion of nucleobases 6523-6552 of SEQ ID NO: 1.

In certain embodiments, oligomeric duplexes comprising antisense oligonucleotides complementary to a portion of nucleobases 6523-6552 of SEQ ID NO: 1 achieve at least 44% reduction of MAPT RNA in a standard in vitro assay. In certain embodiments, oligomeric duplexes comprising antisense oligonucleotides complementary to a portion of nucleobases 6523-6552 of SEQ ID NO: 1 achieve an average of 60% reduction of MAPT RNA in a standard in vitro assay.

In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyyyfyyyyyyyfyfyyyyyyy-3′, wherein each “y” represents a 2′-OMe sugar moiety, and each “f” represents a 2′-F sugar moiety.

### NONLIMITING DISCLOSURE AND INCORPORATION BY REFERENCE

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, unless otherwise stated, including, but not limited to such nucleic acids having modified nucleobases. By way of further example and without limitation, an oligomeric compound having the nucleobase sequence “ATCGATCG” encompasses any compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “AT^{m}CGAUCG,” wherein ^{m}C indicates a cytosine base comprising a methyl group at the 5-position. Finally, for clarity, unless otherwise indicated, the phrase “nucleobase sequence of SEQ ID NO: X”, refers only to the sequence of nucleobases in that SEQ ID NO: X, independent of any sugar or internucleoside linkage modifications also described in such SEQ ID NO.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### Examples

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Design of RNAi Compounds with Antisense RNAi Oligonucleotides Complementary to a Human MAPT Nucleic Acid

RNAi compounds comprising antisense RNAi oligonucleotides complementary to a human MAPT nucleic acid and sense RNAi oligonucleotides complementary to the antisense RNAi oligonucleotides were designed as follows.

The RNAi compounds in the tables below consist of an antisense RNAi oligonucleotide and a sense RNAi oligonucleotide. In each case, the antisense RNAi oligonucleotide is 23 nucleosides in length; has a sugar motif (from 5′ to 3′) of: yfyfyfyfyfyfyfyfyfyfyyy, wherein each “y” represents a 2′-O-methylribosyl sugar, and each “f” represents a 2′-fluororibosyl sugar; and has an internucleoside linkage motif (from 5′ to 3′) of: ssooooooooooooooooooss, wherein each “o” represents a phosphodiester internucleoside linkage, and each “s” represents a phosphorothioate internucleoside linkage. The sense RNAi oligonucleotide in each case is 21 nucleosides in length; has a sugar motif (from 5′ to 3′) of: fyfyfyfyfyfyfyfyfyfyf, wherein each “y” represents a 2′-O-methylribosyl sugar, and each “f” represents a 2′-fluororibosyl sugar; and has an internucleoside linkage motif (from 5′ to 3′) of: ssooooooooooooooooss, wherein each “o” represents a phosphodiester internucleoside linkage, and each “s” represents a phosphorothioate internucleoside linkage. Each antisense RNAi oligonucleotide is complementary to the target nucleic acid (MAPT), and each sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

“Start site” indicates the 5′-most nucleoside to which the antisense RNAi oligonucleotide is complementary target nucleic acid sequence. “Stop site” indicates the 3′-most nucleoside to which the antisense RNAi oligonucleotide is complementary in the target nucleic acid sequence. Each antisense RNAi oligonucleotide listed in the tables below is 100% complementary to SEQ ID NO: 1 (GENBANK Accession No. NM_001377265.1).

TABLE 1
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1613942 | 1613940 | UAAAGUGAGUCA | 11 | 5929 | 5951 | 1613941 | UCAAGCUGCUG | 40 |
| | | GCAGCUUGAAG | | | | | ACUCACUUUA | |
| 1613945 | 1613943 | AAAUGGAACUAU | 12 | 5944 | 5966 | 1613944 | ACUUUAUCAAU | 41 |
| | | UGAUAAAGUGA | | | | | AGUUCCAUUU | |
| 1613951 | 1613949 | AGGAUACAGUCU | 13 | 5974 | 5996 | 1613950 | UCAGUGGUGAG | 42 |
| | | CACCACUGAAG | | | | | ACUGUAUCCU | |
| 1613963 | 1613961 | UCUUACACAUUC | 14 | 6025 | 6047 | 1613962 | AGGGGGGAGGA | 43 |
| | | CUCCCCCCUCC | | | | | AUGUGUAAGA | |
| 1613966 | 1613964 | UGCCCAUGUUAA | 15 | 6040 | 6062 | 1613965 | GUAAGAUAGUU | 44 |
| | | CUAUCUUACAC | | | | | AACAUGGGCA | |
| 1613975 | 1613973 | AAGGGUUACGAG | 16 | 6085 | 6107 | 1613974 | UUAAACUGCCU | 45 |
| | | GCAGUUUAAGU | | | | | CGUAACCCUU | |
| 1613987 | 1613985 | AAACCCCAAGGG | 17 | 6151 | 6173 | 1613986 | AGUUAGAGGCC | 46 |
| | | CCUCUAACUCC | | | | | CUUGGGGUUU | |
| 1613996 | 1613994 | UGAACUAGCCAG | 18 | 6194 | 6216 | 1613995 | CCCAGGCAGCUG | 47 |
| | | CUGCCUGGGAA | | | | | GCUAGUUCA | |
| 1613999 | 1613997 | UGGCUGGGGAGG | 19 | 6209 | 6231 | 1613998 | AGUUCAUUCCC | 48 |
| | | GAAUGAACUAG | | | | | UCCCCAGCCA | |
| 1614005 | 1614003 | UAUUCCUACGCC | 20 | 6227 | 6249 | 1614004 | CCAGGUGCAGG | 49 |
| | | UGCACCUGGCU | | | | | CGUAGGAAUA | |
| 1614020 | 1614018 | UAGGGAGGCAUG | 21 | 6295 | 6317 | 1614019 | GCCCACAAUCAU | 50 |
| | | AUUGUGGGCUU | | | | | GCCUCCCUA | |
| 1614023 | 1614021 | AGGAUGCCAAGG | 22 | 6310 | 6332 | 1614022 | UCCCUAAGACCU | 51 |
| | | UCUUAGGGAGG | | | | | UGGCAUCCU | |
| 1614026 | 1614024 | AACGGCUUAGAG | 23 | 6325 | 6347 | 1614025 | CAUCCUUCCCUC | 52 |
| | | GGAAGGAUGCC | | | | | UAAGCCGUU | |
| 1614032 | 1614030 | AGUGUGAGAGGU | 24 | 6351 | 6373 | 1614031 | CUCUGUGCCACC | 53 |
| | | GGCACAGAGGU | | | | | UCUCACACU | |
| 1614035 | 1614033 | UGUGUGUCUGGA | 25 | 6366 | 6388 | 1614034 | CACACUGGCUCC | 54 |
| | | GCCAGUGUGAG | | | | | AGACACACA | |
| 1614038 | 1614036 | UCCAAAAGCACA | 26 | 6381 | 6403 | 1614037 | CACACAGCCUGU | 55 |
| | | GGCUGUGUGUC | | | | | GCUUUUGGA | |
| 1614077 | 1614065 | UACUUGGAGAAC | 27 | 6426 | 6448 | 1614066 | CUCAUCUUUGU | 56 |
| | | AAAGAUGAGGA | | | | | UCUCCAAGUA | |
| 1614131 | 1614121 | AUGGGACACGCA | 28 | 6475 | 6497 | 1614122 | GUGAUCACCUG | 57 |
| | | GGUGAUCACCU | | | | | CGUGUCCCAU | |
| 1614140 | 1614138 | AAGCUGAAGAGA | 29 | 6520 | 6542 | 1614139 | CUUCUGAUUUC | 58 |
| | | AAUCAGAAGUU | | | | | UCUUCAGCUU | |
| 1614149 | 1614147 | UUAGGAGGUGAG | 30 | 6565 | 6587 | 1614148 | GCCUAGAGCCUC | 59 |
| | | GCUCUAGGCCA | | | | | ACCUCCUAA | |
| 1614152 | 1614150 | AUGGGGCUAAGU | 31 | 6580 | 6602 | 1614151 | UCCUAAUAGAC | 60 |
| | | CUAUUAGGAGG | | | | | UUAGCCCCAU | |
| 1614158 | 1614156 | AGAAAUAGUCCU | 32 | 6610 | 6632 | 1614157 | AUGUUGAGCAG | 61 |
| | | GCUCAACAUGG | | | | | GACUAUUUCU | |
| 1614164 | 1614162 | ACCGAAGAAAUC | 33 | 6640 | 6662 | 1614163 | AAGUCCCAUGA | 62 |
| | | AUGGGACUUGC | | | | | UUUCUUCGGU | |
| 1614173 | 1614171 | UGAUUUCAUGUC | 34 | 6673 | 6695 | 1614172 | GGGGGGAGGGA | 63 |
| | | CCUCCCCCCAC | | | | | CAUGAAAUCA | |
| 1614176 | 1614174 | AAAGCUAAGCUA | 35 | 6688 | 6710 | 1614175 | AAAUCAUCUUA | 64 |
| | | AGAUGAUUUCA | | | | | GCUUAGCUUU | |
| 1614182 | 1614180 | ACAAUACACUAU | 36 | 6718 | 6740 | 1614181 | AAUGUCUAUAU | 65 |
| | | AUAGACAUUCA | | | | | AGUGUAUUGU | |
| 1614188 | 1614186 | ACAGUCAGUGUA | 37 | 6748 | 6770 | 1614187 | CAAAUGAUUUA | 66 |
| | | AAUCAUUUGUU | | | | | CACUGACUGU | |
| 1614191 | 1614189 | UUCACUUUUACA | 38 | 6763 | 6785 | 1614190 | GACUGUUGCUG | 67 |
| | | GCAACAGUCAG | | | | | UAAAAGUGAA | |
| 1614194 | 1614192 | AACUUUAUUUCC | 39 | 6778 | 6800 | 1614193 | AGUGAAUUUGG | 68 |
| | | AAAUUCACUUU | | | | | AAAUAAAGUU | |

TABLE 2
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1613699 | 1613697 | AAACCUCUUUAC | 69 | 4601 | 4623 | 1613698 | GAAAUGCUUGU | 113 |
| | | AAGCAUUUCAA | | | | | AAAGAGGUUU | |
| 1613705 | 1613703 | AGACACCUCGUG | 70 | 4625 | 4647 | 1613704 | ACCCACCCUCAC | 114 |
| | | AGGGUGGGUUA | | | | | GAGGUGUCU | |
| 1613708 | 1613706 | UCAAAACUUGGG | 71 | 4692 | 4714 | 1613707 | CUGGGGCCUCCC | 115 |
| | | AGGCCCCAGCA | | | | | AAGUUUUGA | |
| 1613714 | 1613712 | UUGGGUCCCAGG | 72 | 4720 | 4742 | 1613713 | UCCUCAGCACCU | 116 |
| | | UGCUGAGGAAA | | | | | GGGACCCAA | |
| 1613717 | 1613715 | AGAAGCUGGUCU | 73 | 4735 | 4757 | 1613716 | ACCCAACAGAG | 117 |
| | | CUGUUGGGUCC | | | | | ACCAGCUUCU | |
| 1613726 | 1613724 | UGCUUCAGGCCU | 74 | 4778 | 4800 | 1613725 | CUGUGACGAAG | 118 |
| | | UCGUCACAGCU | | | | | GCCUGAAGCA | |
| 1613729 | 1613727 | UCAGUCCUAAUC | 75 | 4793 | 4815 | 1613728 | GAAGCACAGGA | 119 |
| | | CUGUGCUUCAG | | | | | UUAGGACUGA | |
| 1613735 | 1613733 | AGGGGAAGUAGG | 76 | 4823 | 4845 | 1613734 | GUCCCCUUCCCU | 120 |
| | | GAAGGGGACAU | | | | | ACUUCCCCU | |
| 1613738 | 1613736 | AAGGGGAAGUAG | 77 | 4824 | 4846 | 1613737 | UCCCCUUCCCUA | 121 |
| | | GGAAGGGGACA | | | | | CUUCCCCUU | |
| 1613741 | 1613739 | UAGUCUGUGCCC | 78 | 4852 | 4874 | 1613740 | CCUGUGUCAGG | 122 |
| | | UGACACAGGGA | | | | | GCACAGACUA | |
| 1613744 | 1613742 | ACCAGCCACAAG | 79 | 4867 | 4889 | 1613743 | AGACUAGGUCU | 123 |
| | | ACCUAGUCUGU | | | | | UGUGGCUGGU | |
| 1613747 | 1613745 | AAGCCAGACCAG | 80 | 4874 | 4896 | 1613746 | GUCUUGUGGCU | 124 |
| | | CCACAAGACCU | | | | | GGUCUGGCUU | |
| 1613750 | 1613748 | UGACCAGAGAGA | 81 | 4902 | 4924 | 1613749 | GAGGAUGGUUC | 125 |
| | | ACCAUCCUCGC | | | | | UCUCUGGUCA | |
| 1613753 | 1613751 | UGAGACUUCGGG | 82 | 4917 | 4939 | 1613752 | UGGUCAUAGCC | 126 |
| | | CUAUGACCAGA | | | | | CGAAGUCUCA | |
| 1613759 | 1613757 | AGGAGUUGUAAG | 83 | 4947 | 4969 | 1613758 | CAAAGGAGGCU | 127 |
| | | CCUCCUUUGGG | | | | | UACAACUCCU | |
| 1613762 | 1613760 | UUUUUCUUGUGA | 84 | 4962 | 4984 | 1613761 | ACUCCUGCAUCA | 128 |
| | | UGCAGGAGUUG | | | | | CAAGAAAAA | |
| 1613771 | 1613769 | UUGGAGAGGAAC | 85 | 5046 | 5068 | 1613770 | UCAGACUGGGU | 129 |
| | | CCAGUCUGAGG | | | | | UCCUCUCCAA | |
| 1613777 | 1613775 | UCAUCCCAAUCCC | 86 | 5115 | 5137 | 1613776 | CCACAGCAGGG | 130 |
| | | UGCUGUGGUC | | | | | AUUGGGAUGA | |
| 1613780 | 1613778 | UCCAGGACAGGC | 87 | 5130 | 5152 | 1613779 | GGAUGAAUUGC | 131 |
| | | AAUUCAUCCCA | | | | | CUGUCCUGGA | |
| 1613789 | 1613787 | AUCCUUCCUCAG | 88 | 5169 | 5191 | 1613788 | GCUGCCUGCCUG | 132 |
| | | GCAGGCAGCUU | | | | | AGGAAGGAU | |
| 1613795 | 1613793 | UGGGAACAGUGU | 89 | 5199 | 5221 | 1613794 | AGUCAGGAGAC | 133 |
| | | CUCCUGACUUG | | | | | ACUGUUCCCA | |
| 1613804 | 1613802 | UUUGUGCAAGGU | 90 | 5241 | 5263 | 1613803 | AGCCCGCUGACC | 134 |
| | | CAGCGGGCUGA | | | | | UUGCACAAA | |
| 1613807 | 1613805 | UGGCAGCAGAUG | 91 | 5256 | 5278 | 1613806 | CACAAACUCCAU | 135 |
| | | GAGUUUGUGCA | | | | | CUGCUGCCA | |
| 1613813 | 1613811 | UGUUUUGCAAAG | 92 | 5286 | 5308 | 1613812 | GGAAGCCGCCU | 136 |
| | | GCGGCUUCCCU | | | | | UUGCAAAACA | |
| 1613816 | 1613814 | UUCUUUAGGCAG | 93 | 5301 | 5323 | 1613815 | AAAACAUUGCU | 137 |
| | | CAAUGUUUUGC | | | | | GCCUAAAGAA | |
| 1613822 | 1613820 | AGAAUUGGGCCU | 94 | 5326 | 5348 | 1613821 | AGCAGCCUCAG | 138 |
| | | GAGGCUGCUGA | | | | | GCCCAAUUCU | |
| 1613831 | 1613829 | AAGUCCCUCAGG | 95 | 5371 | 5393 | 1613830 | AAAGGCAACCC | 139 |
| | | GUUGCCUUUAA | | | | | UGAGGGACUU | |
| 1613834 | 1613832 | UGGAUUUCUACU | 96 | 5386 | 5408 | 1613833 | GGACUUGGCAG | 140 |
| | | GCCAAGUCCCU | | | | | UAGAAAUCCA | |
| 1613837 | 1613835 | AGGGGAGGCCCU | 97 | 5397 | 5419 | 1613836 | UAGAAAUCCAG | 141 |
| | | GGAUUUCUACU | | | | | GGCCUCCCCU | |
| 1613840 | 1613838 | UCUAGCUGCACA | 98 | 5425 | 5447 | 1613839 | GCAGCUUCGUG | 142 |
| | | CGAAGCUGCCA | | | | | UGCAGCUAGA | |
| 1613846 | 1613844 | UGGGCCCAGAGA | 99 | 5455 | 5477 | 1613845 | GAAAGGAAGUC | 143 |
| | | CUUCCUUUCAG | | | | | UCUGGGCCCA | |
| 1613855 | 1613853 | AGGGAGGCUCUU | 100 | 5479 | 5501 | 1613854 | CUCUCCACCAAG | 144 |
| | | GGUGGAGAGUU | | | | | AGCCUCCCU | |
| 1613858 | 1613856 | AAUUGCUGGGAC | 101 | 5503 | 5525 | 1613857 | GUUCGCUGAGU | 145 |
| | | UCAGCGAACGG | | | | | CCCAGCAAUU | |
| 1613864 | 1613862 | UCUCCUUCUCAG | 102 | 5533 | 5555 | 1613863 | UGAAGGGAUCU | 146 |
| | | AUCCCUUCAAC | | | | | GAGAAGGAGA | |
| 1613867 | 1613865 | UACCCCACAUUU | 103 | 5548 | 5570 | 1613866 | AGGAGAAGGAA | 147 |
| | | CCUUCUCCUUC | | | | | AUGUGGGGUA | |
| 1613870 | 1613868 | AACCACCACCAA | 104 | 5563 | 5585 | 1613869 | GGGGUAGAUUU | 148 |
| | | AUCUACCCCAC | | | | | GGUGGUGGUU | |
| 1613876 | 1613874 | UGUUGGCAGUAA | 105 | 5593 | 5615 | 1613875 | CCCCCCUCAUUA | 149 |
| | | UGAGGGGGGCA | | | | | CUGCCAACA | |
| 1613879 | 1613877 | AAAUGCAGCCGA | 106 | 5608 | 5630 | 1613878 | CCAACAGUUUC | 150 |
| | | AACUGUUGGCA | | | | | GGCUGCAUUU | |
| 1613897 | 1613895 | AGCAGGGCACAA | 107 | 5653 | 5675 | 1613896 | CUGAAGUUCUU | 151 |
| | | GAACUUCAGGA | | | | | GUGCCCUGCU | |
| 1613900 | 1613898 | AUGGUGCUGAAG | 108 | 5665 | 5687 | 1613899 | UGCCCUGCUCUU | 152 |
| | | AGCAGGGCACA | | | | | CAGCACCAU | |
| 1613912 | 1613910 | UGAUCUUAGGCU | 109 | 5736 | 5758 | 1613911 | CUUGGGGCCAG | 153 |
| | | GGCCCCAAGAG | | | | | CCUAAGAUCA | |
| 1613918 | 1613916 | UUAUCUGCCAGC | 110 | 5766 | 5788 | 1613917 | GUGAUCAGUGC | 154 |
| | | ACUGAUCACCC | | | | | UGGCAGAUAA | |
| 1613924 | 1613922 | UAAGAUCACAAG | 111 | 5796 | 5818 | 1613923 | GCACGCUGGCU | 155 |
| | | CCAGCGUGCCU | | | | | UGUGAUCUUA | |
| 1613933 | 1613931 | AUCUAGCCCACCC | 112 | 5884 | 5906 | 1613932 | GUGUCCUUGGG | 156 |
| | | AAGGACACUG | | | | | UGGGCUAGAU | |

TABLE 3
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1613468 | 1613466 | UCACACUCACAC | 157 | 3135 | 3157 | 1613467 | GUCAACCUUGUG | 205 |
| | | AAGGUUGACAU | | | | | UGAGUGUGA | |
| 1613474 | 1613472 | UCUUGUGCUUCC | 158 | 3211 | 3233 | 1613473 | GAGGGGAGAGGA | 206 |
| | | UCUCCCCUCUG | | | | | AGCACAAGA | |
| 1613477 | 1613475 | UCUCCCACUCCC | 159 | 3226 | 3248 | 1613476 | ACAAGAAGUGGG | 207 |
| | | ACUUCUUGUGC | | | | | AGUGGGAGA | |
| 1613483 | 1613481 | UACUCUCCAGCA | 160 | 3248 | 3270 | 1613482 | GAAGCCACGUGC | 208 |
| | | CGUGGCUUCCU | | | | | UGGAGAGUA | |
| 1613486 | 1613484 | AAGGAGGGGGAU | 161 | 3263 | 3285 | 1613485 | AGAGUAGACAUC | 209 |
| | | GUCUACUCUCC | | | | | CCCCUCCUU | |
| 1613492 | 1613490 | UUUACCCGCUGU | 162 | 3403 | 3425 | 1613491 | GGAGAAGGGACA | 210 |
| | | CCCUUCUCCCA | | | | | GCGGGUAAA | |
| 1613495 | 1613493 | AGCUUGCCUUCU | 163 | 3418 | 3440 | 1613494 | GGUAAAAAGAGA | 211 |
| | | CUUUUUACCCG | | | | | AGGCAAGCU | |
| 1613501 | 1613499 | AGGAGGUCAUCC | 164 | 3451 | 3473 | 1613500 | GCACUUCGUGGA | 212 |
| | | ACGAAGUGCCA | | | | | UGACCUCCU | |
| 1613510 | 1613508 | AAGAGGCCAGCG | 165 | 3493 | 3515 | 1613509 | UCUUGAGAGCGC | 213 |
| | | CUCUCAAGACA | | | | | UGGCCUCUU | |
| 1613513 | 1613511 | UUUCUGUGGAGC | 166 | 3552 | 3574 | 1613512 | CUUCCCUCUGCU | 214 |
| | | AGAGGGAAGCC | | | | | CCACAGAAA | |
| 1613519 | 1613517 | UUCCAACCUUCA | 167 | 3582 | 3604 | 1613518 | AUUGAGUUCUGA | 215 |
| | | GAACUCAAUAA | | | | | AGGUUGGAA | |
| 1613522 | 1613520 | AAAUCAUGGCAG | 168 | 3597 | 3619 | 1613521 | UUGGAACUGCUG | 216 |
| | | CAGUUCCAACC | | | | | CCAUGAUUU | |
| 1613525 | 1613523 | UCUGCAAAGUGG | 169 | 3612 | 3634 | 1613524 | UGAUUUUGGCCA | 217 |
| | | CCAAAAUCAUG | | | | | CUUUGCAGA | |
| 1613531 | 1613529 | AAGAGAACUGGU | 170 | 3642 | 3664 | 1613530 | UUAGGGCUAACC | 218 |
| | | UAGCCCUAAAG | | | | | AGUUCUCUU | |
| 1613540 | 1613538 | AAACGGGUGGAC | 171 | 3681 | 3703 | 1613539 | UUGGGAGACGUC | 219 |
| | | GUCUCCCAAGA | | | | | CACCCGUUU | |
| 1613543 | 1613541 | ACACACUCCAGA | 172 | 3715 | 3737 | 1613542 | ACUGGCAUCUCU | 220 |
| | | GAUGCCAGUGG | | | | | GGAGUGUGU | |
| 1613546 | 1613544 | AGACCCCCACAC | 173 | 3723 | 3745 | 1613545 | CUCUGGAGUGUG | 221 |
| | | ACUCCAGAGAU | | | | | UGGGGGUCU | |
| 1613549 | 1613547 | UCUCACGGCAGA | 174 | 3808 | 3830 | 1613548 | GUGCUGUUGUCU | 222 |
| | | CAACAGCACAG | | | | | GCCGUGAGA | |
| 1613552 | 1613550 | AGUGAUUGGGCU | 175 | 3819 | 3841 | 1613551 | UGCCGUGAGAGC | 223 |
| | | CUCACGGCAGA | | | | | CCAAUCACU | |
| 1613555 | 1613553 | AUGAGGGGUAUA | 176 | 3834 | 3856 | 1613554 | AUCACUGCCUAU | 224 |
| | | GGCAGUGAUUG | | | | | ACCCCUCAU | |
| 1613558 | 1613556 | ACAUUGUGACGU | 177 | 3849 | 3871 | 1613557 | CCUCAUCACACG | 225 |
| | | GUGAUGAGGGG | | | | | UCACAAUGU | |
| 1613564 | 1613562 | AAGGGGUGGUGA | 178 | 3876 | 3898 | 1613563 | UUCCCAGCCUCA | 226 |
| | | GGCUGGGAAUU | | | | | CCACCCCUU | |
| 1613567 | 1613565 | AGGGUCAUUACU | 179 | 3891 | 3913 | 1613566 | CCCCUUCUCAGU | 227 |
| | | GAGAAGGGGUG | | | | | AAUGACCCU | |
| 1613576 | 1613574 | UUAAUUUCACCC | 180 | 3936 | 3958 | 1613575 | CCAUACUGAGGG | 228 |
| | | UCAGUAUGGAG | | | | | UGAAAUUAA | |
| 1613588 | 1613586 | AACUGAGAGUGA | 181 | 3989 | 4011 | 1613587 | CCCAGCCUCUCA | 229 |
| | | GAGGCUGGGGU | | | | | CUCUCAGUU | |
| 1613594 | 1613592 | UGGUGAGGGUCC | 182 | 4016 | 4038 | 1613593 | AUCCAACUGGGA | 230 |
| | | CAGUUGGAUGA | | | | | CCCUCACCA | |
| 1613597 | 1613595 | AGAUCAUGAGAU | 183 | 4031 | 4053 | 1613596 | UCACCACGAAUC | 231 |
| | | UCGUGGUGAGG | | | | | UCAUGAUCU | |
| 1613600 | 1613598 | ACAGGGAACCGA | 184 | 4046 | 4068 | 1613599 | UGAUCUGAUUCG | 232 |
| | | AUCAGAUCAUG | | | | | GUUCCCUGU | |
| 1613606 | 1613604 | UCACAUCUGUGA | 185 | 4070 | 4092 | 1613605 | CUCCUCCCGUCA | 233 |
| | | CGGGAGGAGGA | | | | | CAGAUGUGA | |
| 1613609 | 1613607 | AGUGCCCUGGCU | 186 | 4081 | 4103 | 1613608 | ACAGAUGUGAGC | 234 |
| | | CACAUCUGUGA | | | | | CAGGGCACU | |
| 1613612 | 1613610 | ACCUAGGGUCAC | 187 | 4101 | 4123 | 1613611 | UGCUCAGCUGUG | 235 |
| | | AGCUGAGCAGU | | | | | ACCCUAGGU | |
| 1613624 | 1613622 | ACAACCAAGACA | 188 | 4199 | 4221 | 1613623 | CAGGCUGGGUGU | 236 |
| | | CCCAGCCUGCU | | | | | CUUGGUUGU | |
| 1613630 | 1613628 | UUCCAUCCUGGU | 189 | 4221 | 4243 | 1613629 | AGUGGUGGCACC | 237 |
| | | GCCACCACUGA | | | | | AGGAUGGAA | |
| 1613633 | 1613631 | UACAAGCUAGGG | 190 | 4282 | 4304 | 1613632 | CCACUUGCACCC | 238 |
| | | UGCAAGUGGGG | | | | | UAGCUUGUA | |
| 1613636 | 1613634 | UGGGAGGUUGGC | 191 | 4297 | 4319 | 1613635 | CUUGUAGCUGCC | 239 |
| | | AGCUACAAGCU | | | | | AACCUCCCA | |
| 1613639 | 1613637 | UGUCUGGGAGGU | 192 | 4301 | 4323 | 1613638 | UAGCUGCCAACC | 240 |
| | | UGGCAGCUACA | | | | | UCCCAGACA | |
| 1613642 | 1613640 | UAUGCAUGUGGA | 193 | 4332 | 4354 | 1613641 | CUGCUCAGCUCC | 241 |
| | | GCUGAGCAGCG | | | | | ACAUGCAUA | |
| 1613648 | 1613646 | UUUGUCGGGUGU | 194 | 4357 | 4379 | 1613647 | CAGCCCUCCACA | 242 |
| | | GGAGGGCUGAU | | | | | CCCGACAAA | |
| 1613654 | 1613652 | ACCAUUUCCAAG | 195 | 4383 | 4405 | 1613653 | ACACACCCCCUU | 243 |
| | | GGGGUGUGUUC | | | | | GGAAAUGGU | |
| 1613660 | 1613658 | UUCCAGCUGGGA | 196 | 4408 | 4430 | 1613659 | UUCCCCCAGUCC | 244 |
| | | CUGGGGGAAAA | | | | | CAGCUGGAA | |
| 1613663 | 1613661 | AACAGACAGCAU | 197 | 4423 | 4445 | 1613662 | CUGGAAGCCAUG | 245 |
| | | GGCUUCCAGCU | | | | | CUGUCUGUU | |
| 1613669 | 1613667 | AUCUAUGUAUAU | 198 | 4453 | 4475 | 1613668 | CAGCUGAACAUA | 246 |
| | | GUUCAGCUGCU | | | | | UACAUAGAU | |
| 1613675 | 1613673 | ACUCAACAGGGU | 199 | 4488 | 4510 | 1613674 | CCCAUCUGCACC | 247 |
| | | GCAGAUGGGGA | | | | | CUGUUGAGU | |
| 1613678 | 1613676 | ACAAAUCCAACU | 200 | 4503 | 4525 | 1613677 | UUGAGUUGUAGU | 248 |
| | | ACAACUCAACA | | | | | UGGAUUUGU | |
| 1613681 | 1613679 | UCCAAGCAUAAA | 201 | 4518 | 4540 | 1613680 | AUUUGUCUGUUU | 249 |
| | | CAGACAAAUCC | | | | | AUGCUUGGA | |
| 1613684 | 1613682 | AGUCACUCUGGU | 202 | 4533 | 4555 | 1613683 | CUUGGAUUCACC | 250 |
| | | GAAUCCAAGCA | | | | | AGAGUGACU | |
| 1613687 | 1613685 | UCUUUUCACUAU | 203 | 4548 | 4570 | 1613686 | GUGACUAUGAUA | 251 |
| | | CAUAGUCACUC | | | | | GUGAAAAGA | |
| 1613690 | 1613688 | UUUUUUUCUUUU | 204 | 4554 | 4576 | 1613689 | AUGAUAGUGAAA | 252 |
| | | CACUAUCAUAG | | | | | AGAAAAAAA | |

TABLE 4
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1613225 | 1613223 | UUGCUGGAAUCC | 253 | 1824 | 1846 | 1613224 | ACGCCACCAGG | 291 |
| | | UGGUGGCGUUG | | | | | AUUCCAGCAA | |
| 1613231 | 1613229 | UCUUUGGAGCGG | 254 | 1845 | 1867 | 1613230 | AAACCCCGCCC | 292 |
| | | GCGGGGUUUUU | | | | | GCUCCAAAGA | |
| 1613237 | 1613235 | AUUUUGGAGGUU | 255 | 1875 | 1897 | 1613236 | GCUCUGGUGAA | 293 |
| | | CACCAGAGCUG | | | | | CCUCCAAAAU | |
| 1613243 | 1613241 | UAGCCGCUGCGA | 256 | 1894 | 1916 | 1613242 | AUCAGGGGAUC | 294 |
| | | UCCCCUGAUUU | | | | | GCAGCGGCUA | |
| 1613246 | 1613244 | UACGGACCACUG | 257 | 1995 | 2017 | 1613245 | AGAAGGUGGCA | 295 |
| | | CCACCUUCUUG | | | | | GUGGUCCGUA | |
| 1613249 | 1613247 | ACUUGGGUGGAG | 258 | 2007 | 2029 | 1613248 | UGGUCCGUACU | 296 |
| | | UACGGACCACU | | | | | CCACCCAAGU | |
| 1613252 | 1613250 | AAGACGGCGACU | 259 | 2016 | 2038 | 1613251 | CUCCACCCAAG | 297 |
| | | UGGGUGGAGUA | | | | | UCGCCGUCUU | |
| 1613255 | 1613253 | UUCUUCAGGUCU | 260 | 2068 | 2090 | 1613254 | GCCCAUGCCAG | 298 |
| | | GGCAUGGGCAC | | | | | ACCUGAAGAA | |
| 1613258 | 1613256 | AUCUUGGACUUG | 261 | 2083 | 2105 | 1613257 | GAAGAAUGUCA | 299 |
| | | ACAUUCUUCAG | | | | | AGUCCAAGAU | |
| 1613261 | 1613259 | UUCUCAGUGGAG | 262 | 2098 | 2120 | 1613260 | CAAGAUCGGCU | 300 |
| | | CCGAUCUUGGA | | | | | CCACUGAGAA | |
| 1613267 | 1613265 | AUUAUCUGCACC | 263 | 2137 | 2159 | 1613266 | AGGCGGGAAGG | 301 |
| | | UUCCCGCCUCC | | | | | UGCAGAUAAU | |
| 1613270 | 1613268 | UCCAGCUUCUUA | 264 | 2152 | 2174 | 1613269 | GAUAAUUAAUA | 302 |
| | | UUAAUUAUCUG | | | | | AGAAGCUGGA | |
| 1613273 | 1613271 | UGGACGUUGCUA | 265 | 2167 | 2189 | 1613272 | GCUGGAUCUUA | 303 |
| | | AGAUCCAGCUU | | | | | GCAACGUCCA | |
| 1613279 | 1613277 | UUGAUAUUAUCC | 266 | 2197 | 2219 | 1613278 | UGGCUCAAAGG | 304 |
| | | UUUGAGCCACA | | | | | AUAAUAUCAA | |
| 1613288 | 1613286 | ACUAUUUGCACA | 267 | 2230 | 2252 | 1613287 | AGGCGGCAGUG | 305 |
| | | CUGCCGCCUCC | | | | | UGCAAAUAGU | |
| 1613291 | 1613289 | UCAACUGGUUUG | 268 | 2245 | 2267 | 1613290 | AAUAGUCUACA | 306 |
| | | UAGACUAUUUG | | | | | AACCAGUUGA | |
| 1613300 | 1613298 | UGGAUGUUGCCU | 269 | 2290 | 2312 | 1613299 | UGGCUCAUUAG | 307 |
| | | AAUGAGCCACA | | | | | GCAACAUCCA | |
| 1613345 | 1613343 | UUUGGCGUUCUC | 270 | 2453 | 2475 | 1613344 | ACCUUCCGCGA | 308 |
| | | GCGGAAGGUCA | | | | | GAACGCCAAA | |
| 1613351 | 1613349 | UGGCGACUUGUA | 271 | 2495 | 2517 | 1613350 | GAGAUCGUGUA | 309 |
| | | CACGAUCUCCG | | | | | CAAGUCGCCA | |
| 1613357 | 1613355 | UUGCUGAGAUGC | 272 | 2533 | 2555 | 1613356 | GUCUCCACGGC | 310 |
| | | CGUGGAGACGU | | | | | AUCUCAGCAA | |
| 1613363 | 1613361 | AUGCUGCCGGUG | 273 | 2554 | 2576 | 1613362 | UGUCUCCUCCA | 311 |
| | | GAGGAGACAUU | | | | | CCGGCAGCAU | |
| 1613369 | 1613367 | UGGGGCGAGUCU | 274 | 2575 | 2597 | 1613368 | CGACAUGGUAG | 312 |
| | | ACCAUGUCGAU | | | | | ACUCGCCCCA | |
| 1613372 | 1613370 | AUCACAAACCCU | 275 | 2631 | 2653 | 1613371 | UGGCCAAGCAG | 313 |
| | | GCUUGGCCAGG | | | | | GGUUUGUGAU | |
| 1613378 | 1613376 | ACAAUUAUUGAC | 276 | 2658 | 2680 | 1613377 | CCUGGGGCGGU | 314 |
| | | CGCCCCAGGGG | | | | | CAAUAAUUGU | |
| 1613384 | 1613382 | UUUUCCACACUC | 277 | 2688 | 2710 | 1613383 | AGAAUGAGAGA | 315 |
| | | UCUCAUUCUCU | | | | | GUGUGGAAAA | |
| 1613393 | 1613391 | UAACCGAACUGC | 278 | 2748 | 2770 | 1613392 | CUGCUCCUCGC | 316 |
| | | GAGGAGCAGCU | | | | | AGUUCGGUUA | |
| 1613396 | 1613394 | AAGUGAUUAACC | 279 | 2763 | 2785 | 1613395 | CGGUUAAUUGG | 317 |
| | | AAUUAACCGAA | | | | | UUAAUCACUU | |
| 1613399 | 1613397 | UGACAAAAGCAG | 280 | 2778 | 2800 | 1613398 | UCACUUAACCU | 318 |
| | | GUUAAGUGAUU | | | | | GCUUUUGUCA | |
| 1613417 | 1613415 | UAUUUUAUUACU | 281 | 2868 | 2890 | 1613416 | GGGUGGGCUAG | 319 |
| | | AGCCCACCCAU | | | | | UAAUAAAAUA | |
| 1613420 | 1613418 | UUUAAAUAUUUU | 282 | 2874 | 2896 | 1613419 | GCUAGUAAUAA | 320 |
| | | AUUACUAGCCC | | | | | AAUAUUUAAA | |
| 1613426 | 1613424 | AAAUGUUGGAUG | 283 | 2910 | 2932 | 1613425 | ACAUGGCCACA | 321 |
| | | UGGCCAUGUUU | | | | | UCCAACAUUU | |
| 1613429 | 1613427 | AGGAAUUGCCUG | 284 | 2925 | 2947 | 1613428 | ACAUUUCCUCA | 322 |
| | | AGGAAAUGUUG | | | | | GGCAAUUCCU | |
| 1613435 | 1613433 | UACAUGGAGGGG | 285 | 2955 | 2977 | 1613434 | UUUUUCUUCCC | 323 |
| | | GAAGAAAAAAG | | | | | CCUCCAUGUA | |
| 1613438 | 1613436 | UCCUUCUCCCUC | 286 | 2970 | 2992 | 1613437 | CAUGUAGAAGA | 324 |
| | | UUCUACAUGGA | | | | | GGGAGAAGGA | |
| 1613441 | 1613439 | AGCUUUCAGAGC | 287 | 2985 | 3007 | 1613440 | GAAGGAGAGGC | 325 |
| | | CUCUCCUUCUC | | | | | UCUGAAAGCU | |
| 1613444 | 1613442 | AAAUCCCCCAGA | 288 | 3000 | 3022 | 1613443 | AAAGCUGCUUC | 326 |
| | | AGCAGCUUUCA | | | | | UGGGGGAUUU | |
| 1613450 | 1613448 | UUGGCACCCCCA | 289 | 3020 | 3042 | 1613449 | UCAAGGGACUG | 327 |
| | | GUCCCUUGAAA | | | | | GGGGUGCCAA | |
| 1613453 | 1613451 | UUGCUGCCACUG | 290 | 3067 | 3089 | 1613452 | UCACAGAGGCA | 328 |
| | | CCUCUGUGACA | | | | | GUGGCAGCAA | |

TABLE 5
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1612979 | 1612977 | UGAUAGUCGACA | 329 | 110 | 132 | 1612978 | CCUCGCCUCUG | 376 |
| | | GAGGCGAGGAC | | | | | UCGACUAUCA | |
| 1612988 | 1612986 | UCCAUCACUUCG | 330 | 163 | 185 | 1612987 | CCAGGAGUUCG | 377 |
| | | AACUCCUGGCG | | | | | AAGUGAUGGA | |
| 1612997 | 1612995 | UCUUUCCUGUCC | 331 | 202 | 224 | 1612996 | CGGGUUGGGGG | 378 |
| | | CCCAACCCGUA | | | | | ACAGGAAAGA | |
| 1613003 | 1613001 | UGGUCUUGGUGC | 332 | 232 | 254 | 1613002 | CUACACCAUGC | 379 |
| | | AUGGUGUAGCC | | | | | ACCAAGACCA | |
| 1613006 | 1613004 | UCCGUGUCACCC | 333 | 247 | 269 | 1613005 | AGACCAAGAGG | 380 |
| | | UCUUGGUCUUG | | | | | GUGACACGGA | |
| 1613012 | 1613010 | UUCUUUCAGGCC | 334 | 263 | 285 | 1613011 | ACGGACGCUGG | 381 |
| | | AGCGUCCGUGU | | | | | CCUGAAAGAA | |
| 1613021 | 1613019 | UUCCUCAGAUCC | 335 | 302 | 324 | 1613020 | ACUGAGGACGG | 382 |
| | | GUCCUCAGUGG | | | | | AUCUGAGGAA | |
| 1613030 | 1613028 | UCCGCUGUUGGA | 336 | 346 | 368 | 1613029 | UAAGAGCACUC | 383 |
| | | GUGCUCUUAGC | | | | | CAACAGCGGA | |
| 1613039 | 1613037 | UGACCAGCAGCU | 337 | 403 | 425 | 1613038 | GGAAGACGAAG | 384 |
| | | UCGUCUUCCAG | | | | | CUGCUGGUCA | |
| 1613042 | 1613040 | UUGGGUCACGUG | 338 | 413 | 435 | 1613041 | GCUGCUGGUCA | 385 |
| | | ACCAGCAGCUU | | | | | CGUGACCCAA | |
| 1613045 | 1613043 | AACUCUCAACUC | 339 | 428 | 450 | 1613044 | ACCCAAGAGGA | 386 |
| | | CUCUUGGGUCA | | | | | GUUGAGAGUU | |
| 1613051 | 1613049 | UCUCAUUGGCCA | 340 | 474 | 496 | 1613050 | AAAGGCCCCUG | 387 |
| | | GGGGCCUUUCA | | | | | GCCAAUGAGA | |
| 1613060 | 1613058 | AGCUUCUGGCUC | 341 | 557 | 579 | 1613059 | GGGGAGAAAGA | 388 |
| | | UUUCUCCCCGA | | | | | GCCAGAAGCU | |
| 1613066 | 1613064 | UUUUUGUCCCCA | 342 | 659 | 681 | 1613065 | UCCCUGGAGUG | 389 |
| | | CUCCAGGGAGG | | | | | GGGACAAAAA | |
| 1613072 | 1613070 | UGUGUGGAGAUA | 343 | 725 | 747 | 1613071 | ACCACUGCGUA | 390 |
| | | CGCAGUGGUGG | | | | | UCUCCACACA | |
| 1613075 | 1613073 | ACCACUUUCAGG | 344 | 740 | 762 | 1613074 | CACACAGAGCC | 391 |
| | | CUCUGUGUGGA | | | | | UGAAAGUGGU | |
| 1613078 | 1613076 | UUCCUGGACCAC | 345 | 755 | 777 | 1613077 | AGUGGUAAGGU | 392 |
| | | CUUACCACUUU | | | | | GGUCCAGGAA | |
| 1613081 | 1613079 | AGGAAGCCUUCC | 346 | 763 | 785 | 1613080 | GGUGGUCCAGG | 393 |
| | | UGGACCACCUU | | | | | AAGGCUUCCU | |
| 1613084 | 1613082 | ACAUGAGCUGGU | 347 | 804 | 826 | 1613083 | GUCUGAGCCAC | 394 |
| | | GGCUCAGACCU | | | | | CAGCUCAUGU | |
| 1613087 | 1613085 | AAGCUGGUGCUU | 348 | 929 | 951 | 1613086 | GAGCUGCUCAA | 395 |
| | | GAGCAGCUCAG | | | | | GCACCAGCUU | |
| 1613096 | 1613094 | UUCAUCCACCUC | 349 | 1013 | 1035 | 1613095 | AGCAAGGAGGA | 396 |
| | | CUCCUUGCUCC | | | | | GGUGGAUGAA | |
| 1613099 | 1613097 | AUCGACGUCGCG | 350 | 1028 | 1050 | 1613098 | GAUGAAGACCG | 397 |
| | | GUCUUCAUCCA | | | | | CGACGUCGAU | |
| 1613102 | 1613100 | ACUCAUCGACGU | 351 | 1032 | 1054 | 1613101 | AAGACCGCGAC | 398 |
| | | CGCGGUCUUCA | | | | | GUCGAUGAGU | |
| 1613105 | 1613103 | AGGAAAUCCACA | 352 | 1168 | 1190 | 1613104 | CCCCCUCCCUG | 399 |
| | | GGGAGGGGGAU | | | | | UGGAUUUCCU | |
| 1613114 | 1613112 | UCUGAGGCUGGG | 353 | 1201 | 1223 | 1613113 | CACAGAGAUCC | 400 |
| | | AUCUCUGUGGA | | | | | CAGCCUCAGA | |
| 1613117 | 1613115 | AAACGUGAACUC | 354 | 1262 | 1284 | 1613116 | GCCCCCCUGGA | 401 |
| | | CAGGGGGGCAU | | | | | GUUCACGUUU | |
| 1613120 | 1613118 | UGUGAUUUCCAC | 355 | 1277 | 1299 | 1613119 | ACGUUUCACGU | 402 |
| | | GUGAAACGUGA | | | | | GGAAAUCACA | |
| 1613126 | 1613124 | UGCUCCUUCUGC | 356 | 1297 | 1319 | 1613125 | ACCCAACGUGC | 403 |
| | | ACGUUGGGUGU | | | | | AGAAGGAGCA | |
| 1613129 | 1613127 | UUCCCAAAUGCU | 357 | 1323 | 1345 | 1613128 | ACUCGGAGGAG | 404 |
| | | CCUCCGAGUGC | | | | | CAUUUGGGAA | |
| 1613138 | 1613136 | UUGUGUCCUCUC | 358 | 1392 | 1414 | 1613137 | CCUCUUUGGGA | 405 |
| | | CCAAAGAGGGG | | | | | GAGGACACAA | |
| 1613144 | 1613142 | UUUCAGAGGGCU | 359 | 1422 | 1444 | 1613143 | ACCUUCCAGAG | 406 |
| | | CUGGAAGGUCA | | | | | CCCUCUGAAA | |
| 1613153 | 1613151 | UUUGAGUUGAGG | 360 | 1478 | 1500 | 1613152 | AGCCGGGUCCC | 407 |
| | | GACCCGGCUGA | | | | | UCAACUCAAA | |
| 1613156 | 1613154 | ACUGACCAUGCG | 361 | 1493 | 1515 | 1613155 | CUCAAAGCUCG | 408 |
| | | AGCUUUGAGUU | | | | | CAUGGUCAGU | |
| 1613165 | 1613163 | UGUCUUGGCUUU | 362 | 1538 | 1560 | 1613164 | GAUGACAAAAA | 409 |
| | | UUUGUCAUCGC | | | | | AGCCAAGACA | |
| 1613168 | 1613166 | AGAGGAACGUGU | 363 | 1553 | 1575 | 1613167 | AAGACAUCCAC | 410 |
| | | GGAUGUCUUGG | | | | | ACGUUCCUCU | |
| 1613171 | 1613169 | UUUCAAGGUUUU | 364 | 1568 | 1590 | 1613170 | UCCUCUGCUAA | 411 |
| | | AGCAGAGGAAC | | | | | AACCUUGAAA | |
| 1613174 | 1613172 | AAGGCAAGGCCU | 365 | 1583 | 1605 | 1613173 | UUGAAAAAUAG | 412 |
| | | AUUUUUCAAGG | | | | | GCCUUGCCUU | |
| 1613183 | 1613175 | UGUUUGGGGCUA | 366 | 1594 | 1616 | 1613179 | GCCUUGCCUUA | 413 |
| | | AGGCAAGGCCU | | | | | GCCCCAAACA | |
| 1613189 | 1613187 | AGGAGUGGGGUG | 367 | 1604 | 1626 | 1613188 | AGCCCCAAACA | 414 |
| | | UUUGGGGCUAA | | | | | CCCCACUCCU | |
| 1613192 | 1613190 | UGAGCUACCAGG | 368 | 1613 | 1635 | 1613191 | CACCCCACUCC | 415 |
| | | AGUGGGGUGUU | | | | | UGGUAGCUCA | |
| 1613195 | 1613193 | UUGGAUCAGAGG | 369 | 1628 | 1650 | 1613194 | AGCUCAGACCC | 416 |
| | | GUCUGAGCUAC | | | | | UCUGAUCCAA | |
| 1613198 | 1613196 | UGGAGGGUUGGA | 370 | 1635 | 1657 | 1613197 | ACCCUCUGAUC | 417 |
| | | UCAGAGGGUCU | | | | | CAACCCUCCA | |
| 1613201 | 1613199 | UUAGGAGAGGAA | 371 | 1672 | 1694 | 1613200 | AGAGCCACCUU | 418 |
| | | GGUGGCUCUGG | | | | | CCUCUCCUAA | |
| 1613204 | 1613202 | ACAGAAGAGACG | 372 | 1687 | 1709 | 1613203 | UCCUAAAUACG | 419 |
| | | UAUUUAGGAGA | | | | | UCUCUUCUGU | |
| 1613210 | 1613208 | UUUGCUCCAGAA | 373 | 1717 | 1739 | 1613209 | AACUGGCAGUU | 420 |
| | | CUGCCAGUUCG | | | | | CUGGAGCAAA | |
| 1613213 | 1613211 | UUGAGUUUCAUC | 374 | 1732 | 1754 | 1613212 | AGCAAAGGAGA | 421 |
| | | UCCUUUGCUCC | | | | | UGAAACUCAA | |
| 1613216 | 1613214 | UUACCAUCAGCC | 375 | 1747 | 1769 | 1613215 | ACUCAAGGGGG | 422 |
| | | CCCUUGAGUUU | | | | | CUGAUGGUAA | |

“Start site” indicates the 5′-most nucleoside to which the antisense RNAi oligonucleotide is complementary in the target nucleic acid sequence. “Stop site” indicates the 3′-most nucleoside to which the antisense RNAi oligonucleotide is complementary in the target nucleic acid sequence. Each antisense RNAi oligonucleotide listed in the tables below has a single mismatch to SEQ ID NO: 1 (described herein above), wherein the mismatch is located at position 1 on the 5′ end of the antisense sequence.

TABLE 6
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1613948 | 1613946 | AACUGAAGUCAA | 423 | 5959 | 5980 | 1613947 | CCAUUUAAAUU | 453 |
| | | UUUAAAUGGAA | | | | | GACUUCAGUU | |
| 1613954 | 1613952 | AAAGCAAUAGCA | 424 | 5989 | 6010 | 1613953 | UAUCCUGUUUG | 454 |
| | | AACAGGAUACA | | | | | CUAUUGCUUU | |
| 1613957 | 1613955 | ACCCCCAUAGCAC | 425 | 6004 | 6025 | 1613956 | UGCUUGUUGUG | 455 |
| | | AACAAGCAAU | | | | | CUAUGGGGGU | |
| 1613960 | 1613958 | AUCCCCCCAUAGC | 426 | 6006 | 6027 | 1613959 | CUUGUUGUGCU | 456 |
| | | ACAACAAGCA | | | | | AUGGGGGGAU | |
| 1613969 | 1613967 | ACCAAGAUCUCC | 427 | 6055 | 6076 | 1613968 | UGGGCAAAGGG | 457 |
| | | CUUUGCCCAUG | | | | | AGAUCUUGGU | |
| 1613972 | 1613970 | AUUUAAGUGCUG | 428 | 6070 | 6091 | 1613971 | CUUGGGGUGCA | 458 |
| | | CACCCCAAGAU | | | | | GCACUUAAAU | |
| 1613978 | 1613976 | AGUUGAAAUCAU | 429 | 6100 | 6121 | 1613977 | ACCCUUUUCAU | 459 |
| | | GAAAAGGGUUA | | | | | GAUUUCAACU | |
| 1613981 | 1613979 | ACCUCUAGCAAA | 430 | 6115 | 6136 | 1613980 | UCAACCACAUU | 460 |
| | | UGUGGUUGAAA | | | | | UGCUAGAGGU | |
| 1613984 | 1613982 | ACUGCUCCCUCCC | 431 | 6125 | 6146 | 1613983 | UUGCUAGAGGG | 461 |
| | | UCUAGCAAAU | | | | | AGGGAGCAGU | |
| 1613990 | 1613988 | AUCAGUGGAAAA | 432 | 6166 | 6187 | 1613989 | GGGUUUCUCUU | 462 |
| | | GAGAAACCCCA | | | | | UUCCACUGAU | |
| 1613993 | 1613991 | ACUGGGAAAGCC | 433 | 6179 | 6200 | 1613992 | CCACUGACAGGC | 463 |
| | | UGUCAGUGGAA | | | | | UUUCCCAGU | |
| 1614002 | 1614000 | AUGGCUGGGGAG | 434 | 6210 | 6231 | 1614001 | GUUCAUUCCCUC | 464 |
| | | GGAAUGAACUA | | | | | CCCAGCCAU | |
| 1614008 | 1614006 | ACAACCAGAUGU | 435 | 6242 | 6263 | 1614007 | GGAAUAUGGAC | 465 |
| | | CCAUAUUCCUA | | | | | AUCUGGUUGU | |
| 1614011 | 1614009 | ACAGCAGGCCAA | 436 | 6255 | 6276 | 1614010 | CUGGUUGCUUU | 466 |
| | | AGCAACCAGAU | | | | | GGCCUGCUGU | |
| 1614014 | 1614012 | AUGAAAGAGGGC | 437 | 6265 | 6286 | 1614013 | UGGCCUGCUGCC | 467 |
| | | AGCAGGCCAAA | | | | | CUCUUUCAU | |
| 1614017 | 1614015 | AUGGGCUUAGGA | 438 | 6280 | 6301 | 1614016 | UUUCAGGGGUC | 468 |
| | | CCCCUGAAAGA | | | | | CUAAGCCCAU | |
| 1614029 | 1614027 | AACAGAGGUGCC | 439 | 6337 | 6358 | 1614028 | UAAGCCGUUGG | 469 |
| | | AACGGCUUAGA | | | | | CACCUCUGUU | |
| 1614041 | 1614039 | ACGAGUGAUCUC | 440 | 6396 | 6417 | 1614040 | UUUGGAGCUGA | 470 |
| | | AGCUCCAAAAG | | | | | GAUCACUCGU | |
| 1614054 | 1614042 | AAUGAGGAGGGU | 441 | 6411 | 6432 | 1614043 | ACUCGCUUCACC | 471 |
| | | GAAGCGAGUGA | | | | | CUCCUCAUU | |
| 1614110 | 1614088 | ACGACCUCGUGG | 442 | 6441 | 6462 | 1614099 | CAAGUAAAGCC | 472 |
| | | CUUUACUUGGA | | | | | ACGAGGUCGU | |
| 1614134 | 1614132 | ACUGCAGGUCUG | 443 | 6490 | 6511 | 1614133 | UCCCAUCUACAG | 473 |
| | | UAGAUGGGACA | | | | | ACCUGCAGU | |
| 1614137 | 1614135 | AAGAAGUUUUAU | 444 | 6505 | 6526 | 1614136 | UGCAGCUUCAU | 474 |
| | | GAAGCUGCAGG | | | | | AAAACUUCUU | |
| 1614143 | 1614141 | AGGUAACCCUUU | 445 | 6535 | 6556 | 1614142 | CAGCUUUGAAA | 475 |
| | | UCAAAGCUGAA | | | | | AGGGUUACCU | |
| 1614146 | 1614144 | ACAGUGCCCAGG | 446 | 6545 | 6566 | 1614145 | AAGGGUUACCC | 476 |
| | | GUAACCCUUUU | | | | | UGGGCACUGU | |
| 1614155 | 1614153 | AAACAUGGCAAA | 447 | 6595 | 6616 | 1614154 | CCCCAUGAGUU | 477 |
| | | CUCAUGGGGCU | | | | | UGCCAUGUUU | |
| 1614161 | 1614159 | AGACUUGCAAGU | 448 | 6625 | 6646 | 1614160 | AUUUCUGGCAC | 478 |
| | | GCCAGAAAUAG | | | | | UUGCAAGUCU | |
| 1614167 | 1614165 | ACCACCCUCAGA | 449 | 6655 | 6676 | 1614166 | UUCGGUAAUUC | 479 |
| | | AUUACCGAAGA | | | | | UGAGGGUGGU | |
| 1614170 | 1614168 | ACCCCACCCUCAG | 450 | 6657 | 6678 | 1614169 | CGGUAAUUCUG | 480 |
| | | AAUUACCGAA | | | | | AGGGUGGGGU | |
| 1614179 | 1614177 | AACAUUCACAGA | 451 | 6703 | 6724 | 1614178 | AGCUUUCUGUC | 48 |
| | | CAGAAAGCUAA | | | | | UGUGAAUGUU | |
| 1614185 | 1614183 | AAUUUGUUAAAA | 452 | 6733 | 6754 | 1614184 | UAUUGUGUGUU | 482 |
| | | CACACAAUACA | | | | | UUAACAAAUU | |

TABLE 7
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1613702 | 1613700 | AUGAGGGUGGGU | 483 | 4616 | 4637 | 1613701 | AGGUUUCUAAC | 517 |
| | | UAGAAACCUCU | | | | | CCACCCUCAU | |
| 1613711 | 1613709 | ACUGAGGAAAGC | 484 | 4707 | 4728 | 1613710 | UUUUGAAAGGC | 518 |
| | | CUUUCAAAACU | | | | | UUUCCUCAGU | |
| 1613720 | 1613718 | ACCUCCUUAGCU | 485 | 4750 | 4771 | 1613719 | GCUUCUAGCAG | 519 |
| | | GCUAGAAGCUG | | | | | CUAAGGAGGU | |
| 1613723 | 1613721 | AUCACAGCUGAA | 486 | 4764 | 4785 | 1613722 | AGGAGGCCGUU | 520 |
| | | CGGCCUCCUUA | | | | | CAGCUGUGAU | |
| 1613732 | 1613730 | AGGGACAUCAUC | 487 | 4808 | 4829 | 1613731 | GACUGAAGCGA | 521 |
| | | GCUUCAGUCCU | | | | | UGAUGUCCCU | |
| 1613756 | 1613754 | ACUUUGGGACUG | 488 | 4932 | 4953 | 1613755 | GUCUCAUGGCA | 522 |
| | | CCAUGAGACUU | | | | | GUCCCAAAGU | |
| 1613765 | 1613763 | AUGGCAGUGGCU | 489 | 4977 | 4998 | 1613764 | GAAAAAGGAAG | 523 |
| | | UCCUUUUUCUU | | | | | CCACUGCCAU | |
| 1613768 | 1613766 | ACAGCUGGCAGU | 490 | 4981 | 5002 | 1613767 | AAGGAAGCCAC | 524 |
| | | GGCUUCCUUUU | | | | | UGCCAGCUGU | |
| 1613774 | 1613772 | AAGCUUGGAGAG | 491 | 5050 | 5071 | 1613773 | ACUGGGUUCCU | 525 |
| | | GAACCCAGUCU | | | | | CUCCAAGCUU | |
| 1613783 | 1613781 | ACCUCUAGAGCA | 492 | 5144 | 5165 | 1613782 | UCCUGGAUCUG | 526 |
| | | GAUCCAGGACA | | | | | CUCUAGAGGU | |
| 1613786 | 1613784 | AAGCUUGGGCCU | 493 | 5152 | 5173 | 1613785 | CUGCUCUAGAG | 527 |
| | | CUAGAGCAGAU | | | | | GCCCAAGCUU | |
| 1613792 | 1613790 | AUGACUUGUCAA | 494 | 5184 | 5205 | 1613791 | AAGGAUGACUU | 528 |
| | | GUCAUCCUUCC | | | | | GACAAGUCAU | |
| 1613798 | 1613796 | AUCUGGUCAAGG | 495 | 5214 | 5235 | 1613797 | UUCCCAAAGCC | 529 |
| | | CUUUGGGAACA | | | | | UUGACCAGAU | |
| 1613801 | 1613799 | AUGAGGUGCUCU | 496 | 5222 | 5243 | 1613800 | GCCUUGACCAG | 530 |
| | | GGUCAAGGCUU | | | | | AGCACCUCAU | |
| 1613810 | 1613808 | ACUUCCCUUUUC | 497 | 5271 | 5292 | 1613809 | CUGCCAUGAGA | 531 |
| | | UCAUGGCAGCA | | | | | AAAGGGAAGU | |
| 1613819 | 1613817 | AUGAGGCUGCUG | 498 | 5316 | 5337 | 1613818 | AAAGAAACUCA | 532 |
| | | AGUUUCUUUAG | | | | | GCAGCCUCAU | |
| 1613825 | 1613823 | AAAACCAGAAGU | 499 | 5341 | 5362 | 1613824 | AAUUCUGCCAC | 533 |
| | | GGCAGAAUUGG | | | | | UUCUGGUUUU | |
| 1613828 | 1613826 | ACCUUUAACUGU | 500 | 5356 | 5377 | 1613827 | GGUUUGGGUAC | 534 |
| | | ACCCAAACCAG | | | | | AGUUAAAGGU | |
| 1613843 | 1613841 | ACUUUCAGGUAA | 501 | 5440 | 5461 | 1613842 | GCUAGAGCUUU | 535 |
| | | AGCUCUAGCUG | | | | | ACCUGAAAGU | |
| 1613849 | 1613847 | AAGAGUUCUGGG | 502 | 5463 | 5484 | 1613848 | GUCUCUGGGCC | 536 |
| | | CCCAGAGACUU | | | | | CAGAACUCUU | |
| 1613852 | 1613850 | AGAGGCUCUUGG | 503 | 5477 | 5498 | 1613851 | AACUCUCCACC | 537 |
| | | UGGAGAGUUCU | | | | | AAGAGCCUCU | |
| 1613861 | 1613859 | ACUUCAACUUAG | 504 | 5518 | 5539 | 1613860 | GCAAUUCUCCU | 538 |
| | | GAGAAUUGCUG | | | | | AAGUUGAAGU | |
| 1613873 | 1613871 | AGGGGGCAUAUC | 505 | 5578 | 5599 | 1613872 | GUGGUUAGAGA | 539 |
| | | UCUAACCACCA | | | | | UAUGCCCCCU | |
| 1613891 | 1613880 | ACGAGGUGCGUG | 506 | 5623 | 5644 | 1613887 | GCAUUUCUUCA | 540 |
| | | AAGAAAUGCAG | | | | | CGCACCUCGU | |
| 1613894 | 1613892 | AUUCAGGAAGAG | 507 | 5638 | 5659 | 1613893 | CCUCGGUUCCU | 541 |
| | | GAACCGAGGUG | | | | | CUUCCUGAAU | |
| 1613903 | 1613901 | AGUAUAAGAAGG | 508 | 5680 | 5701 | 1613902 | CACCAUGGGCC | 542 |
| | | CCCAUGGUGCU | | | | | UUCUUAUACU | |
| 1613906 | 1613904 | AAUCCCAGAGCC | 509 | 5695 | 5716 | 1613905 | UAUACGGAAGG | 543 |
| | | UUCCGUAUAAG | | | | | CUCUGGGAUU | |
| 1613909 | 1613907 | AGAGAUCCCAGA | 510 | 5698 | 5719 | 1613908 | ACGGAAGGCUC | 544 |
| | | GCCUUCCGUAU | | | | | UGGGAUCUCU | |
| 1613915 | 1613913 | AAUCACCCUAAA | 511 | 5751 | 5772 | 1613914 | AGAUCAUGGUU | 545 |
| | | CCAUGAUCUUA | | | | | UAGGGUGAUU | |
| 1613921 | 1613919 | ACGUGCCUUUUC | 512 | 5781 | 5802 | 1613920 | AGAUAAAUUGA | 546 |
| | | AAUUUAUCUGC | | | | | AAAGGCACGU | |
| 1613927 | 1613925 | AGGAUUGUCCUC | 513 | 5811 | 5832 | 1613926 | AUCUUAAAUGA | 547 |
| | | AUUUAAGAUCA | | | | | GGACAAUCCU | |
| 1613930 | 1613928 | ACCUGGGGGGAU | 514 | 5818 | 5839 | 1613929 | AUGAGGACAAU | 548 |
| | | UGUCCUCAUUU | | | | | CCCCCCAGGU | |
| 1613936 | 1613934 | AAUACAGUAUAU | 515 | 5899 | 5920 | 1613935 | CUAGAUAGGAU | 549 |
| | | CCUAUCUAGCC | | | | | AUACUGUAUU | |
| 1613939 | 1613937 | ACUUGAAGGAGC | 516 | 5914 | 5935 | 1613938 | UGUAUGCCGGC | 550 |
| | | CGGCAUACAGU | | | | | UCCUUCAAGU | |

TABLE 8
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1613465 | 1613463 | AUUGACAUCGUC | 551 | 3120 | 3141 | 1613464 | CCACAGGCAGAC | 581 |
| | | UGCCUGUGGCU | | | | | GAUGUCAAU | |
| 1613471 | 1613469 | AAACCCCCGUCA | 552 | 3144 | 3165 | 1613470 | GUGUGAGUGUG | 582 |
| | | CACUCACACAA | | | | | ACGGGGGUUU | |
| 1613480 | 1613478 | AUUCCUCUCCCA | 553 | 3231 | 3252 | 1613479 | AAGUGGGAGUG | 583 |
| | | CUCCCACUUCU | | | | | GGAGAGGAAU | |
| 1613489 | 1613487 | ACAAGGAGGGGG | 554 | 3265 | 3286 | 1613488 | AGUAGACAUCCC | 584 |
| | | AUGUCUACUCU | | | | | CCUCCUUGU | |
| 1613498 | 1613496 | AUCCUGCCAGCU | 555 | 3426 | 3447 | 1613497 | GAGAAGGCAAGC | 585 |
| | | UGCCUUCUCUU | | | | | UGGCAGGAU | |
| 1613504 | 1613502 | AGUCAGUCUUUU | 556 | 3466 | 3487 | 1613503 | CCUCCUUAGAAA | 586 |
| | | CUAAGGAGGUC | | | | | AGACUGACU | |
| 1613507 | 1613505 | AUCUCAAGACAU | 557 | 3481 | 3502 | 1613506 | CUGACCUUGAUG | 587 |
| | | CAAGGUCAGUC | | | | | UCUUGAGAU | |
| 1613516 | 1613514 | AUCAAUAAAACA | 558 | 3567 | 3588 | 1613515 | CAGAAACCCUGU | 588 |
| | | GGGUUUCUGUG | | | | | UUUAUUGAU | |
| 1613528 | 1613526 | ACCUAAAGUCCC | 559 | 3627 | 3648 | 1613527 | UGCAGACCUGGG | 589 |
| | | AGGUCUGCAAA | | | | | ACUUUAGGU | |
| 1613534 | 1613532 | ACACAAGUCCUU | 560 | 3657 | 3678 | 1613533 | UCUCUUUGUAAG | 590 |
| | | ACAAAGAGAAC | | | | | GACUUGUGU | |
| 1613537 | 1613535 | AACGUCUCCCAA | 561 | 3672 | 3693 | 1613536 | UUGUGCCUCUUG | 591 |
| | | GAGGCACAAGU | | | | | GGAGACGUU | |
| 1613561 | 1613559 | AGCUGGGAAUUC | 562 | 3864 | 3885 | 1613560 | CAAUGUCCCGAA | 592 |
| | | GGGACAUUGUG | | | | | UUCCCAGCU | |
| 1613570 | 1613568 | AUCCUGCAACCA | 563 | 3906 | 3927 | 1613569 | GACCCUGGUUGG | 593 |
| | | ACCAGGGUCAU | | | | | UUGCAGGAU | |
| 1613573 | 1613571 | AUAUGGAGUAG | 564 | 3921 | 3942 | 1613572 | CAGGAGGUACCU | 594 |
| | | GUACCUCCUGCA | | | | | ACUCCAUAU | |
| 1613579 | 1613577 | AGACUUUGCCUU | 565 | 3951 | 3972 | 1613578 | AAUUAAGGGAA | 595 |
| | | CCCUUAAUUUC | | | | | GGCAAAGUCU | |
| 1613582 | 1613580 | ACCACUCUUGUG | 566 | 3966 | 3987 | 1613581 | AAGUCCAGGCAC | 596 |
| | | CCUGGACUUUG | | | | | AAGAGUGGU | |
| 1613585 | 1613583 | AUCCCACUCUUG | 567 | 3968 | 3989 | 1613584 | GUCCAGGCACAA | 597 |
| | | UGCCUGGACUU | | | | | GAGUGGGAU | |
| 1613591 | 1613589 | AAGUUGGAUGA | 568 | 4004 | 4025 | 1613590 | UCAGUUCCACUC | 598 |
| | | GUGGAACUGAGA | | | | | AUCCAACUU | |
| 1613603 | 1613601 | AAGGAGGAGACA | 569 | 4055 | 4076 | 1613602 | UCGGUUCCCUGU | 599 |
| | | GGGAACCGAAU | | | | | CUCCUCCUU | |
| 1613615 | 1613613 | AAACAAGGCAGA | 570 | 4116 | 4137 | 1613614 | CUAGGUGUUUCU | 600 |
| | | AACACCUAGGG | | | | | GCCUUGUUU | |
| 1613618 | 1613616 | AGGCUCUCUCCA | 571 | 4131 | 4152 | 1613617 | UUGUUGACAUGG | 601 |
| | | UGUCAACAAGG | | | | | AGAGAGCCU | |
| 1613621 | 1613619 | AUUCUCAGGGGA | 572 | 4145 | 4166 | 1613620 | AGAGCCCUUUCC | 602 |
| | | AAGGGCUCUCU | | | | | CCUGAGAAU | |
| 1613627 | 1613625 | AUGGUGCCACCA | 573 | 4214 | 4235 | 1613626 | GGUUGUCAGUGG | 603 |
| | | CUGACAACCAA | | | | | UGGCACCAU | |
| 1613645 | 1613643 | AUGGAGGGCUGA | 574 | 4347 | 4368 | 1613644 | UGCAUAGUAUCA | 604 |
| | | UACUAUGCAUG | | | | | GCCCUCCAU | |
| 1613651 | 1613649 | AUGUGUUCCCCU | 575 | 4368 | 4389 | 1613650 | ACCCGACAAAGG | 605 |
| | | UUGUCGGGUGU | | | | | GGAACACAU | |
| 1613657 | 1613655 | AACUGGGGGAAA | 576 | 4398 | 4419 | 1613656 | AAUGGUUCUUUU | 606 |
| | | AGAACCAUUUC | | | | | CCCCCAGUU | |
| 1613666 | 1613664 | AAGCUGCUCCAG | 577 | 4438 | 4459 | 1613665 | UCUGUUCUGCUG | 607 |
| | | CAGAACAGACA | | | | | GAGCAGCUU | |
| 1613672 | 1613670 | AGAGGGCAGGGC | 578 | 4468 | 4489 | 1613671 | AUAGAUGUUGCC | 608 |
| | | AACAUCUAUGU | | | | | CUGCCCUCU | |
| 1613693 | 1613691 | ACGUCCUUUUUU | 579 | 4571 | 4592 | 1613692 | AAAAAAAAAAA | 609 |
| | | UUUUUUUUUUU | | | | | AAAAGGACGU | |
| 1613696 | 1613694 | AAUUUCAAGAUA | 580 | 4586 | 4607 | 1613695 | GGACGCAUGUAU | 610 |
| | | CAUGCGUCCUU | | | | | CUUGAAAUU | |

TABLE 9
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1613222 | 1613220 | AGCGGUGUGGCG | 611 | 1768 | 1789 | 1613221 | AACGAAGAUCG | 651 |
| | | AUCUUCGUUUU | | | | | CCACACCGCU | |
| 1613228 | 1613226 | AGCGGGGUUUUU | 612 | 1834 | 1855 | 1613227 | GAUUCCAGCAA | 652 |
| | | GCUGGAAUCCU | | | | | AAACCCCGCU | |
| 1613234 | 1613232 | AAGAGCUGGGUG | 613 | 1860 | 1881 | 1613233 | CAAAGACACCA | 653 |
| | | GUGUCUUUGGA | | | | | CCCAGCUCUU | |
| 1613240 | 1613238 | AGCUGCGAUCCC | 614 | 1890 | 1911 | 1613239 | CAAAAUCAGGG | 654 |
| | | CUGAUUUUGGA | | | | | GAUCGCAGCU | |
| 1613264 | 1613262 | AGCUGGUGCUUC | 615 | 2113 | 2134 | 1613263 | UGAGAACCUGA | 655 |
| | | AGGUUCUCAGU | | | | | AGCACCAGCU | |
| 1613276 | 1613274 | AAGCCACACUUG | 616 | 2182 | 2203 | 1613275 | CGUCCAGUCCA | 656 |
| | | GACUGGACGUU | | | | | AGUGUGGCUU | |
| 1613282 | 1613280 | ACUCCCGGGACG | 617 | 2212 | 2233 | 1613281 | UAUCAAACACG | 657 |
| | | UGUUUGAUAUU | | | | | UCCCGGGAGU | |
| 1613285 | 1613283 | ACCUCCCGGGAC | 618 | 2213 | 2234 | 1613284 | AUCAAACACGU | 658 |
| | | GUGUUUGAUAU | | | | | CCCGGGAGGU | |
| 1613294 | 1613292 | AUCACCUUGCUC | 619 | 2260 | 2281 | 1613293 | AGUUGACCUGA | 659 |
| | | AGGUCAACUGG | | | | | GCAAGGUGAU | |
| 1613297 | 1613295 | AAGCCACACUUG | 620 | 2275 | 2296 | 1613296 | GGUGACCUCCA | 660 |
| | | GAGGUCACCUU | | | | | AGUGUGGCUU | |
| 1613303 | 1613301 | ACUCCUGGUUUA | 621 | 2305 | 2326 | 1613302 | CAUCCAUCAUA | 661 |
| | | UGAUGGAUGUU | | | | | AACCAGGAGU | |
| 1613306 | 1613304 | AACCUGGCCACC | 622 | 2315 | 2336 | 1613305 | AAACCAGGAGG | 662 |
| | | UCCUGGUUUAU | | | | | UGGCCAGGUU | |
| 1613309 | 1613307 | AUCAGAUUUUAC | 623 | 2330 | 2351 | 1613308 | CAGGUGGAAGU | 663 |
| | | UUCCACCUGGC | | | | | AAAAUCUGAU | |
| 1613312 | 1613310 | AUUGAAGUCAAG | 624 | 2345 | 2366 | 1613311 | UCUGAGAAGCU | 664 |
| | | CUUCUCAGAUU | | | | | UGACUUCAAU | |
| 1613315 | 1613313 | AGACUGGACUCU | 625 | 2360 | 2381 | 1613314 | UUCAAGGACAG | 665 |
| | | GUCCUUGAAGU | | | | | AGUCCAGUCU | |
| 1613327 | 1613322 | AAGGGACCCAAU | 626 | 2375 | 2396 | 1613323 | CAGUCGAAGAU | 666 |
| | | CUUCGACUGGA | | | | | UGGGUCCCUU | |
| 1613330 | 1613328 | AUGGGUGAUAUU | 627 | 2390 | 2411 | 1613329 | UCCCUGGACAA | 667 |
| | | GUCCAGGGACC | | | | | UAUCACCCAU | |
| 1613333 | 1613331 | ACCAGGGACGUG | 628 | 2399 | 2420 | 1613332 | AAUAUCACCCA | 668 |
| | | GGUGAUAUUGU | | | | | CGUCCCUGGU | |
| 1613336 | 1613334 | AUUUUUAUUUCC | 629 | 2414 | 2435 | 1613335 | CCUGGCGGAGG | 669 |
| | | UCCGCCAGGGA | | | | | AAAUAAAAAU | |
| 1613339 | 1613337 | AUUGUGGGUUUC | 630 | 2429 | 2450 | 1613338 | AAAAAGAUUGA | 670 |
| | | AAUCUUUUUAU | | | | | AACCCACAAU | |
| 1613342 | 1613340 | ACGGAAGGUCAG | 631 | 2441 | 2462 | 1613341 | ACCCACAAGCU | 671 |
| | | CUUGUGGGUUU | | | | | GACCUUCCGU | |
| 1613348 | 1613346 | AUGGUCUGUCUU | 632 | 2468 | 2489 | 1613347 | GCCAAAGCCAA | 672 |
| | | GGCUUUGGCGU | | | | | GACAGACCAU | |
| 1613354 | 1613352 | ACAGACACCACU | 633 | 2506 | 2527 | 1613353 | CAAGUCGCCAG | 673 |
| | | GGCGACUUGUA | | | | | UGGUGUCUGU | |
| 1613360 | 1613358 | AGGUGGAGGAGA | 634 | 2547 | 2568 | 1613359 | UCAGCAAUGUC | 674 |
| | | CAUUGCUGAGA | | | | | UCCUCCACCU | |
| 1613366 | 1613364 | AAGUCUACCAUG | 635 | 2569 | 2590 | 1613365 | CAGCAUCGACA | 675 |
| | | UCGAUGCUGCC | | | | | UGGUAGACUU | |
| 1613375 | 1613373 | AGGCCUGAUCAC | 636 | 2638 | 2659 | 1613374 | GCAGGGUUUGU | 676 |
| | | AAACCCUGCUU | | | | | GAUCAGGCCU | |
| 1613381 | 1613379 | AAUUCUCUCCUC | 637 | 2673 | 2694 | 1613380 | AAUUGUGGAGA | 677 |
| | | UCCACAAUUAU | | | | | GGAGAGAAUU | |
| 1613387 | 1613385 | AUCAUUAUUCUU | 638 | 2703 | 2724 | 1613386 | GGAAAAAAAAA | 678 |
| | | UUUUUUUCCAC | | | | | GAAUAAUGAU | |
| 1613390 | 1613388 | AGGGGGCCGGGU | 639 | 2713 | 2734 | 1613389 | AGAAUAAUGAC | 679 |
| | | CAUUAUUCUUU | | | | | CCGGCCCCCU | |
| 1613402 | 1613400 | AGAGCCAAAGCC | 640 | 2793 | 2814 | 1613401 | UUGUCACUCGG | 680 |
| | | GAGUGACAAAA | | | | | CUUUGGCUCU | |
| 1613405 | 1613403 | AUGAUUUUGAAG | 641 | 2808 | 2829 | 1613404 | GGCUCGGGACU | 681 |
| | | UCCCGAGCCAA | | | | | UCAAAAUCAU | |
| 1613408 | 1613406 | AUCUUACUCCCA | 642 | 2823 | 2844 | 1613407 | AAUCAGUGAUG | 682 |
| | | UCACUGAUUUU | | | | | GGAGUAAGAU | |
| 1613411 | 1613409 | AAAAGAUGAAAU | 643 | 2838 | 2859 | 1613410 | UAAGAGCAAAU | 683 |
| | | UUGCUCUUACU | | | | | UUCAUCUUUU | |
| 1613414 | 1613412 | ACACCCAUCAAU | 644 | 2853 | 2874 | 1613413 | UCUUUCCAAAU | 684 |
| | | UUGGAAAGAUG | | | | | UGAUGGGUGU | |
| 1613423 | 1613421 | ACAUGUUUUUGA | 645 | 2895 | 2916 | 1613422 | AAAAAACAUUC | 685 |
| | | AUGUUUUUUUU | | | | | AAAAACAUGU | |
| 1613432 | 1613430 | AAAAAAAGAAUC | 646 | 2940 | 2961 | 1613431 | AUUCCUUUUGA | 686 |
| | | AAAAGGAAUUG | | | | | UUCUUUUUUU | |
| 1613447 | 1613445 | ACAGUCCCUUGA | 647 | 3011 | 3032 | 1613446 | UGGGGGAUUUC | 687 |
| | | AAUCCCCCAGA | | | | | AAGGGACUGU | |
| 1613456 | 1613454 | AUUUCAAAUCCU | 648 | 3082 | 3103 | 1613455 | CAGCAACAAAG | 688 |
| | | UUGUUGCUGCC | | | | | GAUUUGAAAU | |
| 1613459 | 1613457 | ACACGAACACAC | 649 | 3097 | 3118 | 1613458 | UGAAACUUGGU | 689 |
| | | CAAGUUUCAAA | | | | | GUGUUCGUGU | |
| 1613462 | 1613460 | AUGUGGCUCCAC | 650 | 3105 | 3126 | 1613461 | GGUGUGUUCGU | 690 |
| | | GAACACACCAA | | | | | GGAGCCACAU | |

TABLE 10
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1612982 | 1612980 | AGUUCAAAGUUC | 691 | 125 | 146 | 1612981 | CUAUCAGGUGA | 722 |
| | | ACCUGAUAGUC | | | | | ACUUUGAACU | |
| 1612985 | 1612983 | AGGCUCAGCCAU | 692 | 140 | 161 | 1612984 | UGAACCAGGAU | 723 |
| | | CCUGGUUCAAA | | | | | GGCUGAGCCU | |
| 1612991 | 1612989 | AUCCCAGCGUGA | 693 | 178 | 199 | 1612990 | GAUGGAAGAUC | 724 |
| | | UCUUCCAUCAC | | | | | ACGCUGGGAU | |
| 1612994 | 1612992 | AUACGUCCCAGC | 694 | 182 | 203 | 1612993 | GAAGAUCACGC | 725 |
| | | GUGAUCUUCCA | | | | | UGGGACGUAU | |
| 1613000 | 1612998 | AUGUAGCCCCCC | 695 | 217 | 238 | 1612999 | GAAAGAUCAGG | 726 |
| | | UGAUCUUUCCU | | | | | GGGGCUACAU | |
| 1613009 | 1613007 | AUCCGUGUCACC | 696 | 248 | 269 | 1613008 | GACCAAGAGGG | 727 |
| | | CUCUUGGUCUU | | | | | UGACACGGAU | |
| 1613015 | 1613013 | AGUCUGCAGGGG | 697 | 278 | 299 | 1613014 | AAAGAAUCUCC | 728 |
| | | AGAUUCUUUCA | | | | | CCUGCAGACU | |
| 1613018 | 1613016 | AGGGUCUGCAGG | 698 | 280 | 301 | 1613017 | AGAAUCUCCCCU | 729 |
| | | GGAGAUUCUUU | | | | | GCAGACCCU | |
| 1613024 | 1613022 | AUUUCAGAGCCC | 699 | 316 | 337 | 1613023 | UGAGGAACCGG | 730 |
| | | GGUUCCUCAGA | | | | | GCUCUGAAAU | |
| 1613027 | 1613025 | AUCUUAGCAUCA | 700 | 331 | 352 | 1613026 | UGAAACCUCUG | 731 |
| | | GAGGUUUCAGA | | | | | AUGCUAAGAU | |
| 1613033 | 1613031 | ACUGCUUCUUCA | 701 | 361 | 382 | 1613032 | AGCGGAAGCUG | 732 |
| | | GCUUCCGCUGU | | | | | AAGAAGCAGU | |
| 1613036 | 1613034 | AGGUGUCUCCAA | 702 | 375 | 396 | 1613035 | AAGCAGGCAUU | 733 |
| | | UGCCUGCUUCU | | | | | GGAGACACCU | |
| 1613048 | 1613046 | ACCCGGAACUCU | 703 | 434 | 455 | 1613047 | GAGGAGUUGAG | 734 |
| | | CAACUCCUCUU | | | | | AGUUCCGGGU | |
| 1613054 | 1613052 | AGACGUGGGCGC | 704 | 489 | 510 | 1613053 | AUGAGAUUAGC | 735 |
| | | UAAUCUCAUUG | | | | | GCCCACGUCU | |
| 1613057 | 1613055 | AUGGACGUGGGC | 705 | 491 | 512 | 1613056 | GAGAUUAGCGC | 736 |
| | | GCUAAUCUCAU | | | | | CCACGUCCAU | |
| 1613063 | 1613061 | AACGGGAGCUUC | 706 | 563 | 584 | 1613062 | AAAGAGCCAGA | 737 |
| | | UGGCUCUUUCU | | | | | AGCUCCCGUU | |
| 1613069 | 1613067 | ACUUUUUGUCCC | 707 | 661 | 682 | 1613068 | CCUGGAGUGGG | 738 |
| | | CACUCCAGGGA | | | | | GACAAAAAGU | |
| 1613090 | 1613088 | AUGCAGGUCUCC | 708 | 944 | 965 | 1613089 | CAGCUUCUAGG | 739 |
| | | UAGAAGCUGGU | | | | | AGACCUGCAU | |
| 1613093 | 1613091 | AUCCUGGUGCAG | 709 | 950 | 971 | 1613092 | CUAGGAGACCU | 740 |
| | | GUCUCCUAGAA | | | | | GCACCAGGAU | |
| 1613108 | 1613106 | AUGGAAACUUUG | 710 | 1183 | 1204 | 1613107 | UUUCCUCUCCAA | 741 |
| | | GAGAGGAAAUC | | | | | AGUUUCCAU | |
| 1613111 | 1613109 | AAGGCUGGGAUC | 711 | 1198 | 1219 | 1613110 | UUCCACAGAGA | 742 |
| | | UCUGUGGAAAC | | | | | UCCCAGCCUU | |
| 1613123 | 1613121 | AUUCUGCACGUU | 712 | 1292 | 1313 | 1613122 | AUCACACCCAAC | 743 |
| | | GGGUGUGAUUU | | | | | GUGCAGAAU | |
| 1613132 | 1613130 | AUGGAAAUGCAG | 713 | 1338 | 1359 | 1613131 | UGGGAAGGGCU | 744 |
| | | CCCUUCCCAAA | | | | | GCAUUUCCAU | |
| 1613135 | 1613133 | ACUGGAAAUGCA | 714 | 1339 | 1360 | 1613134 | GGGAAGGGCUG | 745 |
| | | GCCCUUCCCAA | | | | | CAUUUCCAGU | |
| 1613141 | 1613139 | AAAGGUCAGCCU | 715 | 1407 | 1428 | 1613140 | ACACAAAAGAG | 746 |
| | | CUUUUGUGUCC | | | | | GCUGACCUUU | |
| 1613147 | 1613145 | AAGCAGCAGGCU | 716 | 1437 | 1458 | 1613146 | CUGAAAAGCAG | 747 |
| | | GCUUUUCAGAG | | | | | CCUGCUGCUU | |
| 1613150 | 1613148 | AAGCAGCAGCAG | 717 | 1440 | 1461 | 1613149 | AAAAGCAGCCU | 748 |
| | | GCUGCUUUUCA | | | | | GCUGCUGCUU | |
| 1613159 | 1613157 | ACCGUCUUUGCU | 718 | 1508 | 1529 | 1613158 | GUCAGUAAAAG | 749 |
| | | UUUACUGACCA | | | | | CAAAGACGGU | |
| 1613162 | 1613160 | AUCAUCGCUUCC | 719 | 1523 | 1544 | 1613161 | GACGGGACUGG | 750 |
| | | AGUCCCGUCUU | | | | | AAGCGAUGAU | |
| 1613207 | 1613205 | ACAGUUCGGGAA | 720 | 1702 | 1723 | 1613206 | UUCUGUCACUU | 751 |
| | | GUGACAGAAGA | | | | | CCCGAACUGU | |
| 1613219 | 1613217 | AUGGCGAUCUUC | 721 | 1762 | 1783 | 1613218 | UGGUAAAACGA | 752 |
| | | GUUUUACCAUC | | | | | AGAUCGCCAU | |

### Example 2: Effect of RNAi Compounds on Human MAPT RNA In Vitro, Single Dose

RNAi compounds described herein above were tested for their single dose effects on MAPT RNA in vitro in cultured cells that express MAPT. The RNAi compounds were tested in a series of experiments that had the same culture conditions.

Cultured A-172 cells were treated with RNAi compound at a concentration of 15 nM by LipofectAMINE 2000, at a density of 30,000 cells per well. After a treatment period of 24 hours, total RNA was isolated from the cells and MAPT RNA levels were measured by quantitative real-time RTPCR. MAPT RNA was measured by human primer probe set RTS3104 (forward sequence AAGATTGGGTCCCTGGACAAT, designated herein as SEQ ID NO: 3; reverse sequence AGCTTGTGGGTTTCAATCTTTTTATT, designated herein as SEQ ID NO: 4; probe sequence CACCCACGTCCCTGGCGGA, designated herein as SEQ ID NO: 5). MAPT RNA levels were normalized to total RNA content, as measured by RIBOGREENK® Reduction of MAPT RNA is presented in the table below as percent MAPT RNA relative to the amount in untreated control cells (% UTC).

Each separate experiment described in this example is presented in a separate table. The values marked with a “T” indicate that the RNAi compound is complementary to the amplicon region of the primer probe set.

TABLE 11
Reduction of MAPT RNA by RNAi compounds
that target human MAPT nucleic acid

| Compound | MAPT |
|---|---|
| Number | (% UTC) |
| 1612982 | 14 |
| 1612982 | 17 |
| 1612985 | 86 |
| 1612988 | 68 |
| 1612991 | 122 |
| 1612994 | 30 |
| 1612997 | 118 |
| 1613000 | 129 |
| 1613006 | 84 |
| 1613009 | 22 |
| 1613015 | 120 |
| 1613018 | 89 |
| 1613024 | 98 |
| 1613030 | 113 |
| 1613036 | 72 |
| 1613051 | 135 |
| 1613090 | 110 |
| 1613108 | 90 |
| 1613114 | 112 |
| 1613162 | 106 |
| 1613219 | 14 |
| 1613243 | 96 |
| 1613246 | 91 |
| 1613264 | 116 |
| 1613270 | 102 |
| 1613291 | 117 |
| 1613294 | 82 |
| 1613330 | 118† |
| 1613348 | 106 |
| 1613375 | 103 |
| 1613387 | 121 |
| 1613393 | 116 |
| 1613417 | 94 |
| 1613435 | 125 |
| 1613438 | 94 |
| 1613456 | 109 |
| 1613465 | 72 |
| 1613468 | 61 |
| 1613474 | 120 |
| 1613477 | 112 |
| 1613483 | 126 |
| 1613504 | 60 |
| 1613525 | 95 |
| 1613549 | 78 |
| 1613573 | 79 |
| 1613585 | 123 |
| 1613606 | 98 |
| 1613618 | 94 |
| 1613633 | 92 |
| 1613642 | 97 |
| 1613645 | 80 |
| 1613651 | 114 |
| 1613681 | 78 |
| 1613702 | 120 |
| 1613708 | 107 |
| 1613723 | 72 |
| 1613729 | 113 |
| 1613732 | 104 |
| 1613741 | 105 |
| 1613777 | 109 |
| 1613780 | 109 |
| 1613840 | 110 |
| 1613864 | 121 |
| 1613867 | 127 |
| 1613873 | 93 |
| 1613924 | 123 |
| 1613927 | 108 |
| 1613942 | 111 |
| 1613963 | 112 |
| 1613972 | 106 |
| 1613978 | 100 |
| 1613990 | 101 |
| 1614005 | 95 |
| 1614017 | 114 |
| 1614020 | 118 |
| 1614038 | 94 |
| 1614077 | 108 |
| 1614143 | 89 |

TABLE 12
Reduction of MAPT RNA by RNAi compounds
that target human MAPT nucleic acid

| Compound | MAPT |
|---|---|
| Number | (% UTC) |
| 1612979 | 52 |
| 1612982 | 13 |
| 1613003 | 40 |
| 1613009 | 17 |
| 1613012 | 104 |
| 1613021 | 76 |
| 1613039 | 53 |
| 1613042 | 96 |
| 1613066 | 109 |
| 1613072 | 80 |
| 1613078 | 111 |
| 1613096 | 83 |
| 1613120 | 73 |
| 1613126 | 91 |
| 1613129 | 107 |
| 1613138 | 104 |
| 1613144 | 83 |
| 1613153 | 110 |
| 1613165 | 50 |
| 1613171 | 91 |
| 1613183 | 105 |
| 1613192 | 88 |
| 1613195 | 96 |
| 1613198 | 110 |
| 1613201 | 128 |
| 1613210 | 110 |
| 1613213 | 95 |
| 1613216 | 95 |
| 1613225 | 84 |
| 1613231 | 94 |
| 1613255 | 100 |
| 1613261 | 103 |
| 1613273 | 82 |
| 1613279 | 109 |
| 1613300 | 83 |
| 1613345 | 115† |
| 1613351 | 105 |
| 1613357 | 105 |
| 1613369 | 99 |
| 1613384 | 107 |
| 1613399 | 86 |
| 1613420 | 106 |
| 1613450 | 95 |
| 1613453 | 119 |
| 1613492 | 89 |
| 1613513 | 105 |
| 1613519 | 73 |
| 1613576 | 86 |
| 1613594 | 88 |
| 1613630 | 99 |
| 1613636 | 79 |
| 1613639 | 95 |
| 1613648 | 118 |
| 1613660 | 88 |
| 1613687 | 52 |
| 1613690 | 51 |
| 1613714 | 77 |
| 1613726 | 101 |
| 1613750 | 95 |
| 1613753 | 88 |
| 1613762 | 104 |
| 1613771 | 71 |
| 1613795 | 85 |
| 1613804 | 112 |
| 1613807 | 96 |
| 1613813 | 113 |
| 1613816 | 100 |
| 1613834 | 91 |
| 1613846 | 78 |
| 1613876 | 85 |
| 1613912 | 102 |
| 1613918 | 110 |
| 1613966 | 72 |
| 1613996 | 81 |
| 1613999 | 89 |
| 1614035 | 99 |
| 1614149 | 104 |
| 1614173 | 98 |
| 1614191 | 107 |

TABLE 13
Reduction of MAPT RNA by RNAi compounds
that target human MAPT nucleic acid

| | MAPT |
|---|---|
| Compound | (% |
| Number | UTC) |
| 1612982 | 14 |
| 1613009 | 18 |
| 1613045 | 83 |
| 1613060 | 92 |
| 1613075 | 98 |
| 1613081 | 102 |
| 1613084 | 105 |
| 1613087 | 100 |
| 1613102 | 99 |
| 1613105 | 83 |
| 1613117 | 94 |
| 1613156 | 56 |
| 1613168 | 93 |
| 1613174 | 95 |
| 1613189 | 111 |
| 1613204 | 102 |
| 1613249 | 110 |
| 1613252 | 84 |
| 1613288 | 97 |
| 1613378 | 108 |
| 1613396 | 84 |
| 1613426 | 106 |
| 1613429 | 109 |
| 1613441 | 90 |
| 1613444 | 115 |
| 1613486 | 113 |
| 1613495 | 62 |
| 1613501 | 80 |
| 1613510 | 127 |
| 1613522 | 65 |
| 1613531 | 58 |
| 1613540 | 93 |
| 1613543 | 102 |
| 1613546 | 103 |
| 1613558 | 94 |
| 1613564 | 99 |
| 1613588 | 125 |
| 1613597 | 51 |
| 1613600 | 67 |
| 1613612 | 88 |
| 1613624 | 103 |
| 1613654 | 99 |
| 1613663 | 68 |
| 1613675 | 70 |
| 1613678 | 54 |
| 1613699 | 51 |
| 1613705 | 91 |
| 1613717 | 92 |
| 1613735 | 81 |
| 1613738 | 91 |
| 1613744 | 105 |
| 1613747 | 84 |
| 1613759 | 103 |
| 1613822 | 91 |
| 1613831 | 92 |
| 1613837 | 94 |
| 1613855 | 97 |
| 1613858 | 93 |
| 1613870 | 97 |
| 1613879 | 99 |
| 1613897 | 77 |
| 1613945 | 78 |
| 1613951 | 93 |
| 1613975 | 96 |
| 1613987 | 102 |
| 1614023 | 105 |
| 1614026 | 94 |
| 1614140 | 95 |
| 1614158 | 115 |
| 1614164 | 118 |
| 1614176 | 105 |
| 1614182 | 117 |
| 1614188 | 103 |
| 1614194 | 94 |

TABLE 14
Reduction of MAPT RNA by RNAi compounds
that target human MAPT nucleic acid

| | MAPT |
|---|---|
| Compound | (% |
| Number | UTC) |
| 1612982 | 14 |
| 1613009 | 18 |
| 1613033 | 78 |
| 1613069 | 78 |
| 1613099 | 96 |
| 1613135 | 92 |
| 1613147 | 106 |
| 1613159 | 40 |
| 1613207 | 99 |
| 1613222 | 99 |
| 1613234 | 92 |
| 1613237 | 79 |
| 1613240 | 115 |
| 1613258 | 108 |
| 1613267 | 86 |
| 1613282 | 104 |
| 1613303 | 97 |
| 1613306 | 93 |
| 1613309 | 101 |
| 1613315 | 115 |
| 1613327 | 112† |
| 1613354 | 89 |
| 1613360 | 103 |
| 1613363 | 98 |
| 1613372 | 98 |
| 1613381 | 100 |
| 1613390 | 102 |
| 1613402 | 130 |
| 1613414 | 78 |
| 1613423 | 80 |
| 1613447 | 97 |
| 1613459 | 104 |
| 1613471 | 104 |
| 1613516 | 61 |
| 1613528 | 88 |
| 1613552 | 74 |
| 1613555 | 81 |
| 1613567 | 83 |
| 1613582 | 110 |
| 1613591 | 102 |
| 1613609 | 112 |
| 1613615 | 65 |
| 1613666 | 92 |
| 1613669 | 56 |
| 1613684 | 58 |
| 1613696 | 54 |
| 1613756 | 75 |
| 1613768 | 81 |
| 1613786 | 73 |
| 1613789 | 113 |
| 1613825 | 100 |
| 1613843 | 82 |
| 1613861 | 90 |
| 1613891 | 101 |
| 1613900 | 89 |
| 1613903 | 102 |
| 1613930 | 88 |
| 1613933 | 71 |
| 1613936 | 112 |
| 1613948 | 101 |
| 1613954 | 113 |
| 1613957 | 89 |
| 1613960 | 105 |
| 1613969 | 91 |
| 1613981 | 88 |
| 1613993 | 89 |
| 1614029 | 98 |
| 1614032 | 98 |
| 1614110 | 73 |
| 1614131 | 67 |
| 1614137 | 85 |
| 1614146 | 94 |
| 1614152 | 97 |
| 1614155 | 93 |
| 1614167 | 79 |
| 1614170 | 105 |
| 1614185 | 91 |

TABLE 15
Reduction of MAPT RNA by RNAi compounds
that target human MAPT nucleic acid

| Compound | MAPT |
|---|---|
| Number | (% UTC) |
| 1612982 | 12 |
| 1613009 | 16 |
| 1613027 | 72 |
| 1613048 | 80 |
| 1613054 | 90 |
| 1613057 | 104 |
| 1613063 | 121 |
| 1613093 | 106 |
| 1613111 | 97 |
| 1613123 | 134 |
| 1613132 | 110 |
| 1613141 | 117 |
| 1613150 | 136 |
| 1613228 | 107 |
| 1613276 | 94 |
| 1613285 | 124 |
| 1613297 | 102 |
| 1613312 | 128 |
| 1613333 | 117† |
| 1613336 | 100† |
| 1613339 | 117† |
| 1613342 | 109† |
| 1613366 | 107 |
| 1613405 | 87 |
| 1613408 | 104 |
| 1613411 | 91 |
| 1613432 | 93 |
| 1613462 | 111 |
| 1613480 | 110 |
| 1613489 | 108 |
| 1613498 | 101 |
| 1613507 | 62 |
| 1613534 | 87 |
| 1613537 | 115 |
| 1613561 | 102 |
| 1613570 | 85 |
| 1613579 | 75 |
| 1613603 | 106 |
| 1613621 | 119 |
| 1613627 | 86 |
| 1613657 | 107 |
| 1613672 | 118 |
| 1613693 | 63 |
| 1613711 | 96 |
| 1613720 | 125 |
| 1613765 | 96 |
| 1613774 | 89 |
| 1613783 | 120 |
| 1613792 | 122 |
| 1613798 | 108 |
| 1613801 | 116 |
| 1613810 | 99 |
| 1613819 | 121 |
| 1613828 | 101 |
| 1613849 | 136 |
| 1613852 | 125 |
| 1613894 | 88 |
| 1613906 | 101 |
| 1613909 | 65 |
| 1613915 | 138 |
| 1613921 | 106 |
| 1613939 | 71 |
| 1613984 | 110 |
| 1614002 | 87 |
| 1614008 | 106 |
| 1614011 | 120 |
| 1614014 | 98 |
| 1614041 | 76 |
| 1614054 | 152 |
| 1614134 | 94 |
| 1614161 | 110 |
| 1614179 | 99 |

### Example 3: Dose-Dependent Inhibition of Human MAPT in A-172 Cells by RNAi Compounds

RNAi compounds selected from the example above were tested at various doses in A-172 cells. A-172 cells plated at a density of 30,000 cells per well were treated using LipofectAMINE 2000 with various concentrations of RNAi compounds as specified in the tables below. After a treatment period of approximately 24 hours, total RNA was isolated from the cells and MAPT RNA levels were measured by quantitative real-time RTPCR. Human MAPT primer-probe set RTS3104 (described herein above) was used to measure RNA levels as described above. MAPT RNA levels were normalized to total RNA content, as measured by RIBOGREEN®. Reduction of MAPT RNA is presented in the table below as percent MAPT RNA, relative to untreated control cells (% UTC).

The half maximal inhibitory concentration (IC₅₀) of each RNAi compound was calculated using a linear regression on a log/linear plot of the data in Excel or GraphPad Prism (version 9.2.0) and is also presented in the table below. Compound No. 623782, a 5-10-5 MOE gapmer modified oligonucleotide previously described in WO 2015/010135, was added as a positive control.

TABLE 16
Dose-dependent reduction of human MAPT
RNA in A-172 cells by RNAi compounds

| Compound | MAPT RNA (% UTC) | | | | | IC₅₀ |
|---|---|---|---|---|---|---|
| Number | 0.008 nM | 0.04 nM | 0.2 nM | 1 nM | 5 nM | (nM) |
| 623782 | 85 | 59 | 29 | 10 | 5 | 0.07 |
| 1613156 | 99 | 100 | 94 | 72 | 57 | >5 |
| 1613495 | 97 | 83 | 96 | 85 | 83 | >5 |
| 1613501 | 100 | 94 | 87 | 78 | 49 | 5.11 |
| 1613522 | 95 | 65 | 57 | 58 | 64 | >5 |
| 1613531 | 102 | 83 | 101 | 98 | 80 | >5 |
| 1613597 | 101 | 77 | 62 | 65 | 67 | >5 |
| 1613600 | 104 | 96 | 83 | 66 | 65 | >5 |
| 1613663 | 94 | 91 | 77 | 60 | 55 | >5 |
| 1613675 | 92 | 94 | 90 | 73 | 68 | >5 |
| 1613678 | 91 | 74 | 62 | 56 | 69 | >5 |
| 1613699 | 83 | 62 | 61 | 65 | 66 | >5 |
| 1613897 | 105 | 100 | 103 | 104 | 92 | >5 |
| 1613945 | 96 | 94 | 94 | 97 | 93 | >5 |

TABLE 17
Dose-dependent reduction of human MAPT
RNA in A-172 cells by RNAi compounds

| Compound | MAPT RNA (% UTC) | | | | | IC₅₀ |
|---|---|---|---|---|---|---|
| Number | 0.008 nM | 0.04 nM | 0.2 nM | 1 nM | 5 nM | (nM) |
| 623782 | 90 | 67 | 28 | 10 | 5 | 0.08 |
| 1613159 | 113 | 110 | 106 | 69 | 57 | >5 |
| 1613423 | 103 | 102 | 101 | 105 | 103 | >5 |
| 1613516 | 96 | 84 | 66 | 65 | 72 | >5 |
| 1613552 | 96 | 87 | 82 | 68 | 62 | >5 |
| 1613615 | 92 | 79 | 60 | 60 | 73 | >5 |
| 1613669 | 96 | 71 | 56 | 51 | 67 | >5 |
| 1613684 | 98 | 91 | 73 | 67 | 72 | >5 |
| 1613696 | 81 | 58 | 50 | 55 | 59 | 8.36 |
| 1613756 | 103 | 101 | 105 | 105 | 94 | >5 |
| 1613786 | 96 | 96 | 103 | 100 | 91 | >5 |
| 1613933 | 111 | 115 | 106 | 101 | 97 | >5 |
| 1614110 | 106 | 107 | 108 | 110 | 110 | >5 |
| 1614131 | 101 | 104 | 102 | 100 | 97 | >5 |
| 1614167 | 96 | 101 | 104 | 102 | 101 | >5 |

TABLE 18
Dose-dependent reduction of human MAPT
RNA in A-172 cells by RNAi compounds

| Compound | MAPT RNA (% UTC) | | | | | IC₅₀ |
|---|---|---|---|---|---|---|
| Number | 0.008 nM | 0.04 nM | 0.2 nM | 1 nM | 5 nM | (nM) |
| 623782 | 91 | 64 | 26 | 9 | 6 | 0.07 |
| 1613027 | 95 | 88 | 71 | 64 | 62 | >5 |
| 1613048 | 123 | 102 | 90 | 84 | 78 | >5 |
| 1613405 | 100 | 104 | 106 | 101 | 89 | >5 |
| 1613507 | 103 | 85 | 72 | 63 | 66 | >5 |
| 1613534 | 97 | 100 | 96 | 74 | 76 | >5 |
| 1613570 | 99 | 92 | 104 | 88 | 82 | >5 |
| 1613579 | 99 | 92 | 73 | 65 | 65 | >5 |
| 1613627 | 101 | 98 | 96 | 94 | 88 | >5 |
| 1613693 | 104 | 99 | 97 | 85 | 75 | >5 |
| 1613894 | 97 | 101 | 101 | 92 | 72 | >5 |
| 1613909 | 107 | 97 | 107 | 113 | 110 | >5 |
| 1613939 | 115 | 96 | 87 | 79 | 84 | >5 |
| 1614041 | 105 | 101 | 111 | 115 | 128 | >5 |

TABLE 19
Dose-dependent reduction of human MAPT
RNA in A-172 cells by RNAi compounds

| Compound | MAPT RNA (% UTC) | | | | | IC₅₀ |
|---|---|---|---|---|---|---|
| Number | 0.008 nM | 0.04 nM | 0.2 nM | 1 nM | 5 nM | (nM) |
| 623782 | 88 | 64 | 29 | 9 | 6 | 0.08 |
| 1612982 | 95 | 74 | 40 | 24 | 26 | 0.19 |
| 1612988 | 94 | 89 | 81 | 53 | 32 | 1.42 |
| 1612994 | 84 | 50 | 29 | 19 | 24 | 0.06 |
| 1613009 | 94 | 73 | 40 | 25 | 29 | 0.20 |
| 1613036 | 99 | 81 | 72 | 53 | 45 | 2.07 |
| 1613219 | 81 | 46 | 24 | 23 | 22 | 0.05 |
| 1613465 | 93 | 96 | 94 | 73 | 56 | >5 |
| 1613468 | 94 | 92 | 71 | 61 | 63 | >5 |
| 1613504 | 96 | 79 | 62 | 53 | 63 | >5 |
| 1613549 | 99 | 90 | 91 | 73 | 68 | >5 |
| 1613573 | 99 | 97 | 83 | 65 | 67 | >5 |
| 1613645 | 94 | 96 | 97 | 95 | 92 | >5 |
| 1613681 | 99 | 101 | 102 | 98 | 79 | >5 |
| 1613723 | 108 | 110 | 115 | 117 | 125 | >5 |

TABLE 20
Dose-dependent reduction of human MAPT
RNA in A-172 cells by RNAi compounds

| Compound | MAPT RNA (% UTC) | | | | | IC₅₀ |
|---|---|---|---|---|---|---|
| Number | 0.008 nM | 0.04 nM | 0.2 nM | 1 nM | 5 nM | (nM) |
| 623782 | 85 | 56 | 29 | 10 | 6 | 0.06 |
| 1612979 | 114 | 105 | 84 | 47 | 39 | 1.61 |
| 1613003 | 115 | 102 | 71 | 45 | 36 | 1.18 |
| 1613021 | 103 | 107 | 104 | 75 | 58 | >5 |
| 1613039 | 98 | 102 | 96 | 57 | 39 | 2.26 |
| 1613120 | 108 | 92 | 85 | 71 | 77 | >5 |
| 1613165 | 115 | 104 | 101 | 76 | 43 | 3.63 |
| 1613519 | 100 | 98 | 81 | 64 | 60 | >5 |
| 1613636 | 97 | 100 | 96 | 87 | 78 | >5 |
| 1613687 | 103 | 75 | 58 | 59 | 57 | >5 |
| 1613690 | 86 | 64 | 52 | 50 | 56 | 2.64 |
| 1613714 | 98 | 105 | 117 | 105 | 111 | >5 |
| 1613771 | 104 | 106 | 104 | 112 | 117 | >5 |
| 1613846 | 97 | 110 | 105 | 95 | 107 | >5 |
| 1613966 | 109 | 102 | 105 | 111 | 106 | >5 |

### Example 4: Design of RNAi Compounds with Antisense RNAi Oligonucleotides Complementary to a Human MAPT Nucleic Acid

RNAi compounds comprising antisense RNAi oligonucleotides complementary to a human MAPT nucleic acid, and sense RNAi oligonucleotides complementary to the antisense RNAi oligonucleotides were designed as follows.

The RNAi compounds in the tables below consist of an antisense RNAi oligonucleotide and a sense RNAi oligonucleotide. In each case, the antisense RNAi oligonucleotide is 23 nucleosides in length; has a sugar motif (from 5′ to 3′) of: yfyyyfyyyyyyyfyfyyyyyyy, wherein each “y” represents a 2′-O-methylribosyl sugar, and each “f” represents a 2′-fluororibosyl sugar; and has an internucleoside linkage motif (from 5′ to 3′) of: ssooooooooooooooooooss, wherein each “o” represents a phosphodiester internucleoside linkage, and each “s” represents a phosphorothioate internucleoside linkage. Each antisense RNAi oligonucleotide has a terminal phosphate at the 5′ end. The sense RNAi oligonucleotide in each case is 21 nucleosides in length; has a sugar motif (from 5′ to 3′) of yyyyyyfyfffyyyyyyyyyy, wherein each “y” represents a 2′-O-methylribosyl sugar, and each “f” represents a 2′-fluororibosyl sugar; and has an internucleoside linkage motif (from 5′ to 3′) of ssooooooooooooooooss, wherein each “o” represents a phosphodiester internucleoside linkage, and each “s” represents a phosphorothioate internucleoside linkage. Each antisense RNAi oligonucleotide in the table below is complementary to the target nucleic acid (MAPT), and each sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

“Start site” indicates the 5′-most nucleoside to which the antisense RNAi oligonucleotide is complementary target nucleic acid sequence. “Stop site” indicates the 3′-most nucleoside to which the antisense RNAi oligonucleotide is complementary in the target nucleic acid sequence. Each antisense RNAi oligonucleotide listed in the tables below is 100% complementary to SEQ ID NO: 1 (described herein above).

TABLE 21
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1702348 | 1702346 | UUCAAAGUUCAC | 753 | 123 | 145 | 1702347 | GACUAUCAGGU | 899 |
| | | CUGAUAGUCGA | | | | | GAACUUUGAA | |
| 1702366 | 1702364 | UGGCGGGGCUCA | 754 | 145 | 167 | 1702365 | CAGGAUGGCUG | 900 |
| | | GCCAUCCUGGU | | | | | AGCCCCGCCA | |
| 1702375 | 1702373 | UCACUUCGAACU | 755 | 159 | 181 | 1702374 | CCCGCCAGGAGU | 901 |
| | | CCUGGCGGGGC | | | | | UCGAAGUGA | |
| 1702381 | 1702379 | UCCAUCACUUCG | 330 | 163 | 185 | 1702380 | CCAGGAGUUCG | 377 |
| | | AACUCCUGGCG | | | | | AAGUGAUGGA | |
| 1702423 | 1702421 | UUUCAGGCCAGC | 756 | 260 | 282 | 1702422 | GACACGGACGC | 902 |
| | | GUCCGUGUCAC | | | | | UGGCCUGAAA | |
| 1702441 | 1702439 | UGGAGUGCUCUU | 757 | 338 | 360 | 1702440 | UCUGAUGCUAA | 903 |
| | | AGCAUCAGAGG | | | | | GAGCACUCCA | |
| 1702444 | 1702442 | UUCAGCUUCCGC | 758 | 353 | 375 | 1702443 | ACUCCAACAGCG | 904 |
| | | UGUUGGAGUGC | | | | | GAAGCUGAA | |
| 1702447 | 1702445 | UCCAAUGCCUGC | 759 | 368 | 390 | 1702446 | GCUGAAGAAGC | 905 |
| | | UUCUUCAGCUU | | | | | AGGCAUUGGA | |
| 1702453 | 1702451 | UCACGUGACCAG | 760 | 408 | 430 | 1702452 | ACGAAGCUGCU | 906 |
| | | CAGCUUCGUCU | | | | | GGUCACGUGA | |
| 1702462 | 1702460 | UCAGGCGCCUUC | 761 | 454 | 476 | 1702461 | CCGGCAGAGGA | 907 |
| | | CUCUGCCGGCC | | | | | AGGCGCCUGA | |
| 1702471 | 1702469 | UGGACGUGGGCG | 762 | 490 | 512 | 1702470 | UGAGAUUAGCG | 908 |
| | | CUAAUCUCAUU | | | | | CCCACGUCCA | |
| 1702501 | 1702499 | UCAGGCAGGAGG | 763 | 835 | 857 | 1702500 | UGGGGCUCCCCU | 909 |
| | | GGAGCCCCAGG | | | | | CCUGCCUGA | |
| 1702522 | 1702520 | UCAUCGACGUCG | 764 | 1030 | 1052 | 1702521 | UGAAGACCGCG | 910 |
| | | CGGUCUUCAUC | | | | | ACGUCGAUGA | |
| 1702528 | 1702526 | UUUGGAGAGGAA | 765 | 1175 | 1197 | 1702527 | CCUGUGGAUUU | 911 |
| | | AUCCACAGGGA | | | | | CCUCUCCAAA | |
| 1702534 | 1702532 | UGAGGCUGGGAU | 766 | 1199 | 1221 | 1702533 | UCCACAGAGAU | 912 |
| | | CUCUGUGGAAA | | | | | CCCAGCCUCA | |
| 1702537 | 1702535 | UUGGCCCGCCCU | 767 | 1231 | 1253 | 1702536 | GCCCAGUGUAG | 913 |
| | | ACACUGGGCCC | | | | | GGCGGGCCAA | |
| 1702546 | 1702544 | UCCUUCUGCACG | 768 | 1294 | 1316 | 1702545 | CACACCCAACGU | 914 |
| | | UUGGGUGUGAU | | | | | GCAGAAGGA | |
| 1702570 | 1702568 | UGCGAGCUUUGA | 769 | 1485 | 1507 | 1702569 | UCCCUCAACUCA | 915 |
| | | GUUGAGGGACC | | | | | AAGCUCGCA | |
| 1702573 | 1702571 | UGCUUUUACUGA | 770 | 1500 | 1522 | 1702572 | CUCGCAUGGUC | 916 |
| | | CCAUGCGAGCU | | | | | AGUAAAAGCA | |
| 1702576 | 1702574 | UUCCAGUCCCGU | 771 | 1515 | 1537 | 1702575 | AAAGCAAAGAC | 917 |
| | | CUUUGCUUUUA | | | | | GGGACUGGAA | |
| 1702585 | 1702583 | UUUUAGCAGAGG | 772 | 1560 | 1582 | 1702584 | CCACACGUUCCU | 918 |
| | | AACGUGUGGAU | | | | | CUGCUAAAA | |
| 1702594 | 1702592 | UGGGGUGUUUGG | 773 | 1599 | 1621 | 1702593 | GCCUUAGCCCCA | 919 |
| | | GGCUAAGGCAA | | | | | AACACCCCA | |
| 1702597 | 1702595 | UACCAGGAGUGG | 774 | 1608 | 1630 | 1702596 | CCAAACACCCCA | 920 |
| | | GGUGUUUGGGG | | | | | CUCCUGGUA | |
| 1702651 | 1702649 | UCCCCUGAUUUU | 775 | 1882 | 1904 | 1702650 | UGAACCUCCAA | 921 |
| | | GGAGGUUCACC | | | | | AAUCAGGGGA | |
| 1702678 | 1702676 | UUCCCGCCUCCCG | 776 | 2125 | 2147 | 1702677 | GCACCAGCCGGG | 922 |
| | | GCUGGUGCUU | | | | | AGGCGGGAA | |
| 1702699 | 1702697 | UUUGUAGACUAU | 777 | 2237 | 2259 | 1702698 | AGUGUGCAAAU | 923 |
| | | UUGCACACUGC | | | | | AGUCUACAAA | |
| 1702711 | 1702709 | UUUAUGAUGGAU | 778 | 2297 | 2319 | 1702710 | UUAGGCAACAU | 924 |
| | | GUUGCCUAAUG | | | | | CCAUCAUAAA | |
| 1702717 | 1702715 | UUACUUCCACCU | 779 | 2322 | 2344 | 1702716 | GAGGUGGCCAG | 925 |
| | | GGCCACCUCCU | | | | | GUGGAAGUAA | |
| 1702729 | 1702727 | UAUUGUCCAGGG | 780 | 2382 | 2404 | 1702728 | AGAUUGGGUCC | 926 |
| | | ACCCAAUCUUC | | | | | CUGGACAAUA | |
| 1702735 | 1702733 | UUCCUCCGCCAG | 781 | 2406 | 2428 | 1702734 | CCCACGUCCCUG | 927 |
| | | GGACGUGGGUG | | | | | GCGGAGGAA | |
| 1702738 | 1702736 | UUUCAAUCUUUU | 782 | 2421 | 2443 | 1702737 | GAGGAAAUAAA | 928 |
| | | UAUUUCCUCCG | | | | | AAGAUUGAAA | |
| 1702747 | 1702745 | UCUUGGCUUUGG | 783 | 2460 | 2482 | 1702746 | GCGAGAACGCC | 929 |
| | | CGUUCUCGCGG | | | | | AAAGCCAAGA | |
| 1702756 | 1702754 | UGGAGACGUGUC | 784 | 2519 | 2541 | 1702755 | GUGUCUGGGGA | 930 |
| | | CCCAGACACCA | | | | | CACGUCUCCA | |
| 1702762 | 1702760 | UGCCGGUGGAGG | 785 | 2550 | 2572 | 1702761 | GCAAUGUCUCC | 931 |
| | | AGACAUUGCUG | | | | | UCCACCGGCA | |
| 1702777 | 1702775 | UUGGCCAGGGAG | 786 | 2617 | 2639 | 1702776 | GGUGUCUGCCU | 932 |
| | | GCAGACACCUC | | | | | CCCUGGCCAA | |
| 1702792 | 1702790 | UCUUUUUUUUUC | 787 | 2695 | 2717 | 1702791 | GAGAGUGUGGA | 933 |
| | | CACACUCUCUC | | | | | AAAAAAAAGA | |
| 1702822 | 1702820 | UACUAGCCCACCC | 788 | 2860 | 2882 | 1702821 | AAAUUGAUGGG | 934 |
| | | AUCAAUUUGG | | | | | UGGGCUAGUA | |
| 1702861 | 1702859 | UCCUUUGUUGCU | 789 | 3074 | 3096 | 1702860 | GGCAGUGGCAG | 935 |
| | | GCCACUGCCUC | | | | | CAACAAAGGA | |
| 1702891 | 1702889 | UUCUCUUUUUAC | 790 | 3410 | 3432 | 1702890 | GGACAGCGGGU | 936 |
| | | CCGCUGUCCCU | | | | | AAAAAGAGAA | |
| 1702894 | 1702892 | UGCCAGCUUGCC | 791 | 3422 | 3444 | 1702893 | AAAAGAGAAGG | 937 |
| | | UUCUCUUUUUA | | | | | CAAGCUGGCA | |
| 1702900 | 1702898 | UUUUCUAAGGAG | 792 | 3458 | 3480 | 1702899 | GUGGAUGACCU | 938 |
| | | GUCAUCCACGA | | | | | CCUUAGAAAA | |
| 1702909 | 1702907 | UCAGGCCCCCUAC | 793 | 3522 | 3544 | 1702908 | CCUGCAGGGUA | 939 |
| | | CCUGCAGGGA | | | | | GGGGGCCUGA | |
| 1702915 | 1702913 | UUCAGAACUCAA | 794 | 3574 | 3596 | 1702914 | CCUGUUUUAUU | 940 |
| | | UAAAACAGGGU | | | | | GAGUUCUGAA | |
| 1702924 | 1702922 | UCCCAGGUCUGC | 795 | 3619 | 3641 | 1702923 | GGCCACUUUGC | 941 |
| | | AAAGUGGCCAA | | | | | AGACCUGGGA | |
| 1702927 | 1702925 | UGGUUAGCCCUA | 796 | 3634 | 3656 | 1702926 | CUGGGACUUUA | 942 |
| | | AAGUCCCAGGU | | | | | GGGCUAACCA | |
| 1702954 | 1702952 | UGGGCUCUCACG | 797 | 3813 | 3835 | 1702953 | GUUGUCUGCCG | 943 |
| | | GCAGACAACAG | | | | | UGAGAGCCCA | |
| 1702957 | 1702955 | UAUAGGCAGUGA | 798 | 3826 | 3848 | 1702956 | AGAGCCCAAUC | 944 |
| | | UUGGGCUCUCA | | | | | ACUGCCUAUA | |
| 1702966 | 1702964 | UGGUGAGGCUGG | 799 | 3870 | 3892 | 1702965 | CCCGAAUUCCCA | 945 |
| | | GAAUUCGGGAC | | | | | GCCUCACCA | |
| 1702969 | 1702967 | UACUGAGAAGGG | 800 | 3883 | 3905 | 1702968 | CCUCACCACCCC | 946 |
| | | GUGGUGAGGCU | | | | | UUCUCAGUA | |
| 1702975 | 1702973 | UAGGUACCUCCU | 801 | 3913 | 3935 | 1702974 | GUUGGUUGCAG | 947 |
| | | GCAACCAACCA | | | | | GAGGUACCUA | |
| 1702984 | 1702982 | UGUGCCUGGACU | 802 | 3958 | 3980 | 1702983 | GGAAGGCAAAG | 948 |
| | | UUGCCUUCCCU | | | | | UCCAGGCACA | |
| 1702987 | 1702985 | UCCCACUCUUGU | 803 | 3967 | 3989 | 1702986 | AGUCCAGGCAC | 949 |
| | | GCCUGGACUUU | | | | | AAGAGUGGGA | |
| 1702993 | 1702991 | UGAGUGGAACUG | 804 | 3996 | 4018 | 1702992 | UCUCACUCUCAG | 950 |
| | | AGAGUGAGAGG | | | | | UUCCACUCA | |
| 1703020 | 1703018 | UCCAUGUCAACA | 805 | 4123 | 4145 | 1703019 | UUUCUGCCUUG | 951 |
| | | AGGCAGAAACA | | | | | UUGACAUGGA | |
| 1703026 | 1703024 | UGGGCUCAGCAC | 806 | 4172 | 4194 | 1703025 | CCCCUUCCUGUG | 952 |
| | | AGGAAGGGGCC | | | | | CUGAGCCCA | |
| 1703038 | 1703036 | UGGCAGCUACAA | 807 | 4289 | 4311 | 1703037 | CACCCUAGCUUG | 953 |
| | | GCUAGGGUGCA | | | | | UAGCUGCCA | |
| 1703041 | 1703039 | UCUGGGAGGUUG | 808 | 4299 | 4321 | 1703040 | UGUAGCUGCCA | 954 |
| | | GCAGCUACAAG | | | | | ACCUCCCAGA | |
| 1703053 | 1703051 | UCCCCUUUGUCG | 809 | 4362 | 4384 | 1703052 | CUCCACACCCGA | 955 |
| | | GGUGUGGAGGG | | | | | CAAAGGGGA | |
| 1703083 | 1703081 | UAAACAGACAAA | 810 | 4510 | 4532 | 1703082 | GUAGUUGGAUU | 956 |
| | | UCCAACUACAA | | | | | UGUCUGUUUA | |
| 1703086 | 1703084 | UGGUGAAUCCAA | 811 | 4525 | 4547 | 1703085 | UGUUUAUGCUU | 957 |
| | | GCAUAAACAGA | | | | | GGAUUCACCA | |
| 1703092 | 1703090 | UUACAAGCAUUU | 812 | 4593 | 4615 | 1703091 | UGUAUCUUGAA | 958 |
| | | CAAGAUACAUG | | | | | AUGCUUGUAA | |
| 1703134 | 1703132 | UAGGGAAGGGGA | 813 | 4815 | 4837 | 1703133 | GCGAUGAUGUC | 959 |
| | | CAUCAUCGCUU | | | | | CCCUUCCCUA | |
| 1703158 | 1703156 | UAAGCCUCCUUU | 814 | 4939 | 4961 | 1703157 | GGCAGUCCCAA | 960 |
| | | GGGACUGCCAU | | | | | AGGAGGCUUA | |
| 1703182 | 1703180 | UGGUGGGAGGCU | 815 | 5082 | 5104 | 1703181 | GGGCAGCGCAG | 961 |
| | | GCGCUGCCCCU | | | | | CCUCCCACCA | |
| 1703191 | 1703189 | UUGGGCCUCUAG | 816 | 5148 | 5170 | 1703190 | GGAUCUGCUCU | 962 |
| | | AGCAGAUCCAG | | | | | AGAGGCCCAA | |
| 1703197 | 1703195 | UCAAGUCAUCCU | 817 | 5176 | 5198 | 1703196 | GCCUGAGGAAG | 963 |
| | | UCCUCAGGCAG | | | | | GAUGACUUGA | |
| 1703215 | 1703213 | UUUCUCAUGGCA | 818 | 5263 | 5285 | 1703214 | UCCAUCUGCUGC | 964 |
| | | GCAGAUGGAGU | | | | | CAUGAGAAA | |
| 1703227 | 1703225 | UGGGCCUGAGGC | 819 | 5321 | 5343 | 1703226 | AACUCAGCAGCC | 965 |
| | | UGCUGAGUUUC | | | | | UCAGGCCCA | |
| 1703239 | 1703237 | UACUGCCAAGUC | 820 | 5378 | 5400 | 1703238 | ACCCUGAGGGA | 966 |
| | | CCUCAGGGUUG | | | | | CUUGGCAGUA | |
| 1703263 | 1703261 | UCAGCGAACGGC | 821 | 5491 | 5513 | 1703262 | AGCCUCCCUGCC | 967 |
| | | AGGGAGGCUCU | | | | | GUUCGCUGA | |
| 1703269 | 1703267 | UUAGGAGAAUUG | 822 | 5510 | 5532 | 1703268 | GAGUCCCAGCA | 968 |
| | | CUGGGACUCAG | | | | | AUUCUCCUAA | |
| 1703272 | 1703270 | UCAGAUCCCUUC | 823 | 5525 | 5547 | 1703271 | UCCUAAGUUGA | 969 |
| | | AACUUAGGAGA | | | | | AGGGAUCUGA | |
| 1703281 | 1703279 | UAUCUCUAACCA | 824 | 5570 | 5592 | 1703280 | AUUUGGUGGUG | 970 |
| | | CCACCAAAUCU | | | | | GUUAGAGAUA | |
| 1703314 | 1703312 | UAAACCAUGAUC | 825 | 5743 | 5765 | 1703313 | CCAGCCUAAGA | 971 |
| | | UUAGGCUGGCC | | | | | UCAUGGUUUA | |
| 1703320 | 1703318 | UUUCAAUUUAUC | 826 | 5773 | 5795 | 1703319 | GUGCUGGCAGA | 972 |
| | | UGCCAGCACUG | | | | | UAAAUUGAAA | |
| 1703338 | 1703336 | UGGGAGAAGUGA | 827 | 5851 | 5873 | 1703337 | CCCCUCCCCUCA | 973 |
| | | GGGGAGGGGAG | | | | | CUUCUCCCA | |
| 1703356 | 1703354 | UCAAUUUAAAUG | 828 | 5951 | 5973 | 1703355 | CAAUAGUUCCA | 974 |
| | | GAACUAUUGAU | | | | | UUUAAAUUGA | |
| 1703368 | 1703366 | UCCCCCCAUAGCA | 829 | 6005 | 6027 | 1703367 | GCUUGUUGUGC | 975 |
| | | CAACAAGCAA | | | | | UAUGGGGGGA | |
| 1703371 | 1703369 | UCCUCCCCCCUCC | 830 | 6015 | 6037 | 1703370 | CUAUGGGGGGA | 976 |
| | | CCCCAUAGCA | | | | | GGGGGGAGGA | |
| 1703374 | 1703372 | UUAACUAUCUUA | 831 | 6032 | 6054 | 1703373 | AGGAAUGUGUA | 977 |
| | | CACAUUCCUCC | | | | | AGAUAGUUAA | |
| 1703386 | 1703384 | UCAUGAAAAGGG | 832 | 6092 | 6114 | 1703385 | GCCUCGUAACCC | 978 |
| | | UUACGAGGCAG | | | | | UUUUCAUGA | |
| 1703392 | 1703390 | UCCCUCCCUCUAG | 833 | 6120 | 6142 | 1703391 | CACAUUUGCUA | 979 |
| | | CAAAUGUGGU | | | | | GAGGGAGGGA | |
| 1703443 | 1703441 | UGGAGCCAGUGU | 834 | 6358 | 6380 | 1703442 | CCACCUCUCACA | 980 |
| | | GAGAGGUGGCA | | | | | CUGGCUCCA | |
| 1703449 | 1703447 | UCUCAGCUCCAA | 835 | 6388 | 6410 | 1703448 | CCUGUGCUUUU | 981 |
| | | AAGCACAGGCU | | | | | GGAGCUGAGA | |
| 1703461 | 1703459 | UCACCUCUGCCCU | 836 | 6458 | 6480 | 1703460 | UCGGGGCGAGG | 982 |
| | | CGCCCCGACC | | | | | GCAGAGGUGA | |
| 1703464 | 1703462 | UCUGUAGAUGGG | 837 | 6482 | 6504 | 1703463 | CCUGCGUGUCCC | 983 |
| | | ACACGCAGGUG | | | | | AUCUACAGA | |
| 1703467 | 1703465 | UUAUGAAGCUGC | 838 | 6497 | 6519 | 1703466 | UACAGACCUGC | 984 |
| | | AGGUCUGUAGA | | | | | AGCUUCAUAA | |
| 1703488 | 1703486 | UCCUGCUCAACA | 839 | 6602 | 6624 | 1703487 | AGUUUGCCAUG | 985 |
| | | UGGCAAACUCA | | | | | UUGAGCAGGA | |
| 1703503 | 1703501 | UGUCCCUCCCCCC | 840 | 6665 | 6687 | 1703502 | CUGAGGGUGGG | 986 |
| | | ACCCUCAGAA | | | | | GGGAGGGACA | |
| 1703518 | 1703516 | UGUAAAUCAUUU | 841 | 6740 | 6762 | 1703517 | UGUUUUAACAA | 987 |
| | | GUUAAAACACA | | | | | AUGAUUUACA | |
| 1703521 | 1703519 | UACAGCAACAGU | 842 | 6755 | 6777 | 1703520 | UUUACACUGAC | 988 |
| | | CAGUGUAAAUC | | | | | UGUUGCUGUA | |
| 1703524 | 1703522 | UUCCAAAUUCAC | 843 | 6770 | 6792 | 1703523 | GCUGUAAAAGU | 989 |
| | | UUUUACAGCAA | | | | | GAAUUUGGAA | |
| 1703545 | 1703543 | UCUGUGGAAACU | 844 | 1186 | 1208 | 1703544 | CCUCUCCAAAGU | 990 |
| | | UUGGAGAGGAA | | | | | UUCCACAGA | |
| 1703551 | 1703549 | UUUUACUGACCA | 845 | 1497 | 1519 | 1703550 | AAGCUCGCAUG | 991 |
| | | UGCGAGCUUUG | | | | | GUCAGUAAAA | |
| 1703557 | 1703555 | UUGGCUUUUUUG | 846 | 1534 | 1556 | 1703556 | AAGCGAUGACA | 992 |
| | | UCAUCGCUUCC | | | | | AAAAAGCCAA | |
| 1703566 | 1703564 | UCAAGGUUUUAG | 847 | 1566 | 1588 | 1703565 | GUUCCUCUGCU | 993 |
| | | CAGAGGAACGU | | | | | AAAACCUUGA | |
| 1703569 | 1703567 | UAUUUUUCAAGG | 848 | 1572 | 1594 | 1703568 | CUGCUAAAACC | 994 |
| | | UUUUAGCAGAG | | | | | UUGAAAAAUA | |
| 1703581 | 1703579 | UUUCAUCUCCUU | 849 | 1727 | 1749 | 1703580 | UCUGGAGCAAA | 995 |
| | | UGCUCCAGAAC | | | | | GGAGAUGAAA | |
| 1703584 | 1703582 | UGGCGAUCUUCG | 850 | 1761 | 1783 | 1703583 | AUGGUAAAACG | 996 |
| | | UUUUACCAUCA | | | | | AAGAUCGCCA | |
| 1703587 | 1703585 | UUGGACUUGACA | 851 | 2080 | 2102 | 1703586 | CCUGAAGAAUG | 997 |
| | | UUCUUCAGGUC | | | | | UCAAGUCCAA | |
| 1703602 | 1703600 | UGUUUGAUAUUA | 852 | 2200 | 2222 | 1703601 | CUCAAAGGAUA | 998 |
| | | UCCUUUGAGCC | | | | | AUAUCAAACA | |
| 1703614 | 1703612 | UCAGGUCAACUG | 853 | 2250 | 2272 | 1703613 | UCUACAAACCA | 999 |
| | | GUUUGUAGACU | | | | | GUUGACCUGA | |
| 1703617 | 1703615 | UGGUUUAUGAUG | 854 | 2300 | 2322 | 1703616 | GGCAACAUCCA | 1000 |
| | | GAUGUUGCCUA | | | | | UCAUAAACCA | |
| 1703620 | 1703618 | UUCUCAGAUUUU | 855 | 2332 | 2354 | 1703619 | GGUGGAAGUAA | 1001 |
| | | ACUUCCACCUG | | | | | AAUCUGAGAA | |
| 1703632 | 1703630 | UGGGUUUCAAUC | 856 | 2425 | 2447 | 1703631 | AAAUAAAAAGA | 1002 |
| | | UUUUUAUUUCC | | | | | UUGAAACCCA | |
| 1703641 | 1703639 | UCUCAUUCUCUC | 857 | 2676 | 2698 | 1703640 | UGUGGAGAGGA | 1003 |
| | | CUCUCCACAAU | | | | | GAGAAUGAGA | |
| 1703644 | 1703642 | UUUUUUUCCACA | 858 | 2691 | 2713 | 1703643 | AUGAGAGAGUG | 1004 |
| | | CUCUCUCAUUC | | | | | UGGAAAAAAA | |
| 1703647 | 1703645 | UUAUUCUUUUUU | 859 | 2699 | 2721 | 1703646 | GUGUGGAAAAA | 1005 |
| | | UUUCCACACUC | | | | | AAAAGAAUAA | |
| 1703650 | 1703648 | UUAACCAAUUAA | 860 | 2757 | 2779 | 1703649 | GCAGUUCGGUU | 1006 |
| | | CCGAACUGCGA | | | | | AAUUGGUUAA | |
| 1703656 | 1703654 | UGAUUUUGAAGU | 861 | 2807 | 2829 | 1703655 | UGGCUCGGGAC | 1007 |
| | | CCCGAGCCAAA | | | | | UUCAAAAUCA | |
| 1703662 | 1703660 | UUUGCUCUUACU | 862 | 2827 | 2849 | 1703661 | AGUGAUGGGAG | 1008 |
| | | CCCAUCACUGA | | | | | UAAGAGCAAA | |
| 1703671 | 1703669 | UUUGGAAAGAUG | 863 | 2842 | 2864 | 1703670 | AGCAAAUUUCA | 1009 |
| | | AAAUUUGCUCU | | | | | UCUUUCCAAA | |
| 1703680 | 1703678 | UUUUUUUAAAUA | 864 | 2878 | 2900 | 1703679 | GUAAUAAAAUA | 1010 |
| | | UUUUAUUACUA | | | | | UUUAAAAAAA | |
| 1703683 | 1703681 | UUUGAAUGUUUU | 865 | 2888 | 2910 | 1703682 | AUUUAAAAAAA | 1011 |
| | | UUUUUAAAUAU | | | | | AACAUUCAAA | |
| 1703686 | 1703684 | UGUUUUUGAAUG | 866 | 2892 | 2914 | 1703685 | AAAAAAAAACA | 1012 |
| | | UUUUUUUUUAA | | | | | UUCAAAAACA | |
| 1703707 | 1703705 | UGGAGGGGGAAG | 867 | 2951 | 2973 | 1703706 | UUCUUUUUUCU | 1013 |
| | | AAAAAAGAAUC | | | | | UCCCCCUCCA | |
| 1703710 | 1703708 | UUCUACAUGGAG | 868 | 2958 | 2980 | 1703709 | UUCUUCCCCCUC | 1014 |
| | | GGGGAAGAAAA | | | | | CAUGUAGAA | |
| 1703725 | 1703723 | UCAAGACAUCAA | 869 | 3478 | 3500 | 1703724 | AGACUGACCUU | 1015 |
| | | GGUCAGUCUUU | | | | | GAUGUCUUGA | |
| 1703749 | 1703747 | UUCCCUUAAUUU | 870 | 3941 | 3963 | 1703748 | CUGAGGGUGAA | 1016 |
| | | CACCCUCAGUA | | | | | AUUAAGGGAA | |
| 1703791 | 1703789 | UUAGAAACCUCU | 871 | 4605 | 4627 | 1703790 | UGCUUGUAAAG | 1017 |
| | | UUACAAGCAUU | | | | | AGGUUUCUAA | |
| 1703800 | 1703798 | UUCAGUCCUAAU | 872 | 4794 | 4816 | 1703799 | AAGCACAGGAU | 1018 |
| | | CCUGUGCUUCA | | | | | UAGGACUGAA | |
| 1703803 | 1703801 | UUCUUGUGAUGC | 873 | 4959 | 4981 | 1703802 | ACAACUCCUGCA | 1019 |
| | | AGGAGUUGUAA | | | | | UCACAAGAA | |
| 1703806 | 1703804 | UCCUUUUUCUUG | 874 | 4965 | 4987 | 1703805 | CCUGCAUCACAA | 1020 |
| | | UGAUGCAGGAG | | | | | GAAAAAGGA | |
| 1703812 | 1703810 | UUAGGCAGCAAU | 875 | 5297 | 5319 | 1703811 | UUGCAAAACAU | 1021 |
| | | GUUUUGCAAAG | | | | | UGCUGCCUAA | |
| 1703821 | 1703819 | UUUCAGGUAAAG | 876 | 5438 | 5460 | 1703820 | CAGCUAGAGCU | 1022 |
| | | CUCUAGCUGCA | | | | | UUACCUGAAA | |
| 1703824 | 1703822 | UCAACUUAGGAG | 877 | 5515 | 5537 | 1703823 | CCAGCAAUUCUC | 1023 |
| | | AAUUGCUGGGA | | | | | CUAAGUUGA | |
| 1703836 | 1703834 | UCUCUAACCACC | 878 | 5568 | 5590 | 1703835 | AGAUUUGGUGG | 1024 |
| | | ACCAAAUCUAC | | | | | UGGUUAGAGA | |
| 1703839 | 1703837 | UAAUGAGGGGGG | 879 | 5584 | 5606 | 1703838 | AGAGAUAUGCC | 1025 |
| | | CAUAUCUCUAA | | | | | CCCCUCAUUA | |
| 1703842 | 1703840 | UCACCCUAAACC | 880 | 5749 | 5771 | 1703841 | UAAGAUCAUGG | 1026 |
| | | AUGAUCUUAGG | | | | | UUUAGGGUGA | |
| 1703860 | 1703858 | UCAUUUAAGAUC | 881 | 5801 | 5823 | 1703859 | CUGGCUUGUGA | 1027 |
| | | ACAAGCCAGCG | | | | | UCUUAAAUGA | |
| 1703863 | 1703861 | UGUCCUCAUUUA | 882 | 5806 | 5828 | 1703862 | UUGUGAUCUUA | 1028 |
| | | AGAUCACAAGC | | | | | AAUGAGGACA | |
| 1703866 | 1703864 | UAUAUCCUAUCU | 883 | 5892 | 5914 | 1703865 | GGGUGGGCUAG | 1029 |
| | | AGCCCACCCAA | | | | | AUAGGAUAUA | |
| 1703872 | 1703870 | UUGAUAAAGUGA | 884 | 5933 | 5955 | 1703871 | GCUGCUGACUC | 1030 |
| | | GUCAGCAGCUU | | | | | ACUUUAUCAA | |
| 1703887 | 1703885 | UACAGUCUCACC | 885 | 5970 | 5992 | 1703886 | GACUUCAGUGG | 1031 |
| | | ACUGAAGUCAA | | | | | UGAGACUGUA | |
| 1703908 | 1703906 | UGAAAAGGGUUA | 886 | 6089 | 6111 | 1703907 | ACUGCCUCGUA | 1032 |
| | | CGAGGCAGUUU | | | | | ACCCUUUUCA | |
| 1703914 | 1703912 | UGUGGUUGAAAU | 887 | 6103 | 6125 | 1703913 | CUUUUCAUGAU | 1033 |
| | | CAUGAAAAGGG | | | | | UUCAACCACA | |
| 1703929 | 1703927 | UCGUGGCUUUAC | 888 | 6435 | 6457 | 1703928 | GUUCUCCAAGU | 1034 |
| | | UUGGAGAACAA | | | | | AAAGCCACGA | |
| 1703938 | 1703936 | UGAAGAGAAAUC | 889 | 6516 | 6538 | 1703937 | AAAACUUCUGA | 1035 |
| | | AGAAGUUUUAU | | | | | UUUCUCUUCA | |
| 1703941 | 1703939 | UCAAAGCUGAAG | 890 | 6523 | 6545 | 1703940 | CUGAUUUCUCU | 1036 |
| | | AGAAAUCAGAA | | | | | UCAGCUUUGA | |
| 1703947 | 1703945 | UCAUGGGGCUAA | 891 | 6582 | 6604 | 1703946 | CUAAUAGACUU | 1037 |
| | | GUCUAUUAGGA | | | | | AGCCCCAUGA | |
| 1703953 | 1703951 | UUGCAAGUGCCA | 892 | 6621 | 6643 | 1703952 | GACUAUUUCUG | 1038 |
| | | GAAAUAGUCCU | | | | | GCACUUGCAA | |
| 1703956 | 1703954 | UACCGAAGAAAU | 893 | 6641 | 6663 | 1703955 | AGUCCCAUGAU | 1039 |
| | | CAUGGGACUUG | | | | | UUCUUCGGUA | |
| 1703968 | 1703966 | UUCACAGACAGA | 894 | 6699 | 6721 | 1703967 | GCUUAGCUUUC | 1040 |
| | | AAGCUAAGCUA | | | | | UGUCUGUGAA | |
| 1703974 | 1703972 | UACACUAUAUAG | 895 | 6714 | 6736 | 1703973 | UGUGAAUGUCU | 1041 |
| | | ACAUUCACAGA | | | | | AUAUAGUGUA | |
| 1703980 | 1703978 | UGUUAAAACACA | 896 | 6729 | 6751 | 1703979 | AGUGUAUUGUG | 1042 |
| | | CAAUACACUAU | | | | | UGUUUUAACA | |
| 1703986 | 1703984 | UCAGUGUAAAUC | 897 | 6744 | 6766 | 1703985 | UUAACAAAUGA | 1043 |
| | | AUUUGUUAAAA | | | | | UUUACACUGA | |
| 1703998 | 1703996 | UUAUUUCCAAAU | 898 | 6774 | 6796 | 1703997 | UAAAAGUGAAU | 1044 |
| | | UCACUUUUACA | | | | | UUGGAAAUAA | |

“Start site” indicates the 5′-most nucleoside to which the antisense RNAi oligonucleotide is complementary target nucleic acid sequence. “Stop site” indicates the 3′-most nucleoside to which the antisense RNAi oligonucleotide is complementary in the target nucleic acid sequence. Each antisense RNAi oligonucleotide listed in the tables below is complementary to SEQ ID NO: 1 (GENBANK Accession No. NM_001377265.1), with a single mismatch at the 5′ end.

TABLE 22
RNAi compounds targeting human MAPT, SEQ ID NO: 1

| | | | | SEQ ID | SEQ ID | | | |
|---|---|---|---|---|---|---|---|---|
| | | | | NO: 1 | NO: 1 | | | |
| | Anti- | Antisense | SEQ | Antisense | Antisense | | Sense | SEQ |
| Compound | sense | Sequence | ID | Start | Stop | Sense | Sequence | ID |
| Number | ID | (5′ to 3′) | NO. | Site | Site | ID | (5′ to 3′) | NO. |
| 1702345 | 1702343 | UUUCACCUGAUA | 1045 | 117 | 138 | 1702344 | UCUGUCGACUA | 1444 |
| | | GUCGACAGAGG | | | | | UCAGGUGAAA | |
| 1702351 | 1702349 | UGUUCAAAGUUC | 1046 | 125 | 146 | 1702350 | CUAUCAGGUGA | 1445 |
| | | ACCUGAUAGUC | | | | | ACUUUGAACA | |
| 1702354 | 1702352 | UUGGUUCAAAGU | 1047 | 127 | 148 | 1702353 | AUCAGGUGAAC | 1446 |
| | | UCACCUGAUAG | | | | | UUUGAACCAA | |
| 1702357 | 1702355 | UCAUCCUGGUUC | 1048 | 132 | 153 | 1702356 | GUGAACUUUGA | 1447 |
| | | AAAGUUCACCU | | | | | ACCAGGAUGA | |
| 1702360 | 1702358 | UGGGCUCAGCCA | 1049 | 141 | 162 | 1702359 | GAACCAGGAUG | 1448 |
| | | UCCUGGUUCAA | | | | | GCUGAGCCCA | |
| 1702363 | 1702361 | UCGGGGCUCAGC | 1050 | 143 | 164 | 1702362 | ACCAGGAUGGC | 1449 |
| | | CAUCCUGGUUC | | | | | UGAGCCCCGA | |
| 1702369 | 1702367 | UACUCCUGGCGG | 1051 | 151 | 172 | 1702368 | GGCUGAGCCCCG | 1450 |
| | | GGCUCAGCCAU | | | | | CCAGGAGUA | |
| 1702372 | 1702370 | UCUUCGAACUCC | 1052 | 157 | 178 | 1702371 | GCCCCGCCAGGA | 1451 |
| | | UGGCGGGGCUC | | | | | GUUCGAAGA | |
| 1702378 | 1702376 | UAUCACUUCGAA | 1053 | 161 | 182 | 1702377 | CGCCAGGAGUU | 1452 |
| | | CUCCUGGCGGG | | | | | CGAAGUGAUA | |
| 1702384 | 1702382 | UUUCCAUCACUU | 1054 | 165 | 186 | 1702383 | AGGAGUUCGAA | 1453 |
| | | CGAACUCCUGG | | | | | GUGAUGGAAA | |
| 1702387 | 1702385 | UUGAUCUUCCAU | 1055 | 170 | 191 | 1702386 | UUCGAAGUGAU | 1454 |
| | | CACUUCGAACU | | | | | GGAAGAUCAA | |
| 1702390 | 1702388 | UCGUCCCAGCGU | 1056 | 180 | 201 | 1702389 | UGGAAGAUCAC | 1455 |
| | | GAUCUUCCAUC | | | | | GCUGGGACGA | |
| 1702393 | 1702391 | UUACGUCCCAGC | 1057 | 182 | 203 | 1702392 | GAAGAUCACGC | 1456 |
| | | GUGAUCUUCCA | | | | | UGGGACGUAA | |
| 1702396 | 1702394 | UCGUACGUCCCA | 1058 | 184 | 205 | 1702395 | AGAUCACGCUG | 1457 |
| | | GCGUGAUCUUC | | | | | GGACGUACGA | |
| 1702399 | 1702397 | UCCGUACGUCCC | 1059 | 185 | 206 | 1702398 | GAUCACGCUGG | 1458 |
| | | AGCGUGAUCUU | | | | | GACGUACGGA | |
| 1702402 | 1702400 | UCCCCAACCCGUA | 1060 | 192 | 213 | 1702401 | CUGGGACGUAC | 1459 |
| | | CGUCCCAGCG | | | | | GGGUUGGGGA | |
| 1702405 | 1702403 | UCCCUGAUCUUU | 1061 | 209 | 230 | 1702404 | GGGGACAGGAA | 1460 |
| | | CCUGUCCCCCA | | | | | AGAUCAGGGA | |
| 1702408 | 1702406 | UUGCAUGGUGUA | 1062 | 224 | 245 | 1702407 | CAGGGGGGCUA | 1461 |
| | | GCCCCCCUGAU | | | | | CACCAUGCAA | |
| 1702411 | 1702409 | UCCCUCUUGGUC | 1063 | 239 | 260 | 1702410 | AUGCACCAAGA | 1462 |
| | | UUGGUGCAUGG | | | | | CCAAGAGGGA | |
| 1702414 | 1702412 | UUCCGUGUCACC | 1064 | 248 | 269 | 1702413 | GACCAAGAGGG | 1463 |
| | | CUCUUGGUCUU | | | | | UGACACGGAA | |
| 1702417 | 1702415 | UCGUCCGUGUCA | 1065 | 250 | 271 | 1702416 | CCAAGAGGGUG | 1464 |
| | | CCCUCUUGGUC | | | | | ACACGGACGA | |
| 1702420 | 1702418 | UGCCAGCGUCCG | 1066 | 255 | 276 | 1702419 | AGGGUGACACG | 1465 |
| | | UGUCACCCUCU | | | | | GACGCUGGCA | |
| 1702426 | 1702424 | UGGGAGAUUCUU | 1067 | 270 | 291 | 1702425 | CUGGCCUGAAA | 1466 |
| | | UCAGGCCAGCG | | | | | GAAUCUCCCA | |
| 1702429 | 1702427 | UGGUCUGCAGGG | 1068 | 279 | 300 | 1702428 | AAGAAUCUCCCC | 1467 |
| | | GAGAUUCUUUC | | | | | UGCAGACCA | |
| 1702432 | 1702430 | UGUCCUCAGUGG | 1069 | 291 | 312 | 1702431 | UGCAGACCCCCA | 1468 |
| | | GGGUCUGCAGG | | | | | CUGAGGACA | |
| 1702435 | 1702433 | UGCCCGGUUCCU | 1070 | 309 | 330 | 1702434 | ACGGAUCUGAG | 1469 |
| | | CAGAUCCGUCC | | | | | GAACCGGGCA | |
| 1702438 | 1702436 | UUCAGAGGUUUC | 1071 | 323 | 344 | 1702437 | CCGGGCUCUGA | 1470 |
| | | AGAGCCCGGUU | | | | | AACCUCUGAA | |
| 1702450 | 1702448 | UUCUUCCAGGCU | 1072 | 389 | 410 | 1702449 | GACACCCCCAGC | 1471 |
| | | GGGGGUGUCUC | | | | | CUGGAAGAA | |
| 1702456 | 1702454 | UCUCCUCUUGGG | 1073 | 420 | 441 | 1702455 | GUCACGUGACCC | 1472 |
| | | UCACGUGACCA | | | | | AAGAGGAGA | |
| 1702459 | 1702457 | UGGAACUCUCAA | 1074 | 431 | 452 | 1702458 | CAAGAGGAGUU | 1473 |
| | | CUCCUCUUGGG | | | | | GAGAGUUCCA | |
| 1702465 | 1702463 | UAGGGGCCUUUC | 1075 | 464 | 485 | 1702464 | AAGGCGCCUGA | 1474 |
| | | AGGCGCCUUCC | | | | | AAGGCCCCUA | |
| 1702468 | 1702466 | UCGCUAAUCUCA | 1076 | 481 | 502 | 1702467 | CCUGGCCAAUG | 1475 |
| | | UUGGCCAGGGG | | | | | AGAUUAGCGA | |
| 1702474 | 1702472 | UGAGACCCCAGA | 1077 | 524 | 545 | 1702473 | GGAGAGGCCUC | 1476 |
| | | GGCCUCUCCGC | | | | | UGGGGUCUCA | |
| 1702477 | 1702475 | UGGAGCUUCUGG | 1078 | 560 | 581 | 1702476 | GAGAAAGAGCC | 1477 |
| | | CUCUUUCUCCC | | | | | AGAAGCUCCA | |
| 1702480 | 1702478 | UGGCGCUGGGCA | 1079 | 611 | 632 | 1702479 | CGUCCCGUUUGC | 1478 |
| | | AACGGGACGGU | | | | | CCAGCGCCA | |
| 1702483 | 1702481 | UUUUUUGUCCCC | 1080 | 660 | 681 | 1702482 | CCCUGGAGUGG | 1479 |
| | | ACUCCAGGGAG | | | | | GGACAAAAAA | |
| 1702486 | 1702484 | UAAAGGCCGGAC | 1081 | 693 | 714 | 1702485 | CCGAGAAGGGU | 1480 |
| | | CCUUCUCGGCC | | | | | CCGGCCUUUA | |
| 1702489 | 1702487 | UAGGCUCUGUGU | 1082 | 732 | 753 | 1702488 | CGUAUCUCCACA | 1481 |
| | | GGAGAUACGCA | | | | | CAGAGCCUA | |
| 1702492 | 1702490 | UCACCUUACCAC | 1083 | 747 | 768 | 1702491 | AGCCUGAAAGU | 1482 |
| | | UUUCAGGCUCU | | | | | GGUAAGGUGA | |
| 1702495 | 1702493 | UGCCUUCCUGGA | 1084 | 759 | 780 | 1702494 | GUAAGGUGGUC | 1483 |
| | | CCACCUUACCA | | | | | CAGGAAGGCA | |
| 1702498 | 1702496 | UUGGGGGGCCUG | 1085 | 783 | 804 | 1702497 | UCCGAGAGCCA | 1484 |
| | | GCUCUCGGAGG | | | | | GGCCCCCCAA | |
| 1702504 | 1702502 | UGAAGGUUGGCG | 1086 | 866 | 887 | 1702503 | GAGGCCACACGC | 1485 |
| | | UGUGGCCUCUC | | | | | CAACCUUCA | |
| 1702507 | 1702505 | UGCCGCCCUCUG | 1087 | 897 | 918 | 1702506 | CUGAGGACACA | 1486 |
| | | UGUCCUCAGGU | | | | | GAGGGCGGCA | |
| 1702510 | 1702508 | UUCCUAGAAGCU | 1088 | 936 | 957 | 1702509 | UCAAGCACCAGC | 1487 |
| | | GGUGCUUGAGC | | | | | UUCUAGGAA | |
| 1702513 | 1702511 | UUGGUGCAGGUC | 1089 | 947 | 968 | 1702512 | CUUCUAGGAGA | 1488 |
| | | UCCUAGAAGCU | | | | | CCUGCACCAA | |
| 1702516 | 1702514 | UUUUGCCCCCUG | 1090 | 981 | 1002 | 1702515 | UGAAGGGGGCA | 1489 |
| | | CCCCCUUCAGC | | | | | GGGGGCAAAA | |
| 1702519 | 1702517 | UGCGGUCUUCAU | 1091 | 1020 | 1041 | 1702518 | AGGAGGUGGAU | 1490 |
| | | CCACCUCCUCC | | | | | GAAGACCGCA | |
| 1702525 | 1702523 | UGCUGUCUGGGG | 1092 | 1100 | 1121 | 1702524 | GGGCGGCCUCCC | 1491 |
| | | AGGCCGCCCAU | | | | | CAGACAGCA | |
| 1702531 | 1702529 | UAUCUCUGUGGA | 1093 | 1190 | 1211 | 1702530 | UCCAAAGUUUC | 1492 |
| | | AACUUUGGAGA | | | | | CACAGAGAUA | |
| 1702540 | 1702538 | UCACGUGAAACG | 1094 | 1269 | 1290 | 1702539 | UGGAGUUCACG | 1493 |
| | | UGAACUCCAGG | | | | | UUUCACGUGA | |
| 1702543 | 1702541 | UGUUGGGUGUGA | 1095 | 1284 | 1305 | 1702542 | ACGUGGAAAUC | 1494 |
| | | UUUCCACGUGA | | | | | ACACCCAACA | |
| 1702549 | 1702547 | UUCCGAGUGCGC | 1096 | 1310 | 1331 | 1702548 | AAGGAGCAGGC | 1495 |
| | | CUGCUCCUUCU | | | | | GCACUCGGAA | |
| 1702552 | 1702550 | UCAGCCCUUCCCA | 1097 | 1330 | 1351 | 1702551 | GGAGCAUUUGG | 1496 |
| | | AAUGCUCCUC | | | | | GAAGGGCUGA | |
| 1702555 | 1702553 | UCCUCUUUUGUG | 1098 | 1399 | 1420 | 1702554 | GGGAGAGGACA | 1497 |
| | | UCCUCUCCCAA | | | | | CAAAAGAGGA | |
| 1702558 | 1702556 | UGCUCUGGAAGG | 1099 | 1414 | 1435 | 1702557 | AGAGGCUGACC | 1498 |
| | | UCAGCCUCUUU | | | | | UUCCAGAGCA | |
| 1702561 | 1702559 | UGCUGCUUUUCA | 1100 | 1429 | 1450 | 1702560 | AGAGCCCUCUG | 1499 |
| | | GAGGGCUCUGG | | | | | AAAAGCAGCA | |
| 1702564 | 1702562 | UCAGCAGCAGGC | 1101 | 1438 | 1459 | 1702563 | UGAAAAGCAGC | 1500 |
| | | UGCUUUUCAGA | | | | | CUGCUGCUGA | |
| 1702567 | 1702565 | UUGACGGGCUUC | 1102 | 1459 | 1480 | 1702566 | UCCGCGGGGGA | 1501 |
| | | CCCCGCGGAGC | | | | | AGCCCGUCAA | |
| 1702579 | 1702577 | UUUUUUUGUCAU | 1103 | 1530 | 1551 | 1702578 | CUGGAAGCGAU | 1502 |
| | | CGCUUCCAGUC | | | | | GACAAAAAAA | |
| 1702582 | 1702580 | UUGUGGAUGUCU | 1104 | 1545 | 1566 | 1702581 | AAAAAGCCAAG | 1503 |
| | | UGGCUUUUUUG | | | | | ACAUCCACAA | |
| 1702588 | 1702586 | UCCUAUUUUUCA | 1105 | 1575 | 1596 | 1702587 | CUAAAACCUUG | 1504 |
| | | AGGUUUUAGCA | | | | | AAAAAUAGGA | |
| 1702591 | 1702589 | UGGCUAAGGCAA | 1106 | 1588 | 1609 | 1702590 | AAAUAGGCCUU | 1505 |
| | | GGCCUAUUUUU | | | | | GCCUUAGCCA | |
| 1702600 | 1702598 | UAGGGUCUGAGC | 1107 | 1620 | 1641 | 1702599 | CUCCUGGUAGC | 1506 |
| | | UACCAGGAGUG | | | | | UCAGACCCUA | |
| 1702603 | 1702601 | UGGUUGGAUCAG | 1108 | 1631 | 1652 | 1702602 | UCAGACCCUCUG | 1507 |
| | | AGGGUCUGAGC | | | | | AUCCAACCA | |
| 1702606 | 1702604 | UUGGGCACACAG | 1109 | 1653 | 1674 | 1702605 | CCAGCCCUGCUG | 1508 |
| | | CAGGGCUGGAG | | | | | UGUGCCCAA | |
| 1702609 | 1702607 | UACGUAUUUAGG | 1110 | 1679 | 1700 | 1702608 | CCUUCCUCUCCU | 1509 |
| | | AGAGGAAGGUG | | | | | AAAUACGUA | |
| 1702612 | 1702610 | UGAAGUGACAGA | 1111 | 1694 | 1715 | 1702611 | UACGUCUCUUC | 1510 |
| | | AGAGACGUAUU | | | | | UGUCACUUCA | |
| 1702615 | 1702613 | UGAACUGCCAGU | 1112 | 1709 | 1730 | 1702614 | ACUUCCCGAACU | 1511 |
| | | UCGGGAAGUGA | | | | | GGCAGUUCA | |
| 1702618 | 1702616 | UAUCUCCUUUGC | 1113 | 1724 | 1745 | 1702617 | AGUUCUGGAGC | 1512 |
| | | UCCAGAACUGC | | | | | AAAGGAGAUA | |
| 1702621 | 1702619 | UGCCCCCUUGAG | 1114 | 1739 | 1760 | 1702620 | GAGAUGAAACU | 1513 |
| | | UUUCAUCUCCU | | | | | CAAGGGGGCA | |
| 1702624 | 1702622 | UUUCGUUUUACC | 1115 | 1754 | 1775 | 1702623 | GGGGCUGAUGG | 1514 |
| | | AUCAGCCCCCU | | | | | UAAAACGAAA | |
| 1702627 | 1702625 | UGCGAUCUUCGU | 1116 | 1760 | 1781 | 1702626 | GAUGGUAAAAC | 1515 |
| | | UUUACCAUCAG | | | | | GAAGAUCGCA | |
| 1702630 | 1702628 | UUGGCGAUCUUC | 1117 | 1762 | 1783 | 1702629 | UGGUAAAACGA | 1516 |
| | | GUUUUACCAUC | | | | | AGAUCGCCAA | |
| 1702633 | 1702631 | UGUGUGGCGAUC | 1118 | 1765 | 1786 | 1702632 | UAAAACGAAGA | 1517 |
| | | UUCGUUUUACC | | | | | UCGCCACACA | |
| 1702636 | 1702634 | UCCCUUCUGGCC | 1119 | 1796 | 1817 | 1702635 | GCCCCUCCAGGC | 1518 |
| | | UGGAGGGGCUG | | | | | CAGAAGGGA | |
| 1702639 | 1702637 | UGUUUUUGCUGG | 1120 | 1829 | 1850 | 1702638 | ACCAGGAUUCC | 1519 |
| | | AAUCCUGGUGG | | | | | AGCAAAAACA | |
| 1702642 | 1702640 | UAGCGGGCGGGG | 1121 | 1839 | 1860 | 1702641 | CAGCAAAAACCC | 1520 |
| | | UUUUUGCUGGA | | | | | CGCCCGCUA | |
| 1702645 | 1702643 | UGUGGUGUCUUU | 1122 | 1852 | 1873 | 1702644 | GCCCGCUCCAAA | 1521 |
| | | GGAGCGGGCGG | | | | | GACACCACA | |
| 1702648 | 1702646 | UGUUCACCAGAG | 1123 | 1867 | 1888 | 1702647 | ACCACCCAGCUC | 1522 |
| | | CUGGGUGGUGU | | | | | UGGUGAACA | |
| 1702654 | 1702652 | UCCGCUGCGAUC | 1124 | 1892 | 1913 | 1702653 | AAAUCAGGGGA | 1523 |
| | | CCCUGAUUUUG | | | | | UCGCAGCGGA | |
| 1702657 | 1702655 | UCGGGGUGCGGG | 1125 | 1944 | 1965 | 1702656 | GCAGCCGCUCCC | 1524 |
| | | AGCGGCUGCCG | | | | | GCACCCCGA | |
| 1702660 | 1702658 | UUGGAGUACGGA | 1126 | 2001 | 2022 | 1702659 | UGGCAGUGGUC | 1525 |
| | | CCACUGCCACC | | | | | CGUACUCCAA | |
| 1702663 | 1702661 | UGCGACUUGGGU | 1127 | 2011 | 2032 | 1702662 | CCGUACUCCACC | 1526 |
| | | GGAGUACGGAC | | | | | CAAGUCGCA | |
| 1702666 | 1702664 | UGCUGUCUGCAG | 1128 | 2042 | 2063 | 1702665 | AAGAGCCGCCU | 1527 |
| | | GCGGCUCUUGG | | | | | GCAGACAGCA | |
| 1702669 | 1702667 | UUUGACAUUCUU | 1129 | 2075 | 2096 | 1702668 | CCAGACCUGAA | 1528 |
| | | CAGGUCUGGCA | | | | | GAAUGUCAAA | |
| 1702672 | 1702670 | UGAGCCGAUCUU | 1130 | 2090 | 2111 | 1702671 | GUCAAGUCCAA | 1529 |
| | | GGACUUGACAU | | | | | GAUCGGCUCA | |
| 1702675 | 1702673 | UUUCAGGUUCUC | 1131 | 2105 | 2126 | 1702674 | GGCUCCACUGA | 1530 |
| | | AGUGGAGCCGA | | | | | GAACCUGAAA | |
| 1702681 | 1702679 | UUUAUUAAUUAU | 1132 | 2144 | 2165 | 1702680 | AAGGUGCAGAU | 1531 |
| | | CUGCACCUUCC | | | | | AAUUAAUAAA | |
| 1702684 | 1702682 | UCUAAGAUCCAG | 1133 | 2159 | 2180 | 1702683 | AAUAAGAAGCU | 1532 |
| | | CUUCUUAUUAA | | | | | GGAUCUUAGA | |
| 1702687 | 1702685 | UUUGGACUGGAC | 1134 | 2174 | 2195 | 1702686 | CUUAGCAACGU | 1533 |
| | | GUUGCUAAGAU | | | | | CCAGUCCAAA | |
| 1702690 | 1702688 | UUCCUUUGAGCC | 1135 | 2189 | 2210 | 1702689 | UCCAAGUGUGG | 1534 |
| | | ACACUUGGACU | | | | | CUCAAAGGAA | |
| 1702693 | 1702691 | UACGUGUUUGAU | 1136 | 2204 | 2225 | 1702692 | AAGGAUAAUAU | 1535 |
| | | AUUAUCCUUUG | | | | | CAAACACGUA | |
| 1702696 | 1702694 | UCACUGCCGCCUC | 1137 | 2221 | 2242 | 1702695 | CGUCCCGGGAG | 1536 |
| | | CCGGGACGUG | | | | | GCGGCAGUGA | |
| 1702702 | 1702700 | UCUCAGGUCAAC | 1138 | 2252 | 2273 | 1702701 | UACAAACCAGU | 1537 |
| | | UGGUUUGUAGA | | | | | UGACCUGAGA | |
| 1702705 | 1702703 | UUUGGAGGUCAC | 1139 | 2267 | 2288 | 1702704 | CUGAGCAAGGU | 1538 |
| | | CUUGCUCAGGU | | | | | GACCUCCAAA | |
| 1702708 | 1702706 | UCCUAAUGAGCC | 1140 | 2282 | 2303 | 1702707 | UCCAAGUGUGG | 1539 |
| | | ACACUUGGAGG | | | | | CUCAUUAGGA | |
| 1702714 | 1702712 | UGCCACCUCCUG | 1141 | 2310 | 2331 | 1702713 | AUCAUAAACCA | 1540 |
| | | GUUUAUGAUGG | | | | | GGAGGUGGCA | |
| 1702720 | 1702718 | UAAGCUUCUCAG | 1142 | 2337 | 2358 | 1702719 | AAGUAAAAUCU | 1541 |
| | | AUUUUACUUCC | | | | | GAGAAGCUUA | |
| 1702723 | 1702721 | UUCUGUCCUUGA | 1143 | 2352 | 2373 | 1702722 | AGCUUGACUUC | 1542 |
| | | AGUCAAGCUUC | | | | | AAGGACAGAA | |
| 1702726 | 1702724 | UAAUCUUCGACU | 1144 | 2367 | 2388 | 1702725 | ACAGAGUCCAG | 1543 |
| | | GGACUCUGUCC | | | | | UCGAAGAUUA | |
| 1702732 | 1702730 | UGACGUGGGUGA | 1145 | 2394 | 2415 | 1702731 | UGGACAAUAUC | 1544 |
| | | UAUUGUCCAGG | | | | | ACCCACGUCA | |
| 1702741 | 1702739 | UGUCAGCUUGUG | 1146 | 2435 | 2456 | 1702740 | AUUGAAACCCA | 1545 |
| | | GGUUUCAAUCU | | | | | CAAGCUGACA | |
| 1702744 | 1702742 | UUUCUCGCGGAA | 1147 | 2447 | 2468 | 1702743 | AAGCUGACCUU | 1546 |
| | | GGUCAGCUUGU | | | | | CCGCGAGAAA | |
| 1702750 | 1702748 | UGAUCUCCGCCCC | 1148 | 2481 | 2502 | 1702749 | CAGACCACGGG | 1547 |
| | | GUGGUCUGUC | | | | | GCGGAGAUCA | |
| 1702753 | 1702751 | UCCACUGGCGAC | 1149 | 2500 | 2521 | 1702752 | CGUGUACAAGU | 1548 |
| | | UUGUACACGAU | | | | | CGCCAGUGGA | |
| 1702759 | 1702757 | UGAGACAUUGCU | 1150 | 2540 | 2561 | 1702758 | CGGCAUCUCAGC | 1549 |
| | | GAGAUGCCGUG | | | | | AAUGUCUCA | |
| 1702765 | 1702763 | UAUGUCGAUGCU | 1151 | 2561 | 2582 | 1702764 | UCCACCGGCAGC | 1550 |
| | | GCCGGUGGAGG | | | | | AUCGACAUA | |
| 1702768 | 1702766 | UGCGAGUCUACC | 1152 | 2572 | 2593 | 1702767 | CAUCGACAUGG | 1551 |
| | | AUGUCGAUGCU | | | | | UAGACUCGCA | |
| 1702771 | 1702769 | UUAGCGUGGCGA | 1153 | 2589 | 2610 | 1702770 | CGCCCCAGCUCG | 1552 |
| | | GCUGGGGCGAG | | | | | CCACGCUAA | |
| 1702774 | 1702772 | UGACACCUCGUC | 1154 | 2603 | 2624 | 1702773 | ACGCUAGCUGA | 1553 |
| | | AGCUAGCGUGG | | | | | CGAGGUGUCA | |
| 1702780 | 1702778 | UUGAUCACAAAC | 1155 | 2634 | 2655 | 1702779 | CCAAGCAGGGU | 1554 |
| | | CCUGCUUGGCC | | | | | UUGUGAUCAA | |
| 1702783 | 1702781 | UCCGCCCCAGGG | 1156 | 2648 | 2669 | 1702782 | UGAUCAGGCCCC | 1555 |
| | | GCCUGAUCACA | | | | | UGGGGCGGA | |
| 1702786 | 1702784 | UCUCUCCACAAU | 1157 | 2665 | 2686 | 1702785 | CGGUCAAUAAU | 1556 |
| | | UAUUGACCGCC | | | | | UGUGGAGAGA | |
| 1702789 | 1702787 | UCUCUCUCAUUC | 1158 | 2680 | 2701 | 1702788 | GAGAGGAGAGA | 1557 |
| | | UCUCCUCUCCA | | | | | AUGAGAGAGA | |
| 1702795 | 1702793 | UCCGGGUCAUUA | 1159 | 2708 | 2729 | 1702794 | AAAAAAGAAUA | 1558 |
| | | UUCUUUUUUUU | | | | | AUGACCCGGA | |
| 1702798 | 1702796 | UAGCUGGGGGCA | 1160 | 2730 | 2751 | 1702797 | CCCGCCCUCUGC | 1559 |
| | | GAGGGCGGGGG | | | | | CCCCAGCUA | |
| 1702801 | 1702799 | UACCAAUUAACC | 1161 | 2755 | 2776 | 1702800 | UCGCAGUUCGG | 1560 |
| | | GAACUGCGAGG | | | | | UUAAUUGGUA | |
| 1702804 | 1702802 | UCAGGUUAAGUG | 1162 | 2770 | 2791 | 1702803 | UUGGUUAAUCA | 1561 |
| | | AUUAACCAAUU | | | | | CUUAACCUGA | |
| 1702807 | 1702805 | UGCCGAGUGACA | 1163 | 2785 | 2806 | 1702806 | ACCUGCUUUUG | 1562 |
| | | AAAGCAGGUUA | | | | | UCACUCGGCA | |
| 1702810 | 1702808 | UAAGUCCCGAGC | 1164 | 2800 | 2821 | 1702809 | UCGGCUUUGGC | 1563 |
| | | CAAAGCCGAGU | | | | | UCGGGACUUA | |
| 1702813 | 1702811 | UCCAUCACUGAU | 1165 | 2815 | 2836 | 1702812 | GACUUCAAAAU | 1564 |
| | | UUUGAAGUCCC | | | | | CAGUGAUGGA | |
| 1702816 | 1702814 | UAAUUUGCUCUU | 1166 | 2830 | 2851 | 1702815 | GAUGGGAGUAA | 1565 |
| | | ACUCCCAUCAC | | | | | GAGCAAAUUA | |
| 1702819 | 1702817 | UAAUUUGGAAAG | 1167 | 2845 | 2866 | 1702818 | AAAUUUCAUCU | 1566 |
| | | AUGAAAUUUGC | | | | | UUCCAAAUUA | |
| 1702825 | 1702823 | UAAUAUUUUAUU | 1168 | 2871 | 2892 | 1702824 | UGGGCUAGUAA | 1567 |
| | | ACUAGCCCACC | | | | | UAAAAUAUUA | |
| 1702828 | 1702826 | UAUGUUUUUUUU | 1169 | 2884 | 2905 | 1702827 | AAAUAUUUAAA | 1568 |
| | | UAAAUAUUUUA | | | | | AAAAAACAUA | |
| 1702831 | 1702829 | UAUGUGGCCAUG | 1170 | 2902 | 2923 | 1702830 | AUUCAAAAACA | 1569 |
| | | UUUUUGAAUGU | | | | | UGGCCACAUA | |
| 1702834 | 1702832 | UCUGAGGAAAUG | 1171 | 2917 | 2938 | 1702833 | CACAUCCAACAU | 1570 |
| | | UUGGAUGUGGC | | | | | UUCCUCAGA | |
| 1702837 | 1702835 | UAUCAAAAGGAA | 1172 | 2932 | 2953 | 1702836 | CUCAGGCAAUU | 1571 |
| | | UUGCCUGAGGA | | | | | CCUUUUGAUA | |
| 1702840 | 1702838 | UGGGGAAGAAAA | 1173 | 2947 | 2968 | 1702839 | UUGAUUCUUUU | 1572 |
| | | AAGAAUCAAAA | | | | | UUCUUCCCCA | |
| 1702843 | 1702841 | UCUCUUCUACAU | 1174 | 2962 | 2983 | 1702842 | UCCCCCUCCAUG | 1573 |
| | | GGAGGGGGAAG | | | | | UAGAAGAGA | |
| 1702846 | 1702844 | UAGCCUCUCCUU | 1175 | 2977 | 2998 | 1702845 | AAGAGGGAGAA | 1574 |
| | | CUCCCUCUUCU | | | | | GGAGAGGCUA | |
| 1702849 | 1702847 | UAGAAGCAGCUU | 1176 | 2992 | 3013 | 1702848 | AGGCUCUGAAA | 1575 |
| | | UCAGAGCCUCU | | | | | GCUGCUUCUA | |
| 1702852 | 1702850 | UCUUGAAAUCCC | 1177 | 3005 | 3026 | 1702851 | UGCUUCUGGGG | 1576 |
| | | CCAGAAGCAGC | | | | | GAUUUCAAGA | |
| 1702855 | 1702853 | UCCCCCAGUCCCU | 1178 | 3015 | 3036 | 1702854 | GGAUUUCAAGG | 1577 |
| | | UGAAAUCCCC | | | | | GACUGGGGGA | |
| 1702858 | 1702856 | UCCCACAACAGG | 1179 | 3043 | 3064 | 1702857 | ACCUCUGGCCCU | 1578 |
| | | GCCAGAGGUGG | | | | | GUUGUGGGA | |
| 1702864 | 1702862 | UCACCAAGUUUC | 1180 | 3089 | 3110 | 1702863 | AAAGGAUUUGA | 1579 |
| | | AAAUCCUUUGU | | | | | AACUUGGUGA | |
| 1702867 | 1702865 | UGCUCCACGAAC | 1181 | 3101 | 3122 | 1702866 | ACUUGGUGUGU | 1580 |
| | | ACACCAAGUUU | | | | | UCGUGGAGCA | |
| 1702870 | 1702868 | UGUCUGCCUGUG | 1182 | 3112 | 3133 | 1702869 | UCGUGGAGCCA | 1581 |
| | | GCUCCACGAAC | | | | | CAGGCAGACA | |
| 1702873 | 1702871 | UCACAAGGUUGA | 1183 | 3127 | 3148 | 1702872 | CAGACGAUGUC | 1582 |
| | | CAUCGUCUGCC | | | | | AACCUUGUGA | |
| 1702876 | 1702874 | UCUCUCCCACUCC | 1184 | 3228 | 3249 | 1702875 | AAGAAGUGGGA | 1583 |
| | | CACUUCUUGU | | | | | GUGGGAGAGA | |
| 1702879 | 1702877 | UCACGUGGCUUC | 1185 | 3239 | 3260 | 1702878 | GUGGGAGAGGA | 1584 |
| | | CUCUCCCACUC | | | | | AGCCACGUGA | |
| 1702882 | 1702880 | UGAUGUCUACUC | 1186 | 3255 | 3276 | 1702881 | CGUGCUGGAGA | 1585 |
| | | UCCAGCACGUG | | | | | GUAGACAUCA | |
| 1702885 | 1702883 | UAAGGAGGGGGA | 1187 | 3264 | 3285 | 1702884 | GAGUAGACAUC | 1586 |
| | | UGUCUACUCUC | | | | | CCCCUCCUUA | |
| 1702888 | 1702886 | UCCCCCGGCCACC | 1188 | 3334 | 3355 | 1702887 | CUGUCCUUGGU | 1587 |
| | | AAGGACAGGC | | | | | GGCCGGGGGA | |
| 1702897 | 1702895 | UGAAGUGCCACC | 1189 | 3438 | 3459 | 1702896 | UGGCAGGAGGG | 1588 |
| | | CUCCUGCCAGC | | | | | UGGCACUUCA | |
| 1702903 | 1702901 | UCAUCAAGGUCA | 1190 | 3473 | 3494 | 1702902 | AGAAAAGACUG | 1589 |
| | | GUCUUUUCUAA | | | | | ACCUUGAUGA | |
| 1702906 | 1702904 | UCAGCGCUCUCA | 1191 | 3487 | 3508 | 1702905 | UUGAUGUCUUG | 1590 |
| | | AGACAUCAAGG | | | | | AGAGCGCUGA | |
| 1702912 | 1702910 | UACAGGGUUUCU | 1192 | 3559 | 3580 | 1702911 | CUGCUCCACAGA | 1591 |
| | | GUGGAGCAGAG | | | | | AACCCUGUA | |
| 1702918 | 1702916 | UCAGCAGUUCCA | 1193 | 3589 | 3610 | 1702917 | UCUGAAGGUUG | 1592 |
| | | ACCUUCAGAAC | | | | | GAACUGCUGA | |
| 1702921 | 1702919 | UUGGCCAAAAUC | 1194 | 3604 | 3625 | 1702920 | UGCUGCCAUGA | 1593 |
| | | AUGGCAGCAGU | | | | | UUUUGGCCAA | |
| 1702930 | 1702928 | UCUUACAAAGAG | 1195 | 3649 | 3670 | 1702929 | UAACCAGUUCU | 1594 |
| | | AACUGGUUAGC | | | | | CUUUGUAAGA | |
| 1702933 | 1702931 | UCAAGAGGCACA | 1196 | 3664 | 3685 | 1702932 | GUAAGGACUUG | 1595 |
| | | AGUCCUUACAA | | | | | UGCCUCUUGA | |
| 1702936 | 1702934 | UGUGGACGUCUC | 1197 | 3676 | 3697 | 1702935 | GCCUCUUGGGA | 1596 |
| | | CCAAGAGGCAC | | | | | GACGUCCACA | |
| 1702939 | 1702937 | UGGCUUGGAAAC | 1198 | 3689 | 3710 | 1702938 | CGUCCACCCGUU | 1597 |
| | | GGGUGGACGUC | | | | | UCCAAGCCA | |
| 1702942 | 1702940 | UAGUGGCCCAGG | 1199 | 3698 | 3719 | 1702941 | GUUUCCAAGCC | 1598 |
| | | CUUGGAAACGG | | | | | UGGGCCACUA | |
| 1702945 | 1702943 | UGAGAUGCCAGU | 1200 | 3706 | 3727 | 1702944 | GCCUGGGCCACU | 1599 |
| | | GGCCCAGGCUU | | | | | GGCAUCUCA | |
| 1702948 | 1702946 | UCCCACACACUCC | 1201 | 3719 | 3740 | 1702947 | GCAUCUCUGGA | 1600 |
| | | AGAGAUGCCA | | | | | GUGUGUGGGA | |
| 1702951 | 1702949 | UAGUGGCCGUGG | 1202 | 3765 | 3786 | 1702950 | CUGUCCUUCCCA | 1601 |
| | | GAAGGACAGGG | | | | | CGGCCACUA | |
| 1702960 | 1702958 | UCGUGUGAUGAG | 1203 | 3841 | 3862 | 1702959 | CCUAUACCCCUC | 1602 |
| | | GGGUAUAGGCA | | | | | AUCACACGA | |
| 1702963 | 1702961 | UUUCGGGACAUU | 1204 | 3856 | 3877 | 1702962 | ACACGUCACAA | 1603 |
| | | GUGACGUGUGA | | | | | UGUCCCGAAA | |
| 1702972 | 1702970 | UCCAACCAGGGU | 1205 | 3898 | 3919 | 1702971 | UCAGUAAUGAC | 1604 |
| | | CAUUACUGAGA | | | | | CCUGGUUGGA | |
| 1702978 | 1702976 | UCCCUCAGUAUG | 1206 | 3928 | 3949 | 1702977 | UACCUACUCCAU | 1605 |
| | | GAGUAGGUACC | | | | | ACUGAGGGA | |
| 1702981 | 1702979 | UCUUCCCUUAAU | 1207 | 3943 | 3964 | 1702980 | GAGGGUGAAAU | 1606 |
| | | UUCACCCUCAG | | | | | UAAGGGAAGA | |
| 1702990 | 1702988 | UGAGGCUGGGGU | 1208 | 3978 | 3999 | 1702989 | AAGAGUGGGAC | 1607 |
| | | CCCACUCUUGU | | | | | CCCAGCCUCA | |
| 1702996 | 1702994 | UGGUCCCAGUUG | 1209 | 4010 | 4031 | 1702995 | CCACUCAUCCAA | 1608 |
| | | GAUGAGUGGAA | | | | | CUGGGACCA | |
| 1702999 | 1702997 | UGAUUCGUGGUG | 1210 | 4023 | 4044 | 1702998 | UGGGACCCUCAC | 1609 |
| | | AGGGUCCCAGU | | | | | CACGAAUCA | |
| 1703002 | 1703000 | UCGAAUCAGAUC | 1211 | 4038 | 4059 | 1703001 | GAAUCUCAUGA | 1610 |
| | | AUGAGAUUCGU | | | | | UCUGAUUCGA | |
| 1703005 | 1703003 | UGAGACAGGGAA | 1212 | 4050 | 4071 | 1703004 | CUGAUUCGGUU | 1611 |
| | | CCGAAUCAGAU | | | | | CCCUGUCUCA | |
| 1703008 | 1703006 | UUGACGGGAGGA | 1213 | 4062 | 4083 | 1703007 | CCUGUCUCCUCC | 1612 |
| | | GGAGACAGGGA | | | | | UCCCGUCAA | |
| 1703011 | 1703009 | UUGGCUCACAUC | 1214 | 4075 | 4096 | 1703010 | CCCGUCACAGAU | 1613 |
| | | UGUGACGGGAG | | | | | GUGAGCCAA | |
| 1703014 | 1703012 | UCAGCUGAGCAG | 1215 | 4091 | 4112 | 1703013 | GCCAGGGCACU | 1614 |
| | | UGCCCUGGCUC | | | | | GCUCAGCUGA | |
| 1703017 | 1703015 | UAGAAACACCUA | 1216 | 4108 | 4129 | 1703016 | CUGUGACCCUA | 1615 |
| | | GGGUCACAGCU | | | | | GGUGUUUCUA | |
| 1703023 | 1703021 | UGGGAAAGGGCU | 1217 | 4138 | 4159 | 1703022 | CAUGGAGAGAG | 1616 |
| | | CUCUCCAUGUC | | | | | CCCUUUCCCA | |
| 1703029 | 1703027 | UCCACUGACAAC | 1218 | 4206 | 4227 | 1703028 | GGUGUCUUGGU | 1617 |
| | | CAAGACACCCA | | | | | UGUCAGUGGA | |
| 1703032 | 1703030 | UUCCUGGUGCCA | 1219 | 4217 | 4238 | 1703031 | UGUCAGUGGUG | 1618 |
| | | CCACUGACAAC | | | | | GCACCAGGAA | |
| 1703035 | 1703033 | UACUGUGGGCCU | 1220 | 4251 | 4272 | 1703034 | ACCCAGGGCAG | 1619 |
| | | GCCCUGGGUGC | | | | | GCCCACAGUA | |
| 1703044 | 1703042 | UGCAGCGGGCUG | 1221 | 4316 | 4337 | 1703043 | CAGACAGCCCAG | 1620 |
| | | GGCUGUCUGGG | | | | | CCCGCUGCA | |
| 1703047 | 1703045 | UUGAUACUAUGC | 1222 | 4339 | 4360 | 1703046 | GCUCCACAUGCA | 1621 |
| | | AUGUGGAGCUG | | | | | UAGUAUCAA | |
| 1703050 | 1703048 | UGGGUGUGGAGG | 1223 | 4352 | 4373 | 1703049 | AGUAUCAGCCC | 1622 |
| | | GCUGAUACUAU | | | | | UCCACACCCA | |
| 1703056 | 1703054 | UAAGGGGGUGUG | 1224 | 4375 | 4396 | 1703055 | AAAGGGGAACA | 1623 |
| | | UUCCCCUUUGU | | | | | CACCCCCUUA | |
| 1703059 | 1703057 | UAAAAGAACCAU | 1225 | 4390 | 4411 | 1703058 | CCCUUGGAAAU | 1624 |
| | | UUCCAAGGGGG | | | | | GGUUCUUUUA | |
| 1703062 | 1703060 | UCUGGGACUGGG | 1226 | 4403 | 4424 | 1703061 | UUCUUUUCCCCC | 1625 |
| | | GGAAAAGAACC | | | | | AGUCCCAGA | |
| 1703065 | 1703063 | UCAUGGCUUCCA | 1227 | 4415 | 4436 | 1703064 | AGUCCCAGCUG | 1626 |
| | | GCUGGGACUGG | | | | | GAAGCCAUGA | |
| 1703068 | 1703066 | UCAGCAGAACAG | 1228 | 4430 | 4451 | 1703067 | CCAUGCUGUCU | 1627 |
| | | ACAGCAUGGCU | | | | | GUUCUGCUGA | |
| 1703071 | 1703069 | UUAUGUUCAGCU | 1229 | 4445 | 4466 | 1703070 | UGCUGGAGCAG | 1628 |
| | | GCUCCAGCAGA | | | | | CUGAACAUAA | |
| 1703074 | 1703072 | UGGCAACAUCUA | 1230 | 4460 | 4481 | 1703073 | ACAUAUACAUA | 1629 |
| | | UGUAUAUGUUC | | | | | GAUGUUGCCA | |
| 1703077 | 1703075 | UUGCAGAUGGGG | 1231 | 4478 | 4499 | 1703076 | CCCUGCCCUCCC | 1630 |
| | | AGGGCAGGGCA | | | | | CAUCUGCAA | |
| 1703080 | 1703078 | UACUACAACUCA | 1232 | 4495 | 4516 | 1703079 | GCACCCUGUUG | 1631 |
| | | ACAGGGUGCAG | | | | | AGUUGUAGUA | |
| 1703089 | 1703087 | UUAUCAUAGUCA | 1233 | 4540 | 4561 | 1703088 | UCACCAGAGUG | 1632 |
| | | CUCUGGUGAAU | | | | | ACUAUGAUAA | |
| 1703095 | 1703093 | UGGUUAGAAACC | 1234 | 4608 | 4629 | 1703094 | UUGUAAAGAGG | 1633 |
| | | UCUUUACAAGC | | | | | UUUCUAACCA | |
| 1703098 | 1703096 | UCUCGUGAGGGU | 1235 | 4620 | 4641 | 1703097 | UUCUAACCCACC | 1634 |
| | | GGGUUAGAAAC | | | | | CUCACGAGA | |
| 1703101 | 1703099 | UCAGUGUGGGGG | 1236 | 4641 | 4662 | 1703100 | UGUCUCUCACCC | 1635 |
| | | UGAGAGACACC | | | | | CCACACUGA | |
| 1703104 | 1703102 | UACAGGCCACAC | 1237 | 4658 | 4679 | 1703103 | CUGGGACUCGU | 1636 |
| | | GAGUCCCAGUG | | | | | GUGGCCUGUA | |
| 1703107 | 1703105 | UCAGCAGGGUGG | 1238 | 4675 | 4696 | 1703106 | UGUGUGGUGCC | 1637 |
| | | CACCACACAGG | | | | | ACCCUGCUGA | |
| 1703110 | 1703108 | UAGCCUUUCAAA | 1239 | 4699 | 4720 | 1703109 | CUCCCAAGUUU | 1638 |
| | | ACUUGGGAGGC | | | | | UGAAAGGCUA | |
| 1703113 | 1703111 | UCAGGUGCUGAG | 1240 | 4713 | 4734 | 1703112 | AAGGCUUUCCU | 1639 |
| | | GAAAGCCUUUC | | | | | CAGCACCUGA | |
| 1703116 | 1703114 | UUCUCUGUUGGG | 1241 | 4727 | 4748 | 1703115 | CACCUGGGACCC | 1640 |
| | | UCCCAGGUGCU | | | | | AACAGAGAA | |
| 1703119 | 1703117 | UGCUGCUAGAAG | 1242 | 4742 | 4763 | 1703118 | AGAGACCAGCU | 1641 |
| | | CUGGUCUCUGU | | | | | UCUAGCAGCA | |
| 1703122 | 1703120 | UUGAACGGCCUC | 1243 | 4757 | 4778 | 1703121 | GCAGCUAAGGA | 1642 |
| | | CUUAGCUGCUA | | | | | GGCCGUUCAA | |
| 1703125 | 1703123 | UGCCUUCGUCAC | 1244 | 4771 | 4792 | 1703124 | CGUUCAGCUGU | 1643 |
| | | AGCUGAACGGC | | | | | GACGAAGGCA | |
| 1703128 | 1703126 | UAUCCUGUGCUU | 1245 | 4785 | 4806 | 1703127 | GAAGGCCUGAA | 1644 |
| | | CAGGCCUUCGU | | | | | GCACAGGAUA | |
| 1703131 | 1703129 | UAUCGCUUCAGU | 1246 | 4800 | 4821 | 1703130 | AGGAUUAGGAC | 1645 |
| | | CCUAAUCCUGU | | | | | UGAAGCGAUA | |
| 1703137 | 1703135 | UCACAGGGAGCC | 1247 | 4838 | 4859 | 1703136 | UCCCCUUGGGGC | 1646 |
| | | CCAAGGGGAAG | | | | | UCCCUGUGA | |
| 1703140 | 1703138 | UAAGACCUAGUC | 1248 | 4859 | 4880 | 1703139 | CAGGGCACAGA | 1647 |
| | | UGUGCCCUGAC | | | | | CUAGGUCUUA | |
| 1703143 | 1703141 | UAGACCAGCCAC | 1249 | 4870 | 4891 | 1703142 | CUAGGUCUUGU | 1648 |
| | | AAGACCUAGUC | | | | | GGCUGGUCUA | |
| 1703146 | 1703144 | UAUCCUCGCGCC | 1250 | 4888 | 4909 | 1703145 | CUGGCUUGCGG | 1649 |
| | | GCAAGCCAGAC | | | | | CGCGAGGAUA | |
| 1703152 | 1703150 | UGGGCUAUGACC | 1251 | 4909 | 4930 | 1703151 | GUUCUCUCUGG | 1650 |
| | | AGAGAGAACCA | | | | | UCAUAGCCCA | |
| 1703155 | 1703153 | UCUGCCAUGAGA | 1252 | 4924 | 4945 | 1703154 | AGCCCGAAGUC | 1651 |
| | | CUUCGGGCUAU | | | | | UCAUGGCAGA | |
| 1703161 | 1703159 | UUGAUGCAGGAG | 1253 | 4954 | 4975 | 1703160 | GGCUUACAACU | 1652 |
| | | UUGUAAGCCUC | | | | | CCUGCAUCAA | |
| 1703164 | 1703162 | UGCUUCCUUUUU | 1254 | 4969 | 4990 | 1703163 | CAUCACAAGAA | 1653 |
| | | CUUGUGAUGCA | | | | | AAAGGAAGCA | |
| 1703167 | 1703165 | UGCUGGCAGUGG | 1255 | 4979 | 5000 | 1703166 | AAAAGGAAGCC | 1654 |
| | | CUUCCUUUUUC | | | | | ACUGCCAGCA | |
| 1703170 | 1703168 | UGAGCUGCAGAU | 1256 | 4997 | 5018 | 1703169 | GCUGGGGGGAU | 1655 |
| | | CCCCCCAGCUG | | | | | CUGCAGCUCA | |
| 1703173 | 1703171 | UCUCACGGAGCU | 1257 | 5013 | 5034 | 1703172 | GCUCCCAGAAGC | 1656 |
| | | UCUGGGAGCUG | | | | | UCCGUGAGA | |
| 1703176 | 1703174 | UUGAGGGGUGGC | 1258 | 5029 | 5050 | 1703175 | UGAGCCUCAGCC | 1657 |
| | | UGAGGCUCACG | | | | | ACCCCUCAA | |
| 1703179 | 1703177 | UCUUGGAGAGGA | 1259 | 5048 | 5069 | 1703178 | AGACUGGGUUC | 1658 |
| | | ACCCAGUCUGA | | | | | CUCUCCAAGA | |
| 1703185 | 1703183 | UGGCAAUUCAUC | 1260 | 5122 | 5143 | 1703184 | AGGGAUUGGGA | 1659 |
| | | CCAAUCCCUGC | | | | | UGAAUUGCCA | |
| 1703188 | 1703186 | UAGCAGAUCCAG | 1261 | 5137 | 5158 | 1703187 | UUGCCUGUCCU | 1660 |
| | | GACAGGCAAUU | | | | | GGAUCUGCUA | |
| 1703194 | 1703192 | UAGGCAGGCAGC | 1262 | 5160 | 5181 | 1703193 | GAGGCCCAAGC | 1661 |
| | | UUGGGCCUCUA | | | | | UGCCUGCCUA | |
| 1703200 | 1703198 | UUGUCUCCUGAC | 1263 | 5191 | 5212 | 1703199 | ACUUGACAAGU | 1662 |
| | | UUGUCAAGUCA | | | | | CAGGAGACAA | |
| 1703203 | 1703201 | UAGGCUUUGGGA | 1264 | 5206 | 5227 | 1703202 | AGACACUGUUC | 1663 |
| | | ACAGUGUCUCC | | | | | CCAAAGCCUA | |
| 1703206 | 1703204 | UGUGCUCUGGUC | 1265 | 5218 | 5239 | 1703205 | CAAAGCCUUGA | 1664 |
| | | AAGGCUUUGGG | | | | | CCAGAGCACA | |
| 1703209 | 1703207 | UUCAGCGGGCUG | 1266 | 5231 | 5252 | 1703208 | AGAGCACCUCA | 1665 |
| | | AGGUGCUCUGG | | | | | GCCCGCUGAA | |
| 1703212 | 1703210 | UAUGGAGUUUGU | 1267 | 5248 | 5269 | 1703211 | UGACCUUGCAC | 1666 |
| | | GCAAGGUCAGC | | | | | AAACUCCAUA | |
| 1703218 | 1703216 | UAAGGCGGCUUC | 1268 | 5278 | 5299 | 1703217 | GAGAAAAGGGA | 1667 |
| | | CCUUUUCUCAU | | | | | AGCCGCCUUA | |
| 1703221 | 1703219 | UCAGCAAUGUUU | 1269 | 5293 | 5314 | 1703220 | GCCUUUGCAAA | 1668 |
| | | UGCAAAGGCGG | | | | | ACAUUGCUGA | |
| 1703224 | 1703222 | UCUGAGUUUCUU | 1270 | 5308 | 5329 | 1703223 | UGCUGCCUAAA | 1669 |
| | | UAGGCAGCAAU | | | | | GAAACUCAGA | |
| 1703230 | 1703228 | UAGUGGCAGAAU | 1271 | 5333 | 5354 | 1703229 | UCAGGCCCAAU | 1670 |
| | | UGGGCCUGAGG | | | | | UCUGCCACUA | |
| 1703233 | 1703231 | UUGUACCCAAAC | 1272 | 5348 | 5369 | 1703232 | CCACUUCUGGU | 1671 |
| | | CAGAAGUGGCA | | | | | UUGGGUACAA | |
| 1703236 | 1703234 | UAGGGUUGCCUU | 1273 | 5363 | 5384 | 1703235 | GUACAGUUAAA | 1672 |
| | | UAACUGUACCC | | | | | GGCAACCCUA | |
| 1703242 | 1703240 | UGCCCUGGAUUU | 1274 | 5391 | 5412 | 1703241 | UGGCAGUAGAA | 1673 |
| | | CUACUGCCAAG | | | | | AUCCAGGGCA | |
| 1703245 | 1703243 | UAGCUGCCAGCC | 1275 | 5411 | 5432 | 1703244 | CUCCCCUGGGGC | 1674 |
| | | CCAGGGGAGGC | | | | | UGGCAGCUA | |
| 1703248 | 1703246 | UUAAAGCUCUAG | 1276 | 5432 | 5453 | 1703247 | CGUGUGCAGCU | 1675 |
| | | CUGCACACGAA | | | | | AGAGCUUUAA | |
| 1703251 | 1703249 | UAGACUUCCUUU | 1277 | 5447 | 5468 | 1703250 | CUUUACCUGAA | 1676 |
| | | CAGGUAAAGCU | | | | | AGGAAGUCUA | |
| 1703254 | 1703252 | UUUCUGGGCCCA | 1278 | 5459 | 5480 | 1703253 | GGAAGUCUCUG | 1677 |
| | | GAGACUUCCUU | | | | | GGCCCAGAAA | |
| 1703257 | 1703255 | UUUGGUGGAGAG | 1279 | 5470 | 5491 | 1703256 | GGCCCAGAACUC | 1678 |
| | | UUCUGGGCCCA | | | | | UCCACCAAA | |
| 1703260 | 1703258 | UGGAGGCUCUUG | 1280 | 5478 | 5499 | 1703259 | ACUCUCCACCAA | 1679 |
| | | GUGGAGAGUUC | | | | | GAGCCUCCA | |
| 1703275 | 1703273 | UUUUCCUUCUCC | 1281 | 5540 | 5561 | 1703274 | AUCUGAGAAGG | 1680 |
| | | UUCUCAGAUCC | | | | | AGAAGGAAAA | |
| 1703278 | 1703276 | UCAAAUCUACCC | 1282 | 5555 | 5576 | 1703277 | GGAAAUGUGGG | 1681 |
| | | CACAUUUCCUU | | | | | GUAGAUUUGA | |
| 1703284 | 1703282 | UUAAUGAGGGGG | 1283 | 5585 | 5606 | 1703283 | GAGAUAUGCCC | 1682 |
| | | GCAUAUCUCUA | | | | | CCCUCAUUAA | |
| 1703287 | 1703285 | UCGAAACUGUUG | 1284 | 5600 | 5621 | 1703286 | CAUUACUGCCA | 1683 |
| | | GCAGUAAUGAG | | | | | ACAGUUUCGA | |
| 1703290 | 1703288 | UGUGAAGAAAUG | 1285 | 5615 | 5636 | 1703289 | UUUCGGCUGCA | 1684 |
| | | CAGCCGAAACU | | | | | UUUCUUCACA | |
| 1703293 | 1703291 | UGAGGAACCGAG | 1286 | 5630 | 5651 | 1703292 | UUCACGCACCUC | 1685 |
| | | GUGCGUGAAGA | | | | | GGUUCCUCA | |
| 1703296 | 1703294 | UCAAGAACUUCA | 1287 | 5645 | 5666 | 1703295 | UCCUCUUCCUGA | 1686 |
| | | GGAAGAGGAAC | | | | | AGUUCUUGA | |
| 1703299 | 1703297 | UUGAAGAGCAGG | 1288 | 5659 | 5680 | 1703298 | UUCUUGUGCCC | 1687 |
| | | GCACAAGAACU | | | | | UGCUCUUCAA | |
| 1703302 | 1703300 | UAGGCCCAUGGU | 1289 | 5672 | 5693 | 1703301 | CUCUUCAGCACC | 1688 |
| | | GCUGAAGAGCA | | | | | AUGGGCCUA | |
| 1703305 | 1703303 | UGCCUUCCGUAU | 1290 | 5687 | 5708 | 1703304 | GGCCUUCUUAU | 1689 |
| | | AAGAAGGCCCA | | | | | ACGGAAGGCA | |
| 1703308 | 1703306 | UGAUCCCAGAGC | 1291 | 5696 | 5717 | 1703307 | AUACGGAAGGC | 1690 |
| | | CUUCCGUAUAA | | | | | UCUGGGAUCA | |
| 1703311 | 1703309 | UGAGCCUGCCCC | 1292 | 5717 | 5738 | 1703310 | CCCCCUUGUGGG | 1691 |
| | | ACAAGGGGGAG | | | | | GCAGGCUCA | |
| 1703317 | 1703315 | UAGCACUGAUCA | 1293 | 5758 | 5779 | 1703316 | GGUUUAGGGUG | 1692 |
| | | CCCUAAACCAU | | | | | AUCAGUGCUA | |
| 1703323 | 1703321 | UAAGCCAGCGUG | 1294 | 5788 | 5809 | 1703322 | UUGAAAAGGCA | 1693 |
| | | CCUUUUCAAUU | | | | | CGCUGGCUUA | |
| 1703326 | 1703324 | UCUCAUUUAAGA | 1295 | 5803 | 5824 | 1703325 | GGCUUGUGAUC | 1694 |
| | | UCACAAGCCAG | | | | | UUAAAUGAGA | |
| 1703332 | 1703328 | UGGGGGAUUGUC | 1296 | 5814 | 5835 | 1703331 | UUAAAUGAGGA | 1695 |
| | | CUCAUUUAAGA | | | | | CAAUCCCCCA | |
| 1703335 | 1703333 | UGGGAGGAGUGC | 1297 | 5834 | 5855 | 1703334 | CAGGGCUGGGC | 1696 |
| | | CCAGCCCUGGG | | | | | ACUCCUCCCA | |
| 1703341 | 1703339 | UCACUGGCUCUG | 1298 | 5867 | 5888 | 1703340 | UCCCACCUGCAG | 1697 |
| | | CAGGUGGGAGA | | | | | AGCCAGUGA | |
| 1703344 | 1703342 | UUAUCCUAUCUA | 1299 | 5891 | 5912 | 1703343 | UGGGUGGGCUA | 1698 |
| | | GCCCACCCAAG | | | | | GAUAGGAUAA | |
| 1703347 | 1703345 | UAGCCGGCAUAC | 1300 | 5906 | 5927 | 1703346 | GGAUAUACUGU | 1699 |
| | | AGUAUAUCCUA | | | | | AUGCCGGCUA | |
| 1703350 | 1703348 | UUCAGCAGCUUG | 1301 | 5921 | 5942 | 1703349 | CGGCUCCUUCAA | 1700 |
| | | AAGGAGCCGGC | | | | | GCUGCUGAA | |
| 1703353 | 1703351 | UUAUUGAUAAAG | 1302 | 5936 | 5957 | 1703352 | GCUGACUCACU | 1701 |
| | | UGAGUCAGCAG | | | | | UUAUCAAUAA | |
| 1703359 | 1703357 | UUCUCACCACUG | 1303 | 5966 | 5987 | 1703358 | AAUUGACUUCA | 1702 |
| | | AAGUCAAUUUA | | | | | GUGGUGAGAA | |
| 1703362 | 1703360 | UGCAAACAGGAU | 1304 | 5981 | 6002 | 1703361 | UGAGACUGUAU | 1703 |
| | | ACAGUCUCACC | | | | | CCUGUUUGCA | |
| 1703365 | 1703363 | UGCACAACAAGC | 1305 | 5996 | 6017 | 1703364 | UUUGCUAUUGC | 1704 |
| | | AAUAGCAAACA | | | | | UUGUUGUGCA | |
| 1703377 | 1703375 | UUCCCUUUGCCC | 1306 | 6047 | 6068 | 1703376 | AGUUAACAUGG | 1705 |
| | | AUGUUAACUAU | | | | | GCAAAGGGAA | |
| 1703380 | 1703378 | UCUGCACCCCAA | 1307 | 6062 | 6083 | 1703379 | AGGGAGAUCUU | 1706 |
| | | GAUCUCCCUUU | | | | | GGGGUGCAGA | |
| 1703383 | 1703381 | UGAGGCAGUUUA | 1308 | 6077 | 6098 | 1703382 | UGCAGCACUUA | 1707 |
| | | AGUGCUGCACC | | | | | AACUGCCUCA | |
| 1703389 | 1703387 | UAAAUGUGGUUG | 1309 | 6107 | 6128 | 1703388 | UCAUGAUUUCA | 1708 |
| | | AAAUCAUGAAA | | | | | ACCACAUUUA | |
| 1703398 | 1703396 | UUCUAACUCCGU | 1310 | 6138 | 6159 | 1703397 | GGAGCAGCCAC | 1709 |
| | | GGCUGCUCCCU | | | | | GGAGUUAGAA | |
| 1703401 | 1703399 | UAAAGAGAAACC | 1311 | 6158 | 6179 | 1703400 | GGCCCUUGGGG | 1710 |
| | | CCAAGGGCCUC | | | | | UUUCUCUUUA | |
| 1703404 | 1703402 | UAGCCUGUCAGU | 1312 | 6172 | 6193 | 1703403 | CUCUUUUCCACU | 1711 |
| | | GGAAAAGAGAA | | | | | GACAGGCUA | |
| 1703407 | 1703405 | UCAGCUGCCUGG | 1313 | 6186 | 6207 | 1703406 | CAGGCUUUCCCA | 1712 |
| | | GAAAGCCUGUC | | | | | GGCAGCUGA | |
| 1703410 | 1703408 | UAGGGAAUGAAC | 1314 | 6201 | 6222 | 1703409 | AGCUGGCUAGU | 1713 |
| | | UAGCCAGCUGC | | | | | UCAUUCCCUA | |
| 1703413 | 1703411 | UCCUGCACCUGG | 1315 | 6218 | 6239 | 1703412 | CCUCCCCAGCCA | 1714 |
| | | CUGGGGAGGGA | | | | | GGUGCAGGA | |
| 1703416 | 1703414 | UUGUCCAUAUUC | 1316 | 6234 | 6255 | 1703415 | CAGGCGUAGGA | 1715 |
| | | CUACGCCUGCA | | | | | AUAUGGACAA | |
| 1703419 | 1703417 | UCCAAAGCAACC | 1317 | 6248 | 6269 | 1703418 | UGGACAUCUGG | 1716 |
| | | AGAUGUCCAUA | | | | | UUGCUUUGGA | |
| 1703422 | 1703420 | UGAGGGCAGCAG | 1318 | 6260 | 6281 | 1703421 | UGCUUUGGCCU | 1717 |
| | | GCCAAAGCAAC | | | | | GCUGCCCUCA | |
| 1703425 | 1703423 | UGGACCCCUGAA | 1319 | 6272 | 6293 | 1703424 | CUGCCCUCUUUC | 1718 |
| | | AGAGGGCAGCA | | | | | AGGGGUCCA | |
| 1703428 | 1703426 | UAUGAUUGUGGG | 1320 | 6287 | 6308 | 1703427 | GGUCCUAAGCCC | 1719 |
| | | CUUAGGACCCC | | | | | ACAAUCAUA | |
| 1703431 | 1703429 | UAGGUCUUAGGG | 1321 | 6302 | 6323 | 1703430 | AUCAUGCCUCCC | 1720 |
| | | AGGCAUGAUUG | | | | | UAAGACCUA | |
| 1703434 | 1703432 | UGAGGGAAGGAU | 1322 | 6317 | 6338 | 1703433 | GACCUUGGCAU | 1721 |
| | | GCCAAGGUCUU | | | | | CCUUCCCUCA | |
| 1703437 | 1703435 | UGUGCCAACGGC | 1323 | 6331 | 6352 | 1703436 | UCCCUCUAAGCC | 1722 |
| | | UUAGAGGGAAG | | | | | GUUGGCACA | |
| 1703440 | 1703438 | UAGGUGGCACAG | 1324 | 6344 | 6365 | 1703439 | UUGGCACCUCU | 1723 |
| | | AGGUGCCAACG | | | | | GUGCCACCUA | |
| 1703446 | 1703444 | UACAGGCUGUGU | 1325 | 6373 | 6394 | 1703445 | GCUCCAGACACA | 1724 |
| | | GUCUGGAGCCA | | | | | CAGCCUGUA | |
| 1703452 | 1703450 | UGGUGAAGCGAG | 1326 | 6403 | 6424 | 1703451 | CUGAGAUCACU | 1725 |
| | | UGAUCUCAGCU | | | | | CGCUUCACCA | |
| 1703455 | 1703453 | UAACAAAGAUGA | 1327 | 6418 | 6439 | 1703454 | UCACCCUCCUCA | 1726 |
| | | GGAGGGUGAAG | | | | | UCUUUGUUA | |
| 1703458 | 1703456 | UUGGCUUUACUU | 1328 | 6433 | 6454 | 1703457 | UUGUUCUCCAA | 1727 |
| | | GGAGAACAAAG | | | | | GUAAAGCCAA | |
| 1703470 | 1703468 | UAGAAAUCAGAA | 1329 | 6512 | 6533 | 1703469 | UCAUAAAACUU | 1728 |
| | | GUUUUAUGAAG | | | | | CUGAUUUCUA | |
| 1703473 | 1703471 | UUUUUCAAAGCU | 1330 | 6527 | 6548 | 1703472 | UUUCUCUUCAG | 1729 |
| | | GAAGAGAAAUC | | | | | CUUUGAAAAA | |
| 1703476 | 1703474 | UCCCAGGGUAAC | 1331 | 6540 | 6561 | 1703475 | UUGAAAAGGGU | 1730 |
| | | CCUUUUCAAAG | | | | | UACCCUGGGA | |
| 1703479 | 1703477 | UGGCUCUAGGCC | 1332 | 6555 | 6576 | 1703478 | CUGGGCACUGG | 1731 |
| | | AGUGCCCAGGG | | | | | CCUAGAGCCA | |
| 1703482 | 1703480 | UAGUCUAUUAGG | 1333 | 6572 | 6593 | 1703481 | GCCUCACCUCCU | 1732 |
| | | AGGUGAGGCUC | | | | | AAUAGACUA | |
| 1703485 | 1703483 | UAAACUCAUGGG | 1334 | 6587 | 6608 | 1703484 | AGACUUAGCCCC | 1733 |
| | | GCUAAGUCUAU | | | | | AUGAGUUUA | |
| 1703491 | 1703489 | UAGUGCCAGAAA | 1335 | 6617 | 6638 | 1703490 | GCAGGACUAUU | 1734 |
| | | UAGUCCUGCUC | | | | | UCUGGCACUA | |
| 1703494 | 1703492 | UAUCAUGGGACU | 1336 | 6632 | 6653 | 1703493 | GCACUUGCAAG | 1735 |
| | | UGCAAGUGCCA | | | | | UCCCAUGAUA | |
| 1703497 | 1703495 | UAGAAUUACCGA | 1337 | 6647 | 6668 | 1703496 | AUGAUUUCUUC | 1736 |
| | | AGAAAUCAUGG | | | | | GGUAAUUCUA | |
| 1703500 | 1703498 | UCCCACCCUCAGA | 1338 | 6656 | 6677 | 1703499 | UCGGUAAUUCU | 1737 |
| | | AUUACCGAAG | | | | | GAGGGUGGGA | |
| 1703506 | 1703504 | UCUAAGAUGAUU | 1339 | 6680 | 6701 | 1703505 | GGGACAUGAAA | 1738 |
| | | UCAUGUCCCUC | | | | | UCAUCUUAGA | |
| 1703509 | 1703507 | UAGACAGAAAGC | 1340 | 6695 | 6716 | 1703508 | CUUAGCUUAGC | 1739 |
| | | UAAGCUAAGAU | | | | | UUUCUGUCUA | |
| 1703512 | 1703510 | UUAUAUAGACAU | 1341 | 6710 | 6731 | 1703511 | UGUCUGUGAAU | 1740 |
| | | UCACAGACAGA | | | | | GUCUAUAUAA | |
| 1703515 | 1703513 | UAAACACACAAU | 1342 | 6725 | 6746 | 1703514 | AUAUAGUGUAU | 1741 |
| | | ACACUAUAUAG | | | | | UGUGUGUUUA | |
| 1703533 | 1703531 | UAAAGUUCACCU | 1343 | 121 | 142 | 1703532 | UCGACUAUCAG | 1742 |
| | | GAUAGUCGACA | | | | | GUGAACUUUA | |
| 1703536 | 1703534 | UCUGGUUCAAAG | 1344 | 128 | 149 | 1703535 | UCAGGUGAACU | 1743 |
| | | UUCACCUGAUA | | | | | UUGAACCAGA | |
| 1703539 | 1703537 | UUCUUCCAUCAC | 1345 | 167 | 188 | 1703538 | GAGUUCGAAGU | 1744 |
| | | UUCGAACUCCU | | | | | GAUGGAAGAA | |
| 1703542 | 1703540 | UAACUUUGGAGA | 1346 | 1179 | 1200 | 1703541 | UGGAUUUCCUC | 1745 |
| | | GGAAAUCCACA | | | | | UCCAAAGUUA | |
| 1703548 | 1703546 | UAUUUCCACGUG | 1347 | 1274 | 1295 | 1703547 | UUCACGUUUCA | 1746 |
| | | AAACGUGAACU | | | | | CGUGGAAAUA | |
| 1703554 | 1703552 | UUCUUUGCUUUU | 1348 | 1505 | 1526 | 1703553 | AUGGUCAGUAA | 1747 |
| | | ACUGACCAUGC | | | | | AAGCAAAGAA | |
| 1703560 | 1703558 | UGAUGUCUUGGC | 1349 | 1541 | 1562 | 1703559 | GACAAAAAAGC | 1748 |
| | | UUUUUUGUCAU | | | | | CAAGACAUCA | |
| 1703563 | 1703561 | UCGUGUGGAUGU | 1350 | 1547 | 1568 | 1703562 | AAAGCCAAGAC | 1749 |
| | | CUUGGCUUUUU | | | | | AUCCACACGA | |
| 1703572 | 1703570 | UCAAGGCCUAUU | 1351 | 1580 | 1601 | 1703571 | ACCUUGAAAAA | 1750 |
| | | UUUCAAGGUUU | | | | | UAGGCCUUGA | |
| 1703575 | 1703573 | UGACGUAUUUAG | 1352 | 1680 | 1701 | 1703574 | CUUCCUCUCCUA | 1751 |
| | | GAGAGGAAGGU | | | | | AAUACGUCA | |
| 1703578 | 1703576 | UGUGACAGAAGA | 1353 | 1691 | 1712 | 1703577 | AAAUACGUCUC | 1752 |
| | | GACGUAUUUAG | | | | | UUCUGUCACA | |
| 1703590 | 1703588 | UUUAAUUAUCUG | 1354 | 2141 | 2162 | 1703589 | GGGAAGGUGCA | 1753 |
| | | CACCUUCCCGC | | | | | GAUAAUUAAA | |
| 1703593 | 1703591 | UCUUCUUAUUAA | 1355 | 2148 | 2169 | 1703592 | UGCAGAUAAUU | 1754 |
| | | UUAUCUGCACC | | | | | AAUAAGAAGA | |
| 1703596 | 1703594 | UAGAUCCAGCUU | 1356 | 2156 | 2177 | 1703595 | AUUAAUAAGAA | 1755 |
| | | CUUAUUAAUUA | | | | | GCUGGAUCUA | |
| 1703599 | 1703597 | UCGUUGCUAAGA | 1357 | 2164 | 2185 | 1703598 | GAAGCUGGAUC | 1756 |
| | | UCCAGCUUCUU | | | | | UUAGCAACGA | |
| 1703605 | 1703603 | UGGGACGUGUUU | 1358 | 2207 | 2228 | 1703604 | GAUAAUAUCAA | 1757 |
| | | GAUAUUAUCCU | | | | | ACACGUCCCA | |
| 1703611 | 1703609 | UUGGUUUGUAGA | 1359 | 2241 | 2262 | 1703610 | UGCAAAUAGUC | 1758 |
| | | CUAUUUGCACA | | | | | UACAAACCAA | |
| 1703623 | 1703621 | UAGUCAAGCUUC | 1360 | 2341 | 2362 | 1703622 | AAAAUCUGAGA | 1759 |
| | | UCAGAUUUUAC | | | | | AGCUUGACUA | |
| 1703626 | 1703624 | UCUCUGUCCUUG | 1361 | 2353 | 2374 | 1703625 | GCUUGACUUCA | 1760 |
| | | AAGUCAAGCUU | | | | | AGGACAGAGA | |
| 1703629 | 1703627 | UAAUCUUUUUAU | 1362 | 2418 | 2439 | 1703628 | GCGGAGGAAAU | 1761 |
| | | UUCCUCCGCCA | | | | | AAAAAGAUUA | |
| 1703635 | 1703633 | UGCUUGUGGGUU | 1363 | 2431 | 2452 | 1703634 | AAAGAUUGAAA | 1762 |
| | | UCAAUCUUUUU | | | | | CCCACAAGCA | |
| 1703638 | 1703636 | UUCUCCUCUCCAC | 1364 | 2669 | 2690 | 1703637 | CAAUAAUUGUG | 1763 |
| | | AAUUAUUGAC | | | | | GAGAGGAGAA | |
| 1703653 | 1703651 | UUUAAGUGAUUA | 1365 | 2766 | 2787 | 1703652 | UUAAUUGGUUA | 1764 |
| | | ACCAAUUAACC | | | | | AUCACUUAAA | |
| 1703659 | 1703657 | UCUCCCAUCACU | 1366 | 2818 | 2839 | 1703658 | UUCAAAAUCAG | 1765 |
| | | GAUUUUGAAGU | | | | | UGAUGGGAGA | |
| 1703665 | 1703663 | UAUGAAAUUUGC | 1367 | 2834 | 2855 | 1703664 | GGAGUAAGAGC | 1766 |
| | | UCUUACUCCCA | | | | | AAAUUUCAUA | |
| 1703674 | 1703672 | UCAUCAAUUUGG | 1368 | 2849 | 2870 | 1703673 | UUCAUCUUUCC | 1767 |
| | | AAAGAUGAAAU | | | | | AAAUUGAUGA | |
| 1703677 | 1703675 | UUUACUAGCCCA | 1369 | 2862 | 2883 | 1703676 | AUUGAUGGGUG | 1768 |
| | | CCCAUCAAUUU | | | | | GGCUAGUAAA | |
| 1703689 | 1703687 | UUGGCCAUGUUU | 1370 | 2899 | 2920 | 1703688 | AACAUUCAAAA | 1769 |
| | | UUGAAUGUUUU | | | | | ACAUGGCCAA | |
| 1703692 | 1703690 | UUUGGAUGUGGC | 1371 | 2906 | 2927 | 1703691 | AAAAACAUGGC | 1770 |
| | | CAUGUUUUUGA | | | | | CACAUCCAAA | |
| 1703695 | 1703693 | UAUUGCCUGAGG | 1372 | 2922 | 2943 | 1703694 | CCAACAUUUCCU | 1771 |
| | | AAAUGUUGGAU | | | | | CAGGCAAUA | |
| 1703698 | 1703696 | UAAAGGAAUUGC | 1373 | 2928 | 2949 | 1703697 | UUUCCUCAGGC | 1772 |
| | | CUGAGGAAAUG | | | | | AAUUCCUUUA | |
| 1703701 | 1703699 | UAAGAAUCAAAA | 1374 | 2936 | 2957 | 1703700 | GGCAAUUCCUU | 1773 |
| | | GGAAUUGCCUG | | | | | UUGAUUCUUA | |
| 1703704 | 1703702 | UGAAGAAAAAAG | 1375 | 2944 | 2965 | 1703703 | CUUUUGAUUCU | 1774 |
| | | AAUCAAAAGGA | | | | | UUUUUCUUCA | |
| 1703713 | 1703711 | UCAAGUUUCAAA | 1376 | 3086 | 3107 | 1703712 | AACAAAGGAUU | 1775 |
| | | UCCUUUGUUGC | | | | | UGAAACUUGA | |
| 1703716 | 1703714 | UAACACACCAAG | 1377 | 3093 | 3114 | 1703715 | GAUUUGAAACU | 1776 |
| | | UUUCAAAUCCU | | | | | UGGUGUGUUA | |
| 1703719 | 1703717 | UGUCUUUUCUAA | 1378 | 3462 | 3483 | 1703718 | AUGACCUCCUU | 1777 |
| | | GGAGGUCAUCC | | | | | AGAAAAGACA | |
| 1703722 | 1703720 | UAGGUCAGUCUU | 1379 | 3468 | 3489 | 1703721 | UCCUUAGAAAA | 1778 |
| | | UUCUAAGGAGG | | | | | GACUGACCUA | |
| 1703728 | 1703726 | UAUAAAACAGGG | 1380 | 3564 | 3585 | 1703727 | CCACAGAAACCC | 1779 |
| | | UUUCUGUGGAG | | | | | UGUUUUAUA | |
| 1703731 | 1703729 | UGAACUCAAUAA | 1381 | 3571 | 3592 | 1703730 | AACCCUGUUUU | 1780 |
| | | AACAGGGUUUC | | | | | AUUGAGUUCA | |
| 1703734 | 1703732 | UACCUUCAGAAC | 1382 | 3578 | 3599 | 1703733 | UUUUAUUGAGU | 1781 |
| | | UCAAUAAAACA | | | | | UCUGAAGGUA | |
| 1703737 | 1703735 | UAGUUCCAACCU | 1383 | 3585 | 3606 | 1703736 | GAGUUCUGAAG | 1782 |
| | | UCAGAACUCAA | | | | | GUUGGAACUA | |
| 1703740 | 1703738 | UCAAAGAGAACU | 1384 | 3645 | 3666 | 1703739 | GGGCUAACCAG | 1783 |
| | | GGUUAGCCCUA | | | | | UUCUCUUUGA | |
| 1703743 | 1703741 | UAGUCCUUACAA | 1385 | 3653 | 3674 | 1703742 | CAGUUCUCUUU | 1784 |
| | | AGAGAACUGGU | | | | | GUAAGGACUA | |
| 1703746 | 1703744 | UAGAGGCACAAG | 1386 | 3662 | 3683 | 1703745 | UUGUAAGGACU | 1785 |
| | | UCCUUACAAAG | | | | | UGUGCCUCUA | |
| 1703752 | 1703750 | UUCAGAUCAUGA | 1387 | 4034 | 4055 | 1703751 | CCACGAAUCUCA | 1786 |
| | | GAUUCGUGGUG | | | | | UGAUCUGAA | |
| 1703755 | 1703753 | UGAACCGAAUCA | 1388 | 4042 | 4063 | 1703754 | CUCAUGAUCUG | 1787 |
| | | GAUCAUGAGAU | | | | | AUUCGGUUCA | |
| 1703758 | 1703756 | UAAAGAACCAUU | 1389 | 4389 | 4410 | 1703757 | CCCCUUGGAAA | 1788 |
| | | UCCAAGGGGGU | | | | | UGGUUCUUUA | |
| 1703761 | 1703759 | UUAUGUAUAUGU | 1390 | 4451 | 4472 | 1703760 | AGCAGCUGAAC | 1789 |
| | | UCAGCUGCUCC | | | | | AUAUACAUAA | |
| 1703764 | 1703762 | UAACAUCUAUGU | 1391 | 4457 | 4478 | 1703763 | UGAACAUAUAC | 1790 |
| | | AUAUGUUCAGC | | | | | AUAGAUGUUA | |
| 1703767 | 1703765 | UUCCAACUACAA | 1392 | 4499 | 4520 | 1703766 | CCUGUUGAGUU | 1791 |
| | | CUCAACAGGGU | | | | | GUAGUUGGAA | |
| 1703770 | 1703768 | UCAGACAAAUCC | 1393 | 4507 | 4528 | 1703769 | GUUGUAGUUGG | 1792 |
| | | AACUACAACUC | | | | | AUUUGUCUGA | |
| 1703773 | 1703771 | UGCAUAAACAGA | 1394 | 4514 | 4535 | 1703772 | UUGGAUUUGUC | 1793 |
| | | CAAAUCCAACU | | | | | UGUUUAUGCA | |
| 1703776 | 1703774 | UAAUCCAAGCAU | 1395 | 4521 | 4542 | 1703775 | UGUCUGUUUAU | 1794 |
| | | AAACAGACAAA | | | | | GCUUGGAUUA | |
| 1703779 | 1703777 | UCUCUGGUGAAU | 1396 | 4529 | 4550 | 1703778 | UAUGCUUGGAU | 1795 |
| | | CCAAGCAUAAA | | | | | UCACCAGAGA | |
| 1703782 | 1703780 | UAUAGUCACUCU | 1397 | 4536 | 4557 | 1703781 | GGAUUCACCAG | 1796 |
| | | GGUGAAUCCAA | | | | | AGUGACUAUA | |
| 1703785 | 1703783 | UAGCAUUUCAAG | 1398 | 4589 | 4610 | 1703784 | CGCAUGUAUCU | 1797 |
| | | AUACAUGCGUC | | | | | UGAAAUGCUA | |
| 1703788 | 1703786 | UUCUUUACAAGC | 1399 | 4597 | 4618 | 1703787 | UCUUGAAAUGC | 1798 |
| | | AUUUCAAGAUA | | | | | UUGUAAAGAA | |
| 1703794 | 1703792 | UGGGUGGGUUAG | 1400 | 4613 | 4634 | 1703793 | AAGAGGUUUCU | 1799 |
| | | AAACCUCUUUA | | | | | AACCCACCCA | |
| 1703797 | 1703795 | UAGGAAAGCCUU | 1401 | 4704 | 4725 | 1703796 | AAGUUUUGAAA | 1800 |
| | | UCAAAACUUGG | | | | | GGCUUUCCUA | |
| 1703809 | 1703807 | UCUUGUCAAGUC | 1402 | 5181 | 5202 | 1703808 | AGGAAGGAUGA | 1801 |
| | | AUCCUUCCUCA | | | | | CUUGACAAGA | |
| 1703815 | 1703813 | UAGUUUCUUUAG | 1403 | 5305 | 5326 | 1703814 | CAUUGCUGCCU | 1802 |
| | | GCAGCAAUGUU | | | | | AAAGAAACUA | |
| 1703818 | 1703816 | UUUUAACUGUAC | 1404 | 5354 | 5375 | 1703817 | CUGGUUUGGGU | 1803 |
| | | CCAAACCAGAA | | | | | ACAGUUAAAA | |
| 1703827 | 1703825 | UAUCCCUUCAAC | 1405 | 5522 | 5543 | 1703826 | UUCUCCUAAGU | 1804 |
| | | UUAGGAGAAUU | | | | | UGAAGGGAUA | |
| 1703830 | 1703828 | UUUCUCAGAUCC | 1406 | 5529 | 5550 | 1703829 | AAGUUGAAGGG | 1805 |
| | | CUUCAACUUAG | | | | | AUCUGAGAAA | |
| 1703833 | 1703831 | UACAUUUCCUUC | 1407 | 5543 | 5564 | 1703832 | UGAGAAGGAGA | 1806 |
| | | UCCUUCUCAGA | | | | | AGGAAAUGUA | |
| 1703845 | 1703843 | UUGAUCACCCUA | 1408 | 5753 | 5774 | 1703844 | AUCAUGGUUUA | 1807 |
| | | AACCAUGAUCU | | | | | GGGUGAUCAA | |
| 1703848 | 1703846 | UUUUAUCUGCCA | 1409 | 5768 | 5789 | 1703847 | GAUCAGUGCUG | 1808 |
| | | GCACUGAUCAC | | | | | GCAGAUAAAA | |
| 1703851 | 1703849 | UCCUUUUCAAUU | 1410 | 5777 | 5798 | 1703850 | UGGCAGAUAAA | 1809 |
| | | UAUCUGCCAGC | | | | | UUGAAAAGGA | |
| 1703854 | 1703852 | UCAGCGUGCCUU | 1411 | 5784 | 5805 | 1703853 | UAAAUUGAAAA | 1810 |
| | | UUCAAUUUAUC | | | | | GGCACGCUGA | |
| 1703857 | 1703855 | UAGCCAGCGUGC | 1412 | 5787 | 5808 | 1703856 | AUUGAAAAGGC | 1811 |
| | | CUUUUCAAUUU | | | | | ACGCUGGCUA | |
| 1703869 | 1703867 | UGGCAUACAGUA | 1413 | 5902 | 5923 | 1703868 | GAUAGGAUAUA | 1812 |
| | | UAUCCUAUCUA | | | | | CUGUAUGCCA | |
| 1703875 | 1703873 | UGAACUAUUGAU | 1414 | 5940 | 5961 | 1703874 | ACUCACUUUAU | 1813 |
| | | AAAGUGAGUCA | | | | | CAAUAGUUCA | |
| 1703878 | 1703876 | UUUUAAAUGGAA | 1415 | 5948 | 5969 | 1703877 | UAUCAAUAGUU | 1814 |
| | | CUAUUGAUAAA | | | | | CCAUUUAAAA | |
| 1703881 | 1703879 | UAAGUCAAUUUA | 1416 | 5955 | 5976 | 1703880 | AGUUCCAUUUA | 1815 |
| | | AAUGGAACUAU | | | | | AAUUGACUUA | |
| 1703884 | 1703882 | UACCACUGAAGU | 1417 | 5962 | 5983 | 1703883 | UUUAAAUUGAC | 1816 |
| | | CAAUUUAAAUG | | | | | UUCAGUGGUA | |
| 1703890 | 1703888 | UAAUAGCAAACA | 1418 | 5985 | 6006 | 1703889 | ACUGUAUCCUG | 1817 |
| | | GGAUACAGUCU | | | | | UUUGCUAUUA | |
| 1703893 | 1703891 | UAACAAGCAAUA | 1419 | 5992 | 6013 | 1703892 | CCUGUUUGCUA | 1818 |
| | | GCAAACAGGAU | | | | | UUGCUUGUUA | |
| 1703896 | 1703894 | UAUAGCACAACA | 1420 | 5999 | 6020 | 1703895 | GCUAUUGCUUG | 1819 |
| | | AGCAAUAGCAA | | | | | UUGUGCUAUA | |
| 1703899 | 1703897 | UAUGUUAACUAU | 1421 | 6036 | 6057 | 1703898 | AUGUGUAAGAU | 1820 |
| | | CUUACACAUUC | | | | | AGUUAACAUA | |
| 1703902 | 1703900 | UCUUUGCCCAUG | 1422 | 6044 | 6065 | 1703901 | GAUAGUUAACA | 1821 |
| | | UUAACUAUCUU | | | | | UGGGCAAAGA | |
| 1703905 | 1703903 | UUCUCCCUUUGC | 1423 | 6049 | 6070 | 1703904 | UUAACAUGGGC | 1822 |
| | | CCAUGUUAACU | | | | | AAAGGGAGAA | |
| 1703911 | 1703909 | UAAAUCAUGAAA | 1424 | 6096 | 6117 | 1703910 | CGUAACCCUUU | 1823 |
| | | AGGGUUACGAG | | | | | UCAUGAUUUA | |
| 1703917 | 1703915 | UUAGCAAAUGUG | 1425 | 6111 | 6132 | 1703916 | GAUUUCAACCA | 1824 |
| | | GUUGAAAUCAU | | | | | CAUUUGCUAA | |
| 1703920 | 1703918 | UACCAGAUGUCC | 1426 | 6240 | 6261 | 1703919 | UAGGAAUAUGG | 1825 |
| | | AUAUUCCUACG | | | | | ACAUCUGGUA | |
| 1703923 | 1703921 | UAAAGCAACCAG | 1427 | 6246 | 6267 | 1703922 | UAUGGACAUCU | 1826 |
| | | AUGUCCAUAUU | | | | | GGUUGCUUUA | |
| 1703926 | 1703924 | UUUUACUUGGAG | 1428 | 6429 | 6450 | 1703925 | AUCUUUGUUCU | 1827 |
| | | AACAAAGAUGA | | | | | CCAAGUAAAA | |
| 1703932 | 1703930 | UUUUUAUGAAGC | 1429 | 6500 | 6521 | 1703931 | AGACCUGCAGC | 1828 |
| | | UGCAGGUCUGU | | | | | UUCAUAAAAA | |
| 1703935 | 1703933 | UAAUCAGAAGUU | 1430 | 6509 | 6530 | 1703934 | GCUUCAUAAAA | 1829 |
| | | UUAUGAAGCUG | | | | | CUUCUGAUUA | |
| 1703944 | 1703942 | UACCCUUUUCAA | 1431 | 6531 | 6552 | 1703943 | UCUUCAGCUUU | 1830 |
| | | AGCUGAAGAGA | | | | | GAAAAGGGUA | |
| 1703950 | 1703948 | UAUAGUCCUGCU | 1432 | 6607 | 6628 | 1703949 | GCCAUGUUGAG | 1831 |
| | | CAACAUGGCAA | | | | | CAGGACUAUA | |
| 1703959 | 1703957 | UCCUCAGAAUUA | 1433 | 6651 | 6672 | 1703958 | UUUCUUCGGUA | 1832 |
| | | CCGAAGAAAUC | | | | | AUUCUGAGGA | |
| 1703962 | 1703960 | UUAAGCUAAGAU | 1434 | 6684 | 6705 | 1703961 | CAUGAAAUCAU | 1833 |
| | | GAUUUCAUGUC | | | | | CUUAGCUUAA | |
| 1703965 | 1703963 | UCAGAAAGCUAA | 1435 | 6692 | 6713 | 1703964 | CAUCUUAGCUU | 1834 |
| | | GCUAAGAUGAU | | | | | AGCUUUCUGA | |
| 1703971 | 1703969 | UUAGACAUUCAC | 1436 | 6706 | 6727 | 1703970 | UUUCUGUCUGU | 1835 |
| | | AGACAGAAAGC | | | | | GAAUGUCUAA | |
| 1703977 | 1703975 | UACACAAUACAC | 1437 | 6721 | 6742 | 1703976 | GUCUAUAUAGU | 1836 |
| | | UAUAUAGACAU | | | | | GUAUUGUGUA | |
| 1703983 | 1703981 | UAAUCAUUUGUU | 1438 | 6737 | 6758 | 1703982 | GUGUGUUUUAA | 1837 |
| | | AAAACACACAA | | | | | CAAAUGAUUA | |
| 1703989 | 1703987 | UCAACAGUCAGU | 1439 | 6751 | 6772 | 1703988 | AUGAUUUACAC | 1838 |
| | | GUAAAUCAUUU | | | | | UGACUGUUGA | |
| 1703992 | 1703990 | UUUUUACAGCAA | 1440 | 6759 | 6780 | 1703991 | CACUGACUGUU | 1839 |
| | | CAGUCAGUGUA | | | | | GCUGUAAAAA | |
| 1703995 | 1703993 | UAAAUUCACUUU | 1441 | 6767 | 6788 | 1703994 | GUUGCUGUAAA | 1840 |
| | | UACAGCAACAG | | | | | AGUGAAUUUA | |
| 1704001 | 1703999 | UUAAUAACUUUA | 1442 | 6783 | 6804 | 1704000 | AUUUGGAAAUA | 1841 |
| | | UUUCCAAAUUC | | | | | AAGUUAUUAA | |
| 1704004 | 1704002 | UAUCAGAGUAAU | 1443 | 6790 | 6811 | 1704003 | AAUAAAGUUAU | 1842 |
| | | AACUUUAUUUC | | | | | UACUCUGAUA | |

### Example 5: Effect of RNAi Compounds on Human MAPT RNA In Vitro, Single Dose

RNAi compounds described herein above were tested for their single dose effects on MAPT RNA in vitro. The RNAi compounds were tested in a series of experiments that had the same culture conditions.

Cultured A-172 cells were treated with RNAi compound at a concentration of 1 nM by LipofectAMINE RNAiMAX at a density of 10,000 cells per well. After a treatment period of 72 hours, total RNA was isolated from the cells and MAPT RNA levels were measured by quantitative real-time RTPCR. MAPT RNA was measured by human primer probe set RTS3104 (described herein above). MAPT RNA levels were normalized to total RNA content, as measured by RIBOGREEN®. Reduction of MAPT RNA is presented in the table below as percent MAPT RNA relative to the amount of MAPT RNA in untreated control cells (% UTC).

Each separate experiment described in this example is presented in a separate table. The values marked with a “†” indicate that the RNAi compound is complementary to the amplicon region of the primer probe set.

TABLE 23
Reduction of MAPT RNA by RNAi compounds
that target human MAPT nucleic acid

| Compound | MAPT |
|---|---|
| Number | (% UTC) |
| 1612982 | 30 |
| 1702345 | 30 |
| 1702369 | 77 |
| 1702384 | 44 |
| 1702411 | 59 |
| 1702444 | 78 |
| 1702483 | 75 |
| 1702504 | 69 |
| 1702534 | 70 |
| 1702537 | 81 |
| 1702555 | 83 |
| 1702639 | 46 |
| 1702642 | 77 |
| 1702684 | 65 |
| 1702690 | 57 |
| 1702696 | 65 |
| 1702705 | 49 |
| 1702732 | 49† |
| 1702744 | 74 |
| 1702750 | 72 |
| 1702792 | 32 |
| 1702804 | 58 |
| 1702810 | 61 |
| 1702816 | 39 |
| 1702846 | 58 |
| 1702861 | 57 |
| 1702870 | 83 |
| 1702927 | 69 |
| 1702957 | 59 |
| 1702966 | 57 |
| 1702975 | 67 |
| 1703011 | 87 |
| 1703032 | 81 |
| 1703050 | 71 |
| 1703080 | 58 |
| 1703098 | 84 |
| 1703119 | 81 |
| 1703182 | 93 |
| 1703188 | 65 |
| 1703197 | 77 |
| 1703206 | 81 |
| 1703230 | 66 |
| 1703239 | 85 |
| 1703248 | 64 |
| 1703272 | 66 |
| 1703278 | 66 |
| 1703299 | 55 |
| 1703335 | 83 |
| 1703380 | 62 |
| 1703383 | 68 |
| 1703389 | 60 |
| 1703392 | 79 |
| 1703413 | 84 |
| 1703416 | 72 |
| 1703422 | 85 |
| 1703431 | 83 |
| 1703473 | 56 |
| 1703515 | 48 |
| 1703518 | 51 |
| 1703521 | 70 |
| 1703545 | 92 |
| 1703611 | 40 |
| 1703653 | 39 |
| 1703656 | 37 |
| 1703665 | 51 |
| 1703671 | 24 |
| 1703686 | 51 |
| 1703707 | 96 |
| 1703716 | 51 |
| 1703740 | 49 |
| 1703764 | 56 |
| 1703773 | 50 |
| 1703779 | 79 |
| 1703791 | 59 |
| 1703797 | 63 |
| 1703842 | 73 |
| 1703914 | 57 |
| 1703917 | 59 |
| 1703950 | 58 |

TABLE 24
Reduction of MAPT RNA by RNAi compounds
that target human MAPT nucleic acid

| Compound | MAPT |
|---|---|
| Number | (% UTC) |
| 1612982 | 40 |
| 1702405 | 85 |
| 1702417 | 74 |
| 1702420 | 66 |
| 1702426 | 52 |
| 1702429 | 65 |
| 1702435 | 68 |
| 1702450 | 66 |
| 1702453 | 44 |
| 1702459 | 63 |
| 1702489 | 67 |
| 1702501 | 82 |
| 1702516 | 78 |
| 1702528 | 58 |
| 1702600 | 70 |
| 1702651 | 67 |
| 1702660 | 75 |
| 1702678 | 70 |
| 1702687 | 77 |
| 1702726 | 54† |
| 1702789 | 50 |
| 1702822 | 53 |
| 1702843 | 80 |
| 1702864 | 56 |
| 1702876 | 86 |
| 1702879 | 72 |
| 1702891 | 78 |
| 1702894 | 58 |
| 1702903 | 59 |
| 1702912 | 67 |
| 1702945 | 76 |
| 1702948 | 75 |
| 1702954 | 57 |
| 1702963 | 53 |
| 1702999 | 77 |
| 1703017 | 83 |
| 1703023 | 52 |
| 1703056 | 90 |
| 1703086 | 38 |
| 1703089 | 51 |
| 1703101 | 90 |
| 1703110 | 80 |
| 1703185 | 68 |
| 1703215 | 82 |
| 1703218 | 73 |
| 1703233 | 73 |
| 1703236 | 73 |
| 1703260 | 70 |
| 1703284 | 72 |
| 1703287 | 53 |
| 1703314 | 65 |
| 1703317 | 74 |
| 1703374 | 67 |
| 1703404 | 71 |
| 1703419 | 85 |
| 1703461 | 85 |
| 1703470 | 56 |
| 1703500 | 79 |
| 1703560 | 102 |
| 1703602 | 79 |
| 1703632 | 50† |
| 1703680 | 62 |
| 1703704 | 62 |
| 1703755 | 63 |
| 1703782 | 48 |
| 1703800 | 70 |
| 1703824 | 73 |
| 1703845 | 77 |
| 1703857 | 64 |
| 1703875 | 46 |
| 1703878 | 58 |
| 1703881 | 49 |
| 1703905 | 84 |
| 1703923 | 70 |
| 1703926 | 66 |
| 1703959 | 75 |
| 1703965 | 69 |
| 1703992 | 55 |
| 1703995 | 49 |

TABLE 25
Reduction of MAPT RNA by RNAi compounds
that target human MAPT nucleic acid

| Compound | MAPT |
|---|---|
| Number | (% UTC) |
| 1612982 | 83 |
| 1702357 | 52 |
| 1702366 | 52 |
| 1702372 | 86 |
| 1702387 | 42 |
| 1702393 | 94 |
| 1702402 | 88 |
| 1702423 | 69 |
| 1702486 | 73 |
| 1702492 | 75 |
| 1702507 | 83 |
| 1702525 | 155 |
| 1702531 | 94 |
| 1702552 | 95 |
| 1702561 | 56 |
| 1702564 | 75 |
| 1702567 | 81 |
| 1702594 | 71 |
| 1702597 | 34 |
| 1702630 | 86 |
| 1702654 | 75 |
| 1702669 | 83 |
| 1702672 | 56 |
| 1702798 | 85 |
| 1702885 | 72 |
| 1702897 | 82 |
| 1702939 | 76 |
| 1702960 | 77 |
| 1702981 | 72 |
| 1702987 | 72 |
| 1702990 | 89 |
| 1703053 | 76 |
| 1703062 | 67 |
| 1703083 | 61 |
| 1703104 | 68 |
| 1703107 | 83 |
| 1703140 | 72 |
| 1703143 | 88 |
| 1703152 | 83 |
| 1703173 | 84 |
| 1703176 | 71 |
| 1703179 | 55 |
| 1703254 | 80 |
| 1703269 | 54 |
| 1703275 | 72 |
| 1703281 | 52 |
| 1703311 | 54 |
| 1703323 | 88 |
| 1703341 | 71 |
| 1703362 | 72 |
| 1703398 | 94 |
| 1703401 | 65 |
| 1703479 | 82 |
| 1703482 | 47 |
| 1703512 | 70 |
| 1703524 | 38 |
| 1703587 | 75 |
| 1703596 | 95 |
| 1703605 | 92 |
| 1703647 | 94 |
| 1703662 | 94 |
| 1703674 | 71 |
| 1703710 | 51 |
| 1703725 | 41 |
| 1703728 | 71 |
| 1703731 | 56 |
| 1703734 | 45 |
| 1703737 | 75 |
| 1703746 | 75 |
| 1703758 | 50 |
| 1703761 | 61 |
| 1703770 | 63 |
| 1703776 | 65 |
| 1703794 | 60 |
| 1703854 | 57 |
| 1703896 | 46 |
| 1703962 | 49 |
| 1703983 | 68 |
| 1704004 | 57 |

TABLE 26
Reduction of MAPT RNA by RNAi compounds
that target human MAPT nucleic acid

| Compound | MAPT |
|---|---|
| Number | (% UTC) |
| 1612982 | 64 |
| 1702351 | 79 |
| 1702354 | 81 |
| 1702399 | 87 |
| 1702414 | 52 |
| 1702432 | 44 |
| 1702498 | 63 |
| 1702513 | 31 |
| 1702540 | 80 |
| 1702546 | 95 |
| 1702576 | 110 |
| 1702588 | 95 |
| 1702603 | 75 |
| 1702612 | 73 |
| 1702633 | 76 |
| 1702666 | 83 |
| 1702717 | 89 |
| 1702723 | 82 |
| 1702738 | 89† |
| 1702747 | 59 |
| 1702753 | 92 |
| 1702765 | 72 |
| 1702807 | 69 |
| 1702831 | 74 |
| 1702834 | 80 |
| 1702858 | 78 |
| 1702930 | 79 |
| 1702978 | 59 |
| 1703002 | 68 |
| 1703005 | 86 |
| 1703044 | 84 |
| 1703059 | 93 |
| 1703074 | 77 |
| 1703092 | 60 |
| 1703113 | 65 |
| 1703116 | 89 |
| 1703125 | 86 |
| 1703128 | 76 |
| 1703137 | 57 |
| 1703146 | 94 |
| 1703251 | 98 |
| 1703290 | 74 |
| 1703308 | 82 |
| 1703320 | 51 |
| 1703332 | 57 |
| 1703353 | 67 |
| 1703386 | 71 |
| 1703425 | 54 |
| 1703428 | 87 |
| 1703434 | 65 |
| 1703476 | 89 |
| 1703485 | 72 |
| 1703503 | 92 |
| 1703536 | 62 |
| 1703539 | 55 |
| 1703554 | 48 |
| 1703569 | 26 |
| 1703572 | 64 |
| 1703575 | 75 |
| 1703578 | 41 |
| 1703581 | 55 |
| 1703584 | 57 |
| 1703617 | 91 |
| 1703629 | 67† |
| 1703683 | 69 |
| 1703692 | 59 |
| 1703701 | 61 |
| 1703719 | 80 |
| 1703749 | 69 |
| 1703788 | 54 |
| 1703803 | 46 |
| 1703812 | 42 |
| 1703827 | 43 |
| 1703839 | 49 |
| 1703863 | 42 |
| 1703872 | 35 |
| 1703890 | 40 |
| 1703944 | 28 |
| 1703947 | 27 |

TABLE 27
Reduction of MAPT RNA by RNAi compounds
that target human MAPT nucleic acid

| Compound | MAPT |
|---|---|
| Number | (% UTC) |
| 1612982 | 55 |
| 1702348 | 82 |
| 1702378 | 47 |
| 1702390 | 77 |
| 1702396 | 76 |
| 1702447 | 38 |
| 1702456 | 66 |
| 1702468 | 77 |
| 1702474 | 79 |
| 1702558 | 77 |
| 1702585 | 80 |
| 1702618 | 120 |
| 1702621 | 141 |
| 1702648 | 60 |
| 1702708 | 55 |
| 1702711 | 102 |
| 1702756 | 102 |
| 1702759 | 56 |
| 1702780 | 83 |
| 1702783 | 114 |
| 1702786 | 23 |
| 1702795 | 32 |
| 1702801 | 39 |
| 1702819 | 37 |
| 1702840 | 78 |
| 1702867 | 65 |
| 1702873 | 63 |
| 1702882 | 70 |
| 1702900 | 67 |
| 1702909 | 98 |
| 1702918 | 52 |
| 1702951 | 68 |
| 1702969 | 60 |
| 1702972 | 77 |
| 1702993 | 65 |
| 1703014 | 99 |
| 1703038 | 54 |
| 1703071 | 17 |
| 1703122 | 65 |
| 1703131 | 49 |
| 1703155 | 96 |
| 1703158 | 72 |
| 1703161 | 59 |
| 1703164 | 53 |
| 1703194 | 65 |
| 1703203 | 51 |
| 1703209 | 77 |
| 1703221 | 72 |
| 1703227 | 73 |
| 1703296 | 44 |
| 1703356 | 41 |
| 1703365 | 49 |
| 1703443 | 78 |
| 1703449 | 73 |
| 1703458 | 55 |
| 1703467 | 52 |
| 1703494 | 41 |
| 1703533 | 29 |
| 1703557 | 53 |
| 1703593 | 56 |
| 1703599 | 75 |
| 1703614 | 52 |
| 1703635 | 67† |
| 1703641 | 20 |
| 1703644 | 26 |
| 1703689 | 58 |
| 1703695 | 47 |
| 1703713 | 37 |
| 1703722 | 46 |
| 1703743 | 99 |
| 1703836 | 46 |
| 1703848 | 40 |
| 1703851 | 41 |
| 1703902 | 40 |
| 1703908 | 39 |
| 1703968 | 48 |
| 1703974 | 53 |
| 1703986 | 49 |
| 1703998 | 36 |

TABLE 28
Reduction of MAPT RNA by RNAi compounds
that target human MAPT nucleic acid

| Compound | MAPT |
|---|---|
| Number | (% UTC) |
| 1612982 | 29 |
| 1702375 | 78 |
| 1702438 | 87 |
| 1702441 | 61 |
| 1702462 | 98 |
| 1702465 | 101 |
| 1702471 | 96 |
| 1702477 | 91 |
| 1702480 | 147 |
| 1702495 | 101 |
| 1702510 | 94 |
| 1702519 | 66 |
| 1702522 | 128 |
| 1702573 | 81 |
| 1702579 | 71 |
| 1702582 | 114 |
| 1702615 | 138 |
| 1702636 | 137 |
| 1702663 | 57 |
| 1702675 | 103 |
| 1702681 | 61 |
| 1702699 | 63 |
| 1702729 | 48† |
| 1702735 | 92† |
| 1702741 | 43† |
| 1702762 | 40 |
| 1702768 | 38 |
| 1702774 | 99 |
| 1702777 | 102 |
| 1702813 | 46 |
| 1702888 | 81 |
| 1702921 | 62 |
| 1702924 | 47 |
| 1702933 | 58 |
| 1702996 | 89 |
| 1703029 | 80 |
| 1703035 | 93 |
| 1703047 | 46 |
| 1703077 | 68 |
| 1703095 | 49 |
| 1703167 | 95 |
| 1703191 | 71 |
| 1703224 | 40 |
| 1703257 | 63 |
| 1703293 | 44 |
| 1703305 | 92 |
| 1703338 | 75 |
| 1703347 | 41 |
| 1703350 | 44 |
| 1703371 | 92 |
| 1703407 | 74 |
| 1703437 | 94 |
| 1703440 | 77 |
| 1703455 | 46 |
| 1703491 | 41 |
| 1703509 | 44 |
| 1703542 | 45 |
| 1703548 | 52 |
| 1703590 | 72 |
| 1703638 | 38 |
| 1703650 | 25 |
| 1703752 | 46 |
| 1703767 | 43 |
| 1703809 | 39 |
| 1703818 | 55 |
| 1703821 | 41 |
| 1703830 | 49 |
| 1703860 | 45 |
| 1703866 | 42 |
| 1703869 | 47 |
| 1703887 | 40 |
| 1703899 | 40 |
| 1703911 | 40 |
| 1703929 | 41 |
| 1703932 | 41 |
| 1703935 | 33 |
| 1703941 | 36 |
| 1703956 | 40 |
| 1704001 | 43 |

TABLE 29
Reduction of MAPT RNA by RNAi compounds
that target human MAPT nucleic acid

| Compound | MAPT |
|---|---|
| Number | (% UTC) |
| 1612982 | 25 |
| 1702360 | 77 |
| 1702363 | 35 |
| 1702381 | 30 |
| 1702408 | 59 |
| 1702543 | 69 |
| 1702549 | 61 |
| 1702570 | 54 |
| 1702591 | 77 |
| 1702606 | 85 |
| 1702609 | 82 |
| 1702624 | 22 |
| 1702627 | 31 |
| 1702645 | 54 |
| 1702657 | 91 |
| 1702693 | 68 |
| 1702702 | 27 |
| 1702714 | 45 |
| 1702720 | 20 |
| 1702771 | 86 |
| 1702825 | 44 |
| 1702828 | 70 |
| 1702837 | 59 |
| 1702849 | 55 |
| 1702852 | 76 |
| 1702855 | 74 |
| 1702906 | 59 |
| 1702915 | 58 |
| 1702936 | 80 |
| 1702942 | 69 |
| 1702984 | 71 |
| 1703008 | 98 |
| 1703020 | 67 |
| 1703026 | 100 |
| 1703041 | 74 |
| 1703065 | 82 |
| 1703068 | 59 |
| 1703134 | 55 |
| 1703170 | 79 |
| 1703200 | 58 |
| 1703212 | 67 |
| 1703242 | 92 |
| 1703245 | 97 |
| 1703263 | 65 |
| 1703302 | 78 |
| 1703326 | 54 |
| 1703344 | 66 |
| 1703359 | 50 |
| 1703368 | 90 |
| 1703377 | 60 |
| 1703410 | 72 |
| 1703446 | 99 |
| 1703452 | 54 |
| 1703464 | 67 |
| 1703488 | 52 |
| 1703497 | 46 |
| 1703506 | 47 |
| 1703551 | 97 |
| 1703563 | 107 |
| 1703566 | 95 |
| 1703620 | 53 |
| 1703623 | 36 |
| 1703626 | 53 |
| 1703659 | 49 |
| 1703677 | 82 |
| 1703698 | 47 |
| 1703785 | 49 |
| 1703806 | 56 |
| 1703815 | 70 |
| 1703833 | 60 |
| 1703884 | 49 |
| 1703893 | 52 |
| 1703920 | 64 |
| 1703938 | 49 |
| 1703953 | 45 |
| 1703971 | 45 |
| 1703977 | 51 |
| 1703980 | 46 |
| 1703989 | 47 |

1.-7. (canceled)

8. An oligomeric compound, wherein the oligomeric compound comprises a modified oligonucleotide consisting of 12 to 50 linked nucleosides, wherein the nucleobase sequence of the modified oligonucleotide is complementary to at least 12, at least 13, at least 14, at least 15, at least 16, at least 17, at least 18, at least 19, at least 20, at least 21, at least 22, or 23 contiguous nucleobases of:
an equal length portion of nucleobases of 110-142 of SEQ ID NO: 1;
an equal length portion of nucleobases of 1754-1783 SEQ ID NO: 1;
an equal length portion of nucleobases of 2332-2362 SEQ ID NO: 1; or
an equal length portion of nucleobases of 6523-6552 SEQ ID NO: 1.

9. The oligomeric compound of claim 8, wherein the nucleobase sequence of the modified oligonucleotide is complementary to at least 12, at least 13, at least 14, at least 15, at least 16, at least 17, at least 18, at least 19, at least 20, at least 21, at least 22, or 23 contiguous nucleobases of:
SEQ ID NOs: 721, 850, 1115, or 1116;
SEQ ID NOs: 885, 1142, or 1360;
SEQ ID NOs: 329, 1045, or 1343; and
SEQ ID NOs: 890, 1330, or 1431.

10. (canceled)

11. The oligomeric compound of claim 8, wherein at least one nucleoside of the modified oligonucleotide comprises a modified sugar moiety.

12. The oligomeric compound of claim 11, wherein the modified sugar moiety comprises a bicyclic sugar moiety.

13. The oligomeric compound of claim 12, wherein the bicyclic sugar moiety comprises a 2′-4′ bridge, wherein the 2′-4′ bridge is selected from —O—CH2- and —O—CH(CH3)-.

14. (canceled)

15. The oligomeric compound of claim 11, wherein the modified sugar moiety comprises a non-bicyclic modified sugar moiety, and wherein the non-bicyclic modified sugar moiety is a 2′-MOE sugar moiety, a 2′-OMe sugar moiety, or a 2′-F sugar moiety.

16. (canceled)

17. (canceled)

18. The oligomeric compound of claim 8, wherein the modified oligonucleotide comprises at least one modified internucleoside linkage.

19.-29. (canceled)

30. The oligomeric compound of claim 8, wherein the oligomeric compound comprises a conjugate group.

31.-36. (canceled)

37. An oligomeric duplex, comprising a first oligomeric compound comprising a first modified oligonucleotide and a second oligomeric compound comprising a second modified oligonucleotide, wherein the first oligomeric compound is an oligomeric compound of claim 8.

38. The oligomeric duplex of claim 37, wherein the second modified oligonucleotide consists of 8 to 80 linked nucleosides, and wherein the nucleobase sequence of the second modified oligonucleotide comprises a complementary region of at least 8 nucleobases that is at least 90% complementary to an equal length portion of the first modified oligonucleotide.

39. (canceled)

40. (canceled)

41. (canceled)

42. The oligomeric duplex of claim 37, wherein the first modified oligonucleotide comprises a 5′-stabilized phosphate group selected from a cyclopropyl phosphonate or a vinyl phosphonate.

43. (canceled)

44. (canceled)

45. (canceled)

46. The oligomeric duplex of claim 37, wherein at least one nucleoside of the second modified oligonucleotide comprises a modified sugar moiety.

47. The oligomeric duplex of claim 46, wherein the modified sugar moiety of the second modified oligonucleotide comprises a bicyclic sugar moiety.

48. The oligomeric duplex of claim 47, wherein the bicyclic sugar moiety of the second modified oligonucleotide comprises a 2′-4′ bridge selected from —O—CH2- and —O—CH(CH3)-.

49. (canceled)

50. The oligomeric duplex of claim 46, wherein the modified sugar moiety comprises a non-bicyclic modified sugar moiety, and wherein the non-bicyclic modified sugar moiety of the second modified oligonucleotide is a 2′-MOE sugar moiety, a 2′-F sugar moiety, or 2′-OMe sugar moiety.

51. (canceled)

52. (canceled)

53. (canceled)

54. (canceled)

55. (canceled)

56. (canceled)

57. (canceled)

58. (canceled)

59. The oligomeric duplex of claim 37, wherein the second modified oligonucleotide comprises a conjugate group, wherein the conjugate group is attached to the second modified oligonucleotide at the 5′-end or the 3′ end of the second modified oligonucleotide.

60. (canceled)

61. (canceled)

62. (canceled)

63. (canceled)

64. The oligomeric duplex of claim 37, wherein the second modified oligonucleotide comprises a terminal group, wherein the terminal group is an abasic sugar moiety.

65.-71. (canceled)

72. A chirally enriched population of oligomeric duplexes of claim 37, wherein the population is enriched for modified oligonucleotides comprising at least one particular phosphorothioate internucleoside linkage having a particular stereochemical configuration.

73. (canceled)

74. (canceled)

75. (canceled)

76. A pharmaceutical composition comprising the oligomeric compound of claim 8, and a pharmaceutically acceptable diluent or carrier.

77. (canceled)

78. (canceled)

79. A method comprising administering to an animal the oligomeric compound of claim 8.

80. (canceled)

81. (canceled)

82. A method of treating a tau-associated disease comprising administering to an individual having or at risk for developing the tau-associated disease a therapeutically effective amount of the oligomeric compound of claim 8, thereby treating the tau-associated disease.

83. The method of claim 82, wherein the tau-associated disease is a tauopathy, Alzheimer's disease, frontotemporal dementia (FTD), frontotemporal dementia with parkinsonism-17 (FTDP-17), progressive supranuclear palsy (PSP), chronic traumatic encephalopathy (CTE), corticobasal ganglionic degeneration (CBD), epilepsy, or Dravet's Syndrome.

84.-94. (canceled)

95. A method of reducing tau in a cell comprising contacting the cell with the oligomeric compound of claim 8.

96.-103. (canceled)
